(19) United States
(12) Patent Application Publication
(10) Pub. No.: US 2026/0232652 A1
(43) Pub. Date: Aug. 13, 2026

(54) COMBINATIONS OF DGK (DIACYLGLYCEROL KINASE) INHIBITORS

(71) Applicants: Bayer Aktiengesellschaft, Leverkusen (DE); Deutsches Krebsforschungszentrum Stiftung des Öffentlichen Rechts, Heidelberg (DE)

(72) Inventors: Dennis KIRCHHOFF, Berlin (DE); Matyas GORJANACZ, Berlin (DE); Kirstin PETERSEN, Berlin (DE); Norbert SCHMEES, Berlin (DE); Rienk OFFRINGA, Heidelberg (DE); Catherine OLESCH, Heidelberg (DE); Frederik CICHON, Heidelberg (DE); Antje Margret WENGNER, Berlin (DE); Christoph Phillipp HETHEY, Berlin (DE)

(73) Assignees: Bayer Aktiengesellschaft, Leverkusen (DE); Deutsches Krebsforschungszentrum Stiftung des Öffentlichen Rechts, Heidelberg (DE)

(21) Appl. No.: 19/154,274

(22) PCT Filed: Feb. 5, 2024
(86) PCT No.: PCT/EP2024/052714

§ 371 Date: Aug. 6, 2025

(30) Foreign Application Priority Data
Feb. 6, 2023 (EP) 23155228.2
Apr. 14, 2023 (EP) 23168027.3

(51) Int. Cl. A61K 31/4709; A61K 31/426; A61K 31/4439; A61K 39/395; A61P 35/00

(52) U.S. Cl. CPC A61K 31/4709; A61K 31/426; A61K 31/4439; A61K 39/39558; A61P 35/00

(57) ABSTRACT

The present invention covers combinations comprising one or more inhibitors of DGKalpha and one or more inhibitors of DGKzeta Another aspect of the present invention covers the use of such combinations as described herein for the preparation of a medicament for the treatment or prophylaxis of a disease, particularly of a condition with dysregulated immune responses, particularly cancer, or a viral infection or another disorder associated with aberrant DGKalpha and/or DGKzeta signaling.

### TECHNICAL FIELD

The present invention covers combinations comprising one or more inhibitors of DGKalpha and one or more inhibitors of DGKzeta, particularly combinations comprising one DGKalpha inhibitor compound of general formula (I) described herein and one DGKzeta inhibitor compound of general formula (II) described herein, and more particularly, combinations comprising DGKalpha inhibitor A and DGKzeta inhibitor A′ described herein. Further, the present invention relates to an inhibitor of DGKalpha for use in a method of the treatment or prophylaxis of a disease, particularly of a condition with dysregulated immune responses, particularly cancer, or a viral infection or another disorder associated with aberrant DGKalpha and/or DGKzeta signaling, in mammals, including humans, characterized in that the method of the treatment or prophylaxis of said disease comprises administering at least one inhibitor of DGKzeta. Furthermore provided is an inhibitor of DGKzeta for use in a method of the treatment or the prophylaxis of a disease, particularly of a condition with dysregulated immune responses, particularly cancer, or a viral infection or another disorder associated with aberrant DGKalpha and/or DGKzeta signaling, in mammals, including humans, characterized in that the method of the treatment or prophylaxis of said disease comprises administering at least one inhibitor of DGKalpha. Another aspect of the present invention covers the use of said combinations comprising one or more inhibitors of DGKalpha and one or more inhibitors of DGKzeta, for the preparation of a medicament for the treatment or prophylaxis of a disease, particularly of a condition with dysregulated immune responses, particularly cancer, or a viral infection or another disorder associated with aberrant DGKalpha and/or DGKzeta signaling, in mammals, including humans.

### BACKGROUND

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases. In particular there is a need for therapeutic methods for re-activating the patients' immune system, especially exhausted and suppressed T cells, to kill tumors.

Diacylglycerol kinases (DGKs) represent a family of enzymes that catalyse phosphorylation of the membrane lipid sn-1,2 diacylglycerol (DAG) to form phosphatidic acid (PA) (T. O. Eichmann and A. Lass, Cell. Mol. Life Sci. 2015, 72, 3931-3952). In T cells, DAG is formed downstream of the T cell receptor (TCR) after activation of the gamma 1 isoform of phospholipase C (PLCγ1) and cleavage of phosphatidylinositol 4,5-biphosphate (PIP2) into DAG and an additional second messenger, inositol 1,4,5-triphosphate (IP3) (S. Krishna and X.-P. Zhong, Front. Immunol. 2013, 4, 178). Whereas IP3 is important in facilitating release of calcium from the endoplasmic reticulum, DAG interacts with other proteins important in TCR signal transduction, such as protein kinase Cθ (E. J. Quann et al., Nat. Immunol. 2011, 12 (7), 647-654) and the Ras activating protein RasGRP1 (S. Krishna and X.-P. Zhong, Front. Immunol. 2013, 4, 178). Three isoforms of DGK are known to be present within T cells: DGKα (DGKalpha), DGKδ (DGKdelta), and DGKzeta (DGKzeta). Two of these, DGKalpha and DGKzeta, are thought to play an important role in facilitating DAG metabolism downstream of the TCR (R. P. Joshi and G. A. Koretzky, Int. J. Mol. Sci. 2013, 14 (4), 6649-6673).

Targeting the activity of DGKalpha or DGKzeta in T cells, either by germline deletion, or with chemical inhibitors, results in enhanced and sustained signalling downstream of T cells, as assessed by prolonged phosphorylation of downstream molecules, such as extracellular signal-related kinases 1/2 (ERK1/2) and NFκB (X.-P.-Zhong et al., Nat. Immunol. 2003, 4, 882-890; B. A. Olenchock et al., Nat. Immunol. 2006, 7 (11), 1174-1181; M. J. Riese et al., J. Biol. Chem. 2011, 286, 5254-5265; E. M. Wesley et al., ImmunoHorizons 2018, 2 (4), 107-118).

Deletion of DGKzeta or DGKalpha in T cells leads to enhanced production of effector cytokines, such as IL2, IFNγ and enhanced proliferation (X.-P. Zhong et al., Nat. Immunol. 2003, 4, 882-890; B. A. Olenchock et al., Nat. Immunol. 2006, 7 (11), 1174-1181, E. M. Riese et al., J. Biol. Chem. 2011, 286, 5254-5264). Furthermore, the overexpression of DGKalpha induces a state of decreased functional activity resembling an anergy-like state (Zha et al., Nat Immunol 2006, 7, 1166).

Adoptive transfer of DGKzeta deficient T cell reduced leukaemia burden after inoculation of C1498.SIY leukaemia cells compared to control. Also, DGKzeta deficient T cells are at least partially resistant to PD1 mediated inhibitory signals (W. Jing et al., Cancer Res. 2017, 77 (20), 5676-5686). In addition, DGKzeta deficient mice have reduced tumor sizes compared to control after orthotopic tumor injection of a pancreatic tumor model (E. M. Wesley et al., ImmunoHorizons, 2018, 2 (4), 107-118). Also, S. Wee et al. inoculated C57BL/6 mice with a variety of syngeneic tumor cell lines—MC38 colon carcinoma, B16F1 melanoma, and C1498 leukemia—and analysed survival and tumor growth between mice deficient in DGKzeta in the presence or absence of anti-PD1 treatment. DGKzeta−/− mice suppressed growth of subcutaneously implanted tumor cells in the three model systems and the combination of DGKzeta-deficiency and anti-PD1 was additive in tumor control (S. Wee et al., Proceedings of the American Association for Cancer Research Annual Meeting 2019; Cancer Res. 2019, 79 (13 Suppl): Abstract nr 936).

The role of DGKalpha in anti-tumor responses was also studied in human tumor-infiltrating CD8+ T cells (CD8-TILs) from patients with renal cell carcinoma (RCC) (Prinz et al., J. Immunol 2012, 188, 5990). CD8-TILs from RCCs were defective in lytic granule exocytosis and their ability to kill target cells. While proximal signaling events were intact in response to TCR engagement, CD8-TILs exhibited decreased phosphorylation of ERK when compared to non-tumor-infiltrating CD8+ T cells. Treatment of CD8-TILs with an inhibitor of DGKalpha activity rescued killing ability of target cells, increased basal levels of phosphorylation of ERK, and increased PMA/ionomycin-stimulated phosphorylation of ERK.

Consequently, DGKzeta and DGKalpha can be a useful target for enhancing T cell anti-tumor activity.

Additionally, the adoptive transfer of CAR (chimeric antigen receptor)-T cells deficient in DGKzeta and/or DGKalpha demonstrated increased efficacy compared to wild type CAR T cells in the treatment of murine mesothelioma (M. J. Riese et al., Cancer Res. 2013, 73 (12), 3566-3577) and a glioblastoma xenograft mouse model in combination with DGKalpha knockout (I.-Y. Jung et al., Cancer Res. 2018, 78 (16), 4692-4703).

In addition, DGK inhibitors promoted not only Ras/ERK signaling but also AP-1 (Activator protein-1) transcription, facilitated DGKalpha membrane localization, reduced the requirement for costimulation, and cooperated with enhanced activation following DGKzeta silencing/deletion. In contrast with enhanced activation triggered by pharmacological inhibition, DGKalpha silencing/genetic deletion led to impaired Lck (lymphocyte-specific protein tyrosine kinase) activation and limited costimulation responses. (Arranz-Nicolas et al., Canc Immun, Immunother 2018, 67(6), 965).

Furthermore, antigen-specific CD8 positive T cells from DGKalpha^{-/-} and DGKzeta^{-/-} mice show enhanced expansion and increased cytokine production following (Lymphocytic choriomeningitis virus) infection (Shin et al. J. Immunol, 2012). Also, DGKzeta-deficient mice mounted a more robust immune response to lymphocytic choriomeningitis virus infection than did wild-type mice (X.-P. Zhong et al., Nat. Immunol. 2003, 4, 882-890).

DGKzeta is also relevant in natural killer (NK) cells. Upon stimulation through multiple activating receptors, NK cells from mice lacking DGKzeta display increased cytokine production and degranulation in an ERK-dependent manner. Additionally, they have improved cytotoxic functions against tumor cell lines. (E. Yang et al. J. Immunol. 2016, 197(3), 934-41.)

Apart from immune-cell regulation, DGKzeta also plays a role in cancer, mediating numerous aspects of cancer cell progression including proliferation, apoptosis, survival, invasion and tumorigenicity, e.g. in osteosarcoma, colon cancer, breast cancer, prostate cancer, glioma and leukemia models (W. Yu et al., Front. Oncol. 2019, 8:655; K. Cai et al., BMC Cancer 2014, 14:208; J. Diao et al., Mol. Neurobiol. 2016; 53, 5425-35; H. Li et al. Pharmazie 2019, 74(7): 418-422).

DGKalpha also plays a role in cancer, mediating numerous aspects of cancer cell progression including survival (Bacchiocchi et al., Blood, 2005, 106(6), 2175; Yanagisawa et al. Biochim Biophys Acta 2007, 1771, 462), migration and invasion of cancer cells (Baldanzi et al., Oncogene 2008, 27, 942; Filigheddu et al., Anticancer Res 2007, 27, 1489; Rainero et al., J Cell Biol 2012, 196(2): 277). In particular, it has been reported that DGKalpha is overexpressed in hepatocellular carcinoma (Takeishi et al., J Hepatol 2012, 57, 77) and melanoma cells (Yanagisawa et al., Biochim Biophys Acta 2007, 1771, 462) while other reports suggested that the growth of colon and breast cancer cell lines was significantly inhibited by DGKalpha-siRNA16 and DGKalpha/atypical PKC/b1 integrin signalling pathway was crucial for matrix invasion of breast carcinoma cells (Rainero et al., PLoS One 2014, 9(6): e97144) In addition, expression is also higher in lymphonodal metastasis than in breast original tumour (Hao et al., Cancer 2004, 100, 1110).

Additionally, a study testing the importance of DGKalpha in glioblastoma multiforme (GBM) cells found that concurrent administration of the relatively non-specific DGKalpha inhibitor R59022 resulted in decreased growth of intracranially injected GBM tumors. (Dominguez et al. Cancer Discov 2013, 3(7): 782).

Also, DGKalpha promotes esophageal squamous cell carcinoma (ESCC) progression, supporting DGKalpha as a potential target for ESCC therapy (Chen et al., Oncogene, 2019, 38 (14) 2533).

In addition, pharmacological inhibition of DGK diminished both airway inflammation and airway hyperresponsiveness in mice and also reduced bronchoconstriction of human airway samples in vitro by blocking T helper 2 (T_{H}2) differentiation (Singh et al., Sci Signal. 2019, 12, eaax3332).

Furthermore, inhibition of DGKalpha has the potential to reverse the life-threatening Epstein-Barr virus (EBV)-associated immunopathology that occurs in patients X-linked lymphoproliferative disease (XLP-1) patients (Ruffo et al., Sci Transl Med. 2016, 13, 8, 321; Velnati et al., Eur J Med Chem. 2019, 164, 378).

In addition, DGKalpha exacerbates cardiac injury after ischemia/reperfusion cardiac diseases (Sasaki et al., Heart Vessels, 2014, 29, 110).

Taken together, the findings from these studies argue that restraining DGKzeta and/or DGKalpha activity in T cells and tumor cells may prove valuable in generating more vigorous immune responses against tumors and in ameliorating Th2 driven (auto) immune diseases (in re-balancing the immune-system). In addition, inhibiting DGKzeta and/or DGKalpha activity has a therapeutic potential in targeting tumors directly as well as addressing fibrotic disorders, virus infection associated pathologies, cardiac diseases and lymphoproliferative disorders.

Inhibitors of DGK have been disclosed previously, e.g. aminoquinolone-based inhibitors of DGKalpha, see WO2021/105115, WO2021/105116 and WO2021/105117, and aminothiazole-based inhibitors of DGKzeta, see WO2021/214019 and WO2021/214020.

Further inhibitors of DGKalpha are disclosed in WO2021/130638, WO2022/271650, WO2022/271659, WO2022/271677, WO2022/271684, WO2022/114812, WO2023/165504, WO2023/165509 and WO2023/165525.

Further inhibitors of DGKzeta are disclosed in WO2021/132422 and in WO 2022/114164.

Inhibitors of DGKalpha and/or DGKzeta are disclosed in WO2020/006016, WO2020/006018, WO2021/041588, w02021/133748, w02021/133749, w02021/133750, w02021/133751, WO2021/133752, WO2021/258010, WO2022/133083, WO2022/171745, WO2023/011456, WO2023/122772, WO2023/122777, WO2023/122778, WO2023/184327, and WO2023/186060. Yet further inhibitors/modulators of DGK activity are disclosed in WO2023/023376, WO2023/023664, WO2023/150186, CN116969943 and CN117088874.

Combinations of DGK inhibitors and immune checkpoint antagonists are disclosed in WO2021/127554.

Combinations of T-cell therapies with a DGK inhibitor are disclosed in WO2022/187406.

Wichroski et al., Sci. Transl. Med. 15, eadh1892 (2023) 25 Oct. 2023 disclose studies on dual DGKalpha/zeta inhibitors in combination with aPD-(L)1.

Chupak et al., ACS Med. Chem. Lett. 2023, 14, 929-935, disclose the discovery of dual DGKalpha/zeta inhibitors.

### SUMMARY OF THE INVENTION

Surprising effects in vitro and in in vivo tumor models were observed when administering a DGKα inhibitor in combination with a DGKzeta inhibitor.

In accordance with a first aspect, the present invention provides combinations comprising one or more inhibitors of DGKalpha and one or more inhibitors of DGKzeta.

In accordance with a second aspect, the present invention provides a kit comprising:
One DGKalpha inhibitor compound of general formula (I) as described herein, more particularly DGKalpha inhibitor A, or a stereoisomer, a tautomer, an N-oxide, a hydrate, a solvate, or a salt thereof, or a mixture of same,
One DGKzeta inhibitor compound of general formula (II) as described herein, more particularly DGKzeta inhibitor A′, or a stereoisomer, a tautomer, an N-oxide, a hydrate, a solvate, or a salt thereof, or a mixture of same.

In accordance with a third aspect, the present invention provides a pharmaceutical composition comprising a combination of one or more inhibitor(s) of DGKalpha and one or more inhibitor(s) of DGKzeta, together with one or more pharmaceutically acceptable excipients.

In accordance with a fourth aspect there is provided an inhibitor of DGKalpha for use in a method of the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra, characterized in that said method comprises administering at least one inhibitor of DGKzeta.

In accordance with a fifth aspect there is provided an inhibitor of DGKzeta for use in a method of the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra, characterized in that said method comprises administering at least one inhibitor of DGKalpha.

In accordance with a sixth aspect, the present invention provides the combination of the present invention as described supra for use in the treatment or prophylaxis of a disease, preferably a condition with dysregulated immune responses, particularly cancer, or a viral infection or another disorder associated with aberrant DGKalpha and/or DGKzeta signaling, in mammals, including humans, as described infra.

In accordance with a seventh aspect, the present invention provides the kit as described supra for use in the treatment or prophylaxis of a disease, preferably a condition with dysregulated immune responses, particularly cancer, or a viral infection or another disorder associated with aberrant DGKalpha and/or DGKzeta signaling, in mammals, including humans, as described infra.

In accordance with an eighth aspect, the present invention provides the pharmaceutical composition as described supra for use in the treatment or prophylaxis of a disease, preferably a condition with dysregulated immune responses, particularly cancer, or a viral infection or another disorder associated with aberrant DGKalpha and/or DGKzeta signaling, in mammals, including humans, as described infra.

In accordance with a ninth aspect, the present invention provides a method of the treatment or prophylaxis of a disease in a patient, preferably a condition with dysregulated immune responses, particularly cancer, or a viral infection or another disorder associated with aberrant DGKalpha and/or DGKzeta signaling, in mammals, including humans, as described infra, comprising
a) administering DGKalpha inhibitor A as described herein, or a tautomer, an N-oxide, a hydrate, a solvate, or a salt thereof, or a mixture of same, and
b) administering and DGKzeta inhibitor A′, as described herein, or a stereoisomer, a tautomer, an N-oxide, a hydrate, a solvate, or a salt thereof, or a mixture of same.

### Technical Problem

While there has been significant clinical success in treating cancer by stimulating immune cells to act against tumors, e.g. by treatment with humanized antibodies addressing the PD-1/PD-L1 axis in a part of the respective patient population, tumors of many patients develop resistance against the treatment, and many others do not respond at all. Hence there is a need to even further improve cancer therapies, including those which act by activating immune cells against the respective tumor. In addition, there is a need to offer solutions which prevent the re-occurrence of a tumor, e.g. the formation of metastases. As outlined supra, there is a large body of evidence supporting that inhibition of some DGK isoforms, notably DGKalpha and DGKzeta, can effect such stimulation, and multiple clinical studies involving inhibitors of DGKalpha, DGKzeta and dual inhibitors of DGKalpha/zeta have been initiated. However, little is known on how the effects of inhibiting DGKalpha and DGKzeta are best balanced, and “further studies with DGKalpha- and DGKzeta-selective inhibitors will be needed to deconvolute the contribution of components for these DGK isozymes on T cell signaling and tumor immunity” (Wichroski et al., Sci. Transl. Med. 15, eadh1892 (2023) 25 Oct. 2023).

### Solution to Problem

It was now surprisingly found in pre-clinical experiments that combined treatment with inhibitors or DGKalpha and inhibitors of DGKzeta offers advantageous and surprising technical effects. It was found in syngeneic murine tumor models (e.g. EMT6, MC38, Hepa1-6) that the combined treatment with an inhibitor of DGKalpha and an inhibitor of DGKzeta results in a pronounced reduction of tumor growth which clearly exceeds the addition of effects resulting from monotherapy with the respective inhibitor of DGKalpha and the respective inhibitor of DGKzeta alone. Further, increased survival over either monotherapy was found in the MC38 syngeneic murine tumor model upon combined treatment with inhibitors or DGKalpha and inhibitors of DGKzeta, and those animals surviving did not show any substantial tumor growth upon re-challenge by inoculation with MC38 cells after recovery from the experiment described in Example 6.

As any drug likely reaches dose limiting toxicity upon increasing dose at some point, it can be expected in a clinical setting that replacing some of the antitumor effect of a given dose of an DGKzeta inhibitor by combined treatment with a reduced dose of a DGKzeta inhibitor and a DGKalpha inhibitor will result in reduction of a DGKzeta inhibitor associated toxicity whilst efficacy will remain at least unchanged, and vice versa, that is, that replacing some of the antitumor effect of a given dose of an DGKalpha inhibitor by combined treatment with a reduced dose of a DGKalpha inhibitor and with a DGKzeta inhibitor will result in reduction of a DGKalpha inhibitor associated toxicity whilst efficacy will remain at least unchanged.

Further benefits of combining treatment with an inhibitor of DGKalpha and an inhibitor of DGKzeta, as compared to treatment with one dual DGKalpha/zeta inhibitor, include the option to dose them independently from each other flexibly, and following an individual patient's response to treatment, in terms of the amounts dosed, and also in terms of order and timing of the respective dosings, allowing for an optimized treatment of individual patients, maximizing beneficial effects from the inhibition of either DGK isoform whilst minimizing potential undesired effects.

### DETAILED DESCRIPTION OF THE INVENTION

### Definitions

### General Definitions

Unless otherwise defined, all scientific and technical terms used in the description, figures and claims have their ordinary meaning as commonly understood by one of ordinary skill in the art. All publications, patent applications, patents, and other references mentioned herein are incorporated by reference in their entirety. In case of conflict, the present specification, including definitions, will prevail. If two or more documents incorporated by reference include conflicting and/or inconsistent disclosure with respect to each other, then the document having the later effective date shall control. The materials, methods, and examples are illustrative only and are not intended to be limiting. Unless stated otherwise, the following terms used in this document, including the description and claims, have the definitions given below.

The expression “about” or “~” as used herein refers to a value being within an acceptable error range for the particular value as determined by one of ordinary skill in the art, which will depend in part on how the value is measured or determined, i.e., on the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects as described herein.

In this context “about” may refer to a range above and/or below of up to 10%. Wherever the term “about” is specified for a certain assay or embodiment, that definition prevails for the particular context.

If not defined otherwise, the term “approximately” means the provided value +/−10%.

The terms “comprising”, “including”, “containing”, “having” etc. shall be read expansively or open-ended and without limitation. The term comprising when used in the specification includes “consisting of”.

If within the present text any item is referred to as “as mentioned herein”, or as “as described herein”, it means that it may be mentioned anywhere in the present text.

Singular forms such as “a”, “an” or “the” include plural references unless the context clearly indicates otherwise. Thus, for example, reference to “a monoclonal antibody” includes a single monoclonal antibody as well as a plurality of monoclonal antibodies, either the same or different. Likewise reference to “cell” includes a single cell as well as a plurality of cells.

Unless otherwise indicated, the term “at least” preceding a series of elements is to be understood to refer to every element in the series. The terms “at least one” and “at least one of” include for example, one, two, three, four, five or more elements.

It is furthermore understood that slight variations above and below a stated range can be used to achieve substantially the same results as a value within the range. Also, unless indicated otherwise, the disclosure of ranges is intended as a continuous range including every value between the minimum and maximum values.

The term “amino acid” or “amino acid residue” as used herein typically refers to a naturally-occurring amino acid. The one letter code is used herein to refer to the respective amino acid. As used herein, a “charged amino acid” is an amino acid which is negatively charged or positively charged. “Negatively charged amino acids” are aspartic acid (D) and glutamic acid (E). “Positively charged amino acids” are arginine (R) lysine (K) and histidine (H). “Polar amino acids” are all amino acids that form hydrogen bonds as donors or acceptors. These are all charged amino acids and asparagine (N), glutamine (Q), serine (S), threonine (T), tyrosine (Y) and cysteine (C). “Polar uncharged amino acids” are asparagine (N), glutamine (Q), serine (S), threonine (T), tyrosine (Y) and cysteine (C). “Amphiphatic amino acids” are tryptophan (W), tyrosine (Y) and methionine (M). “Aromatic amino acids” are phenylalanine (F), tyrosine (Y), and tryptophan (W). “Hydrophobic amino acids” are glycine (G), alanine (A), valine (V), leucine (L), isoleucine (I), proline (P), phenylalanine (F), methionine (M) and cysteine. “Small amino acids” are glycine (G), alanine (A), serine (S), proline (P), threonine (T), aspartic acid (D) and asparagine (N).

The term “DGK”, also DAGK refers to the Diacylglycerol kinase family, a family of enzymes catalyzing the conversion of diacylglycerol (DAG) to phosphatidic acid (PA). The family members use ATP as a source of the phosphate. DGKs can be classified into the following five groups. The group of Type 1 DGKs comprises DGK-α, DGK-β, and DGK-γ, and members comprise EF-hand motifs and a recoverin homology domain. The group of Type 2 DGKs comprises DGK-δ and DGK-η and members comprise a pleckstrin homology domain. The group of Type 3 DGKs comprises DGK-ε, which has specificity for arachidonate-containing DAG. The group of Type 4 DGKs comprises DGK-ζ, and DGK-ι and members comprise a MARCKS homology domain, ankyrin repeats, a C-terminal nuclear localisation signal, and a PDZ-binding motif. The group of Type 5 DGKs comprises DGK-θ, which comprises a third cysteine-rich domain, a pleckstrin homology domain and a proline rich region. A DGK inhibitor is an inhibitor of at least one or more members of the DGK family. For example, a DGK inhibitor can be an inhibitor of one or more of DGK-α, DGK-β, DGK-γ, DGK-δ, DGK-η, DGK-ε, DGK-ζ, DGK-ι and DGK-θ.

The term “DGKalpha”, also DGK-α or DGKα refers to DGK family member Diacylglycerol kinase alpha. The DGKalpha protein is encoded by the gene DGKA (also DAGK, DAGK1, NCBI gene ID 1606). Synonyms for DGKalpha are inter alia DAG kinase alpha, 80 kDa diacylglycerol kinase, and Diglyceride kinase alpha. The DGKalpha protein comprises human, murine, rat, rhesus macaque and further mammalian and non-mammalian homologues. Sequence(s) for human DGKalpha are accessible via UniProt Identifier P23743 (DGKA_HUMAN), for instance human isoform P23743-1 or P23743-2, or P23743-3 (UniProt, Feb. 1, 2023). Different isoforms and variants may exist for the different species and are all comprised by the term DGKalpha. In addition, synthetic variants of the DGKalpha protein may be generated and are comprised by the term DGKalpha. The protein DGKalpha may furthermore be subject to various modifications, e.g, synthetic or naturally occurring modifications, such as post translational modifications. An inhibitor of DGKalpha is a compound or antibody that at least partially inhibits the functional activity at least one isoform of DGKalpha.

The term “DGKzeta”, also DGK-ζ or DGKζ refers to DGK family member Diacylglycerol kinase zeta. The DGKzeta protein is encoded by the gene DGKZ (also DAGK6, DAGK5, DGK-ZETA, hDGKzeta, NCBI gene ID 8525). Synonyms for DGKzeta are inter alia DAG kinase zeta, and Diglyceride kinase zeta. The DGKzeta protein comprises human, murine, rat, rhesus macaque and further mammalian and non-mammalian homologues. Sequence(s) for human DGKzeta are accessible via UniProt Identifier Q13574 (DGKZ_HUMAN), for instance canonical human isoform Q13574-2 (UniProt, Feb. 1, 2023). Different isoforms and variants exist for the different species and are all comprised by the term DGKzeta. In addition, synthetic variants of the DGKzeta protein may be generated and are comprised by the term DGKzeta. The protein DGKzeta may furthermore be subject to various modifications, e.g, synthetic or naturally occurring modifications, such as post translational modifications. An inhibitor of DGKzeta is a compound or antibody that at least partially inhibits the functional activity at least one isoform of DGKzeta.

An “isotype control” is an antibody or fragment that does not bind a target but has the same class and type as the reference antibody or fragment recognizing the target.

“Half maximal effective concentration” (EC50) refers to the concentration of a drug, antibody, fragment, conjugate or molecule which induces a response halfway between the baseline and maximum after a specified incubation time. In the context of antibody binding, the EC50 thus reflects the antibody concentration needed for half-maximal binding. An EC50 can be determined if an inflection point can be determined by mathematical modeling (e.g., non-linear regression) of the dose-response curve describing the relationship between applied drug, antibody, fragment, conjugate or molecule concentration and signal. For example, if the dose-response curve follows a sigmoidal curve, an EC50 can be determined. Where the response is an inhibition, the EC50 is termed half maximal inhibitory concentration (IC50). EC80 can be determined mutatis mutandis.

The term “linker” or “spacer” as used herein refers to any molecule enabling a direct topological connection between two moieties. A moiety may be inter alia a polypeptide, a protein, an antibody, an antibody fragment, a cytotoxic moiety, a binding moiety, a moiety for detection such as a fluorophore, a moiety for immobilization or retrieval such as beads or magnetic beads, a reactive moiety, or any other molecule. The two moieties may be of the same type or different. Linkers may be part of conjugates and may even contribute to their function. For instance, for a conjugate comprising a polypeptide and a biotin, the presence of a spacer of approximately 4 Å (~5 atoms) between the carboxy group of the biotin and the 1st bulky amino acid of the peptide allows the biotin to reach the (strept)avidin binding pocket. Various linkers are known in the art and can be selected based on the moieties which shall be connected. The linker length typically ranges between 4 atoms and more than 200 atoms. Linkers exceeding 60 atoms in length generally comprise a population of compounds having an average length.

In the context of the present invention, the term “treatment” or “treating” includes inhibition, retardation, checking, alleviating, attenuating, restricting, reducing, suppressing, repelling or healing of a disease or the development, the course or the progression of such states and/or the symptoms of such states. The term “disease” includes but is not limited to a condition, a disorder, an injury or a health problem. The term “therapy” is understood here to be synonymous with the term “treatment”.

The terms “prevention”, “prophylaxis” or “preclusion” are used synonymously in the context of the present invention and refer to the avoidance or reduction of the risk of contracting, experiencing, suffering from or having a disease or a development or advancement of such states and/or the symptoms of such states.

The treatment or prevention of a disease may be partial or complete.

The term “effective amount” or “therapeutically effective amount” are used interchangeably herein and refer to an amount sufficient to achieve a particular biological result or to modulate or ameliorate a symptom in a subject, or the time of onset of a symptom, typically by at least about 10%; usually by at least about 20%, preferably at least about 30%, or more preferably at least about 50%. Efficacy of the use of an antibody in cancer therapy can be assessed based on the change in tumor burden. Both tumor shrinkage (objective response) and time to the development of disease progression are important endpoints in cancer clinical trials. Standardized response criteria, known as RECIST (Response Evaluation Criteria in Solid Tumors), were published in 2000. An update (RECIST 1.1) was released in 2009. RECIST criteria are typically used in clinical trials where objective response is the primary study endpoint, as well as in trials where assessment of stable disease, tumor progression or time to progression analyses are undertaken because these outcome measures are based on an assessment of anatomical tumor burden and its change over the course of the trial. An effective amount for a particular subject may vary depending on factors such as the condition being treated, the overall health of the subject, the method, route, and dose of administration and the severity of side effects. When in combination, an effective amount is in ratio to a combination of components and the effect is not limited to individual components alone. Wherever an antibody is administered as part of a medical use it is clear for the skilled person that the antibody needs to be administered in an effective amount.

If not defined otherwise, “Complete Response” (CR) is defined as disappearance of all target lesions. Any pathological lymph nodes (whether target or non-target) must have reduction in short axis to <10 mm. For “Partial Response” (PR) at least a 30% decrease in the sum of diameters of target lesions has to be reached, taking as reference the baseline sum diameters. For “Progressive Disease” (PD) at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. In “Stable Disease” (SD) neither sufficient shrinkage to qualify for PR nor sufficient increase to qualify for PD is observed, taking as reference the smallest sum diameters while on study.

Secondary outcome measures that can be used to determine the therapeutic benefit of the inventive antibodies described herein include the following: “Objective Response Rate” (ORR) is defined as the proportion of subjects who achieve a complete response (CR) or partial response (PR). “Progression Free Survival” (PFS) is defined as the time from the first dose date of an antibody to either disease progression or death, whichever occurs first. “Overall Survival” (OS) is defined as the length of time from either the date of diagnosis or the start of treatment for a disease, that patients diagnosed with the disease are still alive. “Duration of Overall Response” (DOR) is defined as the time from the participant's initial CR or PR to the time of disease progression. “Depth of Response” (DpR) is defined as the percentage of tumor shrinkage observed at the maximal response point compared to baseline tumor load. Clinical endpoints for both ORR and PFS can be determined based on RECIST 1.1 criteria described above.

Where non-human subjects are analyzed, the aforementioned parameters to determine therapeutic efficacy and benefit have to be adapted.

As used herein, the terms “patient” or “subject” are used interchangeably and mean a mammal, including, but not limited to, a human or non-human mammal, such as a bovine, equine, canine, ovine, or feline. Preferably, the patient is a human.

A “lesion” as used herein refers to an area of abnormal tissue. A lesion may be benign or malignant (“cancer lesion”, also “tumor lesion”).

The term “intra-tumoral”, “intratumoral”, “tumor infiltrating” or “tumoral” in the context of cells, structures, proteins, antibodies, or markers refers to their localization within the tumor tissue.

Cells which are “positive” or “+” for a certain marker or protein are cells characterized by substantial expression of that marker or protein. Marker or protein expression can be determined and quantified as known in the art, e.g. to define different cell populations. For the characterization of (immune) cell populations, the marker expression can be determined by FACS or using any other technique described herein.

“Leukocytes” are immune cells expressing CD45.

“CD45+ cells”, as used herein, refer to all leukocytes. CD45 can be used as a marker to distinguish immune cells and non-immune cells.

The term “lymphocyte” refers to all immature, mature, undifferentiated, and differentiated white lymphocyte populations, including tissue specific and specialized varieties. It encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-I cells, B-2 cells, and anergic AN1/T3 cell populations.

“T cells” are immune cells expressing TCRalphaβ, CD3, and CD8 or CD4. As used herein, the term includes naive T cells, CD4+ T cells, CD8+ T cells, regulatory T cells, memory T cells, activated T cells, anergic T cells, tolerant T cells, chimeric B cells, and antigen-specific T cells and further T cell populations known in the art. In some embodiments, the presence of a T cell receptor (TCR) on the cell surface distinguishes T cells from other lymphocytes.

“CD8+ T cells” (also “cytotoxic T cell”, “TC”, “cytotoxic T lymphocyte”, “CTL”, “T-killer cell”, “cytolytic T cell”, “CD8+ T-cell” or “killer T cell”) are T cells expressing CD3, CD45 and CD8. CD8+ T cells can kill cancer cells, cells that are infected (particularly with viruses), or otherwise damaged cells.

“CD4+ T cells” (also “T helper cells”, “Th cells”) are immune cells expressing CD3, CD4 and CD45. There are several subsets of T helper cells, such as, without limitation, Th1, Th2, and Th17. CD4+ T cells help suppress or regulate immune responses. They are essential in B cell antibody class switching, in the activation and growth of cytotoxic T cells, and in maximizing bactericidal activity of phagocytes such as macrophages.

“NK cells” (also natural killer cells) are immune cells which express CD45, CD16, CD56, NKG2D, but are CD3 negative. NK cells do not require activation to kill cells that are missing “self” markers of MHC class 1. NCR1 (also referred to as CD335 or NKp46) is expressed on NK cells and on a subset of NKT cells.

“Natural killer T (NKT) cells” are a heterogeneous group of T cells that share properties of both T cells and natural killer cells.

“iNKT cells” (also “invariant natural killer T cells”) express invariant alpha TCR (Valpha24-Jalpha18, CD24lo), CD44 hi, NK1.1 (mouse), and NKG2D. The invariant TCR recognizes glycoplipid antigen presented by non-polymorphic MHC class I-like molecule, CD1d. These cells can influence an immune response by rapidly producing large amounts of cytokines, i.e. IFNγ.

As known in the art, “effector cells” are immune cells that actively support immune response after stimulation. As used herein, effector cells refer to immune cells expressing Fcγ receptors and are therefore able to mediate ADCC or ADCP. Non-limiting examples of effector cells are monocytes, neutrophils, mast cells, and, preferably, macrophages, and natural killer cells.

The term “chimeric antigen receptor” or “CAR” as used herein, refers to an artificial T cell surface receptor that is engineered to be expressed on an immune effector cell and specifically bind an antigen. CARs may be used as a therapy with adoptive cell transfer. Monocytes are removed from a patient (blood, tumor or ascites fluid) and modified so that they express the receptors specific to a particular form of antigen. In some embodiments, the CARs have been expressed with specificity to a tumor associated antigen. CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target cancers by redirecting a monocyte/macrophage expressing the CAR specific for tumor associated antigens.

If not stated otherwise, dosing schemes are abbreviated as known in the art, e.g. every day (QD), every 2 days (Q2D), or every 3 days (Q3D). In accordance with this, “QW” means once every week, “Q2W” once every two weeks, “Q3W” once every three weeks, “Q4W” means once every four weeks, “Q5W” once every five weeks, and “Q6W” once every six weeks. “BIW” means biweekly, “BIW×4” means biweekly for four doses, that is, 4 doses in two weeks.

A dose ratio referred to as “dose ratio (mg or mg/kg of DGKalpha inhibitor A:mg or mg/kg of DGKzeta inhibitor A′)” either relates to the ratio mg of DGKalpha inhibitor A:mg of DGKzeta inhibitor A′, or relates to mg/kg of DGKalpha inhibitor A:mg/kg of DGKzeta inhibitor A′.

A “dosing cycle” or “treatment cycle” is a period of treatment followed by a period of rest (no treatment) that is repeated on a regular schedule. When this cycle is repeated multiple times on a regular schedule, it makes up a course of treatment.

An intravenous line or “IV line” is a tube or cannula that can be used for an intravenous infusion.

### Definitions for DGK Inhibitors of Formula (I)

The terms as mentioned in the present text in context with DGK inhibitors of formula (I) have the following meanings:

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3 or 4, in particular 1, 2 or 3.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

Should a composite substituent be composed of more than one part, e.g. (C₁-C₂-alkoxy)-(C₁-C₆-alkyl)-, it is possible for a given part to be attached at any suitable position of said composite substituent, e.g. it is possible for the C₁-C₂-alkoxy part to be attached to any suitable carbon atom of the C₁-C₆-alkyl part of said (C₁-C₂-alkoxy)-(C₁-C₆-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”. If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3, 4 or 5 carbon atoms (“C₁-C₅-alkyl”), e.g. a methyl, ethyl, propyl, isopropyl, butyl, sec-butyl isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl group. More particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group, more particularly 1 or 2 carbon atoms (“C₁-C₂-alkyl”), e.g. a methyl or ethyl group.

The term “C₂-C₄-alkyl” means a linear or branched, saturated, monovalent hydrocarbon group having 2, 3 or 4 carbon atoms, e.g. an ethyl, propyl, isopropyl, butyl, sec-butyl, isobutyl or tert-butyl group.

The term “C₁-C₆-hydroxyalkyl” means a linear or branched, saturated, monovalent hydrocarbon group in which the term “C₁-C₆-alkyl” is defined supra, and in which 1 or 2 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl, 1-hydroxybutyl, 2-hydroxybutyl, 3-hydroxybutyl, 4-hydroxybutyl group, 1-hydroxypentyl, 2-hydroxypentyl, 3-hydroxypentyl, 4-hydroxypentyl, 5-hydroxypentyl, 1-hydroxyhexyl, 2-hydroxyhexyl, 3-hydroxyhexyl, 4-hydroxyhexyl, 5-hydroxyhexyl, 6-hydroxyhexyl group, or an isomer thereof. Particularly, said group has 1, 2, 3 or 4 carbon atoms (“C₁-C₄-hydroxyalkyl”), e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl, 1-hydroxybutyl, 2-hydroxybutyl, 3-hydroxybutyl, 4-hydroxybutyl group, or an isomer thereof.

The term “C₂-C₄-hydroxyalkyl” means a linear or branched, saturated, monovalent hydrocarbon group having 2, 3 or 4 carbon atoms, in which the term “C₂-C₄-alkyl” is defined supra, and in which 1 or 2 hydrogen atoms are replaced with a hydroxy group, e.g. a 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl, 1-hydroxybutyl, 2-hydroxybutyl, 3-hydroxybutyl or 4-hydroxybutyl group, or an isomer thereof.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl, 1,3-difluoropropan-2-yl, 4,4,4-trifluorobutyl, 5,5,5-trifluoropentyl or 6,6,6-trifluorohexyl. Particularly, said group has 1, 2, 3 or 4 carbon atoms (“C₁-C₄-haloalkyl”), e.g. a fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl, 1,3-difluoropropan-2-yl or 4,4,4-trifluorobutyl group.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof. Particularly, said group has 1, 2, 3 or 4 carbon atoms (“C₁-C₄-alkoxy”), e.g. a methoxy, ethoxy, n-propoxy, isopropoxy, n-butoxy, sec-butoxy, isobutoxy or tert-butoxy group.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₂-C₆-alkenyl” means a linear or branched, monovalent hydrocarbon group, which contains one or two double bonds, and which has 2, 3, 4, 5 or 6 carbon atoms, it being understood that in the case in which said alkenyl group contains two double bonds, then it is possible for said double bonds to be conjugated with each other, or to form an allene. Said alkenyl group is, for example, an ethenyl (or “vinyl”), prop-2-en-1-yl (or “allyl”), prop-1-en-1-yl, but-3-enyl, but-2-enyl, but-1-enyl, pent-4-enyl, pent-3-enyl, pent-2-enyl, pent-1-enyl, hex-5-enyl, hex-4-enyl, hex-3-enyl, hex-2-enyl, hex-1-enyl, prop-1-en-2-yl (or “isopropenyl”), 2-methylprop-2-enyl, 1-methylprop-2-enyl, 2-methylprop-1-enyl, 1-methylprop-1-enyl, 3-methylbut-3-enyl, 2-methylbut-3-enyl, 1-methylbut-3-enyl, 3-methylbut-2-enyl, 2-methylbut-2-enyl, 1-methylbut-2-enyl, 3-methylbut-1-enyl, 2-methylbut-1-enyl, 1-methylbut-1-enyl, 1,1-dimethylprop-2-enyl, 1-ethylprop-1-enyl, 1-propylvinyl, 1-isopropylvinyl, 4-methylpent-4-enyl, 3-methylpent-4-enyl, 2-methylpent-4-enyl, 1-methylpent-4-enyl, 4-methylpent-3-enyl, 3-methylpent-3-enyl, 2-methylpent-3-enyl, 1-methylpent-3-enyl, 4-methylpent-2-enyl, 3-methylpent-2-enyl, 2-methylpent-2-enyl, 1-methylpent-2-enyl, 4-methylpent-1-enyl, 3-methylpent-1-enyl, 2-methylpent-1-enyl, 1-methylpent-1-enyl, 3-ethylbut-3-enyl, 2-ethylbut-3-enyl, 1-ethylbut-3-enyl, 3-ethylbut-2-enyl, 2-ethylbut-2-enyl, 1-ethylbut-2-enyl, 3-ethylbut-1-enyl, 2-ethylbut-1-enyl, 1-ethylbut-1-enyl, 2-propylprop-2-enyl, 1-propylprop-2-enyl, 2-isopropylprop-2-enyl, 1-isopropylprop-2-enyl, 2-propylprop-1-enyl, 1-propylprop-1-enyl, 2-isopropylprop-1-enyl, 1-isopropylprop-1-enyl, 3,3-dimethylprop-1-enyl, 1-(1,1-dimethylethyl)ethenyl, buta-1,3-dienyl, penta-1,4-dienyl or hexa-1,5-dienyl group.

The term “C₂-C₆-alkynyl” means a linear or branched, monovalent hydrocarbon group which contains one triple bond, and which contains 2, 3, 4, 5 or 6 carbon atoms, particularly 2, 3 or 4 carbon atoms (“C₂-C₄-alkynyl”). Said C₂-C₆-alkynyl group is, for example, ethynyl, prop-1-ynyl, prop-2-ynyl (or “propargyl”), but-1-ynyl, but-2-ynyl, but-3-ynyl, pent-1-ynyl, pent-2-ynyl, pent-3-ynyl, pent-4-ynyl, hex-1-ynyl, hex-2-ynyl, hex-3-ynyl, hex-4-ynyl, hex-5-ynyl, 1-methylprop-2-ynyl, 2-methylbut-3-ynyl, 1-methylbut-3-ynyl, 1-methylbut-2-ynyl, 3-methylbut-1-ynyl, 1-ethylprop-2-ynyl, 3-methylpent-4-ynyl, 2-methylpent-4-ynyl, 1-methyl-pent-4-ynyl, 2-methylpent-3-ynyl, 1-methylpent-3-ynyl, 4-methylpent-2-ynyl, 1-methyl-pent-2-ynyl, 4-methylpent-1-ynyl, 3-methylpent-1-ynyl, 2-ethylbut-3-ynyl, 1-ethylbut-3-ynyl, 1-ethylbut-2-ynyl, 1-propylprop-2-ynyl, 1-isopropylprop-2-ynyl, 2,2-dimethylbut-3-ynyl, 1,1-dimethylbut-3-ynyl, 1,1-dimethylbut-2-ynyl or 3,3-dimethylbut-1-ynyl group.

The term “C₃-C₆-cycloalkyl” means a saturated, monovalent, monocyclic hydrocarbon ring which contains 3, 4, 5 or 6 carbon atoms. Said C₃-C₆-cycloalkyl group is for example a cyclopropyl, cyclobutyl, cyclopentyl or cyclohexyl group. Particularly, said group has 3, 4 or 5 carbon atoms (“C₃-C₅-cycloalkyl”), e.g. a cyclopropyl, cyclobutyl or cyclopentyl group. Particularly, said group has 3 or 4 carbon atoms (“C₃-C₄-cycloalkyl”), e.g. a cyclopropyl or cyclobutyl group.

The term “C₄-C₆-cycloalkenyl” means a monocyclic hydrocarbon ring which contains 4, 5 or 6 carbon atoms and one double bond. Particularly, said ring contains 5 or 6 carbon atoms (“C₅-C₆-cycloalkenyl”). Said C₄-C₆-cycloalkenyl group is for example, a monocyclic hydrocarbon ring, e.g. a cyclobutenyl, cyclopentenyl, cyclohexenyl or cycloheptenyl group.

The term “C₃-C₆-cycloalkyloxy” means a saturated, monovalent group of formula (C₃-C₆-cycloalkyl)-O—, in which the term “C₃-C₆-cycloalkyl” is as defined supra, e.g. a cyclopropyloxy, cyclobutyloxy, cyclopentyloxy or cyclohexyloxy group.

The term “4- to 7-membered heterocycloalkyl” means a monocyclic, saturated heterocycle with 4, 5, 6 or 7 ring atoms in total, which contains one or two identical or different ring heteroatoms from the series N, O and S.

Said heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

The term “5- to 7-membered heterocycloalkenyl” means a monocyclic, unsaturated, non-aromatic heterocycle with 5, 6 or 7 ring atoms in total, which contains one or two double bonds and one or two identical or different ring heteroatoms from the series N, O and S.

Said heterocycloalkenyl group is, for example, 4H-pyranyl, 2H-pyranyl, 2,5-dihydro-1H-pyrrolyl, [1,3]dioxolyl, 4H-[1,3,4]thiadiazinyl, 2,5-dihydrofuranyl, 2,3-dihydrofuranyl, 2,5-dihydrothiophenyl, 2,3-dihydrothiophenyl, 4,5-dihydrooxazolyl or 4H-[1,4]thiazinyl.

The term “(4- to 7-membered heterocycloalkyl)oxy” means a monocyclic, saturated heterocycloalkyl of formula (4- to 7-membered heterocycloalkyl)-O— in which the term “4- to 7-membered heterocycloalkyl” is as defined supra.

The term “nitrogen containing 4- to 7-membered heterocycloalkyl group” means a monocyclic, saturated heterocycle with 4, 5, 6 or 7 ring atoms in total, which contains one ring nitrogen atom and optionally one further ring heteroatom from the series N, O and S.

Said nitrogen containing 4- to 7-membered heterocycloalkyl group, without being limited thereto, can be a 4-membered ring, such as azetidinyl, for example; or a 5-membered ring, such as pyrrolidinyl, imidazolidinyl, pyrazolidinyl, 1,2-oxazolidinyl, 1,3-oxazolidinyl or 1,3-thiazolidinyl, for example; or a 6-membered ring, such as piperidinyl, morpholinyl, thiomorpholinyl, piperazinyl, or 1,2-oxazinanyl, for example, or a 7-membered ring, such as azepanyl, 1,4-diazepanyl or 1,4-oxazepanyl, for example.

The term “heteroaryl” means a monovalent, monocyclic or bicyclic aromatic ring having 5, 6, 8, 9 or 10 ring atoms (a “5- to 10-membered heteroaryl” group), which contains at least one ring heteroatom and optionally one, two or three further ring heteroatoms from the series: N, O and/or S, and which is bound via a ring carbon atom.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a 9-membered heteroaryl group, such as, for example, benzofuranyl, benzothienyl, benzoxazolyl, benzisoxazolyl, benzimidazolyl, benzothiazolyl, benzotriazolyl, thiazolopyridinyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₆-cycloalkyl”, means a cycloalkyl group having a finite number of carbon atoms of 3 to 6, i.e. 3, 4, 5 or 6 carbon atoms.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₆” encompasses C₃, C₄, C₅, C₆, C₃-C₆, C₃-C₅, C₃-C₄, C₄-C₆, C₄-C₅, and C₅-C₆;

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)-sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butyl-phenyl)sulfonyl]oxy and [(4-methoxyphenyl)sulfonyl]oxy.

### Definitions for DGK Inhibitors of Formula (II)

The terms as mentioned in the present text in context with DGK inhibitors of formula (II) have the following meanings:

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3 or 4, in particular 1, 2 or 3.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one substituent.

As used herein, an oxo substituent represents an oxygen atom, which is bound to a carbon atom or to a sulfur atom via a double bond.

Should a composite substituent be composed of more than one part, e.g. (C₁-C₂-alkoxy)-(C₁-C₆-alkyl)-, it is possible for a given part to be attached at any suitable position of said composite substituent, e.g. it is possible for the C₁-C₂-alkoxy part to be attached to any suitable carbon atom of the C₁-C₆-alkyl part of said (C₁-C₂-alkoxy)-(C₁-C₆-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”. If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group, more particularly 1 or 2 carbon atoms (“C₁-C₂-alkyl”), e.g. a methyl or ethyl group.

The term “C₁-C₄-hydroxyalkyl” means a linear or branched, saturated, monovalent hydrocarbon group in which the term “C₁-C₄-alkyl” is defined supra, and in which 1 or 2 hydrogen atoms are replaced with a hydroxy group, e.g. a hydroxymethyl, 1-hydroxyethyl, 2-hydroxyethyl, 1,2-dihydroxyethyl, 3-hydroxypropyl, 2-hydroxypropyl, 1-hydroxypropyl, 1-hydroxypropan-2-yl, 2-hydroxypropan-2-yl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl, 1-hydroxybutyl, 2-hydroxybutyl, 3-hydroxybutyl, 4-hydroxybutyl group, or an isomer thereof.

The term “C₁-C₆-haloalkyl” means a linear or branched, saturated, monovalent hydrocarbon group in which the term “C₁-C₆-alkyl” is as defined supra, and in which one or more of the hydrogen atoms are replaced, identically or differently, with a halogen atom. Particularly, said halogen atom is a fluorine atom. Said C₁-C₆-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl or 1,3-difluoropropan-2-yl.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Particularly, said halogen atom is a fluorine atom. Said C₁-C₆-haloalkoxy group is, for example, fluoromethoxy, difluoromethoxy, trifluoromethoxy, 2,2,2-trifluoroethoxy or pentafluoroethoxy.

The term “C₃-C₄-alkenyl” means a linear or branched, monovalent hydrocarbon group, which contains one or two double bonds, and which has 3 or 4 carbon atoms. Said alkenyl group is, for example, a prop-2-en-1-yl (or “allyl”), prop-1-en-1-yl, but-3-enyl, but-2-enyl or but-1-enyl group.

The term “C₃-C₄-alkynyl” means a linear or branched, monovalent hydrocarbon group which contains one triple bond, and which contains 3 or 4 carbon atoms. Said C₃-C₄-alkynyl group is, for example, a prop-1-ynyl, prop-2-ynyl (or “propargyl”), but-1-ynyl, but-2-ynyl or but-3-ynyl group.

The term “C₃-C₇-cycloalkyl” means a saturated, monovalent, monocyclic hydrocarbon ring which contains 3, 4, 5, 6 or 7 carbon ring atoms (“C₃-C₇cycloalkyl”). Said C₃-C₇-cycloalkyl group is for example, a cyclopropyl, cyclobutyl, cyclopentyl, cyclohexyl or cycloheptyl group.

The term “bicyclic C₆-C₁₁-cycloalkyl” means a spirocycloalkyl, fused C₆-C₁₀-cycloalkyl or bridged C₇-C₁₀-cycloalkyl group as defined below:

The term “spirocycloalkyl” means a bicyclic, saturated, monovalent C₅-C₁₁ hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom. Said spirocycloalkyl group is, for example, spiro[2.6]nonyl, spiro[3.3]heptyl, spiro[3.4]octyl, spiro[3.5]nonyl, spiro[3.6]decyl, spiro[4.4]nonyl, spiro[4.5]decyl, spiro[4.6]undecyl or spiro[5.5]undecyl.

The term “fused C₆-C₁₀-cycloalkyl” means a bicyclic, saturated, monovalent hydrocarbon group, in which the two rings share two adjacent ring atoms, such as bicyclo[4.2.0]octyl, octahydropentalenyl or decalinyl.

The term “bridged C₇-C₁₀-cycloalkyl” means a bicyclic, saturated, monovalent hydrocarbon group which the two rings share two common ring atoms which are not adjacent, e.g. bicyclo[2.2.1]heptyl (also known as norbornyl).

The term “bicyclic C₅-C₁₁-cycloalkyl” means a spirocycloalkyl, fused C₅-C₁₀-cycloalkyl or bridged C₅-C₁₀-cycloalkyl group as defined below:

The term “spirocycloalkyl” means a bicyclic, saturated, monovalent C₅-C₁₁ hydrocarbon group in which the two rings share one common ring carbon atom, and wherein said bicyclic hydrocarbon group contains 5, 6, 7, 8, 9, 10 or 11 carbon atoms, it being possible for said spirocycloalkyl group to be attached to the rest of the molecule via any one of the carbon atoms except the spiro carbon atom. Said spirocycloalkyl group is, for example, spiro[2.6]nonyl, spiro[3.3]heptyl, spiro[3.4]octyl, spiro[3.5]nonyl, spiro[3.6]decyl, spiro[4.4]nonyl, spiro[4.5]decyl, spiro[4.6]undecyl or spiro[5.5]undecyl.

The term “fused C₅-C₁₀-cycloalkyl” means a bicyclic, saturated, monovalent hydrocarbon group, in which the two rings share two adjacent ring atoms, such as bicyclo[4.2.0]octyl, octahydropentalenyl or decalinyl.

The term “bridged C₅-C₁₀-cycloalkyl” means a bicyclic, saturated, monovalent hydrocarbon group which the two rings share two common ring atoms which are not adjacent, e.g. bicyclo[1.1.1]pentyl or bicyclo[2.2.1]heptyl (also known as norbornyl).

The term “monocyclic 4- to 7-membered heterocycloalkyl” means a monocyclic, saturated heterocycle with 4, 5, 6 or 7 ring atoms in total, which contains one or two identical or different ring heteroatoms from the series N, O and S.

Said monocyclic heterocycloalkyl group, without being limited thereto, can be a 4-membered ring, such as azetidinyl, oxetanyl or thietanyl, for example; or a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example, or a 7-membered ring, such as azepanyl, 1,4-diazepanyl or 1,4-oxazepanyl, for example.

The term “monocyclic nitrogen containing 4- to 7-membered heterocycloalkyl group” means a monocyclic, saturated heterocycle with 4, 5, 6 or 7 ring atoms in total, which contains one ring nitrogen atom and optionally one further ring heteroatom from the series N, O and S.

Said monocyclic nitrogen containing 4- to 7-membered heterocycloalkyl group, without being limited thereto, can be a 4-membered ring, such as azetidinyl, for example; or a 5-membered ring, such as pyrrolidinyl, imidazolidinyl, pyrazolidinyl, 1,2-oxazolidinyl, 1,3-oxazolidinyl or 1,3-thiazolidinyl, for example; or a 6-membered ring, such as piperidinyl, morpholinyl, thiomorpholinyl, piperazinyl, or 1,2-oxazinanyl, for example, or a 7-membered ring, such as azepanyl, 1,4-diazepanyl or 1,4-oxazepanyl, for example.

The term “monocyclic nitrogen containing 4- to 7-membered heterocycloalkyl group which is optionally benzocondensed” means a monocyclic, saturated heterocycle with 4, 5, 6 or 7 ring atoms in total, which contains one ring nitrogen atom and optionally one further ring heteroatom from the series N, O and S, in which two adjacent ring carbon atoms may be shared with a benzene ring optionally fused thereto, such group being one of the aforementioned monocyclic nitrogen containing 4- to 7-membered heterocycloalkyl groups, such as pyrrolidinyl, piperidinyl, and the like, or benzocondensed groups e.g. 3,4-dihydroquinolin-1(2H)-yl, 3,4-dihydroisoquinolin-2(1H)-yl, 1,3-dihydro-2H-isoindol-2-yl or 2,3-dihydro-1H-indol-1-yl.

The term “bicyclic 6-11 membered heterocycloalkyl” means a 6- to 11-membered heterospirocycloalkyl, a 6- to 10-membered fused heterocycloalkyl or a 7- to 10-membered bridged heterocycloalkyl group as defined below:

The term “6- to 11-membered heterospirocycloalkyl” means a bicyclic, saturated heterocycle with 6, 7, 8, 9, 10 or 11 ring atoms in total, in which the two rings share one common ring carbon atom, which “heterospirocycloalkyl” contains one or two identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, azaspiro[4,5]decyl, oxazaspiro[5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “a 6- to 10-membered fused heterocycloalkyl” means a bicyclic, saturated heterocycle with 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, azabicyclo[3.3.0]octyl, azabicyclo[4.3.0]nonyl, diazabicyclo[4.3.0]nonyl, oxazabicyclo[4.3.0]nonyl, thiazabicyclo[4.3.0]nonyl or azabicyclo[4.4.0]decyl.

The term “a 7- to 10-membered bridged heterocycloalkyl” means a bicyclic, saturated heterocycle with 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo-[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]-nonyl, azabicyclo[4.2.1]nonyl, diazabicyclo[4.2.1]nonyl, oxazabicyclo[4.2.1]nonyl, thiazabicyclo[4.2.1]nonyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl.

The term “bicyclic nitrogen containing 6-11 membered heterocycloalkyl” means a 6- to 11-membered heterospirocycloalkyl, 6- to 10-membered fused heterocycloalkyl or 7- to 10-membered bridged heterocycloalkyl group as defined supra, however containing one ring nitrogen atom and optionally one or two further ring heteroatoms from the series N, O and S; it being possible for said bicyclic nitrogen containing 6-11 membered heterocycloalkyl group to be attached to the rest of the molecule via a nitrogen atom or any one of the carbon atoms, except a spiro carbon atom.

The term “bicyclic 5-11 membered heterocycloalkyl” means a 5-11 membered heterospirocycloalkyl, a 5-11 membered fused heterocycloalkyl or a 5-11 membered bridged heterocycloalkyl group as defined below:

The term “5-11 membered heterospirocycloalkyl” means a bicyclic, saturated heterocycle with 5, 6, 7, 8, 9, 10 or 11 ring atoms in total, in which the two rings share one common ring carbon atom, which “heterospirocycloalkyl” contains one or two identical or different ring heteroatoms from the series: N, O, S; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.2]pentyl, azaspiro[2.3]hexyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, azaspiro[4,5]decyl, oxazaspiro[5.5]undecyl, diazaspiro[3.3]heptyl, thiazaspiro[3.3]heptyl, thiazaspiro[4.3]octyl, azaspiro[5.5]undecyl, or one of the further homologous scaffolds such as spiro[3.4]-, spiro[4.4]-, spiro[2.4]-, spiro[2.5]-, spiro[2.6]-, spiro[3.5]-, spiro[3.6]-, spiro[4.5]- and spiro[4.6]-.

The term “5-11 membered fused heterocycloalkyl” means a bicyclic, saturated heterocycle with 5, 6, 7, 8, 9 or 10 ring atoms in total, in which the two rings share two adjacent ring atoms, which “fused heterocycloalkyl” contains one or two identical or different ring heteroatoms from the series: N, O, S; it being possible for said fused heterocycloalkyl group to be attached to the rest of the molecule via any one of the carbon atoms or, if present, a nitrogen atom.

Said fused heterocycloalkyl group is, for example, azabicyclo[3.1.0]hexyl, azabicyclo[3.3.0]octyl, azabicyclo[4.3.0]nonyl, diazabicyclo[4.3.0]nonyl, oxazabicyclo[4.3.0]nonyl, thiazabicyclo[4.3.0]-nonyl or azabicyclo[4.4.0]decyl.

The term “5-11 membered bridged heterocycloalkyl” means a bicyclic, saturated heterocycle with 5, 6, 7, 8, 9 or 10 ring atoms in total, in which the two rings share two common ring atoms which are not adjacent, which “bridged heterocycloalkyl” contains one or two identical or different ring heteroatoms from the series: N, O, S; it being possible for said bridged heterocycloalkyl group to be attached to the rest of the molecule via any one of the carbon atoms, except the spiro carbon atom, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, diazabicyclo[2.2.1]heptyl, azabicyclo[2.2.2]octyl, diazabicyclo[2.2.2]octyl, oxazabicyclo[2.2.2]octyl, thiazabicyclo[2.2.2]octyl, azabicyclo[3.2.1]octyl, diazabicyclo[3.2.1]octyl, oxazabicyclo[3.2.1]octyl, thiazabicyclo[3.2.1]octyl, azabicyclo[3.3.1]nonyl, diazabicyclo[3.3.1]nonyl, oxazabicyclo[3.3.1]nonyl, thiazabicyclo[3.3.1]nonyl, azabicyclo[4.2.1]nonyl, diazabicyclo[4.2.1]nonyl, oxazabicyclo[4.2.1]nonyl, thiazabicyclo[4.2.1]nonyl, azabicyclo[3.3.2]decyl, diazabicyclo[3.3.2]decyl, oxazabicyclo[3.3.2]decyl, thiazabicyclo[3.3.2]decyl or azabicyclo[4.2.2]decyl.

The term “bicyclic nitrogen containing 5-11 membered heterocycloalkyl” means a 5-11 membered heterospirocycloalkyl, 5-11 membered fused heterocycloalkyl or 5-11 membered bridged heterocycloalkyl group as defined supra, however containing one ring nitrogen atom and optionally one or two further ring heteroatoms from the series N, O and S; it being possible for said bicyclic nitrogen containing 5-11 membered heterocycloalkyl group to be attached to the rest of the molecule via a nitrogen atom or any one of the carbon atoms, except a spiro carbon atom.

The term “heteroaryl” means a monovalent, monocyclic or bicyclic aromatic ring having 5, 6, 8, 9 or 10 ring atoms (a “5- to 10-membered heteroaryl” group), which contains at least one ring heteroatom and optionally one, two or three further ring heteroatoms from the series: N, O and/or S, and which is bound via a ring carbon atom, or, if valency allows as e.g. in pyrrol-1-yl, a nitrogen atom.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl (herein also referred to as pyridyl), pyridazinyl, pyrimidinyl, pyrazinyl or triazinyl; or a 9-membered heteroaryl group, such as, for example, benzofuranyl, benzothienyl, benzoxazolyl, benzisoxazolyl, benzimidazolyl, benzothiazolyl, benzotriazolyl, thiazolopyridinyl, indazolyl, indolyl, isoindolyl, indolizinyl or purinyl; or a 10-membered heteroaryl group, such as, for example, quinolinyl, quinazolinyl, isoquinolinyl, cinnolinyl, phthalazinyl, quinoxalinyl or pteridinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₆”, as used in the present text, e.g. in the context of the definition of “C₁-C₆-alkyl”, “C₁-C₆-haloalkyl”, “C₁-C₆-hydroxyalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₇”, as used in the present text, e.g. in the context of the definition of “C₃-C₇cycloalkyl”, means a cycloalkyl group having a finite number of carbon atoms of 3 to 7, i.e. 3, 4, 5, 6 or 7 carbon atoms.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₆” encompasses C₃, C₄, C₅, C₆, C₃-C₆, C₃-C₅, C₃-C₄, C₄-C₆, C₄-C₅, and C₅-C₆.

As used herein, the term “leaving group” means an atom or a group of atoms that is displaced in a chemical reaction as stable species taking with it the bonding electrons. In particular, such a leaving group is selected from the group comprising: a halogen atom, in particular a fluorine atom, a chlorine atom, a bromine atom or an iodide atom, being displaced as halide, in particular fluoride, chloride, bromide or iodide; (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butylphenyl)sulfonyl]oxy and [(4-methoxyphenyl)sulfonyl]oxy.

As used herein, the term “dipolar aprotic solvent” means a solvent selected from acetone, acetonitrile, propionitrile, dimethylsulfoxide, diethylsulfoxide, N,N-dimethylformamide, N,N-dimethylacetamide, N,N-diethylformamide, N,N-diethylacetamide, 1-methyl-2-pyrrolidinone, 1-ethyl-2-pyrrolidinone, 1-methyl-2-piperidinone and 1-ethyl-2-piperidinone, or mixtures thereof. Particularly, said dipolar aprotic solvent is acetonitrile, dimethylsulfoxide, N,N-dimethylformamide, N,N-dimethylacetamide or 1-methyl-2-pyrrolidinone.

As used herein, the term “room temperature” means a temperature in the range from 15° C. to 25° C.

### Further Definitions for DGK Inhibitors

As used herein, the term “one or more”, e.g. in the definition of the substituents of the DGK inhibitors of the general formulae of the present invention, is understood as meaning “one, two, three, four or five, particularly one, two, three or four, more particularly one, two or three, even more particularly one or two”.

The definition of the respective DGK inhibitor of formulae (I) or (II) also includes all suitable isotopic variations of the respective DGK inhibitor of formulae (I) or (II). Accordingly, an “isotopic variation” is defined as one in which at least one atom is replaced by an atom having the same atomic number but an atomic mass different from the atomic mass usually or predominantly found in nature. Examples of isotopes that can be incorporated into the DGK inhibitors of formulae (I) or (II) include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I, and ¹³¹I, respectively. Certain isotopic variations of the DGK inhibitors of formulae (I) or (II), for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of the DGK inhibitors of formulae (I) or (II) can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

The DGK inhibitors of formulae (I) and (II) may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

Substituents on a ring may also be present in either cis or trans form. It is intended that all such configurations (including enantiomers and diastereomers), are included within the definition of the respective DGK inhibitor.

Preferred DGK inhibitors of formulae (I) and (II) are those which produce the more desirable biological activity. Separated, pure or partially purified isomers and stereoisomers or racemic or diastereomeric mixtures of the DGK inhibitors of formulae (I) and (II) are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The definitions of the DGK inhibitors described according to the present invention include without limitation all possible stereoisomers of the DGK inhibitors of formulae (I) and (II) as single stereoisomers, or as any mixture of said stereoisomers, e.g. R- or S-isomers, or E- or Z-isomers, in any ratio. Isolation of a single stereoisomer, e.g. a single enantiomer or a single diastereomer, of a DGK inhibitor of formula (I) or (II) may be achieved by any suitable state of the art method, such as chromatography, especially chiral chromatography, for example.

Further, it is possible for some of the DGK inhibitors of formulae (I) and (II) to exist as “tautomers”. For example, the DGK inhibitors of the present invention may contain a pyridone moiety and can exist as a pyridone, or as an hydroxypyridine, or even a mixture in any amount of the two tautomers, namely:

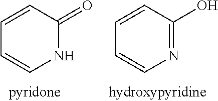

The present definitions of DGK inhibitors include, without limitation, all possible tautomers of DGK inhibitors of formulae (I) and (II) as single tautomers, or as any mixture of said tautomers, in any ratio.

Further, the DGK inhibitors of formulae (I) and (II) can exist as “N-oxides”, which are defined in that at least one nitrogen of the compounds of the present invention is oxidised. The present combination includes, without limitation, all such possible N-oxides of DGK inhibitors of formulae (I) and (II).

The present definition of DGK inhibitors also relates to useful forms of the DGK inhibitors of formulae (I) and (II) as disclosed herein, such as metabolites, hydrates, solvates, prodrugs, salts, in particular pharmaceutically acceptable salts, and co-precipitates.

The DGK inhibitors of formulae (I) and (II) can exist as a “hydrate”, or as a “solvate”, wherein the compounds described herein contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta-etc. solvates or hydrates, respectively, are possible. The DGK inhibitors of formulae (I) and (II) include all such hydrates or solvates.

Further, the DGK inhibitors of formulae (I) and (II) can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The DGK inhibitors of formulae (I) and (II) include all possible salts of the DGK inhibitors of formulae (I) and (II) as single salts, or as any mixture of said salts, in any ratio.

The term “pharmaceutically acceptable salt” of the DGK inhibitors of formulae (I) and (II) refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and chorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

Representative salts of the DGK inhibitors of formulae (I) and (II) include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, sulfate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, or butyl chlorides, bromides and iodides; dialkyl sulfates like dimethyl, diethyl, dibutyl sulfate, or diamyl sulfates, long chain halides such as decyl, lauryl, myristyl and stearyl chlorides, bromides and iodides, aralkyl halides like benzyl and phenethyl bromides and others.

Furthermore, the DGK inhibitors of formulae (I) and (II) include all possible crystalline forms, or polymorphs, of the DGK inhibitors of formulae (I) and (II), either as single polymorphs, or as a mixture of more than one polymorph, in any ratio.

When radicals in the DGK inhibitors of formulae (I) and (II) are substituted, the radicals may be mono- or polysubstituted, unless specified otherwise. In the DGK inhibitors of formulae (I) and (II), all radicals which occur more than once are defined independently of one another. Substitution by one, two or three identical or different substituents is preferred.

### Inhibitors of DGKalpha and Inhibitors of DGKzeta

In the following, the inhibitors of DGKalpha and inhibitors of DGKzeta are described, which can be preferably used for the invention according to any one of aspect 1, aspect 2, aspect 3, aspect 4, aspect 5, aspect 6, aspect 7, aspect 8 or aspect 9 of the invention. In the following, the terms “inhibitors of DGKalpha” and “DGKalpha inhibitors” are used interchangeably. Likewise, in the following, the terms “inhibitors of DGKzeta” and “DGKzeta inhibitors” are used interchangeably.

Inhibitors of DGK have been disclosed previously, as listed supra.

Two members of the DGK family, DGKalpha, and DGKzeta, specifically regulate the pool of DAG that is generated as a second messenger after stimulation of the T cell receptor (TCR). Inhibitors for these two members are therefore preferred.

Further, and as used herein, the term “DGK inhibitor” means a compound which inhibits one or more isoforms of DGK. A DGKalpha inhibitor inhibits the DGKalpha isoform and may be a selective DGKalpha inhibitor or may also, in addition to its DGKalpha inhibitory activity, inhibit other DGK isoforms, such as DGKzeta. Likewise, a DGKzeta inhibitor inhibits the DGKzeta isoform and may be a selective DGKzeta inhibitor or may also, in addition to its DGKzeta inhibitory activity, inhibit other DGK isoforms, such as DGKapha. In the present invention, an inhibitor of DGKalpha is preferably selective with regard to DGKzeta, and an inhibitor of DGKzeta is preferably selective with regard to DGKalpha.

More preferably, an inhibitor of DGKalpha features 20-fold or higher selectivity with regard to DGKzeta, that is, features a 20-fold or higher IC₅₀ value towards DGKzeta as compared to the IC₅₀ value towards DGKalpha, e.g. in the in vitro assays described in the Experimental section, and an inhibitor of DGKzeta features 20-fold or higher selectivity with regard to DGKalpha, that is, features a 20-fold or higher IC₅₀ value towards DGKalpha as compared to the IC₅₀ value towards DGKzeta, e.g. in the in vitro assays described in the Experimental section.

Even more preferably, an inhibitor of DGKalpha features 20-fold or higher selectivity with regard to DGKzeta, that is, features a 100-fold or higher IC₅₀ value towards DGKzeta as compared to the IC₅₀ value towards DGKalpha, e.g. in the in vitro assays described in the Experimental section, and an inhibitor of DGKzeta features 100-fold or higher selectivity with regard to DGKalpha, that is, features a 100-fold or higher IC₅₀ value towards DGKalpha as compared to the IC₅₀ value towards DGKzeta, e.g. in the in vitro assays described in the Experimental section.

Compounds of general formula (I) as used herein are typically DGKalpha inhibitors and compounds of general formula (II) as used herein are typically DGKzeta inhibitors. As shown below, the particularly preferred DGKalpha Inhibitor A features an over 100-fold selectivity over DGKzeta, and the particularly preferred DGKzeta inhibitor A′ features an over 100-fold selectivity over DGKalpha.

### DGKalpha Inhibitors

### DGKalpha Inhibitors Embodiments A

In the following, the inhibitors of DGKalpha are described, which can be preferably used for the invention according to any one of aspect 1, aspect 2, aspect 3, aspect 4, aspect 5, aspect 6, aspect 7, aspect 8 or aspect 9 of the invention.

In some embodiments of the invention, the inhibitor of DGKalpha is a compound of formula (I)

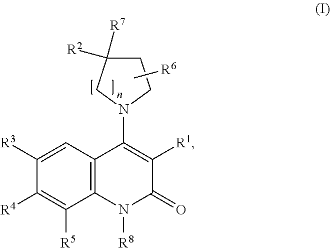

in which:
R¹ represents a group selected from cyano, —C(═O)NH₂, —C(═O)N(H)CH₃, —C(═O)N(H)C₂H₅, —C(═O)N(CH₃)₂ and —C(═O)OR¹⁵,
R² represents a group selected from phenyl, naphthyl and 5- to 10-membered heteroaryl,
   which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁-C₆-alkyl, C₃-C₆-cycloalkyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂ alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   or two substituents of said phenyl group, when they are attached to adjacent ring atoms, are optionally linked to one another in such a way that they jointly form a group selected from
   —(CH₂)₃—, —CH₂—CH(OH)—CH₂—, —(CH₂)₄—, —O—(CH₂)₂—, —(CH₂)₂—O—, —CH₂—CH(CH₃)—O—, —CH₂—O—CH₂—, —O—(CH₂)₃—, —(CH₂)₃—O—, —CH₂—O—(CH₂)₂—, —(CH₂)₂—O—CH₂—, —O—CH₂—O, —O—C(CH₃)₂—O—, —O—(CH₂)₂—O—, —N(R¹⁸)—C(═O)—(C(R¹⁸)(R¹⁹))ₘ—, —N(R¹⁸)—C(═O)—(C(CH₂)₃)—, —N(R¹⁸)—(C(R¹⁸)(R¹⁹))ₘ—, —N(R¹⁸)—C(═O)—O— and —N(R¹⁸)—C(═O)—N(R¹⁸)—,
      wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
      and
      wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      wherein said C₁-C₆-alkyl and C₁-C₆-alkoxy group is optionally substituted with a group selected from C₃-C₄-cycloalkyl, phenyl and 4- to 7-membered heterocycloalkyl,
         wherein said 4- to 7-membered heterocycloalkyl group is connected to the rest of the molecule via a carbon atom of said 4- to 7-membered heterocycloalkyl group,
         and
         wherein said 4- to 7-membered heterocycloalkyl group is optionally substituted, one, two or three times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
         and
         which phenyl group is optionally substituted, one or two times, each substituent independently selected from a halogen atom or a group selected from
         C₁-C₂-alkyl, C₁-C₂-haloalkyl, cyano, hydroxy, C₁-C₂-alkoxy, C₃-C₄-cycloalkyl and —N(R⁹)(R¹⁰),
         and
         which C₃-C₄-cycloalkyl group is optionally substituted, one or two times, each substituent independently selected from a halogen atom or a group selected from cyano and hydroxy,
      and
      wherein said C₃-C₆-cycloalkyl group is optionally substituted, one or two times, each substituent independently selected from a halogen atom or a C₁-C₄-alkyl group,
      and
      wherein said phenyl, phenoxy and 5- or 6-membered heteroaryl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
R³ represents a hydrogen atom or a halogen atom or a group selected from C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂ alkoxy)-(C₁-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
   and
   wherein said C₁-C₆-alkyl, C₂-C₆-alkenyl, C₂-C₆-alkynyl and C₁-C₆-alkoxy group is optionally substituted with a group selected from C₃-C₄-cycloalkyl, phenyl and 4- to 7-membered heterocycloalkyl,
      wherein said 4- to 7-membered heterocycloalkyl group is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₂-C₆-alkenyl group is optionally substituted with a C₁-C₄-haloalkyl group,
   and
   wherein said C₃-C₆-cycloalkyl and C₄-C₆-cycloalkenyl group is optionally substituted, one or two times, each substituent independently selected from a halogen atom or a group selected from C₁-C₄-alkyl and C₁-C₄-haloalkyl,
   and
   wherein said phenyl, phenoxy and 5- or 6-membered heteroaryl group is optionally substituted, one or two times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl and —N(R⁹)(R¹⁰),
R⁴ represents a hydrogen atom or a halogen atom or a group selected from C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂ alkoxy)-(C₁-C₆-alkoxy)-, C₁-C₄-haloalkoxy, —O—(C₁-C₄-alkyl)-C(═O)OR¹⁵, —O—(C₁-C₄-alkyl)-C(═O)N(R⁹)(R¹⁰), C₃-C₆-cycloalkyloxy, —S(═O)R¹⁴, —S(═O)₂R¹⁴, cyano, nitro, hydroxy, —N(R⁹)(R¹⁰), —N(R¹⁶)(R¹⁷), —N(R¹⁶)(R²⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
   wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
   and
   wherein said C₁-C₅-alkyl, C₂-C₆-alkenyl, C₂-C₆-alkynyl and C₁-C₆-alkoxy group is optionally substituted with a group selected from C₃-C₄-cycloalkyl and 4- to 7-membered heterocycloalkyl,
      wherein said 4- to 7-membered heterocycloalkyl group is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from
      cyano and hydroxy,
   and
   wherein said C₁-C₆-alkoxy group is optionally substituted with a oxiran-2-yl group,
   and
   wherein said C₃-C₆-cycloalkyl and C₄-C₆-cycloalkenyl group is optionally substituted, one or two times, each substituent independently selected from a halogen atom or a C₁-C₄-alkyl group,
   and
   wherein said phenyl and 5- or 6-membered heteroaryl group is optionally substituted, one or two times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl and —N(R⁹)(R¹⁰),
R⁵ represents a hydrogen atom or a halogen atom or a group selected from C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂ alkoxy)-(C₂-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl,
   (4- to 7-membered heterocycloalkyl)oxy, phenyl and 5- or 6-membered heteroaryl, wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
   and
   wherein said C₁-C₆-alkyl, C₂-C₆-alkenyl, C₂-C₆-alkynyl and C₁-C₆-alkoxy group is optionally substituted with a group selected from C₃-C₄-cycloalkyl, phenyl and 4- to 7-membered heterocycloalkyl,
      wherein said 4- to 7-membered heterocycloalkyl group is connected to the rest of the molecule via a carbon atom of said 4- to 7-membered heterocycloalkyl group,
      and
      wherein said 4- to 7-membered heterocycloalkyl group is optionally substituted, one, two or three times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
      and
      which phenyl group is optionally substituted, one or two times, each substituent independently selected from a halogen atom or a group selected from
      C₁-C₂-alkyl, C₁-C₂-haloalkyl, cyano, hydroxy, C₁-C₂-alkoxy, C₃-C₄-cycloalkyl and —N(R⁹)(R¹⁰),
      and
      which C₃-C₄-cycloalkyl group is optionally substituted, one or two times, each substituent independently selected from a halogen atom or a group selected from cyano and hydroxy,
   and
   wherein said C₃-C₆-cycloalkyl and C₄-C₆-cycloalkenyl group is optionally substituted, one or two times, each substituent independently selected from a halogen atom or a C₁-C₄-alkyl group,
   and
   wherein said phenyl, phenoxy and 5- or 6-membered heteroaryl group is optionally substituted, one or two times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl and —N(R⁹)(R¹⁰),
R⁶ represents a hydrogen atom, or a fluorine atom or a group selected from C₁-C₄-alkyl, C₁-C₄-hydroxyalkyl, C₁-C₄-alkoxy, hydroxy and oxo,
R⁷ represents a hydrogen atom or a halogen atom or a group selected from C₁-C₄-alkyl, C₁-C₄-alkoxy, hydroxy and cyano,
R⁸ represents a group selected from methyl and ethyl,
R⁹ and R¹⁰ represent, independently from each occurrence, a hydrogen atom or a group selected from C₁-C₄-alkyl, C₂-C₄-hydroxyalkyl, N≡C—(C₁-C₄-alkyl)-, (C₁-C₄-alkoxy)-(C₂-C₄-alkyl)-, C₃-C₄-cycloalkyl and C₂-C₄-haloalkyl,
   or
R⁹ and R¹⁰ together with the nitrogen to which they are attached represent a nitrogen containing 4- to 7-membered heterocycloalkyl group,
   wherein said nitrogen containing 4- to 7-membered heterocycloalkyl group is optionally substituted, one, two or three times, each substituent independently selected from a halogen atom or a group selected from C₁-C₄-alkyl, C₃-C₄-cycloalkyl, hydroxy and oxo,
   or
   two substituents, which are attached to the same carbon atom of said nitrogen containing 4- to 7-membered heterocycloalkyl group, together with the carbon atom to which they are attached, represent a 4- to 7-membered heterocycloalkyl group,
      wherein said 4- to 7-membered heterocycloalkyl group is optionally substituted, one or two times, each substituent independently selected from a halogen atom or a group selected from C₁-C₄-alkyl, C₃-C₄-cycloalkyl, C₁-C₄-haloalkyl, hydroxy and oxo,
R¹¹ represents a hydrogen atom or group selected from C₁-C₄-alkyl, C₁-C₄-hydroxyalkyl, C₁-C₄-haloalkyl, phenyl and 5- or 6-membered heteroaryl,
   wherein said phenyl group and 5- or 6-membered heteroaryl group is optionally substituted, one or two times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl and —N(R⁹)(R¹⁰),
R¹² represents a hydrogen atom or a C₁-C₄-alkyl group,
R¹³ represents a hydrogen atom or a group selected from C₁-C₆-alkyl, phenyl and 5- or 6-membered heteroaryl,
   wherein said phenyl group and 5- or 6-membered heteroaryl group is optionally substituted, one or two times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl and —N(R⁹)(R¹⁰),
R¹⁴ represents a group selected from C₁-C₆-alkyl, C₁-C₆-haloalkyl, C₃-C₆-cycloalkyl, phenyl and 5- or 6-membered heteroaryl,
   wherein said phenyl group and 5- or 6-membered heteroaryl group is optionally substituted, one or two times, each substituent independently selected from a halogen atom or a group selected from C₁-C₂-alkyl, C₁-C₂-haloalkyl, cyano, hydroxy, C₁-C₂-alkoxy, C₃-C₄-cycloalkyl and —N(R⁹)(R¹⁰),
R¹⁵ represents a hydrogen atom or a C₁-C₄-alkyl group,
R¹⁶ represents a hydrogen atom or a group selected from C₁-C₄-alkyl, C₃-C₄-cycloalkyl and C₂-C₄-haloalkyl,
R¹⁷ represents a 4- to 7-membered heterocycloalkyl group,
   wherein said 4- to 7-membered heterocycloalkyl group is optionally substituted, one, two or three times, each substituent independently selected from a halogen atom or a group selected from C₁-C₄-alkyl, C₃-C₄-cycloalkyl, C₁-C₄-alkoxy, hydroxy and oxo,
   and
   wherein said 4- to 7-membered heterocycloalkyl group is connected to the rest of the molecule via a carbon atom of the 4- to 7-membered heterocycloalkyl group,
R¹⁸ represents a hydrogen atom or a group selected from methyl and ethyl,
R¹⁹ represents a hydrogen atom or a group selected from methyl and ethyl,
R²⁰ represents a (4- to 7-membered heterocycloalkyl)-(C₁-C₄-alkyl)- group,
   wherein the (4- to 7-membered heterocycloalkyl) part of said group is optionally substituted, one, two or three times, each substituent independently selected from a halogen atom or a group selected from C₁-C₄-alkyl, C₃-C₄-cycloalkyl, C₁-C₄-alkoxy, hydroxy and oxo,
m represents an integer selected from 1, 2 and 3,
and
n represents an integer selected from 1, 2 and 3,
or a stereoisomer, a tautomer, an N-oxide, a hydrate, a solvate, or a salt thereof, or a mixture of same.

In a preferred embodiment, the at least one inhibitor of DGKalpha is characterized by formula (I) with the restrictions defined under this heading.

### DGKalpha Inhibitors Embodiments B

In some other embodiments of the invention, the inhibitor of DGKalpha is a compound of formula (I), supra, in which
R¹ represents a group selected from cyano, —C(═O)NH₂, —C(═O)N(H)CH₃ and —C(═O)N(CH₃)₂,
R² represents a group selected from phenyl, naphthyl and 5- to 10-membered heteroaryl, which 5- to 10-membered heteroaryl group is connected to the rest of the molecule via a carbon atom of said 5- to 10-membered heteroaryl group,
   and
   which phenyl, naphthyl and 5- to 10-membered heteroaryl group is optionally substituted, one, two, three or four times, each substituent independently selected from a halogen atom or a group selected from
   C₁—C-alkyl, C₃-C₅-cycloalkyl, C₁-C₄-haloalkyl, (C₁-C₂-alkoxy)-(C₁-C₂-alkyl)-, C₁-C₄-alkoxy, C₁-C₄-haloalkoxy, phenoxy, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═O)(R¹⁴)₂, 4- to 7-membered heterocycloalkyl, phenyl and 5- or 6-membered heteroaryl,
   or two substituents of said phenyl group, when they are attached to adjacent ring atoms, are optionally linked to one another in such a way that they jointly form a group selected from
      —CH₂—CH(OH)—CH₂—, —CH₂—CH(CH₃)—O—, —O—C(CH₃)₂—O—, —N(R¹⁸)—C(═O)—(C(R¹⁸)(R¹⁹))ₘ—, —N(R¹⁸)—C(═O)—(C(CH₂)₃)—, —N(R¹⁸)—(C(R¹⁸)(R¹⁹))ₘ—, —N(R¹⁸)—C(═O)—O— and —N(R¹⁸)—C(═O)—N(R¹⁸)—, wherein said 4- to 7-membered heterocycloalkyl group is connected to the rest of the molecule via a carbon atom of said 4- to 7-membered heterocycloalkyl group,
      and
      wherein said C₁-C₄-alkoxy group is optionally substituted with a group selected from 4- to 7-membered heterocycloalkyl and phenyl,
         wherein said 4- to 7-membered heterocycloalkyl group is connected to the rest of the molecule via a carbon atom of said 4- to 7-membered heterocycloalkyl group,
      and
      wherein said phenyl and 5- or 6-membered heteroaryl group is optionally substituted, one or two times, each substituent independently selected from a halogen atom or a group selected from C₁-C₂-alkyl, C₁-C₂-haloalkyl and C₁-C₂-alkoxy,
R³ represents a hydrogen atom or a halogen atom or a group selected from C₁-C₆-alkyl, C₂-C₄-alkenyl, C₃-C₅-cycloalkyl, (C₁-C₂-alkoxy)-(C₁-C₄-alkyl)-, C₁-C₄-alkoxy, C₁-C₄-haloalkoxy, C₃-C₅-cycloalkyloxy, —S(═O)₂R¹⁴, cyano, hydroxy, —N(R⁹)(R¹⁰), —C(═O)N(R⁹)(R¹⁰), —P(═O)(R¹⁴)₂, 4- to 7-membered heterocycloalkyl, 5- to 7-membered heterocycloalkenyl, (4- to 7-membered heterocycloalkyl)oxy and phenyl,
   wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said C₁-C₆-alkyl and C₁-C₄-alkoxy group is optionally substituted with a group selected from C₃-C₄-cycloalkyl and 4- to 7-membered heterocycloalkyl,
      wherein said 4- to 7-membered heterocycloalkyl group is connected to the rest of the molecule via a carbon atom of said 4- to 7-membered heterocycloalkyl group,
      and
      which C₃-C₄-cycloalkyl group is optionally substituted, one or two times, with a cyano group,
   and
   wherein said C₂-C₄-alkenyl group is optionally substituted with a C₁-C₄-haloalkyl group,
   and
   wherein said C₃-C₅-cycloalkyl group is optionally substituted, one or two times, each substituent independently selected from a halogen atom or a group selected from C₁-C₄-alkyl and C₁-C₄-haloalkyl,
R⁴ represents a hydrogen atom or a halogen atom or a group selected from C₁-C₆-alkyl, C₁-C₆-haloalkyl, C₃-C₅-cycloalkyl, (C₁-C₂-alkoxy)-(C₁-C₄-alkyl)-, C₁-C₄-alkoxy, (C₁-C₂ alkoxy)-(C₁-C₄-alkoxy)-, —O—(C₁-C₄-alkyl)-C(═O)OR¹⁵, —O—(C₁-C₄-alkyl)-C(═O)N(R⁹)(R¹⁰), C₃-C₅-cycloalkyloxy, —S(═O)₂R¹⁴, cyano, nitro, hydroxy, —N(R⁹)(R¹⁰), —N(R¹⁶)(R¹⁷), —N(R¹⁶)(R²⁰), —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy and phenyl,
   wherein said 4- to 7-membered heterocycloalkyl group and, 5- to 7-membered heterocycloalkenyl group is connected to the rest of the molecule via a carbon atom of said 4- to 7-membered heterocycloalkyl group and 5- to 7-membered heterocycloalkenyl group,
   and
   wherein said C₁-C₆-alkyl and C₁-C₄-alkoxy group is optionally substituted with a group selected from C₃-C₄-cycloalkyl and 4- to 7-membered heterocycloalkyl,
      wherein said 4- to 7-membered heterocycloalkyl group is connected to the rest of the molecule via a carbon atom of said 4- to 7-membered heterocycloalkyl group,
      and
      which C₃-C₄-cycloalkyl group is optionally substituted, one or two times, each substituent independently selected from a halogen atom or a group selected from cyano and hydroxy,
   and
   wherein said C₁-C₄-alkoxy group is optionally substituted with a oxiran-2-yl group,
   and
   wherein said C₃-C₅-cycloalkyl is optionally substituted, one or two times, each substituent independently selected from a halogen atom or a C₁-C₄-alkyl group,
R⁵ represents a hydrogen atom or a halogen atom or a group selected from C₁-C₅-alkyl, C₃-C₅-cycloalkyl, C₁-C₄-alkoxy, C₃-C₅-cycloalkyloxy, —S(═O)₂R¹⁴, cyano, hydroxy, —N(R⁹)(R¹⁰), 4- to 7-membered heterocycloalkyl and (4- to 7-membered heterocycloalkyl)oxy,
   wherein said 4- to 7-membered heterocycloalkyl group is connected to the rest of the molecule via a carbon atom of said 4- to 7-membered heterocycloalkyl group,
R⁶ represents a hydrogen atom or a group selected from C₁-C₄-alkyl and C₁-C₄-hydroxyalkyl,
R⁷ represents a hydrogen atom or a halogen atom or a group selected from C₁-C₄-alkyl, C₁-C₄-alkoxy and hydroxy,
R⁸ represents a group selected from methyl and ethyl,
R⁹ and R¹⁰ represent, independently from each occurrence, a hydrogen atom or a group selected from C₁-C₄-alkyl, C₂-C₄-hydroxyalkyl, N═C—(C₁-C₄-alkyl)-, (C₁-C₄-alkoxy)-(C₂-C₄-alkyl)- and C₃-C₄-cycloalkyl,
   or
R⁹ and R¹⁰ together with the nitrogen to which they are attached represent a nitrogen containing 4- to 7-membered heterocycloalkyl group,
   wherein said nitrogen containing 4- to 7-membered heterocycloalkyl group is optionally substituted, one, two or three times, each substituent independently selected from a halogen atom or a group selected from C₁-C₄-alkyl, hydroxy and oxo,
   or
   two substituents, which are attached to the same carbon atom of said nitrogen containing 4- to 7-membered heterocycloalkyl group, together with the carbon atom to which they are attached, represent a 4- to 7-membered heterocycloalkyl group,
      wherein said 4- to 7-membered heterocycloalkyl group is optionally substituted, one or two times, with a C₁-C₄-alkyl group,
R¹¹ represents a group selected from C₁-C₄-alkyl and C₁-C₄-haloalkyl,
R¹² represents a hydrogen atom,
R¹³ represents a phenyl group,
R¹⁴ represents a group selected from C₁-C₄-alkyl and phenyl,
R¹⁵ represents a hydrogen atom or a C₁-C₄-alkyl group,
R¹⁶ represents a hydrogen atom or a C₁-C₄-alkyl group,
R¹⁷ represents a 4- to 7-membered heterocycloalkyl group,
   wherein said 4- to 7-membered heterocycloalkyl group is optionally substituted, one or two times, with a C₁-C₄-alkyl group,
   and
   wherein said 4- to 7-membered heterocycloalkyl group is connected to the rest of the molecule via a carbon atom of the 4- to 7-membered heterocycloalkyl group,
R¹⁸ represents a hydrogen atom or a methyl group,
R¹⁹ represents a hydrogen atom or a methyl group,
R²⁰ represents a (4- to 7-membered heterocycloalkyl)-(C₁-C₄-alkyl)- group,
   wherein the (4- to 7-membered heterocycloalkyl) part of said group is optionally substituted, one or two times, with a C₁-C₄-alkyl group,
m represents an integer selected from 1 and 2,
   wherein said 4- to 7-membered heterocycloalkyl group is optionally substituted, one or two times, with a C₁-C₄-alkyl group,
and
n represents an integer selected from 1, 2 and 3,
or a stereoisomer, a tautomer, an N-oxide, a hydrate, a solvate, or a salt thereof, or a mixture of same.

In some preferred embodiments, the inhibitor of DGKalpha is characterized by formula (I) with the restrictions defined under this heading.

### DGKalpha Inhibitors Embodiments C

In some other embodiments of the invention, the inhibitor of DGKalpha is a compound of formula (I), supra, in which
R¹ represents a group selected from cyano, —C(═O)NH₂, —C(═O)N(H)CH₃ and —C(═O)N(CH₃)₂,
R² represents a group selected from phenyl, 1-naphthyl, 2-naphthyl, 1H-pyrazol-3-yl, 1H-pyrazol-4-yl, 1H-pyrazol-5-yl, 1,2,4-oxadiazol-5-yl, 1,3,4-oxadiazol-2-yl, 1H-1,2,3-triazol-4-yl, 2H-1,2,3-triazol-4-yl, 1,3-thiazol-2-yl, pyridin-3-yl, pyrazin-2-yl, 1H-indol-5-yl, 1-benzofuran-4-yl, 1-benzofuran-7-yl, 1H-indol-6-yl, benzothiophen-2-yl, 1,3-benzoxazol-2-yl, 1,3-benzoxazol-5-yl, 1,3-benzoxazol-6-yl, 1,3-benzoxazol-7-yl, 1H-indazol-5-yl, 1H-benzimidazol-2-yl, 1H-benzimidazol-4-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1H-pyrrolo[2,3-b]pyridin-3-yl, quinolin-2-yl, quinolin-4-yl, quinolin-6-yl, quinolin-7-yl, isoquinolin-5-yl, isoquinolin-7-yl, isoquinolin-8-yl, quinoxalin-2-yl, quinoxalin-5-yl and 1,3-thiazolo[5,4-b]pyridin-2-yl,
   which group is optionally substituted, one or two times, each substituent independently selected from a fluorine, chlorine or bromine atom or a group selected from
   methyl, propyl, isopropyl, tert-butyl, cyclopropyl, difluoromethyl, trifluoromethyl, methoxy, ethoxy, propoxy, (propan-2-yl)oxy, methoxymethyl, 2-methoxyethyl, benzyloxy, trifluormethoxy, 2,2,2-trifluoroethoxy, phenoxy, (oxolan-2-yl)methoxy,
   (tetrahydrofuran-2-yl)methoxy, methanesulfonyl, dimethylphosphoryl, cyano, hydroxy, dimethylamino, oxetan-3-yl, 2-oxopyrrolidin-1-yl, 4-methyl-2-oxopiperazin-1-yl, 4-methyl-3-oxopiperazin-1-yl, morpholino-4-yl, 7-oxo-2-oxa-6-azaspiro[3.4]octan-6-yl,
   8-methyl-3-ox-2,8-diazaspiro[4.5]decan-2-yl, carbamoyl, acetyl, trifluoroacetyl, benzamido, benzenesulfonamido, [dimethyl(oxido)-λ⁶-sulfanylidene]amino, phenyl, 3-chlorophenyl, 4-chlorophenyl, 2-methylphenyl, 3-methylphenyl, 4-methylphenyl, 3-trifluoromethylphenyl, 2-methoxyphenyl, 3-methoxyphenyl, 4-methoxyphenyl and pyridin-3-yl,
   or
   two substituents of said phenyl group, when they are attached to adjacent ring atoms, are linked to one another in such a way that they jointly form a group selected from
   —CH₂—CH(OH)—CH₂—, —CH₂—CH(CH₃)—O—, —O—C(CH₃)₂—O—, —NH—C(═O)—CH(CH₃)—, —N(CH₃)—C(═O)—C(CH₃)₂—, —NH—C(═O)—(C(CH₂)₃)—, —NH—CH₂—C(CH₃)₂—, —N(CH₃)—C(═O)—O— and —N(CH₃)—C(═O)—N(CH₃)—,
R³ represents a hydrogen atom or a fluorine, chlorine or bromine atom or a group selected from methyl, sec-butyl, (oxetan-3-yl)methyl, 3,3,3-trifluoroprop-1-en-2-yl, cyclopropyl, (trifluoromethyl)cyclopropyl, cyclobutyl, 2,2-dimethylcyclobutyl, 3,3-difluorocyclobutyl, methoxymethyl, methoxy, ethoxy, propoxy, 2,2-difluoroethoxy, 2,2-difluoropropoxy, cyclopropylmethoxy, (1-cyanocyclopropyl)methoxy, cyclopropyloxy, cyclobutyloxy, methanesulfonyl, cyano, hydroxy, 4-hydroxy-2-oxo-pyrrolidin-1-yl, 7-oxo-2-oxa-6-azaspiro[3.4]octan-6-yl, carbamoyl, dimethylphosphoryl, oxetan-3-yl, 3,6-dihydro-2H-pyran-4-yl, (oxetan-3-yl)oxy and phenyl,
R⁴ represents a hydrogen atom or a fluorine, chlorine or bromine atom or a group selected from methyl, sec-butyl, (oxetan-3-yl)methyl, trifluoromethyl, cyclopropyl, 3,3-difluorocyclobutyl, methoxymethyl, methoxy, propoxy, 2-methoxyethoxy, (1-hydroxycyclopropyl)methoxy, (1-cyanocyclopropyl)methoxy, (oxiran-2-yl)methoxy, carboxymethoxy, 2-tert-butoxy-2-oxo-ethoxy, 2-amino-2-oxo-ethoxy, cyclopropyloxy, cyclobutyloxy, methanesulfonyl, dimethylphosphoryl, cyano, nitro, hydroxy, (cyanomethyl)(methyl)amino, (2-hydroxyethyl)amino, (2-hydroxyethyl)(methyl)amino, (2-methoxyethyl)amino, (2-methoxyethyl)(methyl)amino, cyclopropylamino, (oxetan-3-yl)amino, methyl(oxetan-3-yl)amino, methyl(oxolan-3-yl)amino, 3-hydroxyazetidin-1-yl, 2-oxopyrrolidin-1-yl, morpholino, 1,1-dioxidothiomorpholin-4-yl, 4-hydroxy-2-oxo-pyrrolidin-1-yl, 7-oxo-2-oxa-6-azaspiro[3.4]octan-6-yl, 2,2-dimethyl-2λ⁶-diazathia-1,2-dien-1-yl, [dimethyl(oxido)-λ⁶-sulfanylidene]amino, methyl(tetrahydrofuran-3-yl)amino, tetrahydrofuran-3-ylmethoxy, (tetrahydrofuran-3-ylmethyl)amino, oxetan-3-yl, 3,6-dihydro-2H-pyran-4-yl, (oxetan-3-yl)oxy, (tetrahydrofuran-3-yl)oxy, (tetrahydro-2H-pyran-3-yl)oxy, (tetrahydro-2H-pyran-4-yl)oxy and phenyl,
R⁵ represents a hydrogen atom or a fluorine, chlorine or bromine atom or a group selected from methyl, cyclopropyl, methoxy, propoxy, cyclopropyloxy, methanesulfonyl, cyano, hydroxy, oxetan-3-yl and oxetan-3-yloxy,
R⁶ represents a hydrogen atom or a group selected from methyl and hydroxymethyl,
R⁷ represents a hydrogen atom or a fluorine atom or a group selected from methyl, ethyl, methoxy and hydroxy,
R⁸ represents a group selected from methyl and ethyl,
and
n represents an integer selected from 1, 2 and 3,
or a stereoisomer, a tautomer, an N-oxide, a hydrate, a solvate, or a salt thereof, or a mixture of same.

In some preferred embodiments, the inhibitor of DGKalpha is characterized by formula (I) with the restrictions defined under this heading.

### DGKalpha Inhibitors Embodiments D

In some other embodiments of the invention, the inhibitor of DGKalpha is a compound selected from:
4-[4-(1,3-benzoxazol-2-yl)-4-methylpiperidin-1-yl]-1-methyl-2-oxo-1,2-dihydroquinoline-3-carbonitrile,
4-[4-(1,3-benzoxazol-2-yl)piperidin-1-yl]-1-methyl-2-oxo-1,2-dihydroquinoline-3-carbonitrile,
4-[4-(7-fluoro-1,3-benzoxazol-2-yl)piperidin-1-yl]-1-methyl-2-oxo-1,2-dihydroquinoline-3-carbonitrile,
4-[4-(6-fluoro-1,3-benzoxazol-2-yl)piperidin-1-yl]-1-methyl-2-oxo-1,2-dihydroquinoline-3-carbonitrile,
1-methyl-4-[4-(5-methyl-1,3-benzoxazol-2-yl)piperidin-1-yl]-2-oxo-1,2-dihydroquinoline-3-carbonitrile,
4-[4-(5-fluoro-1,3-benzoxazol-2-yl)piperidin-1-yl]-1-methyl-2-oxo-1,2-dihydroquinoline-3-carbonitrile,
1-methyl-4-[4-(6-methyl-1,3-benzoxazol-2-yl)piperidin-1-yl]-2-oxo-1,2-dihydroquinoline-3-carbonitrile,
1-methyl-2-oxo-4-{4-[5-(propan-2-yl)-1,3-benzoxazol-2-yl]piperidin-1-yl}-1,2-dihydroquinoline-3-carbonitrile,
4-[4-(6-chloro-1,3-benzoxazol-2-yl)piperidin-1-yl]-1-methyl-2-oxo-1,2-dihydroquinoline-3-carbonitrile,
4-[4-(5-bromo-1,3-benzoxazol-2-yl)piperidin-1-yl]-1-methyl-2-oxo-1,2-dihydroquinoline-3-carbonitrile,
4-[4-(5-chloro-1,3-benzoxazol-2-yl)piperidin-1-yl]-1-methyl-2-oxo-1,2-dihydroquinoline-3-carbonitrile,
4-[4-(1,3-benzothiazol-2-yl)piperidin-1-yi]-1-methyl-2-oxo-1,2-dihydroquinoline-3-carbonitrile,
4-[4-(5-fluoro-1,3-benzothiazol-2-yl)piperidin-1-yl]-1-methyl-2-oxo-1,2-dihydroquinoline-3-carbonitrile,
4-[4-(6-bromo-1,3-benzothiazol-2-yl)piperidin-1-yl]-1-methyl-2-oxo-1,2-dihydroquinoline-3-carbonitrile,
1-methyl-4-[4-(7-methyl-1,3-benzoxazol-2-yl)piperidin-1-yl]-2-oxo-1,2-dihydroquinoline-3-carbonitrile,
4-[4-(1,3-benzothiazol-2-yl)-4-fluoropiperidin-1-yl]-1-methyl-2-oxo-1,2-dihydroquinoline-3-carbonitrile,
1-methyl-4-[4-(4-methyl-1,3-benzoxazol-2-yl)piperidin-1-yl]-2-oxo-1,2-dihydroquinoline-3-carbonitrile,
4-[4-(5-chloro-1,3-benzothiazol-2-yl)piperidin-1-yl]-1-methyl-2-oxo-1,2-dihydroquinoline-3-carbonitrile,
4-[4-(5-chloro[1,3]thiazolo[5,4-b]pyridin-2-yl)piperidin-1-yl]-1-methyl-2-oxo-1,2-dihydroquinoline-3-carbonitrile,
1-methyl-4-{4-methyl-4-[6-(trifluoromethoxy)-1,3-benzoxazol-2-yl]piperidin-1-yl}-2-oxo-1,2-dihydroquinoline-3-carbonitrile,
1-methyl-4-{4-methyl-4-[5-(propan-2-yl)-1,3-benzoxazol-2-yl]piperidin-1-yl}-2-oxo-1,2-dihydroquinoline-3-carbonitrile,
4-[4-(5,6-difluoro-1,3-benzoxazol-2-yl)piperidin-1-yl]-1-methyl-2-oxo-1,2-dihydroquinoline-3-carbonitrile,
1-methyl-2-oxo-4-{4-[5-(trifluoromethyl)-1,3-benzothiazol-2-yl]piperidin-1-yl}-1,2-dihydroquinoline-3-carbonitrile,
4-[4-(5-tert-butyl-1,3-benzoxazol-2-yl)-4-methylpiperidin-1-yl]-1-methyl-2-oxo-1,2-dihydroquinoline-3-carbonitrile,
4-{4-[5-(methanesulfonyl)-1,3-benzoxazol-2-yl]-4-methylpiperidin-1-yl}-1-methyl-2-oxo-1,2-dihydroquinoline-3-carbonitrile,
4-[4-(4-fluoro-1,3-benzoxazol-2-yl)piperidin-1-yi]-1-methyl-2-oxo-1,2-dihydroquinoline-3-carbonitrile,
4-[4-(1,3-benzoxazol-2-yl)piperidin-1-yl]-7-bromo-1-methyl-2-oxo-1,2-dihydroquinoline-3-carbonitrile,
7-bromo-1-methyl-4-{4-methyl-4-[6-(trifluoromethoxy)-1,3-benzoxazol-2-yl]piperidin-1-yl}-2-oxo-1,2-dihydroquinoline-3-carbonitrile,
7-bromo-4-[4-(5-chloro-1,3-benzoxazol-2-yl)piperidin-1-yl]-1-methyl-2-oxo-1,2-dihydroquinoline-3-carbonitrile,
7-bromo-1-methyl-2-oxo-4-{4-[5-(propan-2-yl)-1,3-benzoxazol-2-yl]piperidin-1-yl}-1,2-dihydroquinoline-3-carbonitrile,
7-bromo-4-[4-(5,6-difluoro-1,3-benzoxazol-2-yl)piperidin-1-yl]-1-methyl-2-oxo-1,2-dihydroquinoline-3-carbonitrile,
7-bromo-4-[4-(6-chloro-1,3-benzoxazol-2-yl)piperidin-1-yl]-1-methyl-2-oxo-1,2-dihydroquinoline-3-carbonitrile,
4-[4-(1,3-benzothiazol-2-yl)piperidin-1-yl]-7-bromo-1-methyl-2-oxo-1,2-dihydroquinoline-3-carbonitrile,
7-bromo-1-methyl-4-{4-methyl-4-[5-(propan-2-yl)-1,3-benzoxazol-2-yl]piperidin-1-yl}-2-oxo-1,2-dihydroquinoline-3-carbonitrile,
4-[4-(1,3-benzoxazol-2-yl)piperidin-1-yl]-1-methyl-2-oxo-7-(2-oxopyrrolidin-1-yl)-1,2-dihydroquinoline-3-carbonitrile,
4-[4-(1,3-benzothiazol-2-yl)piperidin-1-yl]-1-methyl-2-oxo-7-(2-oxopyrrolidin-1-yl)-1,2-dihydroquinoline-3-carbonitrile,
1-methyl-2-oxo-4-{4-[4-(2,2,2-trifluoroethoxy)phenyl]piperidin-1-yl}-1,2-dihydroquinoline-3-carbonitrile,
1-methyl-2-oxo-4-(4-{4-[(propan-2-yl)oxy]phenyl}piperidin-1-yl)-1,2-dihydroquinoline-3-carbonitrile,
4-[4-(4-ethoxyphenyl)piperidin-1-yl]-1-methyl-2-oxo-1,2-dihydroquinoline-3-carbonitrile,
4-[4-(4-cyclopropylphenyl)piperidin-1-yl]-1-methyl-2-oxo-1,2-dihydroquinoline-3-carbonitrile,
4-[4-(4-methoxyphenyl)piperidin-1-yl]-1-methyl-2-oxo-1,2-dihydroquinoline-3-carbonitrile,
1-methyl-2-oxo-4-[4-(4-propoxyphenyl)piperidin-1-yl]-1,2-dihydroquinoline-3-carbonitrile,
1-methyl-2-oxo-4-{4-[4-(trifluoromethoxy)phenyl]piperidin-1-yl}-1,2-dihydroquinoline-3-carbonitrile,
N-{4-[1-(3-cyano-1-methyl-2-oxo-1,2-dihydroquinolin-4-yl)piperidin-4-yl]phenyl}benzenesulfonamide,
4-[4-(3-cyclopropylphenyl)piperidin-1-yl]-1-methyl-2-oxo-1,2-dihydroquinoline-3-carbonitrile,
4-{4-[4-(dimethylamino)phenyl]piperidin-1-yl}-1-methyl-2-oxo-1,2-dihydroquinoline-3-carbonitrile,
1-methyl-2-oxo-4-{4-[4-(propan-2-yl)phenyl]piperidin-1-yl}-1,2-dihydroquinoline-3-carbonitrile,
4-{4-[4-(benzyloxy)phenyl]piperidin-1-yl}-1-methyl-2-oxo-1,2-dihydroquinoline-3-carbonitrile,
N-{4-[1-(3-cyano-1-methyl-2-oxo-1,2-dihydroquinolin-4-yl)piperidin-4-yl]phenyl}benzamide,
1-methyl-4-[4-(1-methyl-1H-indol-5-yl)piperidin-1-yl]-2-oxo-1,2-dihydroquinoline-3-carbonitrile,
4-[4-(3-fluoro-5-methylphenyl)piperidin-1-yl]-1-methyl-2-oxo-1,2-dihydroquinoline-3-carbonitrile,
4-[4-(2-methoxyphenyl)piperidin-1-yl]-1-methyl-2-oxo-1,2-dihydroquinoline-3-carbonitrile,
4-[4-([1,1′-biphenyl]-4-yl)piperidin-1-yl]-1-methyl-2-oxo-1,2-dihydroquinoline-3-carbonitrile,
4-[4-(4-chlorophenyl)piperidin-1-yl]-1-methyl-2-oxo-1,2-dihydroquinoline-3-carbonitrile,
4-[4-(3-methoxyphenyl)piperidin-1-yl]-1-methyl-2-oxo-1,2-dihydroquinoline-3-carbonitrile,
1-methyl-2-oxo-4-[4-(4-phenoxyphenyl)piperidin-1-yl]-1,2-dihydroquinoline-3-carbonitrile,
1-methyl-2-oxo-4-(4-phenylpiperidin-1-yl)-1,2-dihydroquinoline-3-carbonitrile,
1-methyl-4-(4-methyl-4-phenylpiperidin-1-yl)-2-oxo-1,2-dihydroquinoline-3-carbonitrile,
1-methyl-2-oxo-4-{4-[4-(2-oxopyrrolidin-1-yl)phenyl]piperidin-1-yl}-1,2-dihydroquinoline-3-carbonitrile,
4-[4-(2-fluorophenyl)piperidin-1-yl]-1-methyl-2-oxo-1,2-dihydroquinoline-3-carbonitrile,
1-methyl-2-oxo-4-{4-[4-(trifluoromethyl)phenyl]piperidin-1-yl}-1,2-dihydroquinoline-3-carbonitrile,
4-[4-(3-fluorophenyl)piperidin-1-yl]-1-methyl-2-oxo-1,2-dihydroquinoline-3-carbonitrile,
1-methyl-4-{4-[3-(morpholin-4-yl)phenyl]piperidin-1-yl}-2-oxo-1,2-dihydroquinoline-3-carbonitrile,
4-[4-(3-cyano-2-methylphenyl)piperidin-1-yl]-1-methyl-2-oxo-1,2-dihydroquinoline-3-carbonitrile,
4-{4-[4-(methanesulfonyl)phenyl]piperidin-1-yl}-1-methyl-2-oxo-1,2-dihydroquinoline-3-carbonitrile,
1-methyl-4-{4-[4-(4-methyl-2-oxopiperazin-1-yl)phenyl]piperidin-1-yl}-2-oxo-1,2-dihydroquinoline-3-carbonitrile,
4-[4-(1,3-benzoxazol-5-yl)piperidin-1-yl]-1-methyl-2-oxo-1,2-dihydroquinoline-3-carbonitrile,
N-{3-[1-(3-cyano-1-methyl-2-oxo-1,2-dihydroquinolin-4-yl)piperidin-4-yl]phenyl}benzenesulfonamide,
4-[4-(3-{[dimethyl(oxo)-λ6-sulfanylidene]amino}phenyl)piperidin-1-yl]-1-methyl-2-oxo-1,2-dihydroquinoline-3-carbonitrile,
1-methyl-4-[4-(naphthalen-1-yl)piperidin-1-yl]-2-oxo-1,2-dihydroquinoline-3-carbonitrile,
4-[4-(1,3-benzothiazol-4-yl)piperidin-1-yl]-1-methyl-2-oxo-1,2-dihydroquinoline-3-carbonitrile,
1-methyl-4-{4-[1-methyl-3-(trifluoroacetyl)-1H-indol-5-yl]piperidin-1-yl}-2-oxo-1,2-dihydroquinoline-3-carbonitrile,
4-[4-(1-benzofuran-7-yl)piperidin-1-yl]-1-methyl-2-oxo-1,2-dihydroquinoline-3-carbonitrile,
4-[4-(isoquinolin-7-yl)piperidin-1-yl]-1-methyl-2-oxo-1,2-dihydroquinoline-3-carbonitrile,
4-[4-(1,3-benzothiazol-7-yl)piperidin-1-yl]-1-methyl-2-oxo-1,2-dihydroquinoline-3-carbonitrile,
N-{3-[1-(3-cyano-1-methyl-2-oxo-1,2-dihydroquinolin-4-yl)piperidin-4-yl]phenyl}benzamide,
4-[4-(isoquinolin-8-yl)piperidin-1-yl]-1-methyl-2-oxo-1,2-dihydroquinoline-3-carbonitrile,
4-[4-(isoquinolin-5-yl)piperidin-1-yl]-1-methyl-2-oxo-1,2-dihydroquinoline-3-carbonitrile,
1-methyl-2-oxo-4-[4-(quinoxalin-5-yl)piperidin-1-yl]-1,2-dihydroquinoline-3-carbonitrile,
4-{4-[3-(methanesulfonyl)phenyl]piperidin-1-yl}-1-methyl-2-oxo-1,2-dihydroquinoline-3-carbo nitrile,
4-[4-(4-fluorophenyl)piperidin-1-yl]-1-methyl-2-oxo-1,2-dihydroquinoline-3-carbonitrile,
1-methyl-4-[4-(2-methyl phenyl) piperidin-1-yl]-2-oxo-1,2-dihydroquinoline-3-carbonitrile,
1-methyl-4-[4-(4-methyl phenyl) piperidin-1-yl]-2-oxo-1,2-dihydroquinoline-3-carbonitrile,
4-[4-(3,5-dichlorophenyl) piperidin-1-yl]-1-methyl-2-oxo-1,2-dihydroquinoline-3-carbonitrile,
4-[4-(3-bromophenyl) piperidin-1-yl]-1-methyl-2-oxo-1,2-dihydroquinoline-3-carbonitrile,
4-[4-(4-cyanophenyl)piperidin-1-yl]-1-methyl-2-oxo-1,2-dihydroquinoline-3-carbonitrile,
4-{4-[3-(difluoromethyl)phenyl]piperidin-1-yl}-1-methyl-2-oxo-1,2-dihydroquinoline-3-carbo nitrile,
4-[4-(4-bromophenyl) piperidin-1-yl]-1-methyl-2-oxo-1,2-dihydroquinoline-3-carbonitrile,
7-bromo-4-[4-(4-methoxyphenyl)piperidin-1-yl]-1-methyl-2-oxo-1,2-dihydroquinoline-3-carbo nitrile,
7-bromo-1-methyl-2-oxo-4-(4-phenylpiperidin-1-yl)-1,2-dihydroquinoline-3-carbonitrile,
4-[4-(4-methoxyphenyl)piperidin-1-yl]-1,7-dimethyl-2-oxo-1,2-dihydroquinoline-3-carbonitrile,
4-[4-(4-methoxyphenyl)piperidin-1-yl]-1-methyl-2-oxo-7-phenyl-1,2-dihydroquinoline-3-carbo nitrile,
4-[4-(4-methoxyphenyl)piperidin-1-yl]-1-methyl-2-oxo-1,2-dihydroquinoline-3,7-dicarbonitrile,
7-cyclopropyl-4-[4-(4-methoxyphenyl)piperidin-1-yl]-1-methyl-2-oxo-1,2-dihydroquinoline-3-carbo nitrile,
7-(2,2-dimethyl-2λ⁶-diazathia-1,2-dien-1-yl)-4-[4-(4-methoxyphenyl)piperidin-1-yl]-1-methyl-2-oxo-1,2-dihydroquinoline-3-carbonitrile,
4-[4-(4-methoxyphenyl)piperidin-1-yl]-1-methyl-2-oxo-7-(2-oxopyrrolidin-1-yl)-1,2-dihydroquinoline-3-carbonitrile,
4-[4-(4-methoxyphenyl)piperidin-1-yl]-1-methyl-7-(oxetan-3-yl)-2-oxo-1,2-dihydroquinoline-3-carbonitrile,
7-(methanesulfonyl)-4-[4-(4-methoxyphenyl)piperidin-1-yl]-1-methyl-2-oxo-1,2-dihydroquinoline-3-carbonitrile,
7-{[dimethyl(oxo)-λ6-sulfanylidene]amino}-4-[4-(4-methoxyphenyl)piperidin-1-yl]-1-methyl-2-oxo-1,2-dihydroquinoline-3-carbonitrile,
7-(3,6-dihydro-2-pyran-4-yl)-4-[4-(4-methoxyphenyl)piperidin-1-yl]-1-methyl-2-oxo-1,2-dihydroquinoline-3-carbonitrile,
4-[4-(1-benzofuran-4-yl)piperidin-1-yl]-1-methyl-2-oxo-1,2-dihydroquinoline-3-carbonitrile,
1-methyl-2-oxo-4-(4-{4-[(propan-2-yl)oxy]phenyl}piperidin-1-yl)-1,2-dihydroquinoline-3-carboxamide,
1-methyl-4-{4-methyl-4-[5-(propan-2-yl)-1,3-benzoxazol-2-yl]piperidin-1-yl}-2-oxo-1,2-dihydroquinoline-3-carboxamide,
4-[4-(1,3-benzoxazol-2-yl)piperidin-1-yl]-7-bromo-1-methyl-2-oxo-1,2-dihydroquinoline-3-carboxamide,
4-[4-(1,3-benzoxazol-2-yl)-4-methylpiperidin-1-yl]-1-methyl-2-oxo-1,2-dihydroquinoline-3-carboxamide,
4-[4-(1,3-benzoxazol-2-yl)-4-ethylpiperidin-1-yl]-1-methyl-2-oxo-1,2-dihydroquinoline-3-carbonitrile,
1-methyl-4-[4-(5-methyl-1,3-benzoxazol-2-yl)piperidin-1-yl]-2-oxo-1,2-dihydroquinoline-3-carboxamide,
1-methyl-2-oxo-4-{4-[4-(trifluoromethoxy)phenyl]piperidin-1-yl}-1,2-dihydroquinoline-3-carboxamide,
4-[4-(5,6-difluoro-1,3-benzoxazol-2-yl)piperidin-1-yl]-1-methyl-2-oxo-1,2-dihydroquinoline-3-carboxamide,
7-bromo-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[3-methyl-4-(5-methyl-1,3-benzoxazol-2-yl)piperidin-1-yl]-2-oxo-1,2-dihydroquinoline-3-carbonitrile, mixture of stereoisomers,
4-[4-(4-methoxyphenyl)piperidin-1-yl]-1-methyl-2-oxo-1,2-dihydroquinoline-3-carboxamide,
4-[4-(1,3-benzothiazol-2-yl)piperidin-1-yi]-1-methyl-2-oxo-1,2-dihydroquinoline-3-carboxamide,
1-methyl-2-oxo-4-(4-phenylpiperidin-1-yl)-1,2-dihydroquinoline-3-carboxamide,
4-[4-(4-methoxyphenyl)piperidin-1-yl]-1-methyl-7-(oxetan-3-yl)-2-oxo-1,2-dihydroquinoline-3-carboxamide,
4-[4-(1,3-benzothiazol-2-yl)piperidin-1-yl]-1-methyl-2-oxo-7-(2-oxopyrrolidin-1-yl)-1,2-dihydroquinoline-3-carboxamide,
(rac)-1-methyl-2-oxo-4-{4-[4-(propan-2-yl)phenyl]azepan-1-yl}-1,2-dihydroquinoline-3-carbonitrile,
1-methyl-2-oxo-4-{(4S)-4-[4-(propan-2-yl)phenyl]azepan-1-yl}-1,2-dihydroquinoline-3-carbonitrile,
1-methyl-2-oxo-4-{(4R)-4-[4-(propan-2-yl)phenyl]azepan-1-yl}-1,2-dihydroquinoline-3-carbonitrile,
(rac)-4-[4-(1,3-benzoxazol-2-yl)azepan-1-yl]-1-methyl-2-oxo-1,2-dihydroquinoline-3-carbonitrile,
1-methyl-2-oxo-4-[4-{4-[(propan-2-yl)oxy]phenyl}azepan-1-yl]-1,2-dihydroquinoline-3-carbonitrile,
(rac)-4-[4-(4-methoxyphenyl)azepan-1-yl]-1-methyl-2-oxo-1,2-dihydroquinoline-3-carbonitrile,
4-[(4R)-4-(4-methoxyphenyl)azepan-1-yl]-1-methyl-2-oxo-1,2-dihydroquinoline-3-carbonitrile,
4-[(4S)-4-(4-methoxyphenyl)azepan-1-yl]-1-methyl-2-oxo-1,2-dihydroquinoline-3-carbonitrile,
(rac)-4-[4-(1,3-benzoxazol-2-yl)azepan-1-yl]-7-bromo-1-methyl-2-oxo-1,2-dihydroquinoline-3-carbonitrile,
4-[(4R)-4-(1,3-benzoxazol-2-yl)azepan-1-yl]-7-bromo-1-methyl-2-oxo-1,2-dihydroquinoline-3-carbonitrile,
4-[(4S)-4-(1,3-benzoxazol-2-yl)azepan-1-yl]-7-bromo-1-methyl-2-oxo-1,2-dihydroquinoline-3-carbonitrile,
(rac)-4-[4-(4-chlorophenyl)azepan-1-yl]-1-methyl-2-oxo-1,2-dihydroquinoline-3-carbonitrile,
4-[(4R)-4-(4-chlorophenyl)azepan-1-yl]-1-methyl-2-oxo-1,2-dihydroquinoline-3-carbonitrile,
4-[(4SR)-4-(4-chlorophenyl)azepan-1-yl]-1-methyl-2-oxo-1,2-dihydroquinoline-3-carbonitrile,
(rac)-1-methyl-2-oxo-4-[4-phenylazepan-1-yl]-1,2-dihydroquinoline-3-carbonitrile,
(rac)-7-bromo-1-methyl-2-oxo-4-[4-phenylazepan-1-yl]-1,2-dihydroquinoline-3-carbonitrile,
(rac)-1-methyl-7-(oxetan-3-yl)-2-oxo-4-[4-phenylazepan-1-yl]-1,2-dihydroquinoline-3-carbonitrile,
(rac)-1-methyl-7-(morpholin-4-yl)-2-oxo-4-[4-phenylazepan-1-yl]-1,2-dihydroquinoline-3-carbonitrile,
(rac)-1-methyl-2-oxo-7-(2-oxopyrrolidin-1-yl)-4-[4-phenylazepan-1-yl]-1,2-dihydroquinoline-3-carbonitrile,
(rac)-7-(1,1-dioxo-1λ⁶-thiomorpholin-4-yl)-1-methyl-2-oxo-4-[4-phenylazepan-1-yl]-1,2-dihydroquinoline-3-carbonitrile,
(rac)-7-bromo-1-methyl-2-oxo-4-[4-phenylazepan-1-yl]-1,2-dihydroquinoline-3-carboxamide,
7-bromo-1-methyl-2-oxo-4-[(4S)-4-phenylazepan-1-yl]-1,2-dihydroquinoline-3-carboxamide,
7-bromo-1-methyl-2-oxo-4-[(4S)-4-phenylazepan-1-yl]-1,2-dihydroquinoline-3-carboxamide,
4-[4-ethyl-4-(5-methyl-1,3-benzoxazol-2-yl)piperidin-1-yl]-1-methyl-2-oxo-1,2-dihydroquinoline-3-carbonitrile,
7-bromo-4-[4-ethyl-4-(5-methyl-1,3-benzoxazol-2-yl)piperidin-1-yl]-1-methyl-2-oxo-1,2-dihydroquinoline-3-carbonitrile,
4-[4-(6-methoxypyridin-3-yl)piperidin-1-yl]-1-methyl-2-oxo-1,2-dihydroquinoline-3-carbonitrile,
1-methyl-4-[4-(6-methylpyridin-3-yl)piperidin-1-yl]-2-oxo-1,2-dihydroquinoline-3-carbonitrile,
1-methyl-2-oxo-4-[4-(pyridin-3-yl)piperidin-1-yl]-1,2-dihydroquinoline-3-carbonitrile,
4-[4-(1,3-benzothiazol-2-yl)piperidin-1-yl]-N,1-dimethyl-2-oxo-1,2-dihydroquinoline-3-carboxamide,
4-[4-(1,3-benzothiazol-2-yl)piperidin-1-yl]-N,N,1-trimethyl-2-oxo-1,2-dihydroquinoline-3-carboxamide,
1-methyl-4-[4-(1-methyl-1H-benzimidazol-2-yl)piperidin-1-yl]-2-oxo-1,2-dihydroquinoline-3-carbonitrile,
1-methyl-2-oxo-4-[4-(3-propyl-1,2,4-oxadiazol-5-yl)piperidin-1-yl]-1,2-dihydroquinoline-3-carbonitrile,
1-methyl-4-[4-(1-methyl-1H-pyrazol-5-yl)piperidin-1-yl]-2-oxo-1,2-dihydroquinoline-3-carbonitrile,
1-methyl-2-oxo-4-[4-(pyrazin-2-yl)piperidin-1-yl]-1,2-dihydroquinoline-3-carbonitrile,
4-[4-(4-chlorophenyl)-4-hydroxypiperidin-1-yl]-1-methyl-2-oxo-1,2-dihydroquinoline-3-carbonitrile,
4-{4-hydroxy-4-[3-(trifluoromethyl)phenyl]piperidin-1-yl}-1-methyl-2-oxo-1,2-dihydroquinoline-3-carbonitrile,
8-fluoro-4-[4-(4-methoxyphenyl)piperidin-1-yl]-1-methyl-2-oxo-1,2-dihydroquinoline-3-carbonitrile,
4-[4-(1,3-benzothiazol-2-yl)piperidin-1-yl]-8-fluoro-1-methyl-2-oxo-1,2-dihydroquinoline-3-carbonitrile,
4-[4-(1,3-benzoxazol-2-yl)piperidin-1-yl]-8-fluoro-1-methyl-2-oxo-1,2-dihydroquinoline-3-carbonitrile,
4-[4-(1,3-benzoxazol-2-yl)-4-methylpiperidin-1-yl]-8-bromo-1-methyl-2-oxo-1,2-dihydroquinoline-3-carbonitrile,
8-bromo-4-[4-(6-chloro-1,3-benzoxazol-2-yl)piperidin-1-yl]-1-methyl-2-oxo-1,2-dihydroquinoline-3-carbonitrile,
8-bromo-1-methyl-4-[4-methyl-4-(5-methyl-1,3-benzoxazol-2-yl)piperidin-1-yl]-2-oxo-1,2-dihydroquinoline-3-carbonitrile,
8-bromo-4-[4-(5-chloro-1,3-benzoxazol-2-yl)piperidin-1-yl]-1-methyl-2-oxo-1,2-dihydroquinoline-3-carbonitrile,
8-bromo-4-[4-(5,6-difluoro-1,3-benzoxazol-2-yl)piperidin-1-yl]-1-methyl-2-oxo-1,2-dihydroquinoline-3-carbonitrile,
4-[4-(1,3-benzoxazol-2-yl)piperidin-1-yl]-8-bromo-1-methyl-2-oxo-1,2-dihydroquinoline-3-carbonitrile,
4-[4-(1,3-benzoxazol-2-yl)piperidin-1-yl]-8-chloro-1-methyl-2-oxo-1,2-dihydroquinoline-3-carbonitrile,
1-methyl-4-[4-methyl-4-(5-methyl-1,3-benzoxazol-2-yl)piperidin-1-yl]-2-oxo-1,2-dihydroquinoline-3,8-dicarbonitrile,
8-(methanesulfonyl)-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)piperidin-1-yl]-1,6-dimethyl-2-oxo-1,2-dihydroquinoline-3-carbonitrile,
1,6-dimethyl-4-[4-(5-methyl-1,3-benzoxazol-2-yl)piperidin-1-yl]-2-oxo-1,2-dihydroquinoline-3-carbonitrile,
6-bromo-4-[4-(4-methoxyphenyl)piperidin-1-yl]-1-methyl-2-oxo-1,2-dihydroquinoline-3-carbonitrile,
4-[4-(1,3-benzoxazol-2-yl)piperidin-1-yl]-6-bromo-1-methyl-2-oxo-1,2-dihydroquinoline-3-carbonitrile,
4-[4-(1,3-benzothiazol-2-yl)piperidin-1-yl]-6-bromo-1-methyl-2-oxo-1,2-dihydroquinoline-3-carbonitrile,
6-bromo-1-methyl-2-oxo-4-{4-[5-(propan-2-yl)-1,3-benzoxazol-2-yl]piperidin-1-yl}-1,2-dihydroquinoline-3-carbonitrile,
6-bromo-1-methyl-4-[4-methyl-4-(5-methyl-1,3-benzoxazol-2-yl)piperidin-1-yl]-2-oxo-1,2-dihydroquinoline-3-carbonitrile,
4-[4-(4-methoxyphenyl)piperidin-1-yl]-1,6-dimethyl-2-oxo-1,2-dihydroquinoline-3-carbonitrile,
4-[4-(4-methoxyphenyl)piperidin-1-yl]-1-methyl-2-oxo-1,2-dihydroquinoline-3,6-dicarbonitrile,
4-[4-(1,3-benzothiazol-2-yl)piperidin-1-yl]-1,6-dimethyl-2-oxo-1,2-dihydroquinoline-3-carbonitrile,
4-[4-(1,3-benzoxazol-2-yl)piperidin-1-yl]-1-methyl-2-oxo-1,2-dihydroquinoline-3,6-dicarbonitrile,
4-[4-(1,3-benzothiazol-2-yl)piperidin-1-yl]-1-methyl-2-oxo-1,2-dihydroquinoline-3,6-dicarbonitrile,
6-cyclopropyl-4-[4-(4-methoxyphenyl)piperidin-1-yl]-1-methyl-2-oxo-1,2-dihydroquinoline-3-carbonitrile,
6-(methanesulfonyl)-4-[4-(4-methoxyphenyl)piperidin-1-yl]-1-methyl-2-oxo-1,2-dihydroquinoline-3-carbonitrile,
4-[4-(4-methoxyphenyl)piperidin-1-yl]-1-methyl-6-(oxetan-3-yl)-2-oxo-1,2-dihydroquinoline-3-carbonitrile,
6-(3,6-dihydro-2H-pyran-4-yl)-4-[4-(4-methoxyphenyl)piperidin-1-yl]-1-methyl-2-oxo-1,2-dihydroquinoline-3-carbonitrile,
4-[4-(4-methoxyphenyl)piperidin-1-yl]-1-methyl-2-oxo-6-phenyl-1,2-dihydroquinoline-3-carbonitrile,
6-(methanesulfonyl)-1-methyl-4-[4-methyl-4-(5-methyl-1,3-benzoxazol-2-yl)piperidin-1-yl]-2-oxo-1,2-dihydroquinoline-3-carbonitrile,
4-[4-(6-chloro-1,3-benzoxazol-2-yl)piperidin-1-yl]-1,6-dimethyl-2-oxo-1,2-dihydroquinoline-3-carbonitrile,
1,6-dimethyl-4-[4-(6-methyl-1,3-benzoxazol-2-yl)piperidin-1-yl]-2-oxo-1,2-dihydroquinoline-3-carbonitrile,
4-[4-(5-chloro-1,3-benzoxazol-2-yl)piperidin-1-yl]-1,6-dimethyl-2-oxo-1,2-dihydroquinoline-3-carbonitrile,
1,6-dimethyl-2-oxo-4-{4-[5-(propan-2-yl)-1,3-benzoxazol-2-yl]piperidin-1-yl}-1,2-dihydroquinoline-3-carbonitrile,
1,6-dimethyl-4-[4-methyl-4-(5-methyl-1,3-benzoxazol-2-yl)piperidin-1-yl]-2-oxo-1,2-dihydroquinoline-3-carbonitrile,
1,6-dimethyl-4-[4-methyl-4-(6-methyl-1,3-benzoxazol-2-yl)piperidin-1-yl]-2-oxo-1,2-dihydroquinoline-3-carbonitrile,
1,6-dimethyl-4-{4-methyl-4-[5-(propan-2-yl)-1,3-benzoxazol-2-yl]piperidin-1-yl}-2-oxo-1,2-dihydroquinoline-3-carbonitrile,
6-methoxy-1-methyl-4-[4-(6-methyl-1,3-benzoxazol-2-yl)piperidin-1-yl]-2-oxo-1,2-dihydroquinoline-3-carbonitrile,
6-methoxy-1-methyl-4-[4-methyl-4-(6-methyl-1,3-benzoxazol-2-yl)piperidin-1-yl]-2-oxo-1,2-dihydroquinoline-3-carbonitrile,
6-chloro-1-methyl-4-[4-methyl-4-(6-methyl-1,3-benzoxazol-2-yl)piperidin-1-yl]-2-oxo-1,2-dihydroquinoline-3-carbonitrile,
4-[4-(1,3-benzoxazol-2-yl)piperidin-1-yl]-1,6-dimethyl-2-oxo-1,2-dihydroquinoline-3-carboxamide,
1,6-dimethyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)-4-methylpiperidin-1-yl]-1,6-dimethyl-2-oxo-1,2-dihydroquinoline-3-carboxamide,
1,6-dimethyl-4-[4-(6-methyl-1,3-benzoxazol-2-yl)piperidin-1-yl]-2-oxo-1,2-dihydroquinoline-3-carboxamide,
1,6-dimethyl-4-[4-methyl-4-(6-methyl-1,3-benzoxazol-2-yl)piperidin-1-yl]-2-oxo-1,2-dihydroquinoline-3-carboxamide,
1,6-dimethyl-4-[4-methyl-4-(5-methyl-1,3-benzoxazol-2-yl)piperidin-1-yl]-2-oxo-1,2-dihydroquinoline-3-carboxamide,
1,6-dimethyl-4-{4-methyl-4-[5-(propan-2-yl)-1,3-benzoxazol-2-yl]piperidin-1-yl}-2-oxo-1,2-dihydroquinoline-3-carboxamide,
1,6-dimethyl-2-oxo-4-{4-[5-(propan-2-yl)-1,3-benzoxazol-2-yl]piperidin-1-yl}-1,2-dihydroquinoline-3-carboxamide,
4-[4-(5-chloro-1,3-benzoxazol-2-yl)piperidin-1-yl]-1,6-dimethyl-2-oxo-1,2-dihydroquinoline-3-carboxamide,
4-[4-(6-chloro-1,3-benzoxazol-2-yl)piperidin-1-yl]-1,6-dimethyl-2-oxo-1,2-dihydroquinoline-3-carboxamide,
6-bromo-1-methyl-4-[4-methyl-4-(5-methyl-1,3-benzoxazol-2-yl)piperidin-1-yl]-2-oxo-1,2-dihydroquinoline-3-carboxamide,
4-[4-(1,3-benzoxazol-2-yl)azepan-1-yl]-1,6-dimethyl-2-oxo-1,2-dihydroquinoline-3-carbonitrile,
4-[4-(1,3-benzoxazol-2-yl)azepan-1-yl]-1,6-dimethyl-2-oxo-1,2-dihydroquinoline-3-carboxamide,
4-[4-(1,3-benzoxazol-2-yl)piperidin-1-yl]-6-fluoro-1-methyl-2-oxo-1,2-dihydroquinoline-3-carbonitrile,
1-methyl-4-[4-methyl-4-(5-methyl-1,3-benzoxazol-2-yl)piperidin-1-yl]-2-oxo-1,2-dihydroquinoline-3,6-dicarbonitrile,
6-cyano-1-methyl-4-[4-methyl-4-(5-methyl-1,3-benzoxazol-2-yl)piperidin-1-yl]-2-oxo-1,2-dihydroquinoline-3-carboxamide,
6-(3,3-difluorocyclobutyl)-1-methyl-4-[4-methyl-4-(5-methyl-1,3-benzoxazol-2-yl)piperidin-1-yl]-2-oxo-1,2-dihydroquinoline-3-carbonitrile,
6-cyclopropyl-1-methyl-4-[4-methyl-4-(5-methyl-1,3-benzoxazol-2-yl)piperidin-1-yl]-2-oxo-1,2-dihydroquinoline-3-carbonitrile,
6-(butan-2-yl)-1-methyl-4-[4-methyl-4-(5-methyl-1,3-benzoxazol-2-yl)piperidin-1-yl]-2-oxo-1,2-dihydroquinoline-3-carbonitrile,
7-(methoxymethyl)-1-methyl-4-[4-methyl-4-(5-methyl-1,3-benzoxazol-2-yl)piperidin-1-yl]-2-oxo-1,2-dihydroquinoline-3-carbonitrile,
6-(methoxymethyl)-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-1,6-dimethyl-2-oxo-1,2-dihydroquinoline-3-carbonitrile,
6-fluoro-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-6-fluoro-1-methyl-2-oxo-1,2-dihydroquinoline-3-carbonitrile,
6-fluoro-1-methyl-4-[4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-1,7-dimethyl-2-oxo-1,2-dihydroquinoline-3-carbonitrile,
4-[4-(1,3-benzoxazol-2-yl)piperidin-1-yl]-1,7-dimethyl-2-oxo-1,2-dihydroquinoline-3-carbonitrile,
1,7-dimethyl-4-[4-methyl-4-(5-methyl-1,3-benzoxazol-2-yl)piperidin-1-yl]-2-oxo-1,2-dihydroquinoline-3-carbonitrile,
1,7-dimethyl-4-[4-(5-methyl-1,3-benzoxazol-2-yl)piperidin-1-yl]-2-oxo-1,2-dihydroquinoline-3-carbonitrile,
4-[4-(1,3-benzothiazol-2-yl)piperidin-1-yl]-1,7-dimethyl-2-oxo-1,2-dihydroquinoline-3-carbonitrile,
4-[4-(1,3-benzoxazol-2-yl)-4-ethylpiperidin-1-yl]-7-bromo-1-methyl-2-oxo-1,2-dihydroquinoline-3-carbonitrile,
4-[4-(1,3-benzoxazol-2-yl)-4-methylpiperidin-1-yl]-7-bromo-1-methyl-2-oxo-1,2-dihydroquinoline-3-carbonitrile,
8-bromo-1,6-dimethyl-4-[4-methyl-4-(6-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-7-chloro-1-methyl-2-oxo-1,2-dihydroquinoline-3-carbonitrile,
4-[4-(1,3-benzoxazol-2-yl)piperidin-1-yl]-7-chloro-1-methyl-2-oxo-1,2-dihydroquinoline-3-carbonitrile,
7-chloro-1-methyl-4-[4-methyl-4-(5-methyl-1,3-benzoxazol-2-yl)piperidin-1-yl]-2-oxo-1,2-dihydroquinoline-3-carbonitrile,
7-chloro-1-methyl-4-[4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-8-chloro-1-methyl-2-oxo-1,2-dihydroquinoline-3-carbonitrile,
8-chloro-1-methyl-4-[4-methyl-4-(5-methyl-1,3-benzoxazol-2-yl)piperidin-1-yl]-2-oxo-1,2-dihydroquinoline-3-carbonitrile,
8-chloro-1-methyl-4-[4-(5-methyl-1,3-benzoxazol-2-yl)piperidin-1-yl]-2-oxo-1,2-dihydroquinoline-3-carbonitrile,
8-bromo-1-methyl-4-{4-methyl-4-[5-methyl-4-(trifluoromethyl)-1,3-benzoxazol-2-yl]piperidin-1-yl}-2-oxo-1,2-dihydroquinoline-3-carbonitrile,
1-methyl-4-[(2S,4S)-2-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methyl piperidin-1-yl]-7-fluoro-1-methyl-2-oxo-1,2-dihydroquinoline-3-carbonitrile,
4-[4-(1,3-benzoxazol-2-yl)piperidin-1-yl]-7-fluoro-1-methyl-2-oxo-1,2-dihydroquinoline-3-carbonitrile,
7-fluoro-1-methyl-4-[4-methyl-4-(5-methyl-1,3-benzoxazol-2-yl)piperidin-1-yl]-2-oxo-1,2-dihydroquinoline-3-carbonitrile,
7-fluoro-1-methyl-4-[4-(5-methyl-1,3-benzoxazol-2-yl)piperidin-1-yl]-2-oxo-1,2-dihydroquinoline-3-carbonitrile,
7-[(1-hydroxycyclopropyl)methoxy]-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzothiazol-2-yl)piperidin-1-yl]-7-methoxy-1-methyl-2-oxo-1,2-dihydroquinoline-3-carbonitrile,
1-methyl-4-[4-methyl-4-(5-methyl-1,3-benzoxazol-2-yl)piperidin-1-yl]-7-[(oxetan-3-yl)oxy]-2-oxo-1,2-dihydroquinoline-3-carbonitrile,
6-hydroxy-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)piperidin-1-yl]-7-methoxy-1-methyl-2-oxo-1,2-dihydroquinoline-3-carbonitrile,
7-(cyclopropyloxy)-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-8-(oxetan-3-yl)-2-oxo-1,2-dihydroquinoline-3-carbonitrile,
6-methoxy-1-methyl-4-[4-methyl-4-(5-methyl-1,3-benzoxazol-2-yl)piperidin-1-yl]-2-oxo-1,2-dihydroquinoline-3-carbonitrile,
7-(cyclobutyloxy)-1-methyl-4-[4-methyl-4-(5-methyl-1,3-benzoxazol-2-yl)piperidin-1-yl]-2-oxo-1,2-dihydroquinoline-3-carbonitrile,
7-methoxy-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-2-oxo-1,2-dihydroquinoline-3,7-dicarbonitrile,
7-cyclopropyl-1-methyl-4-[4-methyl-4-(5-methyl-1,3-benzoxazol-2-yl)piperidin-1-yl]-2-oxo-1,2-dihydroquinoline-3-carbonitrile,
7-[(1-cyanocyclopropyl)methoxy]-1-methyl-4-[4-methyl-4-(5-methyl-1,3-benzoxazol-2-yl)piperidin-1-yl]-2-oxo-1,2-dihydroquinoline-3-carbonitrile,
7-(3,3-difluorocyclobutyl)-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-8-[(oxetan-3-yl)oxy]-2-oxo-1,2-dihydroquinoline-3-carbonitrile,
4-[4-(5,6-difluoro-1,3-benzoxazol-2-yl)piperidin-1-yl]-7-methoxy-1-methyl-2-oxo-1,2-dihydroquinoline-3-carbonitrile,
1,7-dimethyl-2-oxo-4-(4-phenylpiperidin-1-yl)-1,2-dihydroquinoline-3-carbonitrile,
7-methoxy-1-methyl-2-oxo-4-(4-phenylpiperidin-1-yl)-1,2-dihydroquinoline-3-carbonitrile,
1-methyl-4-[4-methyl-4-(5-methyl-1,3-benzoxazol-2-yl)piperidin-1-yl]-2-oxo-7-propoxy-1,2-dihydroquinoline-3-carbonitrile,
4-[4-(6-chloro-1,3-benzoxazol-2-yl)piperidin-1-yl]-7-methoxy-1-methyl-2-oxo-1,2-dihydroquinoline-3-carbonitrile,
4-[4-(5-chloro-1,3-benzoxazol-2-yl)piperidin-1-yl]-7-methoxy-1-methyl-2-oxo-1,2-dihydroquinoline-3-carbonitrile,
8-methoxy-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-7-(oxetan-3-yl)-2-oxo-1,2-dihydroquinoline-3-carbonitrile,
8-(cyclopropyloxy)-1-methyl-4-[4-methyl-4-(5-methyl-1,3-benzoxazol-2-yl)piperidin-1-yl]-2-oxo-1,2-dihydroquinoline-3-carbonitrile,
1,8-dimethyl-4-[4-methyl-4-(5-methyl-1,3-benzoxazol-2-yl)piperidin-1-yl]-2-oxo-1,2-dihydroquinoline-3-carbonitrile,
7-(cyclopropyloxy)-1-methyl-2-oxo-4-(4-phenylpiperidin-1-yl)-1,2-dihydroquinoline-3-carbonitrile,
4-{4-[4-(2-methoxyethyl)-1,3-benzoxazol-2-yl]piperidin-1-yl}-1-methyl-2-oxo-1,2-dihydroquinoline-3-carbonitrile,
7-[(2S)-butan-2-yl]-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-6-[(oxetan-3-yl)oxy]-2-oxo-1,2-dihydroquinoline-3-carbonitrile,
6-[(1-cyanocyclopropyl)methoxy]-1-methyl-4-[4-methyl-4-(5-methyl-1,3-benzoxazol-2-yl)piperidin-1-yl]-2-oxo-1,2-dihydroquinoline-3-carbonitrile,
7-[(4R)-4-hydroxy-2-oxopyrrolidin-1-yl]-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzothiazol-2-yl)piperidin-1-yl]-1-methyl-2-oxo-7-propoxy-1,2-dihydroquinoline-3-carbonitrile,
8-hydroxy-1-methyl-4-[4-methyl-4-(5-methyl-1,3-benzoxazol-2-yl)piperidin-1-yl]-2-oxo-1,2-dihydroquinoline-3-carbonitrile,
8-cyclopropyl-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-2-oxo-8-propoxy-1,2-dihydroquinoline-3-carbonitrile,
6-(cyclopropyloxy)-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)piperidin-1-yl]-1-methyl-2-oxo-1,2-dihydroquinoline-3,7-dicarbonitrile,
4-[4-(1,3-benzothiazol-2-yl)piperidin-1-yl]-6-cyclopropyl-1-methyl-2-oxo-1,2-dihydroquinoline-3-carbonitrile,
6-[(4R)-4-hydroxy-2-oxopyrrolidin-1-yl]-1-methyl-4-[4-methyl-4-(5-methyl-1,3-benzoxazol-2-yl)piperidin-1-yl]-2-oxo-1,2-dihydroquinoline-3-carbonitrile,
6-(cyclobutyloxy)-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-2-oxo-6-propoxy-1,2-dihydroquinoline-3-carbonitrile,
6-[(4R)-4-hydroxy-2-oxopyrrolidin-1-yl]-1-methyl-4-[4-methyl-4-(5-methyl-1,3-benzoxazol-2-yl)piperidin-1-yl]-2-oxo-1,2-dihydroquinoline-3-carboxamide,
7-bromo-1-methyl-4-[4-(5-methyl-1,3-benzoxazol-2-yl)piperidin-1-yl]-2-oxo-1,2-dihydroquinoline-3-carbonitrile,
7-(dimethylphosphoryl)-1-methyl-4-[4-(5-methyl-1,3-benzoxazol-2-yl)piperidin-1-yl]-2-oxo-1,2-dihydroquinoline-3-carbonitrile,
4-{4-[5-(2-methoxyethyl)-1,3-benzoxazol-2-yl]piperidin-1-yl}-1-methyl-2-oxo-1,2-dihydroquinoline-3-carbonitrile,
4-{4-[6-(2-methoxyethyl)-1,3-benzoxazol-2-yl]piperidin-1-yl}-1-methyl-2-oxo-1,2-dihydroquinoline-3-carbonitrile,
1-methyl-4-[4-methyl-4-(5-methyl-1,3-benzoxazol-2-yl)piperidin-1-yl]-7-[(oxetan-3-yl)methyl]-2-oxo-1,2-dihydroquinoline-3-carbonitrile,
1-methyl-4-[4-methyl-4-(5-methyl-1,3-benzoxazol-2-yl)piperidin-1-yl]-6-[(oxetan-3-yl)methyl]-2-oxo-1,2-dihydroquinoline-3-carbonitrile,
1-methyl-4-{4-[5-(oxetan-3-yl)-1,3-benzoxazol-2-yl]piperidin-1-yl}-2-oxo-1,2-dihydroquinoline-3-carbonitrile,
1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-2-oxo-7-(trifluoromethyl)-1,2-dihydroquinoline-3-carbonitrile,
1-methyl-4-[4-methyl-4-(5-methyl-1,3-benzoxazol-2-yl)piperidin-1-yl]-2-oxo-7-(trifluoromethyl)-1,2-dihydroquinoline-3-carbonitrile,
4-[4-(1,3-benzoxazol-2-yl)piperidin-1-yl]-1-methyl-2-oxo-7-(trifluoromethyl)-1,2-dihydroquinoline-3-carbonitrile,
4-[4-(1,3-benzoxazol-2-yl)-4-methylpiperidin-1-yl]-1-methyl-2-oxo-7-(trifluoromethyl)-1,2-dihydroquinoline-3-carbonitrile,
7-hydroxy-1-methyl-4-[4-(5-methyl-1,3-benzoxazol-2-yl)piperidin-1-yl]-2-oxo-1,2-dihydroquinoline-3-carbonitrile,
4-[4-(6-bromo-1,3-benzoxazol-2-yl)piperidin-1-yl]-1-methyl-2-oxo-1,2-dihydroquinoline-3-carbonitrile,
4-{4-[5-(2-methoxyethyl)-1,3-benzoxazol-2-yl]piperidin-1-yl}-1-methyl-2-oxo-1,2-dihydroquinoline-3-carboxamide,
4-[4-(1,3-benzoxazol-2-yl)-4-methylpiperidin-1-yl]-6-fluoro-1-methyl-2-oxo-1,2-dihydroquinoline-3-carboxamide,
4-[4-(1,3-benzoxazol-2-yl)-4-methylpiperidin-1-yl]-7-chloro-1-methyl-2-oxo-1,2-dihydroquinoline-3-carboxamide,
6-fluoro-1-methyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)piperidin-1-yl]-6-fluoro-1-methyl-2-oxo-1,2-dihydroquinoline-3-carboxamide,
4-[4-(1,3-benzoxazol-2-yl)piperidin-1-yl]-7-chloro-1-methyl-2-oxo-1,2-dihydroquinoline-3-carboxamide,
7-chloro-1-methyl-4-[4-(5-methyl-1,3-benzoxazol-2-yl)piperidin-1-yl]-2-oxo-1,2-dihydroquinoline-3-carboxamide,
1-methyl-4-[4-methyl-4-(5-methyl-1,3-benzoxazol-2-yl)piperidin-1-yl]-2-oxo-1,2-dihydroquinoline-3-carboxamide,
4-[4-(1,3-benzoxazol-2-yl)-4-methylpiperidin-1-yl]-7-fluoro-1-methyl-2-oxo-1,2-dihydroquinoline-3-carboxamide,
7-fluoro-1-methyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)piperidin-1-yl]-7-fluoro-1-methyl-2-oxo-1,2-dihydroquinoline-3-carboxamide,
4-[4-(1,3-benzoxazol-2-yl)-4-methylpiperidin-1-yl]-1-methyl-2-oxo-7-(trifluoromethyl)-1,2-dihydroquinoline-3-carboxamide,
1-methyl-4-[4-(5-methyl-1,3-benzoxazol-2-yl)piperidin-1-yl]-2-oxo-7-(trifluoromethyl)-1,2-dihydroquinoline-3-carboxamide,
4-[4-(1,3-benzoxazol-2-yl)piperidin-1-yl]-1-methyl-2-oxo-7-(trifluoromethyl)-1,2-dihydroquinoline-3-carboxamide,
4-[4-(1,3-benzoxazol-2-yl)piperidin-1-yl]-8-chloro-1-methyl-2-oxo-1,2-dihydroquinoline-3-carboxamide,
4-[4-(1,3-benzoxazol-2-yl)-4-methylpiperidin-1-yl]-8-chloro-1-methyl-2-oxo-1,2-dihydroquinoline-3-carboxamide,
4-[4-(1,3-benzoxazol-2-yl)-4-methylpiperidin-1-yl]-1,7-dimethyl-2-oxo-1,2-dihydroquinoline-3-carboxamide,
1,7-dimethyl-4-[4-methyl-4-(5-methyl-1,3-benzoxazol-2-yl)piperidin-1-yl]-2-oxo-1,2-dihydroquinoline-3-carboxamide,
1,7-dimethyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)piperidin-1-yl]-1,7-dimethyl-2-oxo-1,2-dihydroquinoline-3-carboxamide,
4-{4-[4-(2-methoxyethyl)-1,3-benzoxazol-2-yl]piperidin-1-yl}-1-methyl-2-oxo-1,2-dihydroquinoline-3-carboxamide,
4-[4-(1,3-benzoxazol-2-yl)piperidin-1-yl]-1-methyl-2-oxo-1,2-dihydroquinoline-3-carboxamide,
4-{4-[6-(2-methoxyethyl)-1,3-benzoxazol-2-yl]piperidin-1-yl}-1-methyl-2-oxo-1,2-dihydroquinoline-3-carboxamide,
4-[4-(1,3-benzoxazol-2-yl)piperidin-1-yi]-1-methyl-2-oxo-7-(2-oxopyrrolidin-1-yl)-1,2-dihydroquinoline-3-carboxamide,
8-chloro-1-methyl-4-[4-(5-methyl-1,3-benzoxazol-2-yl)piperidin-1-yl]-2-oxo-1,2-dihydroquinoline-3-carboxamide,
4-[4-(4,5-dimethyl-1,3-thiazol-2-yl)piperidin-1-yl]-1-methyl-2-oxo-1,2-dihydroquinoline-3-carbonitrile,
1-methyl-2-oxo-4-[4-(quinoxalin-2-yl)piperidin-1-yl]-1,2-dihydroquinoline-3-carbonitrile,
1-methyl-2-oxo-4-[4-(1H-pyrazol-3-yl)piperidin-1-yl]-1,2-dihydroquinoline-3-carbonitrile,
1-methyl-4-[4-(1-methyl-1H-pyrazol-4-yl)piperidin-1-yl]-2-oxo-1,2-dihydroquinoline-3-carbonitrile,
4-[4-(3,4-dimethoxyphenyl)piperidin-1-yl]-1-methyl-2-oxo-1,2-dihydroquinoline-3-carbonitrile,
4-[4-(3-chlorophenyl)piperidin-1-yl]-1-methyl-2-oxo-1,2-dihydroquinoline-3-carbonitrile,
1-methyl-4-[(2S,4S)-2-methyl-4-(5-methyl-1,3-benzoxazol-2-yl)-1-piperidyl]-2-oxo-quinoline-3-carbonitrile,
4-[4-(3-cyanophenyl)piperidin-1-yl]-1-methyl-2-oxo-1,2-dihydroquinoline-3-carbonitrile,
8-fluoro-1-methyl-2-oxo-4-[(4S)-4-phenylazepan-1-yl]-1,2-dihydroquinoline-3-carbonitrile,
1-methyl-4-[rac-(2R,3S)-2-methyl-3-phenylpyrrolidin-1-yl]-2-oxo-1,2-dihydroquinoline-3-carbonitrile,
4-[1-(3-cyano-1-methyl-2-oxo-1,2-dihydroquinolin-4-yl)piperidin-4-yl]benzamide,
4-[4-hydroxy-4-(2-methoxyphenyl)piperidin-1-yl]-1-methyl-2-oxo-1,2-dihydroquinoline-3-carbonitrile,
4-[4-(1,3-benzoxazol-2-yl)piperidin-1-yl]-1-ethyl-2-oxo-1,2-dihydroquinoline-3-carbonitrile,
4-[4-(4-chlorophenyl)piperidin-1-yl]-1-ethyl-2-oxo-1,2-dihydroquinoline-3-carbonitrile,
1-methyl-2-oxo-4-{4-[5-(2-oxopyrrolidin-1-yl)-1,3-benzoxazol-2-yl]piperidin-1-yl}-1,2-dihydroquinoline-3-carbonitrile,
4-[4-(4-acetylphenyl)-4-methylpiperidin-1-yl]-1-methyl-2-oxo-1,2-dihydroquinoline-3-carbonitrile,
4-[4-(3-chlorophenyl)-4-methoxypiperidin-1-yl]-1-methyl-2-oxo-1,2-dihydroquinoline-3-carbonitrile,
6-(dimethylphosphoryl)-1-methyl-4-[4-methyl-4-(5-methyl-1,3-benzoxazol-2-yl)piperidin-1-yl]-2-oxo-1,2-dihydroquinoline-3-carbonitrile,
6-(dimethylphosphoryl)-1-methyl-4-[4-(5-methyl-1,3-benzoxazol-2-yl)piperidin-1-yl]-2-oxo-1,2-dihydroquinoline-3-carbonitrile,
6-(methanesulfonyl)-1-methyl-4-[4-(5-methyl-1,3-benzoxazol-2-yl)piperidin-1-yl]-2-oxo-1,2-dihydroquinoline-3-carbonitrile,
4-[4-(1-benzothiophen-2-yl)-4-hydroxypiperidin-1-yl]-1-methyl-2-oxo-1,2-dihydroquinoline-3-carbonitrile,
4-[4-(1-benzothiophen-2-yl)-4-methoxypiperidin-1-yl]-1-methyl-2-oxo-1,2-dihydroquinoline-3-carbonitrile,
4-[4-(1-benzothiophen-2-yl)-4-methylpiperidin-1-yl]-1-methyl-2-oxo-1,2-dihydroquinoline-3-carbonitrile,
4-[4-(6-methoxynaphthalen-2-yl)piperidin-1-yl]-1-methyl-2-oxo-1,2-dihydroquinoline-3-carbonitrile,
6-[1-(3-cyano-1-methyl-2-oxo-1,2-dihydroquinolin-4-yl)piperidin-4-yl]-2-methylquinoline-4-carbonitrile,
1-methyl-4-[4-(2-methyl-1,3-benzoxazol-5-yl)piperidin-1-yl]-2-oxo-1,2-dihydroquinoline-3-carbonitrile,
1-methyl-4-[4-(2-methyl-1,3-benzothiazol-5-yl)piperidin-1-yl]-2-oxo-1,2-dihydroquinoline-3-carbonitrile,
(rac)-1-methyl-4-[4-(3-methyl-2-oxo-2,3-dihydro-1-indol-5-yl)piperidin-1-yl]-2-oxo-1,2-dihydroquinoline-3-carbonitrile,
1-methyl-4-{4-[3-(2-methylphenyl)-1,2,4-oxadiazol-5-yl]piperidin-1-yl}-2-oxo-1,2-dihydroquinoline-3-carbonitrile,
4-{4-[3-(4-methoxyphenyl)-1,2,4-oxadiazol-5-yl]piperidin-1-yl}-1-methyl-2-oxo-1,2-dihydroquinoline-3-carbonitrile,
1-methyl-4-{4-[3-(3-methylphenyl)-1,2,4-oxadiazol-5-yl]piperidin-1-yl}-2-oxo-1,2-dihydroquinoline-3-carbonitrile,
1-methyl-4-{4-[5-(2-methylphenyl)-1,3,4-oxadiazol-2-yl]piperidin-1-yl}-2-oxo-1,2-dihydroquinoline-3-carbonitrile,
1-methyl-4-{4-[5-(4-methylphenyl)-1,3,4-oxadiazol-2-yl]piperidin-1-yl}-2-oxo-1,2-dihydroquinoline-3-carbonitrile,
1-methyl-4-{4-[5-(3-methylphenyl)-1,3,4-oxadiazol-2-yl]piperidin-1-yl}-2-oxo-1,2-dihydroquinoline-3-carbonitrile,
4-{4-[5-(3-chlorophenyl)-1,3,4-oxadiazol-2-yl]piperidin-1-yl}-1-methyl-2-oxo-1,2-dihydroquinoline-3-carbonitrile,
4-{4-[5-(3-chlorophenyl)-1,3,4-oxadiazol-2-yl]-4-methylpiperidin-1-yl}-1-methyl-2-oxo-1,2-dihydroquinoline-3-carbonitrile,
4-[4-(3,3-dimethyl-2,3-dihydro-1H-indol-5-yl)piperidin-1-yl]-1-methyl-2-oxo-1,2-dihydroquinoline-3-carbonitrile,
1-methyl-4-{4-[(2R)-2-methyl-2,3-dihydro-1-benzofuran-5-yl]piperidin-1-yl}-2-oxo-1,2-dihydroquinoline-3-carbonitrile,
1-methyl-2-oxo-4-[4-(1,3,3-trimethyl-2-oxo-2,3-dihydro-1H-indol-5-yl)piperidin-1-yl]-1,2-dihydroquinoline-3-carbonitrile,
4-[4-(2-hydroxy-2,3-dihydro-1H-inden-5-yl)piperidin-1-yl]-1-methyl-2-oxo-1,2-dihydroquinoline-3-carbonitrile,
4-[4-(2,2-dimethyl-2H-1,3-benzodioxol-5-yl)piperidin-1-yl]-1-methyl-2-oxo-1,2-dihydroquinoline-3-carbonitrile,
1-methyl-2-oxo-4-[4-(2′-oxo-1′,2′-dihydrospiro[cyclobutane-1,3′-indol]-5′-yl)piperidin-1-yl]-1,2-dihydroquinoline-3-carbonitrile,
1-methyl-4-[4-(3-methyl-2-oxo-2,3-dihydro-1,3-benzoxazol-6-yl)piperidin-1-yl]-2-oxo-1,2-dihydroquinoline-3-carbonitrile,
4-[4-(1,3-dimethyl-2-oxo-2,3-dihydro-1H-benzimidazol-5-yl)piperidin-1-yl]-1-methyl-2-oxo-1,2-dihydroquinoline-3-carbonitrile,
1-methyl-4-[4-methyl-4-(4-methylquinolin-2-yl)piperidin-1-yl]-2-oxo-1,2-dihydroquinoline-3-carbonitrile,
4-[4-(4-fluoro-1-methyl-1H-indol-6-yl)piperidin-1-yl]-1-methyl-2-oxo-1,2-dihydroquinoline-3-carbonitrile,
1-methyl-4-{4-[1-(3-methylphenyl)-1H-pyrazol-3-yl]piperidin-1-yl}-2-oxo-1,2-dihydroquinoline-3-carbonitrile,
1-methyl-4-{4-[1-(2-methylphenyl)-1H-pyrazol-3-yl]piperidin-1-yl}-2-oxo-1,2-dihydroquinoline-3-carbonitrile,
4-{4-[1-(3-chlorophenyl)-1H-pyrazol-3-yl]piperidin-1-yl}-1-methyl-2-oxo-1,2-dihydroquinoline-3-carbonitrile,
4-{4-[1-(4-chlorophenyl)-1H-pyrazol-3-yl]piperidin-1-yl}-1-methyl-2-oxo-1,2-dihydroquinoline-3-carbonitrile,
1-methyl-2-oxo-4-{4-[3-(pyridin-3-yl)-1,2,4-oxadiazol-5-yl]piperidin-1-yl}-1,2-dihydroquinoline-3-carbonitrile,
1-methyl-4-[4-(2-methylquinolin-6-yl)piperidin-1-yl]-2-oxo-1,2-dihydroquinoline-3-carbonitrile,
4-{4-[4-(3-methoxyphenyl)-1,3-thiazol-2-yl]piperidin-1-yl}-1-methyl-2-oxo-1,2-dihydroquinoline-3-carbonitrile,
4-{4-[4-(2-methoxyphenyl)-1,3-thiazol-2-yl]piperidin-1-yl}-1-methyl-2-oxo-1,2-dihydroquinoline-3-carbonitrile,
1-methyl-4-{4-[4-(4-methylphenyl)-1,3-thiazol-2-yl]piperidin-1-yl}-2-oxo-1,2-dihydroquinoline-3-carbonitrile,
4-[4-(1,3-dimethyl-1H-indazol-5-yl)piperidin-1-yl]-1-methyl-2-oxo-1,2-dihydroquinoline-3-carbonitrile,
1-methyl-4-[4-(2-methyl-1,3-benzoxazol-6-yl)piperidin-1-yl]-2-oxo-1,2-dihydroquinoline-3-carbonitrile,
1-methyl-4-[4-(2-methyl-1,3-benzothiazol-6-yl)piperidin-1-yl]-2-oxo-1,2-dihydroquinoline-3-carbonitrile,
4-{4-[3-(difluoromethyl)quinolin-7-yl]piperidin-1-yl}-1-methyl-2-oxo-1,2-dihydroquinoline-3-carbonitrile,
(rac)-1-methyl-4-[4-(5-methyl-1,3-benzoxazol-2-yl)piperidin-1-yl]-2-oxo-7-[(oxolan-3-yl)oxy]-1,2-dihydroquinoline-3-carbonitrile,
1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carbonitrile
1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carbonitrile
1-methyl-4-[4-(5-methyl-1,3-benzoxazol-2-yl)piperidin-1-yl]-7-[(oxan-4-yl)oxy]-2-oxo-1,2-dihydroquinoline-3-carbonitrile,
7-(2-methoxyethoxy)-1-methyl-4-[4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-7-{[oxiran-2-yl]methoxy}-2-oxo-1,2-dihydroquinoline-3-carbonitrile,
1-methyl-4-[4-(5-methyl-1,3-benzoxazol-2-yl)piperidin-1-yi]-7-[(oxetan-3-yl)oxy]-2-oxo-1,2-dihydroquinoline-3-carbonitrile,
4-[4-(5-methoxy-1,3-benzoxazol-2-yl)piperidin-1-yl]-1-methyl-2-oxo-1,2-dihydroquinoline-3-carbonitrile,
1-methyl-2-oxo-4-(4-{5-[(oxolan-2-yl)methoxy]-1,3-benzoxazol-2-yl}piperidin-1-yl)-1,2-dihydroquinoline-3-carbonitrile,
1-methyl-4-{4-[6-(oxetan-3-yl)-1,3-benzoxazol-2-yl]piperidin-1-yl}-2-oxo-1,2-dihydroquinoline-3-carbonitrile,
4-[4-(1,3-benzoxazol-2-yl)-4-fluoropiperidin-1-yl]-1-methyl-2-oxo-1,2-dihydroquinoline-3-carbonitrile,
4-[4-(1,3-benzoxazol-2-yl)-4-methoxypiperidin-1-yl]-1-methyl-2-oxo-1,2-dihydroquinoline-3-carbonitrile,
4-{4-[5-(methoxymethyl)-1,3-benzoxazol-2-yl]-4-methylpiperidin-1-yl}-1-methyl-2-oxo-1,2-dihydroquinoline-3-carbonitrile,
4-{4-[6-(methoxymethyl)-1,3-benzoxazol-2-yl]-4-methylpiperidin-1-yl}-1-methyl-2-oxo-1,2-dihydroquinoline-3-carbonitrile,
4-[4-fluoro-4-(5-methoxy-1,3-benzoxazol-2-yl)piperidin-1-yl]-1-methyl-2-oxo-1,2-dihydroquinoline-3-carbonitrile,
4-[4-(5-cyclopropyl-1,3-benzoxazol-2-yl)piperidin-1-yl]-1-methyl-2-oxo-1,2-dihydroquinoline-3-carbonitrile,
4-[4-(6-cyclopropyl-1,3-benzoxazol-2-yl)piperidin-1-yl]-1-methyl-2-oxo-1,2-dihydroquinoline-3-carbonitrile,
6-bromo-1-methyl-4-[4-(5-methyl-1,3-benzoxazol-2-yl)piperidin-1-yl]-2-oxo-1,2-dihydroquinoline-3-carbonitrile,
8-bromo-1-methyl-4-[4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-7-[methyl(oxetan-3-yl)amino]-2-oxo-1,2-dihydroquinoline-3-carbonitrile,
7-[(2-hydroxyethyl)(methyl)amino]-1-methyl-4-[4-(5-methyl-1,3-benzoxazol-2-yl)piperidin-1-yl]-2-oxo-1,2-dihydroquinoline-3-carbonitrile,
(rac)-1-methyl-4-[4-(5-methyl-1,3-benzoxazol-2-yl)piperidin-1-yl]-7-[methyl(oxolan-3-yl)amino]-2-oxo-1,2-dihydroquinoline-3-carbonitrile,
7-[(cyanomethyl)(methyl)amino]-1-methyl-4-[4-(5-methyl-1,3-benzoxazol-2-yl)piperidin-1-yl]-2-oxo-1,2-dihydroquinoline-3-carbonitrile,
6-methoxy-1-methyl-4-[4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-2-oxo-1,2-dihydroquinoline-3,6-dicarbonitrile,
3-cyano-1-methyl-4-[4-(5-methyl-1,3-benzoxazol-2-yl)piperidin-1-yl]-2-oxo-1,2-dihydroquinoline-6-carboxamide,
6-ethoxy-1-methyl-4-[4-(5-methyl-1,3-benzoxazol-2-yl)piperidin-1-yl]-2-oxo-1,2-dihydroquinoline-3-carbonitrile,
6-(2,2-difluoropropoxy)-1-methyl-4-[4-(5-methyl-1,3-benzoxazol-2-yl)piperidin-1-yl]-2-oxo-1,2-dihydroquinoline-3-carbonitrile,
6-(2,2-difluoroethoxy)-1-methyl-4-[4-(5-methyl-1,3-benzoxazol-2-yl)piperidin-1-yl]-2-oxo-1,2-dihydroquinoline-3-carbonitrile,
6-(cyclopropylmethoxy)-1-methyl-4-[4-(5-methyl-1,3-benzoxazol-2-yl)piperidin-1-yl]-2-oxo-1,2-dihydroquinoline-3-carbonitrile,
6-cyclobutyl-1-methyl-4-[4-(5-methyl-1,3-benzoxazol-2-yl)piperidin-1-yl]-2-oxo-1,2-dihydroquinoline-3-carbonitrile,
6-[2,2-dimethylcyclobutyl]-1-methyl-4-[4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-2-oxo-6-(3,3,3-trifluoroprop-1-en-2-yl)-1,2-dihydroquinoline-3-carbonitrile,
1-methyl-4-[4-(5-methyl-1,3-benzoxazol-2-yl)piperidin-1-yl]-2-oxo-6-[1-(trifluoromethyl)cyclopropyl]-1,2-dihydroquinoline-3-carbonitrile,
2-({3-cyano-1-methyl-4-[4-(5-methyl-1,3-benzoxazol-2-yl)piperidin-1-yl]-2-oxo-1,2-dihydroquinolin-7-yl}oxy)acetamide,
1-methyl-4-[4-(5-methyl-1,3-benzoxazol-2-yl)piperidin-1-yl]-7-{[oxan-3-yl]oxy}-2-oxo-1,2-dihydroquinoline-3-carbonitrile,
(rac)-tert-butyl ({3-cyano-1-methyl-4-[4-(5-methyl-1,3-benzoxazol-2-yl)piperidin-1-yl]-2-oxo-1,2-dihydroquinolin-7-yl}oxy)acetate,
({3-cyano-1-methyl-4-[4-(5-methyl-1,3-benzoxazol-2-yl)piperidin-1-yl]-2-oxo-1,2-dihydroquinolin-7-yl}oxy)acetic acid,
(rac)-4-[4-fluoro-4-(5-methyl-1,3-benzoxazol-2-yl)piperidin-1-yl]-1-methyl-2-oxo-7-(tetrahydrofuran-3-yloxy)-1,2-dihydroquinoline-3-carbonitrile,
7-(cyclopropylamino)-1-methyl-4-[4-methyl-4-(5-methyl-1,3-benzoxazol-2-yl)piperidin-1-yl]-2-oxo-1,2-dihydroquinoline-3-carbonitrile,
7-methoxy-1,6-dimethyl-4-[4-(5-methyl-1,3-benzoxazol-2-yl)piperidin-1-yl]-2-oxo-1,2-dihydroquinoline-3-carbonitrile,
6-bromo-4-[4-fluoro-4-(5-methyl-1,3-benzoxazol-2-yl)piperidin-1-yl]-7-methoxy-1-methyl-2-oxo-1,2-dihydroquinoline-3-carbonitrile,
1,6-dimethyl-4-[4-(5-methyl-1,3-benzoxazol-2-yl)piperidin-1-yl]-7-nitro-2-oxo-1,2-dihydroquinoline-3-carbonitrile,
6-bromo-7-hydroxy-1-methyl-4-[4-(5-methyl-1,3-benzoxazol-2-yl)piperidin-1-yl]-2-oxo-1,2-dihydroquinoline-3-carbonitrile,
6-bromo-7-hydroxy-1-methyl-4-[4-methyl-4-(5-methyl-1,3-benzoxazol-2-yl)piperidin-1-yl]-2-oxo-1,2-dihydroquinoline-3-carbonitrile,
(rac)-6-bromo-1-methyl-4-[4-(5-methyl-1,3-benzoxazol-2-yl)piperidin-1-yl]-2-oxo-7-[(oxolan-3-yl)oxy]-1,2-dihydroquinoline-3-carbonitrile,
(rac)-1-methyl-4-[4-(5-methyl-1,3-benzoxazol-2-yl)piperidin-1-yl]-2-oxo-7-[tetrahydrofuran-3-yloxy]-1,2-dihydroquinoline-3,6-dicarbonitrile,
7-hydroxy-1-methyl-4-[4-methyl-4-(5-methyl-1,3-benzoxazol-2-yl)piperidin-1-yl]-2-oxo-1,2-dihydroquinoline-3,6-dicarbonitrile,
(rac)-6-ethoxy-1-methyl-4-[4-(5-methyl-1,3-benzoxazol-2-yl)piperidin-1-yl]-2-oxo-7-[tetrahydrofuran-3-yloxy]-1,2-dihydroquinoline-3-carbonitrile,
7-hydroxy-1,6-dimethyl-4-[4-(5-methyl-1,3-benzoxazol-2-yl)piperidin-1-yl]-2-oxo-1,2-dihydroquinoline-3-carbonitrile,
(rac)-1,6-dimethyl-4-[4-(5-methyl-1,3-benzoxazol-2-yl)piperidin-1-yl]-2-oxo-7-[tetrahydrofuran-3-yloxy]-1,2-dihydroquinoline-3-carbonitrile,
(rac)-6-(2,2-difluoroethoxy)-1-methyl-4-[4-(5-methyl-1,3-benzoxazol-2-yl)piperidin-1-yl]-2-oxo-7-[tetrahydrofuran-3-yloxy]-1,2-dihydroquinoline-3-carbonitrile,
(rac)-4-[4-fluoro-4-(5-methyl-1,3-benzoxazol-2-yl)piperidin-1-yl]-6-methoxy-1-methyl-2-oxo-7-[tetrahydrofuran-3-yloxy]-1,2-dihydroquinoline-3-carbonitrile,
(rac)-1-methyl-4-[4-methyl-4-(5-methyl-1,3-benzoxazol-2-yl)piperidin-1-yl]-2-oxo-7-[(oxolan-3-yl)oxy]-1,2-dihydroquinoline-3,6-dicarbonitrile,
(rac)-1-methyl-4-[4-(5-methyl-1,3-benzoxazol-2-yl)piperidin-1-yl]-2-oxo-7-[(oxolan-3-yl)oxy]-6-(3,3,3-trifluoroprop-1-en-2-yl)-1,2-dihydroquinoline-3-carbonitrile,
(rac)-1-methyl-4-[4-(5-methyl-1,3-benzoxazol-2-yl)piperidin-1-yl]-2-oxo-7-[(oxolan-3-yl)oxy]-6-[1-(trifluoromethyl)cyclopropyl]-1,2-dihydroquinoline-3-carbonitrile,
6-bromo-1-methyl-2-oxo-4-(4-{5-[3-(trifluoromethyl)phenyl]-1,3,4-oxadiazol-2-yl}piperidin-1-yl)-1,2-dihydroquinoline-3-carbonitrile,
6-methoxy-1-methyl-4-{4-[3-(3-methylphenyl)-1,2,4-oxadiazol-5-yl]piperidin-1-yl}-2-oxo-1,2-dihydroquinoline-3-carbonitrile,
6-methoxy-1-methyl-2-oxo-4-[4-(3-phenyl-1,2,4-oxadiazol-5-yl)piperidin-1-yl]-1,2-dihydroquinoline-3-carbonitrile,
6-methoxy-1-methyl-4-{4-[3-(2-methylphenyl)-1,2,4-oxadiazol-5-yl]piperidin-1-yl}-2-oxo-1,2-dihydroquinoline-3-carbonitrile,
6-methoxy-1-methyl-2-oxo-4-[4-(5-phenyl-1,3,4-oxadiazol-2-yl)piperidin-1-yl]-1,2-dihydroquinoline-3-carbonitrile,
6-bromo-1-methyl-2-oxo-4-[4-(3-phenyl-1,2,4-oxadiazol-5-yl)piperidin-1-yl]-1,2-dihydroquinoline-3-carbonitrile,
6-bromo-1-methyl-2-oxo-4-[4-(5-phenyl-1,3,4-oxadiazol-2-yl)piperidin-1-yl]-1,2-dihydroquinoline-3-carbonitrile,
6-bromo-1-methyl-4-{4-[5-(2-methylphenyl)-1,3,4-oxadiazol-2-yl]piperidin-1-yl}-2-oxo-1,2-dihydroquinoline-3-carbonitrile,
6-bromo-1-methyl-4-{4-[5-(3-methylphenyl)-1,3,4-oxadiazol-2-yl]piperidin-1-yl}-2-oxo-1,2-dihydroquinoline-3-carbonitrile,
7-hydroxy-1-methyl-4-{4-[5-(2-methylphenyl)-1,3,4-oxadiazol-2-yl]piperidin-1-yl}-2-oxo-1,2-dihydroquinoline-3-carbonitrile,
6-bromo-7-hydroxy-1-methyl-4-{4-[5-(2-methylphenyl)-1,3,4-oxadiazol-2-yl]piperidin-1-yl}-2-oxo-1,2-dihydroquinoline-3-carbonitrile,
(rac)-1-methyl-4-{4-[5-(2-methylphenyl)-1,3,4-oxadiazol-2-yl]piperidin-1-yl}-7-{[oxan-3-yl]oxy}-2-oxo-1,2-dihydroquinoline-3-carbonitrile,
4-[4-(1,3-benzoxazol-2-yl)piperidin-1-yl]-N,1-dimethyl-2-oxo-1,2-dihydroquinoline-3-carboxamide,
N,1-dimethyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)-4-methylpiperidin-1-yl]-N,1-dimethyl-2-oxo-1,2-dihydroquinoline-3-carboxamide,
1-methyl-2-oxo-4-{4-[5-(2-oxopyrrolidin-1-yl)-1,3-benzoxazol-2-yl]piperidin-1-yl}-1,2-dihydroquinoline-3-carboxamide,
4-[4-(3-chlorophenyl)-4-methoxypiperidin-1-yl]-1-methyl-2-oxo-1,2-dihydroquinoline-3-carboxamide,
6-(dimethylphosphoryl)-1-methyl-4-[4-methyl-4-(5-methyl-1,3-benzoxazol-2-yl)piperidin-1-yl]-2-oxo-1,2-dihydroquinoline-3-carboxamide,
6-(dimethylphosphoryl)-1-methyl-4-[4-(5-methyl-1,3-benzoxazol-2-yl)piperidin-1-yl]-2-oxo-1,2-dihydroquinoline-3-carboxamide,
6-(methanesulfonyl)-1-methyl-4-[4-(5-methyl-1,3-benzoxazol-2-yl)piperidin-1-yl]-2-oxo-1,2-dihydroquinoline-3-carboxamide,
4-[4-(1-benzothiophen-2-yl)-4-methoxypiperidin-1-yl]-1-methyl-2-oxo-1,2-dihydroquinoline-3-carboxamide,
4-[4-(1-benzothiophen-2-yl)-4-hydroxypiperidin-1-yl]-1-methyl-2-oxo-1,2-dihydroquinoline-3-carboxamide,
4-[4-(6-methoxynaphthalen-2-yl)piperidin-1-yl]-1-methyl-2-oxo-1,2-dihydroquinoline-3-carboxamide,
1-methyl-4-{4-[3-(2-methylphenyl)-1,2,4-oxadiazol-5-yl]piperidin-1-yl}-2-oxo-1,2-dihydroquinoline-3-carboxamide,
4-{4-[3-(4-methoxyphenyl)-1,2,4-oxadiazol-5-yl]piperidin-1-yl}-1-methyl-2-oxo-1,2-dihydroquinoline-3-carboxamide,
1-methyl-4-{4-[3-(3-methylphenyl)-1,2,4-oxadiazol-5-yl]piperidin-1-yl}-2-oxo-1,2-dihydroquinoline-3-carboxamide,
1-methyl-4-{4-[5-(2-methylphenyl)-1,3,4-oxadiazol-2-yl]piperidin-1-yl}-2-oxo-1,2-dihydroquinoline-3-carboxamide,
1-methyl-4-{4-[5-(4-methylphenyl)-1,3,4-oxadiazol-2-yl]piperidin-1-yl}-2-oxo-1,2-dihydroquinoline-3-carboxamide,
1-methyl-4-{4-[5-(3-methylphenyl)-1,3,4-oxadiazol-2-yl]piperidin-1-yl}-2-oxo-1,2-dihydroquinoline-3-carboxamide,
4-{4-[5-(3-chlorophenyl)-1,3,4-oxadiazol-2-yl]piperidin-1-yl}-1-methyl-2-oxo-1,2-dihydroquinoline-3-carboxamide,
4-{4-[5-(3-chlorophenyl)-1,3,4-oxadiazol-2-yl]-4-methylpiperidin-1-yl}-1-methyl-2-oxo-1,2-dihydroquinoline-3-carboxamide,
1-methyl-4-{4-[2-methyl-2,3-dihydro-1-benzofuran-5-yl]piperidin-1-yl}-2-oxo-1,2-dihydroquinoline-3-carboxamide,
4-[4-(2-hydroxy-2,3-dihydro-1H-inden-5-yl)piperidin-1-yl]-1-methyl-2-oxo-1,2-dihydroquinoline-3-carboxamide,
4-[4-(2,2-dimethyl-2H-1,3-benzodioxol-5-yl)piperidin-1-yl]-1-methyl-2-oxo-1,2-dihydroquinoline-3-carboxamide,
1-methyl-2-oxo-4-[4-(2′-oxo-1′,2′-dihydrospiro[cyclobutane-1,3′-indol]-5′-yl)piperidin-1-yl]-1,2-dihydroquinoline-3-carboxamide,
1-methyl-4-[4-methyl-4-(4-methylquinolin-2-yl)piperidin-1-yl]-2-oxo-1,2-dihydroquinoline-3-carboxamide,
4-[4-(4-fluoro-1-methyl-1H-indol-6-yl)piperidin-1-yl]-1-methyl-2-oxo-1,2-dihydroquinoline-3-carboxamide,
1-methyl-4-{4-[1-(3-methylphenyl)-1H-pyrazol-3-yl]piperidin-1-yl}-2-oxo-1,2-dihydroquinoline-3-carboxamide,
1-methyl-4-{4-[1-(2-methylphenyl)-1H-pyrazol-3-yl]piperidin-1-yl}-2-oxo-1,2-dihydroquinoline-3-carboxamide,
4-{4-[1-(3-chlorophenyl)-1H-pyrazol-3-yl]piperidin-1-yl}-1-methyl-2-oxo-1,2-dihydroquinoline-3-carboxamide,
1-methyl-2-oxo-4-{4-[3-(pyridin-3-yl)-1,2,4-oxadiazol-5-yl]piperidin-1-yl}-1,2-dihydroquinoline-3-carboxamide,
4-{4-[4-(3-methoxyphenyl)-1,3-thiazol-2-yl]piperidin-1-yl}-1-methyl-2-oxo-1,2-dihydroquinoline-3-carboxamide,
4-{4-[4-(2-methoxyphenyl)-1,3-thiazol-2-yl]piperidin-1-yl}-1-methyl-2-oxo-1,2-dihydroquinoline-3-carboxamide,
1-methyl-4-{4-[4-(4-methylphenyl)-1,3-thiazol-2-yl]piperidin-1-yl}-2-oxo-1,2-dihydroquinoline-3-carboxamide,
1-methyl-4-[4-(5-methyl-1,3-benzoxazol-2-yl)piperidin-1-yl]-7-[(oxan-4-yl)oxy]-2-oxo-1,2-dihydroquinoline-3-carboxamide,
7-(2-methoxyethoxy)-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yl]-7-{[oxiran-2-yl]methoxy}-2-oxo-1,2-dihydroquinoline-3-carboxamide,
(rac)-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
(−)-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carboxamide,
(+)-1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carboxamide,
1-methyl-4-[4-(5-methyl-1,3-benzoxazol-2-yl)piperidin-1-yl]-7-[(oxetan-3-yl)oxy]-2-oxo-1,2-dihydroquinoline-3-carboxamide,
4-[4-(5-methoxy-1,3-benzoxazol-2-yl)piperidin-1-yl]-1-methyl-2-oxo-1,2-dihydroquinoline-3-carboxamide,
1-methyl-2-oxo-4-(4-{5-[(oxolan-2-yl)methoxy]-1,3-benzoxazol-2-yl}piperidin-1-yl)-1,2-dihydroquinoline-3-carboxamide,
4-[4-(1,3-benzoxazol-2-yl)-4-fluoropiperidin-1-yl]-1-methyl-2-oxo-1,2-dihydroquinoline-3-carboxamide,
4-[4-(1,3-benzoxazol-2-yl)-4-methoxypiperidin-1-yl]-1-methyl-2-oxo-1,2-dihydroquinoline-3-carboxamide,
4-{4-[5-(methoxymethyl)-1,3-benzoxazol-2-yl]-4-methylpiperidin-1-yl}-1-methyl-2-oxo-1,2-dihydroquinoline-3-carboxamide,
4-{4-[6-(methoxymethyl)-1,3-benzoxazol-2-yl]-4-methylpiperidin-1-yl}-1-methyl-2-oxo-1,2-dihydroquinoline-3-carboxamide,
6-bromo-1-methyl-4-[4-(5-methyl-1,3-benzoxazol-2-yl)piperidin-1-yl]-2-oxo-1,2-dihydroquinoline-3-carboxamide,
8-bromo-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yi]-7-[methyl(oxetan-3-yl)amino]-2-oxo-1,2-dihydroquinoline-3-carboxamide,
7-[(2-hydroxyethyl)(methyl)amino]-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yl]-7-{methyl[(3R)-oxolan-3-yl]amino}-2-oxo-1,2-dihydroquinoline-3-carboxamide,
1-methyl-4-[4-(5-methyl-1,3-benzoxazol-2-yl)piperidin-1-yl]-7-{methyl[(3S)-oxolan-3-yl]amino}-2-oxo-1,2-dihydroquinoline-3-carboxamide,
6-cyano-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yl]-2-oxo-1,2-dihydroquinoline-3,6-dicarboxamide,
7-[(cyanomethyl)(methyl)amino]-1-methyl-4-[4-(5-methyl-1,3-benzoxazol-2-yl)piperidin-1-yl]-2-oxo-1,2-dihydroquinoline-3-carboxamide,
6-methoxy-1-methyl-4-[4-(5-methyl-1,3-benzoxazol-2-yl)piperidin-1-yl]-2-oxo-1,2-dihydroquinoline-3-carboxamide,
6-ethoxy-1-methyl-4-[4-(5-methyl-1,3-benzoxazol-2-yl)piperidin-1-yl]-2-oxo-1,2-dihydroquinoline-3-carboxamide,
6-(2,2-difluoropropoxy)-1-methyl-4-[4-(5-methyl-1,3-benzoxazol-2-yl)piperidin-1-yl]-2-oxo-1,2-dihydroquinoline-3-carboxamide,
6-(2,2-difluoroethoxy)-1-methyl-4-[4-(5-methyl-1,3-benzoxazol-2-yl)piperidin-1-yl]-2-oxo-1,2-dihydroquinoline-3-carboxamide,
6-(cyclopropylmethoxy)-1-methyl-4-[4-(5-methyl-1,3-benzoxazol-2-yl)piperidin-1-yl]-2-oxo-1,2-dihydroquinoline-3-carboxamide,
6-cyclobutyl-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yl]-2-oxo-6-[1-(trifluoromethyl)cyclopropyl]-1,2-dihydroquinoline-3-carboxamide,
7-(2-amino-2-oxoethoxy)-1-methyl-4-[4-(5-methyl-1,3-benzoxazol-2-yl)piperidin-1-yl]-2-oxo-1,2-dihydroquinoline-3-carboxamide,
4-[4-fluoro-4-(5-methyl-1,3-benzoxazol-2-yl)piperidin-1-yl]-1-methyl-2-oxo-7-[(oxolan-3-yl)oxy]-1,2-dihydroquinoline-3-carboxamide,
1,6-dimethyl-4-[4-(5-methyl-1,3-benzoxazol-2-yl)piperidin-1-yl]-7-nitro-2-oxo-1,2-dihydroquinoline-3-carboxamide,
6-bromo-7-hydroxy-1-methyl-4-[4-(5-methyl-1,3-benzoxazol-2-yl)piperidin-1-yl]-2-oxo-1,2-dihydroquinoline-3-carboxamide,
6-bromo-7-hydroxy-1-methyl-4-[4-methyl-4-(5-methyl-1,3-benzoxazol-2-yl)piperidin-1-yl]-2-oxo-1,2-dihydroquinoline-3-carboxamide,
(rac)-6-bromo-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
(rac)-6-cyano-1-methyl-4-[4-methyl-4-(5-methyl-1,3-benzoxazol-2-yl)piperidin-1-yl]-2-oxo-7-[(oxolan-3-yl)oxy]-1,2-dihydroquinoline-3-carboxamide,
6-cyano-1-methyl-4-[4-methyl-4-(5-methyl-1,3-benzoxazol-2-yl)piperidin-1-yl]-2-oxo-7-{[(3R)-oxolan-3-yl]oxy}-1,2-dihydroquinoline-3-carboxamide,
6-cyano-1-methyl-4-[4-methyl-4-(5-methyl-1,3-benzoxazol-2-yl)piperidin-1-yl]-2-oxo-7-{[(3S)-oxolan-3-yl]oxy}-1,2-dihydroquinoline-3-carboxamide,
(rac)-6-(2,2-difluoroethoxy)-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
(rac)-1-methyl-4-[4-(5-methyl-1,3-benzoxazol-2-yl)piperidin-1-yl]-2-oxo-7-[(oxolan-3-yl)oxy]-6-[1-(trifluoromethyl)cyclopropyl]-1,2-dihydroquinoline-3-carboxamide,
6-bromo-1-methyl-2-oxo-4-(4-{5-[3-(trifluoromethyl)phenyl]-1,3,4-oxadiazol-2-yl}piperidin-1-yl)-1,2-dihydroquinoline-3-carboxamide,
6-methoxy-1-methyl-4-{4-[3-(3-methylphenyl)-1,2,4-oxadiazol-5-yl]piperidin-1-yl}-2-oxo-1,2-dihydroquinoline-3-carboxamide,
6-methoxy-1-methyl-2-oxo-4-[4-(3-phenyl-1,2,4-oxadiazol-5-yl)piperidin-1-yl]-1,2-dihydroquinoline-3-carboxamide,
6-methoxy-1-methyl-4-{4-[3-(2-methylphenyl)-1,2,4-oxadiazol-5-yl]piperidin-1-yl}-2-oxo-1,2-dihydroquinoline-3-carboxamide,
6-bromo-1-methyl-2-oxo-4-[4-(3-phenyl-1,2,4-oxadiazol-5-yl)piperidin-1-yl]-1,2-dihydroquinoline-3-carboxamide,
6-bromo-1-methyl-2-oxo-4-[4-(5-phenyl-1,3,4-oxadiazol-2-yl)piperidin-1-yl]-1,2-dihydroquinoline-3-carboxamide,
6-bromo-1-methyl-4-{4-[5-(2-methylphenyl)-1,3,4-oxadiazol-2-yl]piperidin-1-yl}-2-oxo-1,2-dihydroquinoline-3-carboxamide,
6-bromo-1-methyl-4-{4-[5-(3-methylphenyl)-1,3,4-oxadiazol-2-yl]piperidin-1-yl}-2-oxo-1,2-dihydroquinoline-3-carboxamide,
1-methyl-4-{4-[5-(2-methylphenyl)-1,3,4-oxadiazol-2-yl]piperidin-1-yl}-7-{[(3R)-oxan-3-yl]oxy}-2-oxo-1,2-dihydroquinoline-3-carboxamide,
1-methyl-4-{4-[5-(2-methylphenyl)-1,3,4-oxadiazol-2-yl]piperidin-1-yl}-7-{[(3S)-oxan-3-yl]oxy}-2-oxo-1,2-dihydroquinoline-3-carboxamide,
6-bromo-7-hydroxy-1-methyl-4-{4-[5-(2-methylphenyl)-1,3,4-oxadiazol-2-yl]piperidin-1-yl}-2-oxo-1,2-dihydroquinoline-3-carboxamide,
6-bromo-7-methoxy-1-methyl-4-{4-[3-(2-methylphenyl)-1,2,4-oxadiazol-5-yl]piperidin-1-yl}-2-oxo-1,2-dihydroquinoline-3-carbonitrile,
6-bromo-7-hydroxy-1-methyl-4-{4-[3-(2-methylphenyl)-1,2,4-oxadiazol-5-yl]piperidin-1-yl}-2-oxo-1,2-dihydroquinoline-3-carbonitrile,
(rac)-6-bromo-1-methyl-4-{4-[3-(2-methylphenyl)-1,2,4-oxadiazol-5-yl]piperidin-1-yl}-2-oxo-7-[(oxolan-3-yl)oxy]-1,2-dihydroquinoline-3-carbonitrile,
(rac)-6-bromo-1-methyl-4-{4-[3-(2-methylphenyl)-1,2,4-oxadiazol-5-yl]piperidin-1-yl}-2-oxo-7-[(oxolan-3-yl)oxy]-1,2-dihydroquinoline-3-carboxamide,
(rac)-6-cyano-1-methyl-4-{4-[3-(2-methylphenyl)-1,2,4-oxadiazol-5-yl]piperidin-1-yl}-2-oxo-7-[(oxolan-3-yl)oxy]-1,2-dihydroquinoline-3-carboxamide,
6-cyano-1-methyl-4-{4-[3-(2-methylphenyl)-1,2,4-oxadiazol-5-yl]piperidin-1-yl}-2-oxo-7-{[(3R)-oxolan-3-yl]oxy}-1,2-dihydroquinoline-3-carboxamide,
6-cyano-1-methyl-4-{4-[3-(2-methylphenyl)-1,2,4-oxadiazol-5-yl]piperidin-1-yl}-2-oxo-7-{[(3S)-oxolan-3-yl]oxy}-1,2-dihydroquinoline-3-carboxamide,
(−)-6-bromo-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carbonitrile,
(rac)-6-cyano-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
6-cyano-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carboxamide,
6-cyano-1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carboxamide,
6-methoxy-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carbonitrile,
6-methoxy-1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carbonitrile,
6-methoxy-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
(rac)-1,6-dimethyl-4-[4-(5-methyl-1,3-benzoxazol-2-yl)piperidin-1-yl]-2-oxo-7-[(oxolan-3-yl)oxy]-1,2-dihydroquinoline-3-carboxamide,
1,6-dimethyl-4-[4-(5-methyl-1,3-benzoxazol-2-yl)piperidin-1-yl]-2-oxo-7-{[(3R)-oxolan-3-yl]oxy}-1,2-dihydroquinoline-3-carboxamide,
1,6-dimethyl-4-[4-(5-methyl-1,3-benzoxazol-2-yl)piperidin-1-yl]-2-oxo-7-{[(3S)-oxolan-3-yl]oxy}-1,2-dihydroquinoline-3-carboxamide,
7-[(2-methoxyethyl)amino]-1,6-dimethyl-4-[4-(5-methyl-1,3-benzoxazol-2-yl)piperidin-1-yl]-2-oxo-1,2-dihydroquinoline-3-carbonitrile,
7-(3-hydroxyazetidin-1-yl)-1,6-dimethyl-4-[4-(5-methyl-1,3-benzoxazol-2-yl)piperidin-1-yl]-2-oxo-1,2-dihydroquinoline-3-carbonitrile,
1,6-dimethyl-4-[4-(5-methyl-1,3-benzoxazol-2-yl)piperidin-1-yl]-7-[(oxetan-3-yl)amino]-2-oxo-1,2-dihydroquinoline-3-carbonitrile,
7-[(2-hydroxyethyl)amino]-1,6-dimethyl-4-[4-(5-methyl-1,3-benzoxazol-2-yl)piperidin-1-yl]-2-oxo-1,2-dihydroquinoline-3-carbonitrile,
7-[(2-hydroxyethyl)(methyl)amino]-1,6-dimethyl-4-[4-(5-methyl-1,3-benzoxazol-2-yl)piperidin-1-yl]-2-oxo-1,2-dihydroquinoline-3-carbonitrile,
7-[(2-methoxyethyl)amino]-1,6-dimethyl-4-[4-(5-methyl-1,3-benzoxazol-2-yl)piperidin-1-yl]-2-oxo-1,2-dihydroquinoline-3-carboxamide,
7-(3-hydroxyazetidin-1-yl)-1,6-dimethyl-4-[4-(5-methyl-1,3-benzoxazol-2-yl)piperidin-1-yl]-2-oxo-1,2-dihydroquinoline-3-carboxamide,
(rac)-1,6-dimethyl-4-{4-[3-(2-methylphenyl)-1,2,4-oxadiazol-5-yl]piperidin-1-yl}-2-oxo-7-[(oxolan-3-yl)oxy]-1,2-dihydroquinoline-3-carbonitrile,
1,6-dimethyl-4-{4-[3-(2-methylphenyl)-1,2,4-oxadiazol-5-yl]piperidin-1-yl}-2-oxo-7-{[(3R)-oxolan-3-yl]oxy}-1,2-dihydroquinoline-3-carbonitrile,
1,6-dimethyl-4-{4-[3-(2-methylphenyl)-1,2,4-oxadiazol-5-yl]piperidin-1-yl}-2-oxo-7-{[(3S)-oxolan-3-yl]oxy}-1,2-dihydroquinoline-3-carbonitrile,
1,6-dimethyl-4-{4-[3-(2-methylphenyl)-1,2,4-oxadiazol-5-yl]piperidin-1-yl}-2-oxo-7-{[(3R)-oxolan-3-yl]oxy}-1,2-dihydroquinoline-3-carboxamide,
1,6-dimethyl-4-{4-[3-(2-methylphenyl)-1,2,4-oxadiazol-5-yl]piperidin-1-yl}-2-oxo-7-{[(3S)-oxolan-3-yl]oxy}-1,2-dihydroquinoline-3-carboxamide,
7-[(2-hydroxyethyl)amino]-1,6-dimethyl-4-{4-[3-(2-methylphenyl)-1,2,4-oxadiazol-5-yl]piperidin-1-yl}-2-oxo-1,2-dihydroquinoline-3-carbonitrile,
7-bromo-1,6-dimethyl-4-{4-[1-(2-methylphenyl)-1H-pyrazol-3-yl]piperidin-1-yl}-2-oxo-1,2-dihydroquinoline-3-carbonitrile,
7-[(2-hydroxyethyl)amino]-1,6-dimethyl-4-{4-[1-(2-methylphenyl)-1H-pyrazol-3-yl]piperidin-1-yl}-2-oxo-1,2-dihydroquinoline-3-carbonitrile,
6-bromo-7-fluoro-1-methyl-4-[4-(5-methyl-1,3-benzoxazol-2-yl)piperidin-1-yl]-2-oxo-1,2-dihydroquinoline-3-carboxamide,
6-cyano-7-fluoro-1-methyl-4-[4-(5-methyl-1,3-benzoxazol-2-yl)piperidin-1-yl]-2-oxo-1,2-dihydroquinoline-3-carboxamide,
(rac)-6-cyano-1-methyl-4-[4-(5-methyl-1,3-benzoxazol-2-yl)piperidin-1-yl]-2-oxo-7-{[(oxolan-3-yl)methyl]amino}-1,2-dihydroquinoline-3-carboxamide,
6-cyano-1-methyl-4-[4-(5-methyl-1,3-benzoxazol-2-yl)piperidin-1-yl]-2-oxo-7-({[(3R)-oxolan-3-yl]methyl}amino)-1,2-dihydroquinoline-3-carboxamide,
6-cyano-1-methyl-4-[4-(5-methyl-1,3-benzoxazol-2-yl)piperidin-1-yl]-2-oxo-7-({[(3S)-oxolan-3-yl]methyl}amino)-1,2-dihydroquinoline-3-carboxamide,
6-chloro-7-methoxy-1-methyl-4-[4-(5-methyl-1,3-benzoxazol-2-yl)piperidin-1-yl]-2-oxo-1,2-dihydroquinoline-3-carbonitrile,
6-chloro-7-hydroxy-1-methyl-4-[4-(5-methyl-1,3-benzoxazol-2-yl)piperidin-1-yl]-2-oxo-1,2-dihydroquinoline-3-carbonitrile,
(rac)-6-chloro-1-methyl-4-[4-(5-methyl-1,3-benzoxazol-2-yl)piperidin-1-yl]-2-oxo-7-[(oxolan-3-yl)oxy]-1,2-dihydroquinoline-3-carbonitrile,
(rac)-6-chloro-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
6-chloro-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carboxamide,
6-chloro-1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carboxamide,
(rac)-6-chloro-1-methyl-4-[4-(5-methyl-1,3-benzoxazol-2-yl)piperidin-1-yl]-2-oxo-7-[(oxolan-3-yl)methoxy]-1,2-dihydroquinoline-3-carbonitrile,
(rac)-6-chloro-1-methyl-4-[4-(5-methyl-1,3-benzoxazol-2-yl)piperidin-1-yl]-2-oxo-7-[(oxolan-3-yl)methoxy]-1,2-dihydroquinoline-3-carboxamide,
6-chloro-1-methyl-4-[4-(5-methyl-1,3-benzoxazol-2-yl)piperidin-1-yl]-2-oxo-7-{[(3R)-oxolan-3-yl]methoxy}-1,2-dihydroquinoline-3-carboxamide,
6-chloro-1-methyl-4-[4-(5-methyl-1,3-benzoxazol-2-yl)piperidin-1-yl]-2-oxo-7-{[(3S)-oxolan-3-yl]methoxy}-1,2-dihydroquinoline-3-carboxamide,
(rac)-6-bromo-1-methyl-2-oxo-7-[(oxolan-3-yl)oxy]-4-{4-[2-(pyridin-3-yl)-2H-1,2,3-triazol-4-yl]piperidin-1-yl}-1,2-dihydroquinoline-3-carbonitrile,
(rac)-6-bromo-1-methyl-4-{4-[1-(2-methylphenyl)-1H-1,2,3-triazol-4-yl]piperidin-1-yl}-2-oxo-7-[(oxolan-3-yl)oxy]-1,2-dihydroquinoline-3-carbonitrile,
(rac)-1,6-dimethyl-4-{4-[1-(2-methylphenyl)-1H-1,2,3-triazol-4-yl]piperidin-1-yl}-2-oxo-7-[(oxolan-3-yl)oxy]-1,2-dihydroquinoline-3-carbonitrile,
7-bromo-1,6-dimethyl-4-{4-[1-(2-methylphenyl)-1-pyrazol-4-yl]piperidin-1-yl}-2-oxo-1,2-dihydroquinoline-3-carbonitrile, and
(rac)-1,6-dimethyl-4-{4-[1-(2-methylphenyl)-1H-pyrazol-4-yl]piperidin-1-yl}-2-oxo-7-[(oxolan-3-yl)oxy]-1,2-dihydroquinoline-3-carbonitrile,
   or a stereoisomer, a tautomer, an N-oxide, a hydrate, a solvate, or a salt thereof, or a mixture of same.

In some preferred embodiments, the inhibitor of DGKalpha is a compound according to this list.

### DGKalpha Inhibitor A

In a preferred embodiment of the present invention, the inhibitor of DGKalpha is 6-fluoro-1-methyl-4-[4-(5-methyl-1,3-benzoxazol-2-yl)piperidin-1-yl]-2-oxo-1,2-dihydroquinoline-3-carboxamide, or a tautomer, an N-oxide, a hydrate, a solvate, or a salt thereof, or a mixture of same.

In another preferred embodiment of the present invention, the inhibitor of DGKalpha is 6-fluoro-1-methyl-4-[4-(5-methyl-1,3-benzoxazol-2-yl)piperidin-1-yl]-2-oxo-1,2-dihydroquinoline-3-carboxamide.

In another preferred embodiment of the present invention, the inhibitor of DGKalpha is DGKalpha inhibitor A of structure

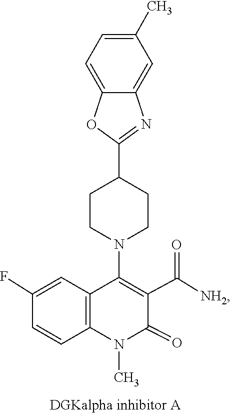 [SMILES: Cc1ccc2oc(C3CCN(c4c(C(N)=O)c(=O)n(C)c5ccc(F)cc45)CC3)nc2c1]

or a tautomer, an N-oxide, a hydrate, a solvate, or a salt thereof, or a mixture of same.

In another preferred embodiment of the present invention, the inhibitor of DGKalpha is DGKalpha inhibitor A of structure

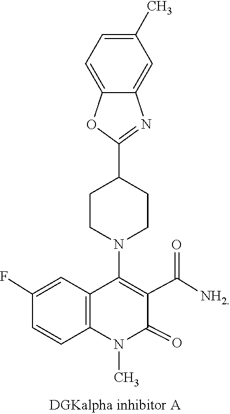 [SMILES: Cc1ccc2oc(C3CCN(c4c(C(N)=O)c(=O)n(C)c5ccc(F)cc45)CC3)nc2c1]

The synthesis of DGKalpha inhibitor A is described in the International Patent Application PCT/EP2020/083198, published as WO 2021/105117 A1, Example 298. International Patent Application PCT/EP2020/083198 also discloses methods of preparing other compounds of formula (I) mentioned herein.

### DGKzeta Inhibitors

### DGKzeta Inhibitors Embodiments A

In the following, the inhibitors of DGKzeta are described, which can be preferably used for the invention according to any one of aspect 1, aspect 2, aspect 3, aspect 4, aspect 5, aspect 6, aspect 7, aspect 8 or aspect 9 of the invention.

In some embodiments of the invention, the inhibitor of DGKzeta is a compound of formula (II)

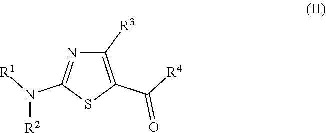

in which
R¹ represents a phenyl or 6-membered heteroaryl group optionally substituted, one, two, or three times, each substituent independently selected from a halogen atom or a group selected from hydroxy, cyano, nitro, C₁-C₆-alkyl, (phenyl)-(C₁-C₃-alkyl)-, C₁-C₆-haloalkyl, C₁-C₆-alkoxy, (phenyl)-(C₁-C₃-alkoxy)-, C₁-C₆-haloalkoxy, —N(R⁶)(R⁶),
   wherein the phenyl groups in said (phenyl)-(C₁-C₃-alkyl)- and (phenyl)-(C₁-C₃-alkoxy)- groups are optionally substituted one or two times, each substituent independently selected from a halogen atom or a group selected from cyano, methyl, ethyl, trifluoromethyl, methoxy, ethoxy, dimethylamino and trifluoromethoxy,
   or two substituents attached to adjacent carbon atoms of said phenyl or 6-membered heteroaryl group together form a bivalent group selected from —(CH₂)₃—, —(CH₂)₄—, —(CH₂)₂—O—, —(CH₂)₃—O—, —CH₂—O—CH₂—, —(CH₂)₂—O—CH₂—, —O—CH₂—O—, —O—CH₂—CH₂—O—, —O—CF₂—O—, —O—CH₂—CF₂—O—, and —O—CF₂—CF₂—O—,
   or
R¹ represents a 5-membered heteroaryl group optionally substituted one or two times, each substituent independently selected from a halogen atom or a group selected from cyano, C₁-C₃-alkyl, and C₁-C₃-alkoxy;
R² represents a group

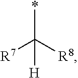

wherein “*” indicates the point of attachment to the nitrogen atom to which R² is attached;
R³ represents a group selected from methyl and —NH₂;
R⁴ represents a phenyl or 6-membered heteroaryl group optionally substituted, one, two, or three times, each substituent independently selected from a halogen atom or a group selected from cyano, nitro, C₁-C₆-alkyl, (phenyl)-(C₁-C₃-alkyl)-, (5- or 6-membered heteroaryl)-(C₁-C₃-alkyl)-, (C₃-C₇-cycloalkyl)-(C₁-C₃-alkyl)-, ((R⁹)O)—(C₁-C₆-alkyl)-, C₁-C₆-haloalkyl, C₃-C₇-cycloalkyl, —OR⁹, —N(R¹⁰)(R¹¹), ((R¹⁰)(R¹¹)N)—(C₁-C₃-alkyl)-, —C(═O)—N(R¹²)(R¹³), —S(═O)ₙ—R¹⁴, —C(═O)R¹⁴, —C(═O)—OR¹⁷, and a 5- or 6-membered heteroaryl group which itself is optionally substituted with one or two substituents selected from a halogen atom and a methyl group,
   or two substituents attached to adjacent carbon atoms of said phenyl or 6-membered heteroaryl group together form a bivalent group selected from —(CH₂)₃—, —(CH₂)₄—, —(CH₂)₂O—, —(CH₂)₃—O—, —CH₂—O—CH₂—, —(CH₂)₂—O—CH₂—, —O—CH₂—O—, —O—CH₂—CH₂—O—, —O—CF₂—CF₂—O—, —O—CH₂—CF₂—O—, and —O—CF₂—CF₂—O—;
R⁵ and R⁶ represent, independently from each occurrence, a hydrogen atom or a group selected from C₁-C₄-alkyl, (C₁-C₄-alkyl)-C(═O)—, C₃-C₄-cycloalkyl and (phenyl)-(C₁-C₃-alkyl)-,
   or
R⁵ and R⁶, together with the nitrogen atom to which they are attached, represent a monocyclic nitrogen containing 4- to 7-membered heterocycloalkyl group which is optionally substituted one, two or three times, each substituent independently selected from a halogen atom or a group selected from oxo, hydroxy, C₁-C₄-alkyl, (C₁-C₄-alkyl)-C(═O)—, C₃-C₄-cycloalkyl and C₁-C₄-alkoxy;
R⁷ represents a hydrogen atom or a C₁-C₂-alkyl group;
R⁸ represents a group selected from —C(═O)—NH₂ and —S(═O)₂—NH₂;
R⁹ represents a hydrogen atom or a group selected from C₁-C₆-alkyl, (5- or 6-membered heteroaryl)-(C₁-C₃-alkyl)-, (phenyl)-(C₁-C₃-alkyl)-, C₁-C₆-haloalkyl, C₂-C₄-hydroxyalkyl, (C₁-C₃-alkoxy)-C₂-C₃-alkyl-, ((C₁-C₃-alkyl)-C(═O)—O)—C₂-C₃-alkyl-, —C(R¹⁸)(R¹⁹)—C(═O)—OR¹⁷, —C(R¹⁸)(R¹⁰)—C(═O)—N(R²⁰)(R²¹), —C(═O)—N(R²⁰)(R²¹), phenyl and 5- or 6-membered heteroaryl group,
   wherein the phenyl group within said (phenyl)-(C₁-C₃-alkyl)- group and said phenyl group itself, and the 5- or 6-membered heteroaryl group within said (5- or 6-membered heteroaryl)-(C₁-C₃-alkyl)- group and said 5- or 6-membered heteroaryl group itself are optionally substituted one or two times, each substituent independently selected from a halogen atom or a group selected from cyano, methyl, ethyl, trifluoromethyl, methoxy, ethoxy, dimethylamino and trifluoromethoxy;
R¹⁰ and R¹¹ represent, independently from each occurrence, a hydrogen atom or a group selected from C₁-C₄-alkyl, C₁-C₄-haloalkyl, C₂-C₄-hydroxyalkyl, (C₁-C₃-alkoxy)-C₂-C₃-alkyl-, ((R²²)(R²³)N)—C₂-C₃-alkyl, (C₃-C₇-cycloalkyl)-(C₁-C₃-alkyl)-, (C₁-C₄-alkyl)-C(═O)—, C₃-C₇-cycloalkyl, (C₃-C₇-cycloalkyl)-C(═O)—, (phenyl)-(C₁-C₃-alkyl)-, (phenyl)-(C₁-C₃-alkyl)-C(═O)—, (phenyl)-(C₁-C₃-alkyl)-O—C(═O)—, phenyl and a 5- or 6-membered heteroaryl group,
   wherein C₃-C₇-cycloalkyl, and the C₃-C₇-cycloalkyl within said (C₃-C₇-cycloalkyl)-(C₁-C₃-alkyl)- and (C₃-C₇-cycloalkyl)-C(═O)— groups are optionally substituted one or two times, each substituent independently selected from a fluorine atom or a group selected from cyano, C₁-C₂-alkyl and C₁-C₂-haloalkyl,
   and wherein said phenyl and said 5- or 6-membered heteroaryl group, and the phenyl groups within said (phenyl)-(C₁-C₃-alkyl)-, (phenyl)-(C₁-C₃-alkyl)-C(═O)— and (phenyl)-(C₁-C₃-alkyl)-O—C(═O)— groups, are optionally substituted one or two times, each substituent independently selected from a halogen atom or a group selected from cyano, methyl, ethyl, trifluoromethyl, methoxy, ethoxy, dimethylamino and trifluoromethoxy,
   or
R¹⁰ and R¹¹, together with the nitrogen atom to which they are attached, represent a monocyclic nitrogen containing 4- to 7-membered heterocycloalkyl group, or a bicyclic nitrogen containing 5- to 11-membered heterocycloalkyl group, which are optionally substituted one, two or three times, each substituent independently selected from a halogen atom or a group selected from cyano, oxo, hydroxy, C₁-C₄-alkyl, C₁-C₄-haloalkyl, (C₁-C₄-alkyl)-C(═O)—, C₃-C₇-cycloalkyl, C₁-C₄-alkoxy, —N(R²²)(R²³), and a monocyclic 4- to 7-membered heterocycloalkyl group;
R¹² and R¹³ represent, independently from each occurrence, a hydrogen atom or a group selected from C₁-C₄-alkyl, C₁-C₄-haloalkyl, C₁-C₄-hydroxyalkyl, (C₁-C₄-alkoxy)-C₂-C₃-alkyl-, (C₁-C₄-haloalkoxy)-C₂-C₃-alkyl-, (phenoxy)-C₂-C₃-alkyl-, C₃-C₇cycloalkyl, monocyclic 4- to 7-membered heterocycloalkyl and (phenyl)-(C₁-C₃-alkyl)-,
   wherein C₃-C₇cycloalkyl and monocyclic 4- to 7-membered heterocycloalkyl are optionally substituted one, two or three times, each substituent independently selected from a halogen atom or a group selected from cyano, oxo, hydroxy, C₁-C₄-alkyl, (C₁-C₄-alkyl)-C(═O)—, C₃-C₄-cycloalkyl and C₁-C₄-alkoxy,
   and wherein the phenyl groups within said (phenoxy)-C₂-C₃-alkyl- group and said (phenyl)-(C₁-C₃-alkyl)- group are optionally substituted one or two times, each substituent independently selected from a halogen atom or a group selected from cyano, methyl, ethyl, trifluoromethyl, methoxy, ethoxy, dimethylamino and trifluoromethoxy,
   or
R² and R³, together with the nitrogen atom to which they are attached, represent a monocyclic nitrogen containing 4- to 7-membered heterocycloalkyl group which is optionally substituted one, two or three times, each substituent independently selected from a halogen atom or a group selected from cyano, oxo, hydroxy, C₁-C₄-alkyl, (C₁-C₄-alkyl)-C(═O)—, C₃-C₄-cycloalkyl and C₁-C₄-alkoxy;
R¹⁴ represents a group selected from C₁-C₄-alkyl, C₁-C₄-haloalkyl and phenyl,
   wherein the phenyl group is optionally substituted one or two times, each substituent independently selected from a halogen atom or a group selected from cyano, methyl, ethyl, trifluoromethyl, methoxy, ethoxy, dimethylamino and trifluoromethoxy;
R¹⁷ represents a C₁-C₄-alkyl group;
R¹⁸ and R¹⁹ represent, independently from each occurrence, a hydrogen atom or a C₁-C₄-alkyl group;
R²⁰ represents a hydrogen atom or a group selected from C₁-C₆-alkyl, C₃-C₄-alkenyl, C₃-C₄-alkynyl, C₁-C₃-alkoxy, C₃-C₇cycloalkyl, bicyclic C₅-C₁₁cycloalkyl, adamantyl, monocyclic 4- to 7-membered heterocycloalkyl, bicyclic 5- to 11-membered heterocycloalkyl, phenyl, naphthyl, and 5- to 10-membered heteroaryl,
   wherein said C₁-C₆-alkyl group is optionally substituted one, two or three times, each substituent independently selected from a halogen atom or a group selected from hydroxy, cyano, C₁-C₃-alkoxy, —N(R²¹)(R²³), C₃-C₇-cycloalkyl, bicyclic C₅-C₁₁-cycloalkyl, adamantyl, monocyclic 4- to 7-membered heterocycloalkyl, bicyclic 5- to 11-membered heterocycloalkyl, phenyl, and 5- to 10-membered heteroaryl, said phenyl and 5- to 10-membered heteroaryl substituents themselves being optionally substituted one or two times, each substituent independently selected from a halogen atom or a group selected from cyano, methyl, ethyl, trifluoromethyl, methoxy, ethoxy, dimethylamino and trifluoromethoxy,
   and wherein C₃-C₇-cycloalkyl, bicyclic C₆-C₁₁-cycloalkyl, adamantyl, monocyclic 4- to 7-membered heterocycloalkyl and bicyclic 5- to 11-membered heterocycloalkyl are optionally substituted one, two or three times, each substituent independently selected from a halogen atom or a group selected from cyano, oxo, hydroxy, C₁-C₄-alkyl, (C₁-C₄-alkyl)-C(═O)—, C₃-C₄-cycloalkyl and C₁-C₄-alkoxy,
   and wherein said phenyl, naphthyl and 5- to 10-membered heteroaryl groups are optionally substituted one, two or three times, each substituent independently selected from a halogen atom or a group selected from cyano, C₁-C₄-alkyl, C₁-C₄-haloalkyl, C₁-C₄-alkoxy, C₁-C₄-haloalkoxy, —N(R²²)(R²¹) and —C(═O)—N(R²⁴)(R²⁵),
R²¹ represents a hydrogen atom or a C₁-C₄-alkyl group,
   or
R²⁰ and R²¹, together with the nitrogen atom to which they are attached, represent a monocyclic nitrogen containing 4- to 7-membered heterocycloalkyl group which is optionally benzocondensed, and which is optionally substituted one, two or three times, each substituent independently selected from a halogen atom or a group selected from cyano, oxo, hydroxy, C₁-C₄-alkyl, C₁-C₄-haloalkyl, (phenyl)-(C₁-C₃-alkyl)-, (C₁-C₄-alkyl)-C(═O)—, C₃-C₄-cycloalkyl, C₁-C₄-alkoxy, C₁-C₃-haloalkoxy, —N(R²²)(R²¹) and —C(═O)—N(R²⁴)(R²⁵);
R²² and R²³ represent, independently from each occurrence, a hydrogen atom or a group selected from C₁-C₂-alkyl and (C₁-C₂-alkyl)-C(═O)—;
R²⁴ and R²⁵ represent, independently from each occurrence, a hydrogen atom or a C₁-C₄-alkyl group, and
n represents an integer 0, 1, or 2,
or a stereoisomer, a tautomer, an N-oxide, a hydrate, a solvate, or a salt thereof, or a mixture of same.

### DGKzeta Inhibitors Embodiments B

In some embodiments of the invention, the inhibitor of DGKzeta is a compound of formula (II), supra, in which
R¹ represents a phenyl or pyridinyl group optionally substituted, one, two, or three times, each substituent independently selected from a fluorine atom, a chlorine atom and a bromine atom, or a group selected from hydroxy, cyano, C₁-C₄-alkyl, C₁-C₂-fluoroalkyl, C₁-C₂-alkoxy, (phenyl)-(C₁-C₂-alkoxy)-, C₁-C₂-fluoroalkoxy and —N(R⁵)(R⁶),
   or two substituents attached to adjacent carbon atoms of said phenyl or pyridinyl group together form a bivalent group selected from —(CH₂)₃—, —O—CH₂—O— and —O—CF₂—O—,
   or
R¹ represents a pyrazolyl group optionally substituted with one methyl group;
R² represents a group

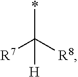

wherein “*” indicates the point of attachment to the nitrogen atom to which R² is attached;
R³ represents a group selected from methyl and —NH₂;
R⁴ represents a phenyl or pyridinyl group optionally substituted, one, two, or three times, each substituent independently selected from a halogen atom or a group selected from cyano, C₁-C₃-alkyl, ((R⁹)O)—(C₁-C₃-alkyl)-, C₁-C₃-fluoroalkyl, —OR⁹, —N(R¹⁰)(R¹¹), —C(═O)—N(R¹²)(R¹³), S(═O)ₙ—R¹⁴ and —C(═O)—OR¹⁷,
   or two substituents attached to adjacent carbon atoms of said phenyl or pyridinyl group together form a bivalent group selected from —(CH₂)₃—, —O—CH₂—O— and —O—CF₂—O—;
R⁵ and R⁶ represent, independently from each occurrence, a hydrogen atom or a C₁-C₂-alkyl group,
   or
R⁵ and R⁶, together with the nitrogen atom to which they are attached, represent a monocyclic nitrogen containing 4- to 7-membered heterocycloalkyl group which is optionally substituted one or two times, each substituent independently selected from a fluorine atom or a group selected from hydroxy and C₁-C₂-alkyl;
R⁷ represents a hydrogen atom or a C₁-C₂-alkyl group;
R⁸ represents a —C(═O)—NH₂ group;
R⁹ represents a hydrogen atom or a group selected from C₁-C₂-alkyl, benzyl, C₁-C₂-fluoroalkyl, C₂-hydroxyalkyl, (C₁-C₂-alkoxy)-C₂-alkyl-, ((C₁-C₂-alkyl)-C(═O)—O)—C₂-alkyl-, —C(R¹⁸)(R¹⁹)—C(═O)—OR¹⁷, —C(R¹⁸)(R¹⁹)—C(═O)—N(R²⁰)(R²¹), —C(═O)—N(R²⁰)(R²¹) and phenyl,
   wherein the phenyl group within said benzyl group and said phenyl group itself are optionally substituted one or two times, each substituent independently selected from a fluorine atom and a chlorine atom, or a group selected from cyano and methyl;
R¹⁰ and R¹¹ represent, independently from each occurrence, a hydrogen atom or a group selected from C₁-C₂-alkyl, C₁-C₂-fluoroalkyl, (C₃-C₅-cycloalkyl)-(C₁-C₂-alkyl)- (C₁-C₂-alkyl)-C(═O)—, C₃-C₇-cycloalkyl, C₃-C₇-cycloalkyl-(C═O)—, (phenyl)-(C₁-C₂-alkyl)-, (phenyl)-(C₁-C₂-alkyl)-C(═O)— and (phenyl)-(C₁-C₂-alkyl)-O—C(═O)—,
   wherein C₃-C₇cycloalkyl, and the C₃-C₅-cycloalkyl within said (C₃-C₅-cycloalkyl)-(C₁-C₂ alkyl)- and the C₃-C₇cycloalkyl within the C₃-C₇cycloalkyl-(C═O)— groups are optionally substituted one or two times, each substituent independently selected from a fluorine atom or a group selected from cyano, C₁-C₂-alkyl and C₁-C₂-fluoroalkyl,
   and wherein the phenyl groups within said (phenyl)-(C₁-C₂-alkyl)-, (phenyl)-(C₁-C₂-alkyl)-C(═O)— and (phenyl)-(C₁-C₂-alkyl)-O—C(═O)— groups are optionally substituted one or two times, each substituent independently selected from a fluorine atom, a chlorine atom and a methyl group,
   or
R¹⁰ and R¹¹, together with the nitrogen atom to which they are attached, represent a monocyclic nitrogen containing 4- to 7-membered heterocycloalkyl group which is optionally substituted one or two times, each substituent independently selected from a fluorine atom or a group selected from cyano, oxo, C₁-C₂-alkyl, C₁-C₂-fluoroalkyl and (C₁-C₂-alkyl)-C(═O)—;
R¹² and R¹³ represent, independently from each occurrence, a hydrogen atom or a group selected from C₁-C₄-alkyl, C₁-C₄-fluoroalkyl, C₁-C₄-hydroxyalkyl, (C₁-C₄-alkoxy)-C₂-C₃-alkyl-, (C₁-C₂-fluoroalkoxy)-C₂-C₃-alkyl-, (phenoxy)-C₂-C₃-alkyl-, C₃-C₇cycloalkyl, monocyclic 4- to 7-membered heterocycloalkyl and (phenyl)-(C₁-C₂-alkyl)-,
   wherein C₃-C₇cycloalkyl and monocyclic 4- to 7-membered heterocycloalkyl are optionally substituted one or two times, each substituent independently selected from a fluorine atom or a group selected from oxo, C₁-C₂-alkyl and (C₁-C₂-alkyl)-C(═O)—,
   and wherein the phenyl groups within said (phenoxy)-C₂-C₃-alkyl- group and said (phenyl)-(C₁-C₂-alkyl)- group are optionally substituted one or two times, each substituent independently selected from a fluorine atom and a chlorine atom, or a group selected from methyl, trifluoromethyl and methoxy,
   or
R¹² and R¹³, together with the nitrogen atom to which they are attached, represent a monocyclic nitrogen containing 4- to 7-membered heterocycloalkyl group which is optionally substituted one or two times, each substituent independently selected from a halogen atom or a group selected from oxo, C₁-C₂-alkyl and (C₁-C₂-alkyl)-C(═O)—;
R¹⁴ represents a group selected from methyl and trifluoromethyl;
R¹⁷ represents a C₁-C₂-alkyl group;
R¹⁸ and R¹⁹ represent, independently from each occurrence, a hydrogen atom or a methyl group;
R²⁰ represents a hydrogen atom or a group selected from optionally substituted C₁-C₃-alkyl, unsubstituted C₄-C₆-alkyl, prop-2-ynyl, methoxy, C₃-C₆-cycloalkyl, adamantyl, monocyclic 4- to 7-membered heterocycloalkyl, phenyl, and 5- to 10-membered heteroaryl,
   wherein said C₁-C₃-alkyl group is optionally substituted one, two or three times, each substituent independently selected from a halogen atom or a group selected from hydroxy, cyano, C₁-C₃-alkoxy, —N(R²²)(R²³), C₃-C₆-cycloalkyl, adamantyl, monocyclic 4- to 7-membered heterocycloalkyl, phenyl, and 5- to 10-membered heteroaryl, said phenyl and 5- to 10-membered heteroaryl substituents themselves being optionally substituted one or two times, each substituent independently selected from a fluorine atom, a chlorine atom and a methyl group,
   and wherein said C₃-C₆-cycloalkyl, adamantyl and monocyclic 4- to 7-membered heterocycloalkyl groups are optionally substituted one or two or three times, each substituent independently selected from a fluorine atom or a group selected from oxo, C₁-C₂-alkyl and (C₁-C₂-alkyl)-C(═O)—,
   and wherein said phenyl and 5- to 10-membered heteroaryl groups are optionally substituted one, two or three times, each substituent independently selected from a fluorine atom and a chlorine atom or a group selected from cyano, C₁-C₂-alkyl, C₁-C₂-fluoroalkyl, C₁-C₂-alkoxy, C₁-C₂-fluoroalkoxy, —N(R²²)(R²³) and —C(═O)—N(R²⁴)(R²⁵),
R²¹ represents a hydrogen atom or a C₁-C₂-alkyl group,
   or
R²⁰ and R²¹, together with the nitrogen atom to which they are attached, represent a monocyclic nitrogen containing 4- to 7-membered heterocycloalkyl group which is optionally benzocondensed, and which is optionally substituted one, two or three times, each substituent independently selected from a halogen atom or a group selected from cyano, oxo, hydroxy, C₁-C₂-alkyl, C₁-C₂-fluoroalkyl, benzyl, (C₁-C₂-alkyl)-C(═O)—, C₃-C₄-cycloalkyl, C₁-C₂-alkoxy, C₁-C₂-fluoroalkoxy, —N(R²²)(R²³) and —C(═O)—N(R²⁴)(R²⁵);
R²² and R²³ represent, independently from each occurrence, a hydrogen atom or a group selected from C₁-C₂-alkyl and (C₁-C₂-alkyl)-C(═O)—;
R²⁴ and R²⁵ represent, independently from each occurrence, a hydrogen atom or a C₁-C₂-alkyl group, and
n represents an integer 2,
or a stereoisomer, a tautomer, an N-oxide, a hydrate, a solvate, or a salt thereof, or a mixture of same.

### DGKzeta Inhibitors Embodiments C

In some embodiments of the invention, the inhibitor of DGKzeta is a compound of formula (II), supra, in which
R¹ represents a group

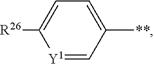

wherein “**” indicates the point of attachment to the nitrogen atom to which R¹ is attached;
R² represents a group

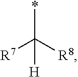

wherein “*” indicates the point of attachment to the nitrogen atom to which R² is attached;
R³ represents a group selected from methyl and —NH₂;
R⁴ represents a group

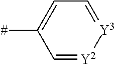

wherein “#” indicates the point of attachment to the carbonyl group to which R⁴ is attached;
R⁷ represents a hydrogen atom or a C₁-C₂-alkyl group;
R⁸ represents a —C(═O)—NH₂ group;
R⁹ represents a hydrogen atom or a group selected from C₁-C₂-alkyl, benzyl, C₁-C₂-fluoroalkyl, (C₁-C₂-alkoxy)-C₂-alkyl-, ((C₁-C₂-alkyl)-C(═O)—O)—C₂-alkyl-, —C(R¹⁸)(R¹⁹)—C(═O)—N(R²⁰)(R²¹), —C(═O)—N(R²⁰)(R²¹) and phenyl,
   wherein the phenyl group within said benzyl group and said phenyl group itself are optionally substituted one or two times, each substituent independently selected from a fluorine atom and a chlorine atom, or a group selected from cyano and methyl;
R¹⁰ and R¹¹ represent, independently from each occurrence, a hydrogen atom or a group selected from C₁-C₂-alkyl, C₃-C₇cycloalkyl and (benzyl)-O—C(═O)—,
   wherein C₃-C₇cycloalkyl is optionally substituted one or two times, each substituent independently selected from a fluorine atom or a group selected from methyl and trifluoromethyl,
   and wherein the phenyl group within said (benzyl)-O—C(═O)— group is optionally substituted one or two times, each substituent independently selected from a fluorine atom, a chlorine atom and a methyl group,
   or
R¹⁰ and R¹¹, together with the nitrogen atom to which they are attached, represent a monocyclic nitrogen containing 4- to 7-membered heterocycloalkyl group which is optionally substituted one or two times, each substituent independently selected from a fluorine atom or a group selected from cyano, methyl and trifluoromethyl;
R¹² and R¹³ represent, independently from each occurrence, a hydrogen atom or a group selected from C₁-C₄-alkyl, C₁-C₂-fluoroalkyl, C₁-C₂-hydroxyalkyl, (C₁-C₄-alkoxy)-C₂-alkyl-, (C₁-C₂-fluoroalkoxy)-C₂-alkyl-, (phenoxy)-C₂-alkyl-, C₃-C₇-cycloalkyl and (phenyl)-(C₁-C₂-alkyl)-,
   wherein the phenyl groups within said (phenoxy)-C₂-alkyl- group and said (phenyl)-(C₁-C₂-alkyl)- group are optionally substituted one or two times, each substituent independently selected from fluorine atom and a chlorine atom, or a group selected from methyl, trifluoromethyl and methoxy;
R¹⁷ represents a C₁-C₂-alkyl group;
R¹⁸ and R¹⁹ represent, independently from each occurrence, a hydrogen atom or a methyl group;
R²⁰ represents a group selected from benzyl and phenyl,
   wherein said phenyl group, and the phenyl group within said benzyl group, is optionally substituted one or two times, each substituent independently selected from a fluorine atom, a chlorine atom and a methyl group,
R²¹ represents a hydrogen atom or a methyl group,
Y¹ represents —C(H)═, —C(F)═, —C(Cl)═, —C(CN)═ or —N═;
Y² represents —C(H)═ or —N═;
Y³ represents —C(R²⁷)═ or —N═,
   with the proviso that if Y² represents —N═, Y³ represents —C(R²⁷)═, and if Y³ represents —N═, Y² represents —C(H)═;
R²⁶ represents a fluorine atom, a chlorine atom or a bromine atom, or a group selected from methyl, difluoromethyl, trifluoromethyl, methoxy, benzyloxy, difluoromethoxy and trifluoromethoxy, and
R²⁷ represents a halogen atom or a group selected from C₁-C₂-alkyl, C₁-C₂-fluoroalkyl, —OR⁹, —N(R¹⁰)(R¹), —C(═O)—N(R¹²)(R¹³) and —C(═O)—OR¹⁷,
or a stereoisomer, a tautomer, an N-oxide, a hydrate, a solvate, or a salt thereof, or a mixture of same.

### DGKzeta Inhibitors Embodiments D

In some embodiments of the present invention, the inhibitor of DGKzeta is a compound selected from:
rac-2-(N-(4-amino-5-benzoyl-thiazol-2-yl)-4-methoxy-2-methyl-anilino)propanamide,
rac-2-[N-(4-amino-5-benzoyl-thiazol-2-yl)-4-(dimethylamino)anilino]propanamide,
rac-2-(N-(4-amino-5-benzoyl-thiazol-2-yl)-4-isopropoxy-anilino)propanamide,
rac-2-(N-(4-amino-5-benzoyl-thiazol-2-yl)-2,4,6-trifluoro-anilino)propanamide,
rac-2-(N-(4-amino-5-benzoyl-thiazol-2-yl)-2-bromo-4-fluoro-anilino)propanamide,
rac-2-(N-[4-amino-5-(6-methylpyridine-3-carbonyl)thiazol-2-yl]-4-fluoro-anilino)propanamide,
rac-2-(N-[4-amino-5-(pyridine-4-carbonyl)thiazol-2-yl]-4-fluoro-anilino)propanamide,
rac-2-(N-[4-amino-5-(2-fluorobenzoyl)thiazol-2-yl]-4-fluoro-anilino)propanamide,
rac-2-(N-(4-amino-5-benzoyl-thiazol-2-yl)-4-cyano-anilino)propanamide,
rac-2-(N-[4-amino-5-[4-chloro-3-(trifluoromethyl)benzoyl]thiazol-2-yl]-4-fluoro-anilino)propanamide,
rac-2-(N-(4-amino-5-benzoyl-thiazol-2-yl)-2-chloro-4-fluoro-anilino)propanamide,
rac-2-(N-[4-amino-5-(3-cyanobenzoyl)thiazol-2-yl]-4-fluoro-anilino)propanamide,
rac-2-[N-(4-amino-5-benzoyl-thiazol-2-yl)-3-chloro-4-(dimethylamino)anilino]propanamide,
rac-2-(N-[4-amino-5-(6-methoxypyridine-3-carbonyl)thiazol-2-yl]-4-fluoro-anilino)propanamide,
rac-4-[4-amino-2-(N-[2-amino-1-methyl-2-oxo-ethyl]-4-fluoro-anilino)thiazole-5-carbonyl]-N-methyl-benzamide,
rac-2-(N-[4-amino-5-(3-fluorobenzoyl)thiazol-2-yl]-4-fluoro-anilino)propanamide,
rac-2-(N-(4-amino-5-benzoyl-thiazol-2-yl)-3-chloro-4-methoxy-anilino)propanamide,
rac-2-(N-[4-amino-5-(4-fluorobenzoyl)thiazol-2-yl]-4-methoxy-anilino)propanamide,
rac-2-(N-[4-amino-5-(4-methylsulfonyl benzoyl)thiazol-2-yl]-4-fluoro-anilino)propanamide,
rac-2-(N-[4-amino-5-(4-imidazol-1-ylbenzoyl)thiazol-2-yl]-4-fluoro-anilino)propanamide,
rac-2-(N-[4-amino-5-(4-cyano-3-fluoro-benzoyl)thiazol-2-yl]-4-fluoro-anilino)propanamide,
(R)-2-(N-[4-amino-5-(4-cyano-3-fluoro-benzoyl)thiazol-2-yl]-4-fluoro-anilino)propanamide,
(S)-2-(N-[4-amino-5-(4-cyano-3-fluoro-benzoyl)thiazol-2-yl]-4-fluoro-anilino)propanamide,
rac-2-(N-[4-amino-5-(4-cyano-2-fluoro-benzoyl)thiazol-2-yl]-4-fluoro-anilino)propanamide,
rac-2-(N-[4-amino-5-(2-fluoro-4-methoxy-benzoyl)thiazol-2-yl]-4-fluoro-anilino)propanamide,
rac-2-[N-(4-amino-5-benzoyl-thiazol-2-yl)-4-(trifluoromethoxy)anilino]propanamide,
rac-2-(N-[4-amino-5-(3,4-difluorobenzoyl)thiazol-2-yl]-4-fluoro-anilino)propanamide,
rac-2-(N-[4-amino-5-(3,4-dichlorobenzoyl)thiazol-2-yl]-4-fluoro-anilino)propanamide,
rac-2-[N-[4-amino-5-(4-fluorobenzoyl)thiazol-2-yl]-4-(trifluoromethoxy)anilino]propanamide,
(R)-2-[N-[4-amino-5-(4-fluorobenzoyl)thiazol-2-yl]-4-(trifluoromethoxy)anilino]propanamide,
(S)-2-[N-[4-amino-5-(4-fluorobenzoyl)thiazol-2-yl]-4-(trifluoromethoxy)anilino]propanamide,
rac-2-[N-(4-amino-5-benzoyl-thiazol-2-yl)-4-(trifluoromethyl)anilino]propanamide,
rac-2-(N-[4-amino-5-[6-(trifluoromethyl)pyridine-3-carbonyl]thiazol-2-yl]-4-fluoro-anilino)propanamide,
(R)-2-(N-[4-amino-5-[6-(trifluoromethyl)pyridine-3-carbonyl]thiazol-2-yl]-4-fluoro-anilino)propanamide,
(S)-2-(N-[4-amino-5-[6-(trifluoromethyl)pyridine-3-carbonyl]thiazol-2-yl]-4-fluoro-anilino)propanamide,
rac-2-(N-(4-amino-5-benzoyl-thiazol-2-yl)-4-chloro-2-fluoro-anilino)propanamide,
rac-2-(N-[4-amino-5-(indane-5-carbonyl)thiazol-2-yl]-4-fluoro-anilino)propanamide,
rac-2-(N-(4-amino-5-benzoyl-thiazol-2-yl)-3,4-difluoro-anilino)propanamide,
rac-2-(N-[4-amino-5-(4-cyanobenzoyl)thiazol-2-yl]-4-fluoro-anilino)propanamide,
(R)-2-(N-[4-amino-5-(4-cyanobenzoyl)thiazol-2-yl]-4-fluoro-anilino)propanamide,
(S)-2-(N-[4-amino-5-(4-cyanobenzoyl)thiazol-2-yl]-4-fluoro-anilino)propanamide,
rac-2-(N-(4-amino-5-benzoyl-thiazol-2-yl)-4-chloro-anilino)propanamide,
rac-2-(N-(4-amino-5-benzoyl-thiazol-2-yl)-3,4-dichloro-anilino)propanamide,
rac-2-(N-(4-amino-5-benzoyl-thiazol-2-yl)-4-chloro-3-fluoro-anilino)propanamide,
rac-2-(N-[4-amino-5-(4-fluorobenzoyl)thiazol-2-yl]-4-chloro-anilino)propanamide,
(R)-2-(N-[4-amino-5-(4-fluorobenzoyl)thiazol-2-yl]-4-chloro-anilino)propanamide,
(S)-2-(N-[4-amino-5-(4-fluorobenzoyl)thiazol-2-yl]-4-chloro-anilino)propanamide,
rac-2-(N-[4-amino-5-(4-chlorobenzoyl)thiazol-2-yl]-4-fluoro-anilino)propanamide,
(R)-2-(N-[4-amino-5-(4-chlorobenzoyl)thiazol-2-yl]-4-fluoro-anilino)propanamide,
(S)-2-(N-[4-amino-5-(4-chlorobenzoyl)thiazol-2-yl]-4-fluoro-anilino)propanamide,
rac-ethyl 2-[4-[4-amino-2-(N-[2-amino-1-methyl-2-oxo-ethyl]-4-fluoro-anilino)thiazole-5-carbonyl]phenoxy]-2-methyl-propanoate,
rac-2-(N-[4-amino-5-[4-(trifluoromethoxy)benzoyl]thiazol-2-yl]-4-fluoro-anilino)propanamide,
(R)-2-(N-[4-amino-5-[4-(trifluoromethoxy)benzoyl]thiazol-2-yl]-4-fluoro-anilino)propanamide,
(S)-2-(N-[4-amino-5-[4-(trifluoromethoxy)benzoyl]thiazol-2-yl]-4-fluoro-anilino)propanamide,
rac-2-(N-[4-amino-5-(4-chlorobenzoyl)thiazol-2-yl]-4-chloro-anilino)propanamide,
(R)-2-(N-[4-amino-5-(4-chlorobenzoyl)thiazol-2-yl]-4-chloro-anilino)propanamide,
(S)-2-(N-[4-amino-5-(4-chlorobenzoyl)thiazol-2-yl]-4-chloro-anilino)propanamide,
rac-2-(N-[4-amino-5-[4-(trifluoromethyl)benzoyl]thiazol-2-yl]-4-fluoro-anilino)propanamide,
rac-2-[N-[4-amino-5-[4-(difluoromethoxy)benzoyl]thiazol-2-yl]-4-(trifluoromethyl)anilino]propanamide,
(R)-2-[N-[4-amino-5-[4-(difluoromethoxy)benzoyl]thiazol-2-yl]-4-(trifluoromethyl)anilino]propanamide,
(S)-2-[N-[4-amino-5-[4-(difluoromethoxy)benzoyl]thiazol-2-yl]-4-(trifluoromethyl)anilino]propanamide,
rac-2-(N-[4-amino-5-[4-(difluoromethoxy)benzoyl]thiazol-2-yl]-4-fluoro-anilino)propanamide,
(R)-2-(N-[4-amino-5-[4-(difluoromethoxy)benzoyl]thiazol-2-yl]-4-fluoro-anilino)propanamide,
(S)-2-(N-[4-amino-5-[4-(difluoromethoxy)benzoyl]thiazol-2-yl]-4-fluoro-anilino)propanamide,
2-(N-[4-amino-5-[4-[2-amino-1-methyl-2-oxo-ethoxy]benzoyl]thiazol-2-yl]anilino)propanamide (mixture of stereoisomers),
(2R)—(N-[4-amino-5-[4-[2-amino-(1R)-methyl-2-oxo-ethoxy]benzoyl]thiazol-2-yl]anilino)propanamide,
(2R)—(N-[4-amino-5-[4-[2-amino-(1S)-methyl-2-oxo-ethoxy]benzoyl]thiazol-2-yl]anilino)propanamide,
(2S)—(N-[4-amino-5-[4-[2-amino-(1R)-methyl-2-oxo-ethoxy]benzoyl]thiazol-2-yl]anilino)propanamide,
(2S)—(N-[4-amino-5-[4-[2-amino-(1S)-methyl-2-oxo-ethoxy]benzoyl]thiazol-2-yl]anilino)propanamide,
rac-2-(N-[4-amino-5-[4-(difluoromethoxy)benzoyl]thiazol-2-yl]-4-chloro-2-fluoro-anilino)propanamide,
(R)-2-(N-[4-amino-5-[4-(difluoromethoxy)benzoyl]thiazol-2-yl]-4-chloro-2-fluoro-anilino)propanamide,
(S)-2-(N-[4-amino-5-[4-(difluoromethoxy)benzoyl]thiazol-2-yl]-4-chloro-2-fluoro-anilino)propanamide,
rac-2-(N-[4-amino-5-[4-(difluoromethoxy)benzoyl]thiazol-2-yl]-4-chloro-anilino)propanamide,
(R)-2-(N-[4-amino-5-[4-(difluoromethoxy)benzoyl]thiazol-2-yl]-4-chloro-anilino)propanamide,
(S)-2-(N-[4-amino-5-[4-(difluoromethoxy)benzoyl]thiazol-2-yl]-4-chloro-anilino)propanamide,
rac-2-(N-[4-amino-5-[4-(difluoromethoxy)benzoyl]thiazol-2-yl]-4-chloro-3-fluoro-anilino)propanamide,
(R)-2-(N-[4-amino-5-[4-(difluoromethoxy)benzoyl]thiazol-2-yl]-4-chloro-3-fluoro-anilino)propanamide,
(S)-2-(N-[4-amino-5-[4-(difluoromethoxy)benzoyl]thiazol-2-yl]-4-chloro-3-fluoro-anilino)propanamide,
rac-2-(N-[4-amino-5-[4-(difluoromethoxy)benzoyl]thiazol-2-yl]-3,4-difluoro-anilino)propanamide,
(R)-2-(N-[4-amino-5-[4-(difluoromethoxy)benzoyl]thiazol-2-yl]-3,4-difluoro-anilino)propanamide,
(S)-2-(N-[4-amino-5-[4-(difluoromethoxy)benzoyl]thiazol-2-yl]-3,4-difluoro-anilino)propanamide,
rac-2-(N-[4-amino-5-(pyridine-4-carbonyl)thiazol-2-yl]-3,4-difluoro-anilino)propanamide
(R)-2-(N-[4-amino-5-(pyridine-4-carbonyl)thiazol-2-yl]-3,4-difluoro-anilino)propanamide,
(S)-2-(N-[4-amino-5-(pyridine-4-carbonyl)thiazol-2-yl]-3,4-difluoro-anilino)propanamide,
rac-2-(N-[4-amino-5-(4-methoxybenzoyl)thiazol-2-yl]-3,4-difluoro-anilino)propanamide,
(R)-2-(N-[4-amino-5-(4-methoxybenzoyl)thiazol-2-yl]-3,4-difluoro-anilino)propanamide,
(S)-2-(N-[4-amino-5-(4-methoxybenzoyl)thiazol-2-yl]-3,4-difluoro-anilino)propanamide,
rac-2-(N-[4-amino-5-(6-methoxypyridine-3-carbonyl)thiazol-2-yl]-3,4-difluoro-anilino)propanamide,
(R)-2-(N-[4-amino-5-(6-methoxypyridine-3-carbonyl)thiazol-2-yl]-3,4-difluoro-anilino)propanamide,
(S)-2-(N-[4-amino-5-(6-methoxypyridine-3-carbonyl)thiazol-2-yl]-3,4-difluoro-anilino)propanamide,
rac-2-(N-[4-amino-5-[6-(trifluoromethyl)pyridine-3-carbonyl]thiazol-2-yl]-3,4-difluoro-anilino)propanamide,
(R)-2-(N-[4-amino-5-[6-(trifluoromethyl)pyridine-3-carbonyl]thiazol-2-yl]-3,4-difluoro-anilino)propanamide,
(S)-2-(N-[4-amino-5-[6-(trifluoromethyl)pyridine-3-carbonyl]thiazol-2-yl]-3,4-difluoro-anilino)propanamide,
rac-2-(N-[5-[4-(difluoromethoxy)benzoyl]-4-methyl-thiazol-2-yl]-4-fluoro-anilino)propanamide,
rac-2-(N-[5-[4-(difluoromethoxy)benzoyl]-4-methyl-thiazol-2-yl]-3,4-difluoro-anilino)propanamide,
(R)-2-(N-[5-[4-(difluoromethoxy)benzoyl]-4-methyl-thiazol-2-yl]-3,4-difluoro-anilino)propanamide,
(S)-2-(N-[5-[4-(difluoromethoxy)benzoyl]-4-methyl-thiazol-2-yl]-3,4-difluoro-anilino)propanamide,
rac-2-(N-[4-amino-5-(pyridine-4-carbonyl)thiazol-2-yl]-4-chloro-3-fluoro-anilino)propanamide,
(R)-2-(N-[4-amino-5-(pyridine-4-carbonyl)thiazol-2-yl]-4-chloro-3-fluoro-anilino)propanamide,
(S)-2-(N-[4-amino-5-(pyridine-4-carbonyl)thiazol-2-yl]-4-chloro-3-fluoro-anilino)propanamide,
rac-2-(N-[4-amino-5-(pyridine-4-carbonyl)thiazol-2-yl]-4-chloro-anilino)propanamide,
(R)-2-(N-[4-amino-5-(pyridine-4-carbonyl)thiazol-2-yl]-4-chloro-anilino)propanamide,
(S)-2-(N-[4-amino-5-(pyridine-4-carbonyl)thiazol-2-yl]-4-chloro-anilino)propanamide,
rac-2-(N-[4-amino-5-(4-methoxybenzoyl)thiazol-2-yl]-4-chloro-anilino)propanamide,
(R)-2-(N-[4-amino-5-(4-methoxybenzoyl)thiazol-2-yl]-4-chloro-anilino)propanamide,
(S)-2-(N-[4-amino-5-(4-methoxybenzoyl)thiazol-2-yl]-4-chloro-anilino)propanamide,
rac-2-(N-[4-amino-5-(4-methoxybenzoyl)thiazol-2-yl]-4-chloro-3-fluoro-anilino)propanamide,
(R)-2-(N-[4-amino-5-(4-methoxybenzoyl)thiazol-2-yl]-4-chloro-3-fluoro-anilino)propanamide,
(S)-2-(N-[4-amino-5-(4-methoxybenzoyl)thiazol-2-yl]-4-chloro-3-fluoro-anilino)propanamide
rac-2-(N-(4-amino-5-benzoyl-thiazol-2-yl)anilino)propanamide,
rac-2-(N-(4-amino-5-benzoyl-thiazol-2-yl)-4-fluoro-anilino)propanamide,
(R)-2-(N-(4-amino-5-benzoyl-thiazol-2-yl)-4-fluoro-anilino)propanamide,
(S)-2-(N-(4-amino-5-benzoyl-thiazol-2-yl)-4-fluoro-anilino)propanamide,
rac-2-(N-[4-amino-5-(4-methoxybenzoyl)thiazol-2-yl]-4-fluoro-anilino)propanamide,
(R)-2-(N-[4-amino-5-(4-methoxybenzoyl)thiazol-2-yl]-4-fluoro-anilino)propanamide,
(S)-2-(N-[4-amino-5-(4-methoxybenzoyl)thiazol-2-yl]-4-fluoro-anilino)propanamide,
rac-2-(N-[4-amino-5-(4-methylbenzoyl)thiazol-2-yl]-4-fluoro-anilino)propanamide,
(R)-2-(N-[4-amino-5-(4-methylbenzoyl)thiazol-2-yl]-4-fluoro-anilino)propanamide,
(S)-2-(N-[4-amino-5-(4-methylbenzoyl)thiazol-2-yl]-4-fluoro-anilino)propanamide,
rac-2-(N-[4-amino-5-(4-fluorobenzoyl)thiazol-2-yl]-4-fluoro-anilino)propanamide,
rac-2-(N-(4-amino-5-benzoyl-thiazol-2-yl)-2,4-difluoro-anilino)propanamide,
rac-2-(N-(4-amino-5-benzoyl-thiazol-2-yl)-4-methoxy-anilino)propanamide,
rac-2-[(4-amino-5-benzoyl-1,3-thiazol-2-yl)(phenyl)amino]butanamide,
rac-2-[(4-amino-5-benzoyl-1,3-thiazol-2-yl)(4-fluorophenyl)amino]butanamide,
2-(N-[4-amino-5-[4-(2-amino-1-methyl-2-oxo-ethoxy)benzoyl]thiazol-2-yl]-4-fluoro-anilino)propanamide (mixture of stereoisomers),
rac-2-{[4-amino-5-(4-methoxybenzoyl)-1,3-thiazol-2-yl](4-fluorophenyl)amino}butanamide,
2-(N-(4-amino-5-benzoyl-thiazol-2-yl)-4-fluoro-anilino)acetamide,
2-(N-[4-amino-5-(4-methylbenzoyl)thiazol-2-yl]-4-fluoro-anilino)acetamide,
rac-2-(N-(4-amino-5-benzoyl-thiazol-2-yl)-4-methyl-anilino)propanamide,
(R)-2-(N-(4-amino-5-benzoyl-thiazol-2-yl)-4-methyl-anilino)propanamide,
(S)-2-(N-(4-amino-5-benzoyl-thiazol-2-yl)-4-methyl-anilino)propanamide,
rac-2-(N-(4-amino-5-benzoyl-thiazol-2-yl)-3-fluoro-anilino)propanamide,
(R)-2-(N-(4-amino-5-benzoyl-thiazol-2-yl)-3-fluoro-anilino)propanamide,
(S)-2-(N-(4-amino-5-benzoyl-thiazol-2-yl)-3-fluoro-anilino)propanamide,
rac-2-(N-(4-amino-5-benzoyl-thiazol-2-yl)-2-fluoro-anilino)propanamide,
(R)-2-(N-(4-amino-5-benzoyl-thiazol-2-yl)-2-fluoro-anilino)propanamide,
(S)-2-(N-(4-amino-5-benzoyl-thiazol-2-yl)-2-fluoro-anilino)propanamide,
rac-2-(N-(4-amino-5-benzoyl-thiazol-2-yl)-3-methyl-anilino)propanamide,
(R)-2-(N-(4-amino-5-benzoyl-thiazol-2-yl)-3-methyl-anilino)propanamide,
(S)-2-(N-(4-amino-5-benzoyl-thiazol-2-yl)-3-methyl-anilino)propanamide,
rac-2-(N-(4-amino-5-benzoyl-thiazol-2-yl)-2-methyl-anilino)propanamide,
(R)-2-(N-(4-amino-5-benzoyl-thiazol-2-yl)-2-methyl-anilino)propanamide,
(S)-2-(N-(4-amino-5-benzoyl-thiazol-2-yl)-2-methyl-anilino)propanamide,
rac-2-[(4-amino-5-benzoyl-thiazol-2-yl)-(1-methylpyrazol-4-yl)amino]propanamide,
(R)-2-[(4-amino-5-benzoyl-thiazol-2-yl)-(1-methylpyrazol-4-yl)amino]propanamide,
(S)-2-[(4-amino-5-benzoyl-thiazol-2-yl)-(1-methylpyrazol-4-yl)amino]propanamide,
rac-2-[(4-amino-5-benzoyl-thiazol-2-yl)-(3-pyridyl)amino]propanamide,
(R)-2-[(4-amino-5-benzoyl-thiazol-2-yl)-(3-pyridyl)amino]propanamide,
(S)-2-[(4-amino-5-benzoyl-thiazol-2-yl)-(3-pyridyl)amino]propanamide,
rac-2-(N-[4-amino-5-(4-bromobenzoyl)thiazol-2-yl]-4-fluoro-anilino)propanamide,
rac-ethyl 2-[4-[4-amino-2-(4-fluoro-N-[2-amino-1-methyl-2-oxo-ethyl]anilino)thiazole-5-carbonyl]phenoxy]acetate,
(R)-ethyl 2-[4-[4-amino-2-(4-fluoro-N-[2-amino-1-methyl-2-oxo-ethyl]anilino)thiazole-5-carbonyl]phenoxy]acetate,
(S)-ethyl 2-[4-[4-amino-2-(4-fluoro-N-[2-amino-1-methyl-2-oxo-ethyl]anilino)thiazole-5-carbonyl]phenoxy]acetate,
rac-2-(N-[4-amino-5-[4-[2-(isopropylamino)-2-oxo-ethoxy]benzoyl]thiazol-2-yl]-4-fluoro-anilino)propanamide,
rac-2-(N-[4-amino-5-[4-[2-(m-tolylmethylamino)-2-oxo-ethoxy]benzoyl]thiazol-2-yl]-4-fluoro-anilino)propanamide,
rac-2-(N-[4-amino-5-[4-[2-(o-tolylmethylamino)-2-oxo-ethoxy]benzoyl]thiazol-2-yl]-4-fluoro-anilino)propanamide,
rac-2-(N-[4-amino-5-[4-[2-[(3-chlorophenyl)methyl amino]-2-oxo-ethoxy]benzoyl]thiazol-2-yl]-4-fluoro-anilino)propanamide,
rac-2-(N-[4-amino-5-[4-[2-(4-methylpiperazin-1-yl)-2-oxo-ethoxy]benzoyl]thiazol-2-yl]-4-fluoro-anilino)propanamide,
rac-2-(N-[4-amino-5-[4-[2-(3-methylanilino)-2-oxo-ethoxy]benzoyl]thiazol-2-yl]-4-fluoro-anilino)propanamide,
rac-2-(N-[4-amino-5-[4-(2-morpholino-2-oxo-ethoxy)benzoyl]thiazol-2-yl]-4-fluoro-anilino)propanamide,
rac-2-(N-[4-amino-5-[4-[2-oxo-2-[2-(1-piperidyl)ethylamino]ethoxy]benzoyl]thiazol-2-yl]-4-fluoro-anilino)propanamide,
rac-2-(N-[4-amino-5-[4-[2-(4-benzyl-1-piperidyl)-2-oxo-ethoxy]benzoyl]thiazol-2-yl]-4-fluoro-anilino)propanamide,
rac-2-(N-[4-amino-5-[4-[2-(2-methoxyethylamino)-2-oxo-ethoxy]benzoyl]thiazol-2-yl]-4-fluoro-anilino)propanamide,
rac-2-(N-[4-amino-5-[4-[2-(4-cyanoanilino)-2-oxo-ethoxy]benzoyl]thiazol-2-yl]-4-fluoro-anilino)propanamide,
rac-2-(N-[4-amino-5-[4-[2-[ethyl(prop-2-ynyl)amino]-2-oxo-ethoxy]benzoyl]thiazol-2-yl]-4-fluoro-anilino)propanamide,
rac-2-(N-[4-amino-5-[4-[2-[(2-methoxyphenyl)methylamino]-2-oxo-ethoxy]benzoyl]thiazol-2-yl]-4-fluoro-anilino)propanamide,
rac-2-(N-[4-amino-5-[4-[2-[(3-methoxyphenyl)methylamino]-2-oxo-ethoxy]benzoyl]thiazol-2-yl]-4-fluoro-anilino)propanamide,
rac-2-(N-[4-amino-5-[4-[2-[(2-fluorophenyl)methylamino]-2-oxo-ethoxy]benzoyl]thiazol-2-yl]-4-fluoro-anilino)propanamide,
rac-2-(N-[4-amino-5-[4-[2-[(4-fluorophenyl)methylamino]-2-oxo-ethoxy]benzoyl]thiazol-2-yl]-4-fluoro-anilino)propanamide,
rac-2-(N-[4-amino-5-[4-[2-(1H-benzimidazol-2-ylmethylamino)-2-oxo-ethoxy]benzoyl]thiazol-2-yl]-4-fluoro-anilino)propanamide,
rac-2-(N-[4-amino-5-[4-[2-oxo-2-(2,2,2-trifluoroethylamino)ethoxy]benzoyl]thiazol-2-yl]-4-fluoro-anilino)propanamide,
rac-2-(N-[4-amino-5-[4-[2-[methyl (2-pyridyl)amino]-2-oxo-ethoxy]benzoyl]thiazol-2-yl]-4-fluoro-anilino)propanamide,
rac-2-(N-[4-amino-5-[4-[2-[methyl-(1-methyl-4-piperidyl)amino]-2-oxo-ethoxy]benzoyl]thiazol-2-yl]-4-fluoro-anilino)propanamide,
rac-2-(N-[4-amino-5-[4-[2-(methoxyamino)-2-oxo-ethoxy]benzoyl]thiazol-2-yl]-4-fluoro-anilino)propanamide,
rac-2-(N-[4-amino-5-[4-[2-[(5-methylisoxazol-3-yl)amino]-2-oxo-ethoxy]benzoyl]thiazol-2-yl]-4-fluoro-anilino)propanamide,
rac-2-(N-[4-amino-5-[4-[2-(ethylamino)-2-oxo-ethoxy]benzoyl]thiazol-2-yl]-4-fluoro-anilino)propanamide,
rac-2-(N-[4-amino-5-[4-[2-(4-methylanilino)-2-oxo-ethoxy]benzoyl]thiazol-2-yl]-4-fluoro-anilino)propanamide,
rac-2-(N-[4-amino-5-[4-[2-(cyclohexylamino)-2-oxo-ethoxy]benzoyl]thiazol-2-yl]-4-fluoro-anilino)propanamide,
rac-3-[[2-[4-[4-amino-2-(N-[2-amino-1-methyl-2-oxo-ethyl]-4-fluoro-anilino)thiazole-5-carbonyl]phenoxy]acetyl]amino]benzamide,
rac-2-(N-[4-amino-5-[4-[2-oxo-2-(6-quinolylamino)ethoxy]benzoyl]thiazol-2-yl]-4-fluoro-anilino)propanamide,
rac-4-[[2-[4-[4-amino-2-(N-[2-amino-1-methyl-2-oxo-ethyl]-4-fluoro-anilino)thiazole-5-carbonyl]phenoxy]acetyl]amino]benzamide,
(2S)-1-[2-[4-[4-amino-2-(N-[2-amino-(1RS)-methyl-2-oxo-ethyl]-4-fluoro-anilino)thiazole-5-carbonyl]phenoxy]acetyl]pyrrolidine-2-carboxamide (mixture of two diastereomers),
rac-2-(N-[4-amino-5-[4-[2-[ethyl(methyl)amino]-2-oxo-ethoxy]benzoyl]thiazol-2-yl]-4-fluoro-anilino)propanamide,
rac-2-(N-[4-amino-5-[4-[2-[(3-methylisoxazol-5-yl)amino]-2-oxo-ethoxy]benzoyl]thiazol-2-yl]-4-fluoro-anilino)propanamide,
rac-2-(N-[4-amino-5-[4-[2-[3-(dimethylamino)propyl-methyl-amino]-2-oxo-ethoxy]benzoyl]thiazol-2-yl]-4-fluoro-anilino)propanamide,
rac-2-(N-[5-[4-[2-(4-acetylpiperazin-1-yl)-2-oxo-ethoxy]benzoyl]-4-amino-thiazol-2-yl]-4-fluoro-anilino)propanamide,
rac-2-(N-[4-amino-5-[4-[2-oxo-2-(3-pyridylmethylamino)ethoxy]benzoyl]thiazol-2-yl]-4-fluoro-anilino)propanamide,
2-(N-[4-amino-5-[4-[2-(2,3-dihydroxypropylamino)-2-oxo-ethoxy]benzoyl]thiazol-2-yl]-4-fluoro-anilino)propanamide (mixture of stereoisomers),
rac-2-(N-[4-amino-5-[4-[2-(4-methoxyanilino)-2-oxo-ethoxy]benzoyl]thiazol-2-yl]-4-fluoro-anilino)propanamide,
rac-2-(N-[4-amino-5-[4-[2-[benzyl(methyl)amino]-2-oxo-ethoxy]benzoyl]thiazol-2-yl]-4-fluoro-anilino)propanamide,
rac-2-(N-[4-amino-5-[4-[2-(4-chloroanilino)-2-oxo-ethoxy]benzoyl]thiazol-2-yl]-4-fluoro-anilino)propanamide,
rac-2-(N-[4-amino-5-[4-[2-[(2-chlorophenyl)methylamino]-2-oxo-ethoxy]benzoyl]thiazol-2-yl]-4-fluoro-anilino)propanamide,
rac-2-(N-[4-amino-5-[4-[2-[(4-chlorophenyl)methylamino]-2-oxo-ethoxy]benzoyl]thiazol-2-yl]-4-fluoro-anilino)propanamide,
rac-2-(N-[4-amino-5-[4-[2-(4-fluoroanilino)-2-oxo-ethoxy]benzoyl]thiazol-2-yl]-4-fluoro-anilino)propanamide,
rac-2-(N-[4-amino-5-[4-[2-(azepan-1-yl)-2-oxo-ethoxy]benzoyl]thiazol-2-yl]-4-fluoro-anilino)propanamide,
rac-2-(N-[4-amino-5-[4-[2-[(4-methoxyphenyl)methylamino]-2-oxo-ethoxy]benzoyl]thiazol-2-yl]-4-fluoro-anilino)propanamide,
2-(N-[4-amino-5-[4-[2-oxo-2-(1-phenylethylamino)ethoxy]benzoyl]thiazol-2-yl]-4-fluoro-anilino)propanamide (mixture of stereoisomers),
rac-2-(N-[4-amino-5-[4-[2-oxo-2-(p-tolylmethylamino)ethoxy]benzoyl]thiazol-2-yl]-4-fluoro-anilino)propanamide,
rac-2-(N-[4-amino-5-[4-[2-[methyl(2-phenylethyl)amino]-2-oxo-ethoxy]benzoyl]thiazol-2-yl]-4-fluoro-anilino)propanamide,
2-(N-[4-amino-5-[4-[2-(3-methyl-1-piperidyl)-2-oxo-ethoxy]benzoyl]thiazol-2-yl]-4-fluoro-anilino)propanamide (mixture of stereoisomers),
rac-2-(N-[4-amino-5-[4-[2-(4-methyl-1-piperidyl)-2-oxo-ethoxy]benzoyl]thiazol-2-yl]-4-fluoro-anilino)propanamide,
rac-2-(N-[5-[4-[2-(4-acetamidoanilino)-2-oxo-ethoxy]benzoyl]-4-amino-thiazol-2-yl]-4-fluoro-anilino)propanamide,
rac-2-(N-[4-amino-5-[4-[2-oxo-2-(1H-pyrazolo[3,4-d]pyrimidin-4-ylamino)ethoxy]benzoyl]thiazol-2-yl]-4-fluoro-anilino)propanamide,
rac-2-(N-[4-amino-5-[4-[2-(cyclopentylamino)-2-oxo-ethoxy]benzoyl]thiazol-2-yl]-4-fluoro-anilino)propanamide,
rac-2-(N-[4-amino-5-[4-[2-(3,4-dihydro-1H-isoquinolin-2-yl)-2-oxo-ethoxy]benzoyl]thiazol-2-yl]-4-fluoro-anilino)propanamide,
rac-2-(N-[4-amino-5-[4-(2-isoindolin-2-yl-2-oxo-ethoxy)benzoyl]thiazol-2-yl]-4-fluoro-anilino)propanamide,
rac-2-(N-[4-amino-5-[4-[2-[2-furylmethyl(methyl)amino]-2-oxo-ethoxy]benzoyl]thiazol-2-yl]-4-fluoro-anilino)propanamide,
rac-2-(N-[4-amino-5-[4-[2-[4-(dimethylamino)-1-piperidyl]-2-oxo-ethoxy]benzoyl]thiazol-2-yl]-4-fluoro-anilino)propanamide,
rac-2-(N-[4-amino-5-[4-[2-[methyl (3-pyridylmethyl)amino]-2-oxo-ethoxy]benzoyl]thiazol-2-yl]-4-fluoro-anilino)propanamide,
rac-2-(N-[4-amino-5-[4-[2-(N,2-dimethylanilino)-2-oxo-ethoxy]benzoyl]thiazol-2-yl]-4-fluoro-anilino)propanamide,
rac-2-(N-[4-amino-5-[4-[2-(N,4-dimethylanilino)-2-oxo-ethoxy]benzoyl]thiazol-2-yl]-4-fluoro-anilino)propanamide,
rac-2-(N-[4-amino-5-[4-[2-(N,3-dimethylanilino)-2-oxo-ethoxy]benzoyl]thiazol-2-yl]-4-fluoro-anilino)propanamide,
rac-2-(N-[4-amino-5-[4-[2-(2,2-dimethylpropylamino)-2-oxo-ethoxy]benzoyl]thiazol-2-yl]-4-fluoro-anilino)propanamide,
2-(N-[5-[4-[2-(1-adamantylamino)-2-oxo-ethoxy]benzoyl]-4-amino-thiazol-2-yl]-4-fluoro-anilino)propanamide (single stereoisomer),
2-(N-[5-[4-[2-(1-adamantylmethylamino)-2-oxo-ethoxy]benzoyl]-4-amino-thiazol-2-yl]-4-fluoro-anilino)propanamide (single stereoisomer),
2-(N-[5-[4-[2-[2-(1-adamantyl)ethylamino]-2-oxo-ethoxy]benzoyl]-4-amino-thiazol-2-yl]-4-fluoro-anilino)propanamide (single stereoisomer),
2-(N-[4-amino-5-[4-[2-(4-chloroanilino)-2-oxo-ethoxy]benzoyl]thiazol-2-yl]-4-fluoro-anilino)propanamide (single stereoisomer),
4-[[2-[4-[4-amino-2-(4-fluoro-N-[2-amino-1-methyl-2-oxo-ethyl]anilino)thiazole-5-carbonyl]phenoxy]acetyl]amino]benzamide (single stereoisomer),
2-(N-[4-amino-5-[4-[2-((2RS),3-dihydroxypropylamino)-2-oxo-ethoxy]benzoyl]thiazol-2-yl]-4-fluoro-anilino)propanamide (mixture of two diastereomers),
2-(N-[4-amino-5-[4-[2-oxo-2-[2-(1-piperidyl)ethylamino]ethoxy]benzoyl]thiazol-2-yl]-4-fluoro-anilino)propanamide (single stereoisomer),
2-(N-[4-amino-5-[4-(2-amino-2-oxo-ethoxy)benzoyl]thiazol-2-yl]-4-fluoro-anilino)propanamide (single stereoisomer),
(R)-2-(N-[4-amino-5-[4-[2-(methylamino)-2-oxo-ethoxy]benzoyl]thiazol-2-yl]-4-fluoro-anilino)propanamide,
(S)-2-(N-[4-amino-5-[4-[2-(methyl amino)-2-oxo-ethoxy]benzoyl]thiazol-2-yl]-4-fluoro-anilino)propanamide,
(R)-2-(N-[4-amino-5-[4-[2-(isopropylamino)-2-oxo-ethoxy]benzoyl]thiazol-2-yi]-4-fluoro-anilino)propanamide,
(S)-2-(N-[4-amino-5-[4-[2-(isopropylamino)-2-oxo-ethoxy]benzoyl]thiazol-2-yl]-4-fluoro-anilino)propanamide,
rac-2-[4-[4-amino-2-(N-(2-amino-1-methyl-2-oxo-ethyl)-4-fluoro-anilino)thiazole-5-carbonyl]phenoxy]-N-isopropyl-2-methyl-propanamide,
rac-2-[4-[4-amino-2-(N-(2-amino-1-methyl-2-oxo-ethyl)-4-fluoro-anilino)thiazole-5-carbonyl]phenoxy]-2-methyl-propanamide,
rac-2-(N-(5-benzoyl-4-methyl-thiazol-2-yl)-4-fluoro-anilino)propanamide,
rac-2-(N-[4-amino-5-(4-hydroxybenzoyl)thiazol-2-yl]-4-fluoro-anilino)propanamide,
rac-2-(N-[4-amino-5-(4-benzyloxybenzoyl)thiazol-2-yl]-4-fluoro-anilino)propanamide,
rac-2-(N-[4-amino-5-[6-(difluoromethyl)pyridine-3-carbonyl]thiazol-2-yl]-4-fluoro-anilino)propanamide,
rac-2-(N-[4-amino-5-(pyridine-4-carbonyl)thiazol-2-yl]-3-chloro-4-fluoro-anilino)propanamide,
(R)-2-(N-[4-amino-5-(pyridine-4-carbonyl)thiazol-2-yl]-3-chloro-4-fluoro-anilino)propanamide,
(S)-2-(N-[4-amino-5-(pyridine-4-carbonyl)thiazol-2-yl]-3-chloro-4-fluoro-anilino)propanamide,
rac-2-(N-[4-amino-5-(4-methoxybenzoyl)thiazol-2-yl]-3-chloro-4-fluoro-anilino)propanamide,
(R)-2-(N-[4-amino-5-(4-methoxybenzoyl)thiazol-2-yl]-3-chloro-4-fluoro-anilino)propanamide,
(S)-2-(N-[4-amino-5-(4-methoxybenzoyl)thiazol-2-yl]-3-chloro-4-fluoro-anilino)propanamide,
rac-2-(N-[4-amino-5-[6-(difluoromethoxy)pyridine-3-carbonyl]thiazol-2-yl]-4-fluoro-anilino)propanamide,
(R)-2-(N-[4-amino-5-[6-(difluoromethoxy)pyridine-3-carbonyl]thiazol-2-yl]-4-fluoro-anilino)propanamide,
(S)-2-(N-[4-amino-5-[6-(difluoromethoxy)pyridine-3-carbonyl]thiazol-2-yl]-4-fluoro-anilino)propanamide,
rac-2-(N-[4-amino-5-[6-(difluoromethoxy)pyridine-3-carbonyl]thiazol-2-yl]-3,4-difluoro-anilino)propanamide,
(R)-(N-[4-amino-5-[6-(difluoromethoxy)pyridine-3-carbonyl]thiazol-2-yl]-3,4-difluoro-anilino)propanamide,
(S)-(N-[4-amino-5-[6-(difluoromethoxy)pyridine-3-carbonyl]thiazol-2-yl]-3,4-difluoro-anilino)propanamide,
rac-2-(N-[4-amino-5-[6-(difluoromethoxy)pyridine-3-carbonyl]thiazol-2-yl]-4-chloro-3-fluoro-anilino)propanamide,
(R)-2-(N-[4-amino-5-[6-(difluoromethoxy)pyridine-3-carbonyl]thiazol-2-yl]-4-chloro-3-fluoro-anilino)propanamide,
(S)-2-(N-[4-amino-5-[6-(difluoromethoxy)pyridine-3-carbonyl]thiazol-2-yl]-4-chloro-3-fluoro-anilino)propanamide,
rac-2-(N-[4-amino-5-(4-benzyloxybenzoyl)thiazol-2-yl]-3,4-difluoro-anilino)propanamide,
(R)-2-(N-[4-amino-5-(4-benzyloxybenzoyl)thiazol-2-yl]-3,4-difluoro-anilino)propanamide,
(S)-2-(N-[4-amino-5-(4-benzyloxybenzoyl)thiazol-2-yl]-3,4-difluoro-anilino)propanamide,
rac-2-(N-[4-amino-5-(4-benzyloxybenzoyl)thiazol-2-yl]-4-chloro-3-fluoro-anilino)propanamide,
(R)-2-(N-[4-amino-5-(4-benzyloxybenzoyl)thiazol-2-yl]-4-chloro-3-fluoro-anilino)propanamide,
(S)-2-(N-[4-amino-5-(4-benzyloxybenzoyl)thiazol-2-yl]-4-chloro-3-fluoro-anilino)propanamide,
rac-benzyl N-[4-[4-amino-2-(N-(2-amino-1-methyl-2-oxo-ethyl)-3,4-difluoro-anilino)thiazole-5-carbonyl]phenyl]carbamate,
(R)-benzyl N-[4-[4-amino-2-(N-(2-amino-1-methyl-2-oxo-ethyl)-3,4-difluoro-anilino)thiazole-5-carbonyl]phenyl]carbamate,
(S)-benzyl N-[4-[4-amino-2-(N-(2-amino-1-methyl-2-oxo-ethyl)-3,4-difluoro-anilino)thiazole-5-carbonyl]phenyl]carbamate,
rac-2-[4-[4-amino-2-(N-(2-amino-1-methyl-2-oxo-ethyl)-4-fluoro-anilino)thiazole-5-carbonyl]phenoxy]ethyl acetate,
rac-benzyl N-[4-[4-amino-2-(N-(2-amino-1-methyl-2-oxo-ethyl)-4-fluoro-anilino)thiazole-5-carbonyl]phenyl]carbamate,
(R)-benzyl N-[4-[4-amino-2-(N-(2-amino-1-methyl-2-oxo-ethyl)-4-fluoro-anilino)thiazole-5-carbonyl]phenyl]carbamate,
(S)-benzyl N-[4-[4-amino-2-(N-(2-amino-1-methyl-2-oxo-ethyl)-4-fluoro-anilino)thiazole-5-carbonyl]phenyl]carbamate,
rac-2-(N-[4-amino-5-(4-benzyloxybenzoyl)thiazol-2-yl]-4-fluoro-anilino)propanamide,
(R)-2-(N-[4-amino-5-(4-benzyloxybenzoyl)thiazol-2-yl]-4-fluoro-anilino)propanamide,
(S)-2-(N-[4-amino-5-(4-benzyloxybenzoyl)thiazol-2-yl]-4-fluoro-anilino)propanamide,
rac-2-(N-[4-amino-5-(4-iodobenzoyl)thiazol-2-yl]-4-fluoro-anilino)propanamide,
rac-2-(N-[4-amino-5-(6-methoxypyridine-3-carbonyl)thiazol-2-yl]-4-chloro-3-fluoro-anilino)propanamide,
(R)-2-(N-[4-amino-5-(6-methoxypyridine-3-carbonyl)thiazol-2-yl]-4-chloro-3-fluoro-anilino)propanamide,
(S)-2-(N-[4-amino-5-(6-methoxypyridine-3-carbonyl)thiazol-2-yl]-4-chloro-3-fluoro-anilino)propanamide,
rac-2-(N-[4-amino-5-(4-phenoxybenzoyl)thiazol-2-yl]-4-fluoro-anilino)propanamide,
rac-2-(N-[4-amino-5-[4-(difluoromethoxy)benzoyl]thiazol-2-yl]-4-methoxy-anilino)propanamide,
(R)-2-(N-[4-amino-5-[4-(difluoromethoxy)benzoyl]thiazol-2-yl]-4-methoxy-anilino)propanamide,
(S)-2-(N-[4-amino-5-[4-(difluoromethoxy)benzoyl]thiazol-2-yl]-4-methoxy-anilino)propanamide,
rac-2-(N-[4-amino-5-(4-nitrobenzoyl)thiazol-2-yl]-4-fluoro-anilino)propanamide,
rac-2-(4-fluoro-N-[5-(4-methoxybenzoyl)-4-methyl-thiazol-2-yl]anilino)propanamide,
(R)-2-(4-fluoro-N-[5-(4-methoxybenzoyl)-4-methyl-thiazol-2-yl]anilino)propanamide,
(S)-2-(4-fluoro-N-[5-(4-methoxybenzoyl)-4-methyl-thiazol-2-yl]anilino)propanamide,
rac-4-[4-[4-amino-2-(N-(2-amino-1-methyl-2-oxo-ethyl)-4-fluoro-anilino)thiazole-5-carbonyl]phenyl]cyclopropanecarboxamide,
rac-2-(N-[4-amino-5-(4-morpholinobenzoyl)thiazol-2-yl]-4-fluoro-anilino)propanamide,
rac-2-(N-[4-amino-5-[4-(pyrazol-1-ylmethyl)benzoyl]thiazol-2-yl]-4-fluoro-anilino)propanamide,
rac-2-(N-[4-amino-5-[4-(dimethylamino)benzoyl]thiazol-2-yl]-4-fluoro-anilino)propanamide,
rac-2-(N-[4-amino-5-(4-pyrrolidin-1-ylbenzoyl)thiazol-2-yl]-4-fluoro-anilino)propanamide,
rac-2-(N-[4-amino-5-[4-(difluoromethoxy)benzoyl]thiazol-2-yl]-4-benzyloxy-anilino)propanamide,
(R)-2-(N-[4-amino-5-[4-(difluoromethoxy)benzoyl]thiazol-2-yl]-4-benzyloxy-anilino)propanamide,
(S)-2-(N-[4-amino-5-[4-(difluoromethoxy)benzoyl]thiazol-2-yl]-4-benzyloxy-anilino)propanamide,
rac-2-(N-[4-amino-5-[3-(difluoromethoxy)benzoyl]thiazol-2-yl]-4-fluoro-anilino)propanamide,
rac-2-(N-[4-amino-5-(pyridine-3-carbonyl)thiazol-2-yl]-3,4-difluoro-anilino)propanamide,
(R)-2-(N-[4-amino-5-(pyridine-3-carbonyl)thiazol-2-yl]-3,4-difluoro-anilino)propanamide,
(S)-2-(N-[4-amino-5-(pyridine-3-carbonyl)thiazol-2-yl]-3,4-difluoro-anilino)propanamide,
rac-2-(N-[5-(4-acetamidobenzoyl)-4-amino-thiazol-2-yl]-4-fluoro-anilino)propanamide,
rac-2-(N-[4-amino-5-(2-chloropyridine-4-carbonyl)thiazol-2-yl]-4-fluoro-anilino)propanamide,
rac-2-(N-[4-amino-5-(2-methylpyridine-4-carbonyl)thiazol-2-yl]-4-fluoro-anilino)propanamide,
(R)-2-(N-[4-amino-5-(2-methylpyridine-4-carbonyl)thiazol-2-yl]-4-fluoro-anilino)propanamide,
(S)-2-(N-[4-amino-5-(2-methylpyridine-4-carbonyl)thiazol-2-yl]-4-fluoro-anilino)propanamide,
rac-2-(N-[4-amino-5-[2-(difluoromethyl)pyridine-4-carbonyl]thiazol-2-yl]-4-fluoro-anilino)propanamide,
rac-2-[(4-amino-5-benzoyl-thiazol-2-yl)-(4-pyridyl)amino]propanamide,
rac-2-(N-[4-amino-5-(2-methoxypyridine-4-carbonyl)thiazol-2-yl]-4-fluoro-anilino)propanamide,
rac-2-(N-(4-amino-5-benzoyl-thiazol-2-yl)-3-fluoro-4-methoxy-anilino)propanamide,
rac-2-(N-(4-amino-5-benzoyl-thiazol-2-yl)-4-cyano-3-fluoro-anilino)propanamide,
rac-2-(N-(4-amino-5-benzoyl-thiazol-2-yl)-4-bromo-anilino)propanamide,
rac-2-[N-(4-amino-5-benzoyl-thiazol-2-yl)-3-chloro-4-(difluoromethoxy)anilino]propanamide,
rac-2-(N-(4-amino-5-benzoyl-thiazol-2-yl)-4-ethoxy-anilino)propanamide,
rac-2-[(4-amino-5-benzoyl-thiazol-2-yl)-(1,3-benzodioxol-5-yl)amino]propanamide,
rac-2-[(4-amino-5-benzoyl-thiazol-2-yl)-(2,2-difluoro-1,3-benzodioxol-5-yl)amino]propanamide,
rac-2-[N-(4-amino-5-benzoyl-thiazol-2-yl)-4-(difluoromethoxy)-3-fluoro-anilino]propanamide,
rac-2-(N-(4-amino-5-benzoyl-thiazol-2-yl)-4-benzyloxy-anilino)propanamide,
rac-2-[N-[4-amino-5-(4-methoxybenzoyl)thiazol-2-yl]-3-chloro-4-(difluoromethoxy)anilino]propanamide,
rac-2-[N-[4-amino-5-[4-(difluoromethoxy)benzoyl]thiazol-2-yl]-3-chloro-4-(difluoromethoxy)anilino]propanamide,
rac-2-[N-[4-amino-5-(4-chlorobenzoyl)thiazol-2-yl]-3-chloro-4-(difluoromethoxy)anilino]propanamide,
rac-2-[N-[4-amino-5-(pyridine-4-carbonyl)thiazol-2-yl]-3-chloro-4-(difluoromethoxy)anilino]propanamide,
rac-2-[N-[4-amino-5-[6-(difluoromethoxy)pyridine-3-carbonyl]thiazol-2-yl]-3-chloro-4-(difluoromethoxy)anilino]propanamide,
rac-2-[N-[4-amino-5-(4-methoxybenzoyl)thiazol-2-yl]-4-(difluoromethoxy)-3-fluoro-anilino]propanamide,
rac-2-[N-[4-amino-5-(4-chlorobenzoyl)thiazol-2-yl]-4-(difluoromethoxy)-3-fluoro-anilino]propanamide,
rac-2-[N-[4-amino-5-[4-(difluoromethoxy)benzoyl]thiazol-2-yl]-4-(difluoromethoxy)-3-fluoro-anilino]propanamide,
rac-2-[N-[4-amino-5-(pyridine-4-carbonyl)thiazol-2-yl]-4-(difluoromethoxy)-3-fluoro-anilino]propanamide,
rac-2-[N-[4-amino-5-[6-(difluoromethoxy)pyridine-3-carbonyl]thiazol-2-yl]-4-(difluoromethoxy)-3-fluoro-anilino]propanamide,
rac-2-[N-(4-amino-5-benzoyl-thiazol-2-yl)-3-chloro-4-(trifluoromethoxy)anilino]propanamide,
rac-2-[[4-amino-5-(4-methoxybenzoyl)thiazol-2-yl]-(2,2-difluoro-1,3-benzodioxol-5-yl)amino]propanamide,
rac-2-[[4-amino-5-(4-chlorobenzoyl)thiazol-2-yl]-(2,2-difluoro-1,3-benzodioxol-5-yl)amino]propanamide,
rac-2-[[4-amino-5-[4-(difluoromethoxy)benzoyl]thiazol-2-yl]-(2,2-difluoro-1,3-benzodioxol-5-yl)amino]propanamide,
rac-2-[[4-amino-5-(pyridine-4-carbonyl)thiazol-2-yl]-(2,2-difluoro-1,3-benzodioxol-5-yl)amino]propanamide,
rac-2-[[4-amino-5-[6-(difluoromethoxy)pyridine-3-carbonyl]thiazol-2-yl]-(2,2-difluoro-1,3-benzodioxol-5-yl)amino]propanamide,
rac-2-[N-[4-amino-5-(4-methoxybenzoyl)thiazol-2-yl]-3-fluoro-4-(trifluoromethoxy)anilino]propanamide,
rac-2-[N-[4-amino-5-(4-chlorobenzoyl)thiazol-2-yl]-3-fluoro-4-(trifluoromethoxy)anilino]propanamide,
rac-2-[N-[4-amino-5-[4-(difluoromethoxy)benzoyl]thiazol-2-yl]-3-fluoro-4-(trifluoromethoxy)anilino]propanamide,
rac-2-[N-[4-amino-5-(pyridine-4-carbonyl)thiazol-2-yl]-3-fluoro-4-(trifluoromethoxy)anilino]propanamide,
rac-2-[N-[4-amino-5-[6-(difluoromethoxy)pyridine-3-carbonyl]thiazol-2-yl]-3-fluoro-4-(trifluoromethoxy)anilino]propanamide,
rac-2-[N-[4-amino-5-(4-methoxybenzoyl)thiazol-2-yl]-3-chloro-4-(trifluoromethoxy)anilino]propanamide,
rac-2-[N-[4-amino-5-(4-chlorobenzoyl)thiazol-2-yl]-3-chloro-4-(trifluoromethoxy)anilino]propanamide,
rac-2-[N-[4-amino-5-[4-(difluoromethoxy)benzoyl]thiazol-2-yl]-3-chloro-4-(trifluoromethoxy)anilino]propanamide,
rac-2-[N-[4-amino-5-(pyridine-4-carbonyl)thiazol-2-yl]-3-chloro-4-(trifluoromethoxy)anilino]propanamide,
rac-2-[N-[4-amino-5-[6-(difluoromethoxy)pyridine-3-carbonyl]thiazol-2-yl]-3-chloro-4-(trifluoromethoxy)anilino]propanamide,
rac-2-[(4-amino-5-benzoyl-thiazol-2-yl)-(6-methoxy-3-pyridyl)amino]propanamide,
rac-2-[(4-amino-5-benzoyl-thiazol-2-yl)-[6-(trifluoromethoxy)-3-pyridyl]amino]propanamide,
rac-2-[(4-amino-5-benzoyl-thiazol-2-yl)-[6-(difluoromethoxy)-3-pyridyl]amino]propanamide,
rac-2-[(4-amino-5-benzoyl-thiazol-2-yl)-[6-(trifluoromethyl)-3-pyridyl]amino]propanamide,
rac-2-[(4-amino-5-benzoyl-thiazol-2-yl)-[6-(difluoromethyl)-3-pyridyl]amino]propanamide,
rac-2-[(4-amino-5-benzoyl-thiazol-2-yl)-(6-chloro-3-pyridyl)amino]propanamide,
rac-2-[(4-amino-5-benzoyl-thiazol-2-yl)-(6-fluoro-3-pyridyl)amino]propanamide,
rac-2-[(4-amino-5-benzoyl-thiazol-2-yl)-(6-methyl-3-pyridyl)amino]propanamide,
rac-2-[N-(4-amino-5-benzoyl-thiazol-2-yl)-4-fluoro-3-(trifluoromethoxy)anilino]propanamide,
rac-2-[N-(4-amino-5-benzoyl-thiazol-2-yl)-4-chloro-3-(trifluoromethoxy)anilino]propanamide,
rac-2-[N-(4-amino-5-benzoyl-thiazol-2-yl)-3-(difluoromethoxy)-4-fluoro-anilino]propanamide,
rac-2-[N-(4-amino-5-benzoyl-thiazol-2-yl)-4-chloro-3-(difluoromethoxy)anilino]propanamide,
rac-2-[N-(4-amino-5-benzoyl-thiazol-2-yl)-2-fluoro-4-(trifluoromethoxy)anilino]propanamide,
rac-2-[N-(4-amino-5-benzoyl-thiazol-2-yl)-2-chloro-4-(trifluoromethoxy)anilino]propanamide,
rac-2-[N-(4-amino-5-benzoyl-thiazol-2-yl)-4-(difluoromethoxy)-2-fluoro-anilino]propanamide,
rac-2-[N-(4-amino-5-benzoyl-thiazol-2-yl)-2-chloro-4-(difluoromethoxy)anilino]propanamide,
rac-2-[N-[4-amino-5-(4-methoxybenzoyl)thiazol-2-yl]-4-(difluoromethyl)anilino]propanamide,
rac-2-[N-[4-amino-5-(4-chlorobenzoyl)thiazol-2-yl]-4-(difluoromethyl)anilino]propanamide,
rac-2-[N-[4-amino-5-[4-(difluoromethoxy)benzoyl]thiazol-2-yl]-4-(difluoromethyl)anilino]propanamide,
rac-2-[N-[4-amino-5-(pyridine-4-carbonyl)thiazol-2-yl]-4-(difluoromethyl)anilino]propanamide,
rac-2-[N-[4-amino-5-[6-(difluoromethoxy)pyridine-3-carbonyl]thiazol-2-yl]-4-(difluoromethyl)anilino]propanamide,
rac-2-[[4-amino-5-(4-methoxybenzoyl)thiazol-2-yl]-[6-(trifluoromethoxy)-3-pyridyl]amino]propanamide,
rac-2-[[4-amino-5-(4-chlorobenzoyl)thiazol-2-yl]-[6-(trifluoromethoxy)-3-pyridyl]amino]propanamide,
rac-2-[[4-amino-5-[4-(difluoromethoxy)benzoyl]thiazol-2-yl]-[6-(trifluoromethoxy)-3-pyridyl]amino]propanamide,
rac-2-[[4-amino-5-[6-(difluoromethoxy)pyridine-3-carbonyl]thiazol-2-yl]-[6-(trifluoromethoxy)-3-pyridyl]amino]propanamide,
rac-2-[[4-amino-5-(4-methoxybenzoyl)thiazol-2-yl]-[6-(difluoromethoxy)-3-pyridyl]amino]propanamide,
rac-2-[[4-amino-5-(4-chlorobenzoyl)thiazol-2-yl]-[6-(difluoromethoxy)-3-pyridyl]amino]propanamide,
rac-2-[[4-amino-5-[4-(difluoromethoxy)benzoyl]thiazol-2-yl]-[6-(difluoromethoxy)-3-pyridyl]amino]propanamide,
rac-2-[[4-amino-5-[6-(difluoromethoxy)pyridine-3-carbonyl]thiazol-2-yl]-[6-(difluoromethoxy)-3-pyridyl]amino]propanamide,
rac-benzyl N-[5-[4-amino-2-(N-(2-amino-1-methyl-2-oxo-ethyl)-4-fluoro-anilino)thiazole-5-carbonyl]-2-pyridyl]carbamate,
rac-Ethyl 4-[4-amino-2-(N-(2-amino-1-methyl-2-oxo-ethyl)-4-fluoro-anilino)thiazole-5-carbonyl]benzoate,
rac-Ethyl 4-[4-amino-2-(N-(2-amino-1-methyl-2-oxo-ethyl)-4-chloro-3-fluoro-anilino)thiazole-5-carbonyl]benzoate,
rac-Ethyl 2-[4-[4-amino-2-(N-(2-amino-1-methyl-2-oxo-ethyl)-4-fluoro-anilino)thiazole-5-carbonyl]phenoxy]-2-methyl-propanoate,
rac-4-[4-amino-2-(N-(2-amino-1-methyl-2-oxo-ethyl)-4-fluoro-anilino)thiazole-5-carbonyl]-N-cyclohexyl-benzamide,
rac-4-[4-amino-2-(N-(2-amino-1-methyl-2-oxo-ethyl)-4-fluoro-anilino)thiazole-5-carbonyl]-N-isopropyl-benzamide,
rac-4-[4-amino-2-(N-(2-amino-1-methyl-2-oxo-ethyl)-4-fluoro-anilino)thiazole-5-carbonyl]-N-benzyl-benzamide,
4-[4-amino-2-(N-(2-amino-1-methyl-2-oxo-ethyl)-4-fluoro-anilino)thiazole-5-carbonyl]-N-[(2S)-2-hydroxypropyl]benzamide (mixture of stereoisomers),
rac-4-[4-amino-2-(N-(2-amino-1-methyl-2-oxo-ethyl)-4-fluoro-anilino)thiazole-5-carbonyl]-N-(2-methoxyethyl)benzamide,
4-[4-amino-2-(N-(2-amino-1-methyl-2-oxo-ethyl)-4-fluoro-anilino)thiazole-5-carbonyl]-N-[(2R)-2-hydroxypropyl]benzamide (mixture of stereoisomers),
rac-4-[4-amino-2-(N-(2-amino-1-methyl-2-oxo-ethyl)-4-fluoro-anilino)thiazole-5-carbonyl]-N-cyclopropyl-benzamide,
rac-4-[4-amino-2-(N-(2-amino-1-methyl-2-oxo-ethyl)-4-fluoro-anilino)thiazole-5-carbonyl]-N-cyclopentyl-benzamide,
rac-4-[4-amino-2-(N-(2-amino-1-methyl-2-oxo-ethyl)-4-chloro-3-fluoro-anilino)thiazole-5-carbonyl]-N-(2-phenoxyethyl)benzamide,
rac-4-[4-amino-2-(N-(2-amino-1-methyl-2-oxo-ethyl)-4-chloro-3-fluoro-anilino)thiazole-5-carbonyl]-N-[2-(trifluoromethoxy)ethyl]benzamide,
rac-4-[4-amino-2-(N-(2-amino-1-methyl-2-oxo-ethyl)-4-chloro-3-fluoro-anilino)thiazole-5-carbonyl]-N-[2-(difluoromethoxy)ethyl]benzamide,
rac-4-[4-amino-2-(N-(2-amino-1-methyl-2-oxo-ethyl)-4-chloro-3-fluoro-anilino)thiazole-5-carbonyl]-N-(2-tert-butoxyethyl)benzamide,
rac-4-[4-amino-2-(N-(2-amino-1-methyl-2-oxo-ethyl)-4-chloro-3-fluoro-anilino)thiazole-5-carbonyl]-N-(2-methoxyethyl)benzamide,
rac-2-[4-[4-amino-2-(N-(2-amino-1-methyl-2-oxo-ethyl)-4-fluoro-anilino)thiazole-5-carbonyl]phenoxy]-N-[(4-chlorophenyl)methyl]-2-methyl-propanamide,
rac-2-(N-[4-amino-5-(6-bromopyridine-3-carbonyl)thiazol-2-yl]-4-fluoro-anilino)propanamide,
rac-2-(N-[4-amino-5-[6-[4-(trifluoromethyl)-1-piperidyl]pyridine-3-carbonyl]thiazol-2-yl]-4-fluoro-anilino)propanamide,
rac-2-(N-[4-amino-5-[6-(4-methyl-1-piperidyl)pyridine-3-carbonyl]thiazol-2-yl]-4-fluoro-anilino)propanamide,
rac-2-(N-[4-amino-5-[6-[4-(oxetan-3-yl)-1-piperidyl]pyridine-3-carbonyl]thiazol-2-yl]-4-fluoro-anilino)propanamide,
rac-2-(N-[4-amino-5-[6-(dimethylamino)pyridine-3-carbonyl]thiazol-2-yl]-4-fluoro-anilino)propanamide,
rac-2-(N-[4-amino-5-[6-(4,4-dimethyl-1-piperidyl)pyridine-3-carbonyl]thiazol-2-yl]-4-fluoro-anilino)propanamide,
rac-2-(N-[4-amino-5-[6-(3-azabicyclo[3.2.1]octan-3-yl)pyridine-3-carbonyl]thiazol-2-yl]-4-fluoro-anilino)propanamide,
rac-2-(N-[4-amino-5-[6-(3,5-dimethyl-1-piperidyl)pyridine-3-carbonyl]thiazol-2-yl]-4-fluoro-anilino)propanamide,
rac-2-(N-[4-amino-5-[6-(3-azabicyclo[3.1.0]hexan-3-yl)pyridine-3-carbonyl]thiazol-2-yl]-4-fluoro-anilino)propanamide,
rac-2-(N-[4-amino-5-[6-(1-piperidyl)pyridine-3-carbonyl]thiazol-2-yl]-4-fluoro-anilino)propanamide,
rac-2-(N-[4-amino-5-[6-(4,4-difluoro-1-piperidyl)pyridine-3-carbonyl]thiazol-2-yl]-4-fluoro-anilino)propanamide,
rac-2-(N-[4-amino-5-[6-(4-cyano-4-methyl-1-piperidyl)pyridine-3-carbonyl]thiazol-2-yl]-4-fluoro-anilino)propanamide,
(R)-2-(N-[4-amino-5-[6-(4-cyano-4-methyl-1-piperidyl)pyridine-3-carbonyl]thiazol-2-yl]-4-fluoro-anilino)propanamide,
(S)-2-(N-[4-amino-5-[6-(4-cyano-4-methyl-1-piperidyl)pyridine-3-carbonyl]thiazol-2-yl]-4-fluoro-anilino)propanamide,
2-(N-[4-amino-5-(4-hydroxybenzoyl)thiazol-2-yl]-4-fluoro-anilino)propanamide (single enantiomer),
(R)-2-(N-[4-amino-5-(4-hydroxybenzoyl)thiazol-2-yl]-3,4-difluoro-anilino)propanamide,
(S)-2-(N-[4-amino-5-(4-hydroxybenzoyl)thiazol-2-yl]-3,4-difluoro-anilino)propanamide,
2-(N-[4-amino-5-(4-hydroxybenzoyl)thiazol-2-yl]-4-chloro-3-fluoro-anilino)propanamide (enantiomer 1),
2-(N-[4-amino-5-(4-hydroxybenzoyl)thiazol-2-yl]-4-chloro-3-fluoro-anilino)propanamide (enantiomer 2),
2-(N-[4-amino-5-[4-(difluoromethoxy)benzoyl]thiazol-2-yl]-4-hydroxy-anilino)propanamide (single enantiomer), and
rac-2-(N-[4-amino-5-[4-(2-hydroxyethoxy)benzoyl]thiazol-2-yl]-4-fluoro-anilino)propanamide, or a stereoisomer, a tautomer, an N-oxide, a hydrate, a solvate, or a salt thereof, or a mixture of same.

In some preferred embodiments, the inhibitor of DGKzeta is a compound according to this list.

### DGKzeta Inhibitor A′

In a preferred embodiment of the present invention, the inhibitor of DGKzeta is (R)-2-(N-[4-amino-5-(4-methoxybenzoyl)thiazol-2-yl]-4-fluoro-anilino)propanamide, or a stereoisomer, a tautomer, an N-oxide, a hydrate, a solvate, or a salt thereof, or a mixture of same.

In another preferred embodiment of the present invention, the inhibitor of DGKzeta is (R)-2-(N-[4-amino-5-(4-methoxybenzoyl)thiazol-2-yl]-4-fluoro-anilino)propanamide, or a tautomer, an N-oxide, a hydrate, a solvate, or a salt thereof, or a mixture of same.

In another preferred embodiment of the present invention, the inhibitor of DGKzeta is (R)-2-(N-[4-amino-5-(4-methoxybenzoyl)thiazol-2-yl]-4-fluoro-anilino)propanamide.

In another preferred embodiment of the present invention, the inhibitor of DGKzeta is DGKzeta inhibitor A′ of structure

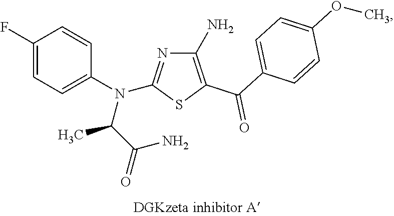 [SMILES: COc1ccc(C(=O)c2sc(N(c3ccc(F)cc3)[C@H](C)C(N)=O)nc2N)cc1]

or a stereoisomer, a tautomer, an N-oxide, a hydrate, a solvate, or a salt thereof, or a mixture of same.

In another preferred embodiment of the present invention, the inhibitor of DGKzeta is DGKzeta inhibitor A′ of structure

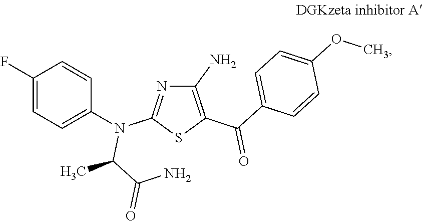 [SMILES: COc1ccc(C(=O)c2sc(N(c3ccc(F)cc3)[C@H](C)C(N)=O)nc2N)cc1]

or a tautomer, an N-oxide, a hydrate, a solvate, or a salt thereof, or a mixture of same.

In another preferred embodiment of the present invention, the DGKzeta inhibitor is DGKzeta inhibitor A′ of structure

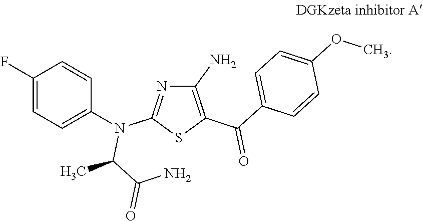 [SMILES: COc1ccc(C(=O)c2sc(N(c3ccc(F)cc3)[C@H](C)C(N)=O)nc2N)cc1]

The synthesis of DGKzeta inhibitor A′ is described in the International Patent Application PCT/EP2021/060167, Example 62.2. International Patent Application PCT/EP2021/060167 also discloses methods of preparing other compounds of formula (II) mentioned herein.

### EMBODIMENTS

### Embodiments of Combinations

### Aspect 1

In accordance with a first aspect, the present invention provides combinations comprising one or more inhibitors of DGKalpha and one or more inhibitors of DGKzeta.

In another embodiment, the present invention provides combinations comprising one inhibitor of DGKalpha and one inhibitor of DGKzeta.

In another embodiment, the present invention provides combinations comprising one DGKalpha inhibitor compound of general formula (I) described herein and one DGKzeta inhibitor compound of general formula (II) described herein, or a stereoisomer, a tautomer, an N-oxide, a hydrate, a solvate, or a salt thereof, or a mixture of same.

In another embodiment, the present invention provides combinations comprising one DGKalpha inhibitor compound of general formula (I) described herein and one DGKzeta inhibitor compound of general formula (II) described herein.

In another embodiment, the present invention provides combinations comprising DGKalpha inhibitor A and DGKzeta inhibitor A′ described herein, or a stereoisomer, a tautomer, an N-oxide, a hydrate, a solvate, or a salt thereof, or a mixture of same.

In another embodiment, the present invention provides combinations comprising DGKalpha inhibitor A and DGKzeta inhibitor A′ described herein.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 20:1 to 1:20.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 12:1 to 1:12.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 12:1 to 8:1.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 10:1 to 1:1.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 10:1.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 4:1 to 2:1.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 3:1.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 2:1 to 1:2.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 1:1.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 1:2 to 1:4.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 1:3.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 1:8 to 1:12.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 1:1 to 1:10.

In another embodiment, the present invention provides combinations comprising DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 1:10.

In another embodiment, the present invention provides combinations consisting of one or more inhibitors of DGKalpha and one or more inhibitors of DGKzeta.

In another embodiment, the present invention provides combinations consisting of one inhibitor of DGKalpha and one inhibitor of DGKzeta.

In another embodiment, the present invention provides combinations consisting of one DGKalpha inhibitor compound of general formula (I) described herein and one DGKzeta inhibitor compound of general formula (II) described herein, or a stereoisomer, a tautomer, an N-oxide, a hydrate, a solvate, or a salt thereof, or a mixture of same.

In another embodiment, the present invention provides combinations consisting of one DGKalpha inhibitor compound of general formula (I) described herein and one DGKzeta inhibitor compound of general formula (II) described herein.

In a preferred embodiment, the present invention provides combinations consisting of DGKalpha inhibitor A and DGKzeta inhibitor A′ described herein, or a stereoisomer, a tautomer, an N-oxide, a hydrate, a solvate, or a salt thereof, or a mixture of same.

In another preferred embodiment, the present invention provides combinations consisting of DGKalpha inhibitor A and DGKzeta inhibitor A′ described herein, or a tautomer, an N-oxide, a hydrate, a solvate, or a salt thereof, or a mixture of same.

In another preferred embodiment, the present invention provides combinations consisting of DGKalpha inhibitor A and DGKzeta inhibitor A′ described herein.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 20:1 to 1:20.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 12:1 to 1:12.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 12:1 to 8:1.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′ is in the range from 10:1 to 1:1.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 10:1.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 4:1 to 2:1.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 3:1.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 2:1 to 1:2.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 1:1.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 1:2 to 1:4.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 1:3.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 1:8 to 1:12.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is in the range from 1:1 to 1:10.

In another embodiment, the present invention provides combinations consisting of DGKalpha inhibitor A and DGKzeta inhibitor A′ described herein, in which the dose ratio (mg or mg/kg of DGKalpha inhibitor A:mg or mg/kg of DGKzeta inhibitor A′) is approximately 1:10.

In another preferred embodiment, the combination of the present invention comprises DGKalpha inhibitor A and DGKzeta inhibitor A′, or a stereoisomer, a tautomer, an N-oxide, a hydrate, a solvate, or a salt thereof, or a mixture of same.

In another preferred embodiment, the combination of the present invention consists of DGKalpha inhibitor A and DGKzeta inhibitor A′, or a stereoisomer, a tautomer, an N-oxide, a hydrate, a solvate, or a salt thereof, or a mixture of same.

In another preferred embodiment, the combination of the present invention comprises DGKalpha inhibitor A and DGKzeta inhibitor A′.

In another preferred embodiment, the combination of the present invention consists of DGKalpha inhibitor A and DGKzeta inhibitor A′.

In another preferred embodiment, the combination of the present invention comprises 6-fluoro-1-methyl-4-[4-(5-methyl-1,3-benzoxazol-2-yl)piperidin-1-yl]-2-oxo-1,2-dihydroquinoline-3-carboxamide and (R)-2-(N-[4-amino-5-(4-methoxybenzoyl)thiazol-2-yl]-4-fluoro-anilino)propanamide, or a stereoisomer, a tautomer, an N-oxide, a hydrate, a solvate, or a salt thereof, or a mixture of same.

In another preferred embodiment, the combination of the present invention consists of 6-fluoro-1-methyl-4-[4-(5-methyl-1,3-benzoxazol-2-yl)piperidin-1-yl]-2-oxo-1,2-dihydroquinoline-3-carboxamide and (R)-2-(N-[4-amino-5-(4-methoxybenzoyl)thiazol-2-yl]-4-fluoro-anilino)propanamide, or a stereoisomer, a tautomer, an N-oxide, a hydrate, a solvate, or a salt thereof, or a mixture of same.

In another preferred embodiment, the combination of the present invention comprises 6-fluoro-1-methyl-4-[4-(5-methyl-1,3-benzoxazol-2-yl)piperidin-1-yl]-2-oxo-1,2-dihydroquinoline-3-carboxamide and (R)-2-(N-[4-amino-5-(4-methoxybenzoyl)thiazol-2-yl]-4-fluoro-anilino)propanamide.

In another preferred embodiment, the combination of the present invention consists of 6-fluoro-1-methyl-4-[4-(5-methyl-1,3-benzoxazol-2-yl)piperidin-1-yl]-2-oxo-1,2-dihydroquinoline-3-carboxamide and (R)-2-(N-[4-amino-5-(4-methoxybenzoyl)thiazol-2-yl]-4-fluoro-anilino)propanamide.

The combinations of the present invention may be administered by the oral, intravenous, topical, local installations, intraperitoneal or nasal route.

The inhibitor of DGKalpha may be administered by the oral, intravenous, topical, local installations, intraperitoneal or nasal route.

The inhibitor of DGKzeta may be administered by the oral, intravenous, topical, local installations, intraperitoneal or nasal route.

The inhibitor of DGKalpha may be in the form of a pharmaceutical formulation which is ready for use to be administered simultaneously, concurrently, separately or sequentially with the inhibitor of DGKzeta and optionally component C as further described infro. The inhibitor of DGKalpha and the inhibitor of DGKzeta and optionally component C may be administered independently of one another by the oral, intravenous, topical, local installations, intraperitoneal or nasal route.

The combinations comprising one or more inhibitors of DGKalpha and one or more inhibitors of DGKzeta, as described and defined herein, are also referred to as “combinations of the present invention”.

The surprising behavior of a combination of the present invention is demonstrated herein with DGK inhibitors (DGKalpha inhibitor A and DGKzeta inhibitor A′), specifically disclosed in the Examples section.

### Embodiments of Kits

### Aspect 2

In accordance with a second aspect, the present invention provides a kit comprising:
One DGKalpha inhibitor compound of general formula (I) as described herein, more particularly DGKalpha inhibitor A, or a stereoisomer, a tautomer, an N-oxide, a hydrate, a solvate, or a salt thereof, or a mixture of same,
One DGKzeta inhibitor compound of general formula (II) as described herein, more particularly DGKzeta inhibitor A′, or a stereoisomer, a tautomer, an N-oxide, a hydrate, a solvate, or a salt thereof, or a mixture of same.

In another preferred embodiment, the present invention provides a kit comprising:
DGKalpha inhibitor A, or a tautomer, an N-oxide, a hydrate, a solvate, or a salt thereof, or a mixture of same,
DGKzeta inhibitor A′, or a stereoisomer, a tautomer, an N-oxide, a hydrate, a solvate, or a salt thereof, or a mixture of same.

In another preferred embodiment, the present invention provides a kit comprising:
DGKalpha inhibitor A, or a tautomer, an N-oxide, a hydrate, a solvate, or a salt thereof, or a mixture of same,
DGKzeta inhibitor A′, or a tautomer, an N-oxide, a hydrate, a solvate, or a salt thereof, or a mixture of same.

In another preferred embodiment, the present invention provides a kit comprising:
DGKalpha inhibitor A,
DGKzeta inhibitor A′.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is in the range from 20:1 to 1:20.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is in the range from 12:1 to 1:12.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is in the range from 12:1 to 8:1.

In another preferred embodiment, the present invention provides a kit comprising: DGKalpha inhibitor A,
DGKzeta inhibitor A′,
in which the dose ratio (mg or mg/kg of DGKalpha inhibitor A:mg or mg/kg of DGKzeta inhibitor A′) is in the range from 10:1 to 1:1.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is approximately 10:1.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is in the range from 4:1 to 2:1.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is approximately 3:1.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is in the range from 2:1 to 1:2.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is approximately 1:1.

In another preferred embodiment, the present invention provides a kit comprising: DGKalpha inhibitor A,
DGKzeta inhibitor A′,
in which the dose ratio (mg or mg/kg of DGKalpha inhibitor A:mg or mg/kg of DGKzeta inhibitor A′) is in the range from 1:2 to 1:4.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is approximately 1:3.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is in the range from 1:8 to 1:12.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is in the range from 1:1 to 1:10.

In another preferred embodiment, the present invention provides a kit comprising:
DGKalpha inhibitor A,
DGKzeta inhibitor A′,
in which the dose ratio (mg or mg/kg of DGKalpha inhibitor A:mg or mg/kg of DGKzeta inhibitor A′) is approximately 1:10.

In the kit optionally either or both of said one DGKalpha inhibitor compound and said one DGKzeta inhibitor compound in any of the above-mentioned combinations are in the form of a pharmaceutical composition which is ready for use to be administered simultaneously, concurrently, separately or sequentially. Said one DGKalpha inhibitor compound and said one DGKzeta inhibitor compound may be administered independently of one another by the oral, intravenous, topical, local installations, intraperitoneal or nasal route. Preferably said one DGKalpha inhibitor compound and said one DGKzeta inhibitor compound are both administered by the oral route or said one DGKalpha inhibitor compound is administered by the oral route and said one DGKzeta inhibitor compound is administered by the intravenous route, or vice versa.

In another embodiment, the present invention provides a kit comprising:
One DGKalpha inhibitor compound of general formula (I) as described herein, more particularly DGKalpha inhibitor A, or a stereoisomer, a tautomer, an N-oxide, a hydrate, a solvate, or a salt thereof, or a mixture of same,
One DGKzeta inhibitor compound of general formula (II) as described herein, more particularly DGKzeta inhibitor A′, or a stereoisomer, a tautomer, an N-oxide, a hydrate, a solvate, or a salt thereof, or a mixture of same, and, optionally,
component C: one or more, preferably one, further pharmaceutical agent(s), and/or CAR-T cells, in which optionally either or all of said one DGKalpha inhibitor compound, said one DGKzeta inhibitor compound, and said component C in any of the above-mentioned combinations are in the form of a pharmaceutical composition which is ready for use to be administered simultaneously, concurrently, separately or sequentially. Said one DGKalpha inhibitor compound, said one DGKzeta inhibitor compound, and optionally component C, may be administered independently of one another by the oral, intravenous, topical, local installations, intraperitoneal or nasal route.

In another embodiment, the present invention provides a kit comprising:
DGKalpha inhibitor A, or a tautomer, an N-oxide, a hydrate, a solvate, or a salt thereof, or a mixture of same,
DGKzeta inhibitor A′, or a stereoisomer, a tautomer, an N-oxide, a hydrate, a solvate, or a salt thereof, or a mixture of same, and, optionally,
component C: one or more, preferably one, further pharmaceutical agent(s), and/or CAR-T cells, in which optionally either or all of said DGKalpha inhibitor A, or a tautomer, an N-oxide, a hydrate, a solvate, or a salt thereof, or a mixture of same, said DGKzeta inhibitor A′, or a stereoisomer, a tautomer, an N-oxide, a hydrate, a solvate, or a salt thereof, or a mixture of same, and said component C in any of the above-mentioned combinations are in the form of a pharmaceutical composition which is ready for use to be administered simultaneously, concurrently, separately or sequentially. Said DGKalpha inhibitor A, or a tautomer, an N-oxide, a hydrate, a solvate, or a salt thereof, or a mixture of same, said DGKzeta inhibitor A′, or a stereoisomer, a tautomer, an N-oxide, a hydrate, a solvate, or a salt thereof, or a mixture of same, and optionally component C, may be administered independently of one another by the oral, intravenous, topical, local installations, intraperitoneal or nasal route.

In another embodiment, the present invention provides a kit comprising:
DGKalpha inhibitor A,
DGKzeta inhibitor A′, and, optionally,
component C: one or more, preferably one, further pharmaceutical agent(s), and/or CAR-T cells, in which optionally either or all of said DGKalpha inhibitor A, said DGKzeta inhibitor A′, and said component C in any of the above-mentioned combinations are in the form of a pharmaceutical composition which is ready for use to be administered simultaneously, concurrently, separately or sequentially. Said DGKalpha inhibitor A, said DGKzeta inhibitor A′, and optionally component C, may be administered independently of one another by the oral, intravenous, topical, local installations, intraperitoneal or nasal route.

In another embodiment the present invention provides a kit, in which said one DGKalpha inhibitor compound, said one DGKzeta inhibitor compound, and optionally component C each are in the form of a pharmaceutical composition and in which said one DGKalpha inhibitor compound is administered prior to said one DGKzeta inhibitor compound and optionally said one DGKalpha inhibitor compound is administered prior to component C.

In another embodiment the present invention provides a kit, in which said one DGKalpha inhibitor compound and said one DGKzeta inhibitor compound are in the form of two or more pharmaceutical compositions and in which said one DGKalpha inhibitor compound is administered prior to said one DGKzeta inhibitor compound.

In another embodiment the present invention provides a kit, in which said one DGKalpha inhibitor compound and said one DGKzeta inhibitor compound are in the form of two or more pharmaceutical compositions and in which said one DGKzeta inhibitor compound is administered prior to said one DGKalpha inhibitor compound.

The term “component C” means a further optional component comprising at least one pharmaceutical agent, including the effective compound itself as well as its pharmaceutically acceptable salts, solvates, hydrates or stereoisomers as well as any pharmaceutical composition comprising such effective compound or its pharmaceutically acceptable salts, solvates, hydrates or stereoisomers, and/or chimeric antigen receptor T cells (CAR-T cells), such as Axicabtagen-Ciloleucel or Tisagenlecleucel. The activity of CAR-T cells can be suppressed by the tumor micro environment (TME). Knock out of DGKs by techniques such as Crispr had been shown to enhance CAR-T cell activity in a suppressive TME (I. Y. Jung et al., Mol. Cells 2018, 41 (8), 717-723). As used herein, the term “CAR-T cells” includes chimeric antigen receptor natural killer T-cells (CAR-NKT cells) and chimeric antigen receptor natural killer cells (CAR-NK cells).

A list of said pharmaceutical agents of component C is being provided further below. Preferably, the chimeric antigen receptor T cells (CAR-T cells) of component C are Axicabtagen-Ciloleucel or Tisagenlecleucel.

The combinations of inhibitors of DGK of this invention can be administered as the sole pharmaceutical agent or in combination with one or more further pharmaceutical agents C where the resulting combination of the inhibitors of DGK and component C causes no unacceptable adverse effects. For example, the combinations of inhibitors of DGK of this invention, such as combinations of one inhibitor of DGKalpha and one inhibitor of DGKzeta can be combined with component C, i.e. one or more further pharmaceutical agents, such as known anti-angiogenesis, anti-hyper-proliferative, anti-inflammatory, analgesic, immunoregulatory, diuretic, antiarrhythmic, anti-hypercholesterolemia, anti-dyslipidemia, anti-diabetic or antiviral agents, and the like, as well as with admixtures and combinations thereof.

Optional pharmaceutical agents which can be added as component C to the combinations inhibitors of DGK of this invention, such as combinations of one inhibitor of DGKalpha and one inhibitor of DGKzeta, can be one or more pharmaceutical agents such as 131I-chTNT, abarelix, abiraterone, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin Ill, aprepitant, arcitumomab, arglabin, arsenic trioxide, asparaginase, axitinib, azacitidine, basiliximab, belotecan, bendamustine, besilesomab, belinostat, bevacizumab, bexarotene, bicalutamide, bisantrene, bleomycin, blinatumomab, bortezomib, buserelin, bosutinib, brentuximab vedotin, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, eculizumab, edrecolomab, elliptinium acetate, elotuzumab, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, necitumumab, nedaplatin, nelarabine, neridronic acid, netupitant/palonosetron, pentetreotide, nilotinib, nilutamide, nimorazole, nimotuzumab, nimustine, nintedanib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pegfilgrastim, peginterferon alfa-2b, pemetrexed, pentazocine, pentostatin, peplomycin, perflubutane, perfosfamide, pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, rolapitant, romidepsin, romiplostim, romurtide, rucaparib, samarium (153Sm) lexidronam, sargramostim, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

Generally, the use of pharmaceutical agents as component C in combination with a combination of inhibitors of DGK of this invention, such as combinations of one inhibitor of DGKalpha and one inhibitor of DGKzeta will serve to:
(1) yield better efficacy in reducing the growth of a tumor and/or metastasis or even eliminate the tumor and/or metastasis as compared to administration of either agent alone,
(2) provide for treating a broader spectrum of different cancer (sub)types in mammals, especially humans,
(3) provide for a higher response rate among treated patients,
(4) provide for a longer survival time among treated patients compared to standard chemotherapy treatments,
(5) provide a longer time for tumor progression, and/or
(6) yield efficacy and tolerability results at least as good as those of the agents used alone, compared to known instances where other cancer agent combinations produce antagonistic effects.

### Embodiments of Pharmaceutical Compositions

### Aspect 3

Further, the present invention provides a pharmaceutical composition comprising a combination of the present invention as described herein together with one or more pharmaceutically acceptable excipients.

In accordance with a third aspect, the present invention provides a pharmaceutical composition comprising a combination of one or more inhibitor(s) of DGKalpha and one or more inhibitor(s) of DGKzeta, together with one or more pharmaceutically acceptable excipients.

In another embodiment, the present invention provides a pharmaceutical composition comprising a combination of one or more inhibitor(s) of DGKalpha and one or more inhibitor(s) of DGKzeta, optionally with any component C mentioned herein, together with one or more pharmaceutically acceptable excipients.

In another embodiment, the present invention provides a pharmaceutical composition comprising a combination of one inhibitor of DGKalpha and one inhibitor of DGKzeta, together with one or more pharmaceutically acceptable excipients.

In another embodiment, the present invention provides a pharmaceutical composition comprising a combination of one inhibitor of DGKalpha and one inhibitor of DGKzeta, optionally with any component C mentioned herein, together with one or more pharmaceutically acceptable excipients.

In another embodiment, the present invention provides a pharmaceutical composition comprising a combination of one DGKalpha inhibitor compound of general formula (I), as described herein, and one DGKzeta inhibitor compound of general formula (II), as described herein, or a stereoisomer, a tautomer, an N-oxide, a hydrate, a solvate, or a salt thereof, or a mixture of same, together with one or more pharmaceutically acceptable excipients.

In another embodiment, the present invention provides a pharmaceutical composition comprising a combination of one DGKalpha inhibitor compound of general formula (I), as described herein, and one DGKzeta inhibitor compound of general formula (II), as described herein, or a stereoisomer, a tautomer, an N-oxide, a hydrate, a solvate, or a salt thereof, or a mixture of same, optionally with any component C mentioned herein, together with one or more pharmaceutically acceptable excipients.

In another embodiment, the present invention provides a pharmaceutical composition comprising a combination of one DGKalpha inhibitor compound of general formula (I), as described herein, and one DGKzeta inhibitor compound of general formula (II), as described herein, together with one or more pharmaceutically acceptable excipients.

In another embodiment, the present invention provides a pharmaceutical composition comprising a combination of one DGKalpha inhibitor compound of general formula (I), as described herein, and one DGKzeta inhibitor compound of general formula (II), as described herein, optionally with any component C mentioned herein, together with one or more pharmaceutically acceptable excipients.

In a preferred embodiment, the present invention provides a pharmaceutical composition comprising a combination of DGKalpha inhibitor A and DGKzeta inhibitor A′, as described herein, or a stereoisomer, a tautomer, an N-oxide, a hydrate, a solvate, or a salt thereof, or a mixture of same, together with one or more pharmaceutically acceptable excipients.

In another preferred embodiment, the present invention provides a pharmaceutical composition comprising a combination consisting of DGKalpha inhibitor A and DGKzeta inhibitor A′, as described herein, or a stereoisomer, a tautomer, an N-oxide, a hydrate, a solvate, or a salt thereof, or a mixture of same, together with one or more pharmaceutically acceptable excipients.

In another preferred embodiment, the present invention provides a pharmaceutical composition comprising a combination of DGKalpha inhibitor A and DGKzeta inhibitor A′, as described herein, or a tautomer, an N-oxide, a hydrate, a solvate, or a salt thereof, or a mixture of same, together with one or more pharmaceutically acceptable excipients.

In another preferred embodiment, the present invention provides a pharmaceutical composition comprising a combination consisting of DGKalpha inhibitor A and DGKzeta inhibitor A′, as described herein, or a tautomer, an N-oxide, a hydrate, a solvate, or a salt thereof, or a mixture of same, together with one or more pharmaceutically acceptable excipients.

In another preferred embodiment, the present invention provides a pharmaceutical composition comprising a combination of DGKalpha inhibitor A and DGKzeta inhibitor A′, as described herein, or a stereoisomer, a tautomer, an N-oxide, a hydrate, a solvate, or a salt thereof, or a mixture of same, optionally with any component C mentioned herein, together with one or more pharmaceutically acceptable excipients.

In another preferred embodiment, the present invention provides a pharmaceutical composition comprising a combination consisting of DGKalpha inhibitor A and DGKzeta inhibitor A′, as described herein, or a stereoisomer, a tautomer, an N-oxide, a hydrate, a solvate, or a salt thereof, or a mixture of same, optionally with any component C mentioned herein, together with one or more pharmaceutically acceptable excipients.

In another preferred embodiment, the present invention provides a pharmaceutical composition comprising a combination of DGKalpha inhibitor A and DGKzeta inhibitor A′, as described herein, or a tautomer, an N-oxide, a hydrate, a solvate, or a salt thereof, or a mixture of same, optionally with any component C mentioned herein, together with one or more pharmaceutically acceptable excipients.

In another preferred embodiment, the present invention provides a pharmaceutical composition comprising a combination consisting of DGKalpha inhibitor A and DGKzeta inhibitor A′, as described herein, or a tautomer, an N-oxide, a hydrate, a solvate, or a salt thereof, or a mixture of same, optionally with any component C mentioned herein, together with one or more pharmaceutically acceptable excipients.

In another preferred embodiment, the present invention provides a pharmaceutical composition comprising a combination of DGKalpha inhibitor A and DGKzeta inhibitor A′, as described herein, together with one or more pharmaceutically acceptable excipients.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is in the range from 20:1 to 1:20.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is in the range from 12:1 to 1:12.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is in the range from 12:1 to 8:1.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is in the range from 10:1 to 1:1.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is approximately 10:1.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is in the range from 4:1 to 2:1.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is approximately 3:1.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is in the range from 2:1 to 1:2.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is approximately 1:1.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is in the range from 1:2 to 1:4.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is approximately 1:3.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is in the range from 1:8 to 1:12.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is in the range from 1:1 to 1:10.

In another preferred embodiment, the present invention provides a pharmaceutical composition comprising a combination of DGKalpha inhibitor A and DGKzeta inhibitor A′, as described herein, together with one or more pharmaceutically acceptable excipients, in which the dose ratio (mg or mg/kg of DGKalpha inhibitor A:mg or mg/kg of DGKzeta inhibitor A′) is approximately 1:10.

In another preferred embodiment, the present invention provides a pharmaceutical composition comprising a combination consisting of DGKalpha inhibitor A and DGKzeta inhibitor A′, as described herein, together with one or more pharmaceutically acceptable excipients.

In another preferred embodiment, the present invention provides a pharmaceutical composition comprising a combination of DGKalpha inhibitor A and DGKzeta inhibitor A′, as described herein, optionally with any component C mentioned herein, together with one or more pharmaceutically acceptable excipients.

In another preferred embodiment, the present invention provides a pharmaceutical composition comprising a combination consisting of DGKalpha inhibitor A and DGKzeta inhibitor A′, as described herein, optionally with any component C mentioned herein, together with one or more pharmaceutically acceptable excipients.

In another embodiment the inhibitor(s) of DGKalpha and the inhibitor(s) of DGKzeta, and optionally component C, are present in separate formulations.

In another embodiment the inhibitor(s) of DGKalpha and the inhibitor(s) of DGKzeta, and optionally component C, are present in a joint formulation.

Pharmaceutically acceptable excipients are non-toxic, preferably they are non-toxic and inert.

Pharmaceutically acceptable excipients include, inter alia,
fillers and excipients (for example cellulose, microcrystalline cellulose, such as, for example, Avicel©, lactose, mannitol, starch, calcium phosphate such as, for example, Di-Cafos©),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat)
solvents (for example water, ethanol, Isopropanol, glycerol, propylene glycol, medium chain-length triglycerides fatty oils, liquid polyethylene glycols, paraffins),
surfactants, emulsifiers, dispersants or wetters (for example sodium dodecyle sulphate, lecithin, phospholipids, fatty alcohols such as, for example, Lanette®, sorbitan fatty acid esters such as, for example, Span®, polyoxyethylene sorbitan fatty acid esters such as, for example, Tween®, polyoxyethylene fatty acid glycerides such as, for example, Cremophor®, polyoxethylene fatty acid esters, polyoxyethylene fatty alcohol ethers, glycerol fatty acid esters, poloxamers such as, for example, Pluronic©),
buffers and also acids and bases (for example phosphates, carbonates, citric acid, acetic acid, hydrochloric acid, sodium hydroxide solution, ammonium carbonate, trometamol, triethanolamine)
isotonicity agents (for example glucose, sodium chloride),
adsorbents (for example highly-disperse silicas)
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids such as, for example, Carbopol®, alginates, gelatine),
disintegrants (for example modified starch, carboxymethylcellulose-sodium, sodium starch glycolate such as, for example, Explotab®, cross-linked polyvinylpyrrolidon, croscarmellose-sodium such as, for example, AcDiSol®),
flow regulators, lubricants, glidant and mould release agents (for example magnesium stearate, stearic acid, talc, highly-disperse silicas such as, for example, Aerosil®),
coating materials (for example sugar, shellac) and film formers for films or diffusion membranes which dissolve rapidly or in a modified manner (for example polyvinylpyrrolidones such as, for example, Kollidon®, polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®),
capsule materials (for example gelatine, hydroxypropylmethylcellulose),
synthetic polymers (for example polylactides, polyglycolides, polyacrylates, polymethacrylates such as, for example, Eudragit®, polyvinylpyrrolidones such as, for example, Kollidon®, polyvinyl alcohols, polyvinyl acetates, polyethylene oxides, polyethylene glycols and their copolymers and block copolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

Further excipients and procedures are described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)—Part-1” PDA Journal of Pharmaceutical Science &Technology 1999, 53(6), 324-349; and Nema, S. et al., “Excipients and Their Use in Injectable Products” PDA Journal of Pharmaceutical Science & Technology 1997, 51(4), 166-171.

The inhibitors of DGK of this invention, such as combinations of one inhibitor of DGKalpha and one inhibitor of DGKzeta, and optionally component C may be administered independently of one another by the oral, intravenous, topical, local installations, intraperitoneal or nasal route.

Preferably, the inhibitors of DGK of this invention, such as combinations of one inhibitor of DGKalpha and one inhibitor of DGKzeta, are administered orally and optionally component C as required.

The pharmaceutical composition (formulation) varies by the route of administration. Components of this invention can be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

Components of this invention can also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions can be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs can be formulated with sweetening agents such as, for example, glycerol, propylene glycol, sorbitol or sucrose. Such formulations may also contain a demulcent, and preservative, such as methyl and propyl parabens and flavoring and coloring agents.

Components of this invention can also be administered parenterally, that is, subcutaneously, intravenously, intraocularly, intrasynovially, intramuscularly, or interperitoneally, as injectable dosages of the compound in preferably a pharmaceutically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions of the present invention can be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation can also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

Components of the invention can also be administered in the form of suppositories for rectal administration of the drug. These components can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It can be desirable or necessary to introduce a component of the present invention to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

### Embodiments of DGK Inhibitors, Combinations Thereof, Kits and Pharmaceutical Compositions for Use in Methods of the Treatment or Prophylaxis of a Disease

Diseases, disorders and conditions particularly suitable for treatment with a combination, a kit, a pharmaceutical composition, and inhibitors of DGKalpha and inhibitors of DGKzeta for use in such treatment, of the present invention are liquid and solid tumors, such as cancers of the breast, respiratory tract, brain, reproductive organs, digestive tract, urinary tract, eye, mesothelium, liver, skin, head and neck, thyroid, thymus, parathyroid and their distant metastases. Those disorders also include squamous cell carcinomas, lymphomas, sarcomas, and leukaemias.

Examples of breast cancers include, but are not limited to, triple negative breast cancer, triple positive breast cancer, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypophtalmic glioma, cerebellar and cerebral astrocytoma, glioblastoma, medulloblastoma, ependymoma, as well as neuroectodermal and pineal tumor.

Tumors of the male reproductive organs include, but are not limited to, prostate and testicular cancer.

Tumors of the female reproductive organs include, but are not limited to, endometrial, cervical, ovarian, vaginal, and vulvar cancer, as well as sarcoma of the uterus.

Examples of ovarian cancer include, but are not limited to serous tumor, endometrioid tumor, mucinous cystadenocarcinoma, granulosa cell tumor, Sertoli-Leydig cell tumor and arrhenoblastoma.

Examples of cervical cancer include, but are not limited to squamous cell carcinoma, adenocarcinoma, adenosquamous carcinoma, small cell carcinoma, neuroendocrine tumor, glassy cell carcinoma and villoglandular adenocarcinoma.

Tumors of the digestive tract include, but are not limited to, anal, colon, colorectal (including microsatellite instability high (MSI H) colorectal carcinomas), esophageal, gallbladder, gastric, pancreatic, rectal, small-intestine, gastroesophageal junction adenocarcinomas, and salivary gland cancers.

Examples of esophageal cancer include, but are not limited to esophageal cell carcinomas and adenocarcinomas, as well as squamous cell carcinomas, leiomyosarcoma, malignant melanoma, rhabdomyosarcoma and lymphoma.

Examples of gastric cancer include, but are not limited to intestinal type and diffuse type gastric adenocarcinoma.

Examples of pancreatic cancer include, but are not limited to pancreatic adenocarcinoma, such as ductal adenocarcinoma, and adenosquamous carcinomas and pancreatic endocrine tumors.

Tumors of the urinary tract include, but are not limited to, bladder, penile, kidney, renal pelvis, ureter, urethral and human papillary renal cancers.

Examples of kidney cancer include, but are not limited to renal cell carcinoma, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor.

Examples of bladder cancer include, but are not limited to transitional cell carcinoma, squamous cell carcinoma, adenocarcinoma, sarcoma and small cell carcinoma.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, squamous cell carcinoma, Kaposi's sarcoma, malignant melanoma, Merkel cell skin cancer, and non-melanoma skin cancer.

Head-and-neck cancers include, but are not limited to, squamous cell cancer of the head and neck (HNSCC), laryngeal, hypopharyngeal, nasopharyngeal, oropharyngeal cancer, salivary gland cancer, lip and oral cavity cancer and squamous cell.

Lymphomas include, but are not limited to, AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to, sarcoma of the soft tissue, osteosarcoma, malignant fibrous histiocytoma, lymphosarcoma, and rhabdomyosarcoma.

Leukemias include, but are not limited to, acute myeloid leukemia, acute lymphoblastic leukemia (including T-cell acute lymphoblastic leukemia), chronic lymphocytic leukemia, chronic myelogenous leukemia, and hairy cell leukemia.

The combinations of the present invention can be used in particular in therapy and prevention, i.e. prophylaxis, of tumor growth and metastases, especially in solid tumors of all indications and stages with or without pre-treatment of the tumor growth.

The present invention also provides methods of treating a variety of other disorders wherein DGKalpha and/or DGKzeta is involved such as, but not limited to, disorders with dysregulated immune responses, inflammation, vaccination for infection & cancer, virus infections, lymphoproliferative disorders, asthma, eye diseases, and type 2 diabetes/insulin resistance.

### Embodiments of Inhibitors of DGKalpha for Use in Methods of the Treatment or Prophylaxis of a Disease

### Aspect 4

According to a fourth aspect there is provided an inhibitor of DGKalpha for use in a method of the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra, characterized in that said method comprises administering at least one inhibitor of DGKzeta.

Preferably the inhibitor of DGKalpha is characterized by formula (I) as defined under Section DGKalpha inhibitors Embodiments A. Preferably the inhibitor of DGKalpha is characterized by formula (I) as defined under Section DGKalpha inhibitors Embodiments B. Preferably the inhibitor of DGKalpha is characterized by formula (I) as defined under Section DGKalpha inhibitors Embodiments C. Preferably the inhibitor of DGKalpha is a compound as listed in Section DGKalpha inhibitors Embodiments D. Preferably the inhibitor of DGKalpha is DGKalpha inhibitor A.

Preferably the inhibitor of DGKzeta is characterized by formula (II) as defined under Section DGKzeta inhibitors Embodiments A. Preferably the inhibitor of DGKzeta is characterized by formula (II) as defined under Section DGKzeta inhibitors Embodiments B. Preferably the inhibitor of DGKzeta is characterized by formula (II) as defined under Section DGKzeta inhibitors Embodiments C. Preferably the inhibitor of DGKzeta is a compound as listed in Section DGKzeta inhibitors Embodiments D. Preferably the inhibitor of DGKzeta is DGKzeta inhibitor A′.

In a highly preferred embodiment, the inhibitor of DGKalpha is characterized by formula (I) as defined under Section DGKalpha inhibitors Embodiments A and the inhibitor of DGKzeta is characterized by formula (II) as defined under Section DGKzeta inhibitors Embodiments A.

In another preferred embodiment, the inhibitor of DGKalpha is characterized by formula (I) as defined under Section DGKalpha inhibitors Embodiments B and the inhibitor of DGKzeta is characterized by formula (II) as defined under Section DGKzeta inhibitors Embodiments B.

In another preferred embodiment, the inhibitor of DGKalpha is characterized by formula (I) as defined under Section DGKalpha inhibitors Embodiments C and the inhibitor of DGKzeta is characterized by formula (II) as defined under Section DGKzeta inhibitors Embodiments C.

In another preferred embodiment, the inhibitor of DGKalpha is a compound as listed in Section DGKalpha inhibitors Embodiments D and the inhibitor of DGKzeta is a compound as listed in Section DGKzeta inhibitors Embodiments D.

In a highly preferred embodiment, the inhibitor of DGKalpha is DGKalpha inhibitor A and the inhibitor of DGKzeta is DGKzeta inhibitor A′. For example, the inhibitor of DGKalpha is DGKalpha inhibitor A, or a tautomer, an N-oxide, a hydrate, a solvate, or a salt thereof, or a mixture of same and/or the inhibitor of DGKzeta is DGKzeta inhibitor A′, or a stereoisomer, a tautomer, an N-oxide, a hydrate, a solvate, or a salt thereof, or a mixture of same.

In another highly preferred embodiment, the inhibitor of DGKalpha is DGKalpha inhibitor A and the inhibitor of DGKzeta is DGKzeta inhibitor A′.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 20:1 to 1:20.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 12:1 to 1:12.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 12:1 to 8:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 10:1 to 1:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 10:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 4:1 to 2:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 3:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 2:1 to 1:2.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 1:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 1:2 to 1:4.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 1:3.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 1:8 to 1:12.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 1:1 to 1:10.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 1:10.

### Embodiments of Inhibitors of DGKzeta for Use in Methods of the Treatment or Prophylaxis of a Disease

### Aspect 5

According to a fifth aspect there is provided an inhibitor of DGKzeta for use in a method of the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra, characterized in that said method comprises administering at least one inhibitor of DGKalpha.

Preferably the inhibitor of DGKalpha is characterized by formula (I) as defined under Section DGKalpha inhibitors Embodiments A. Preferably the inhibitor of DGKalpha is characterized by formula (I) as defined under Section DGKalpha inhibitors Embodiments B. Preferably the inhibitor of DGKalpha is characterized by formula (I) as defined under Section DGKalpha inhibitors Embodiments C. Preferably the inhibitor of DGKalpha is a compound as listed in Section DGKalpha inhibitors Embodiments D. Preferably the inhibitor of DGKalpha is DGKalpha inhibitor A.

Preferably the inhibitor of DGKzeta is characterized by formula (II) as defined under Section DGKzeta inhibitors Embodiments A. Preferably the inhibitor of DGKzeta is characterized by formula (II) as defined under Section DGKzeta inhibitors Embodiments B. Preferably the inhibitor of DGKzeta is characterized by formula (II) as defined under Section DGKzeta inhibitors Embodiments C. Preferably the inhibitor of DGKzeta is a compound as listed in Section DGKzeta inhibitors Embodiments D. Preferably the inhibitor of DGKzeta is DGKzeta inhibitor A′.

Preferably, the inhibitor of DGKalpha is characterized by formula (I) as defined under Section DGKalpha inhibitors Embodiments A and the inhibitor of DGKzeta is characterized by formula (II) as defined under Section DGKzeta inhibitors Embodiments A.

In a preferred embodiment, the inhibitor of DGKalpha is characterized by formula (I) as defined under Section DGKalpha inhibitors Embodiments B and the inhibitor of DGKzeta is characterized by formula (II) as defined under Section DGKzeta inhibitors Embodiments B.

In another preferred embodiment, the inhibitor of DGKalpha is characterized by formula (I) as defined under Section DGKalpha inhibitors Embodiments C and the inhibitor of DGKzeta is characterized by formula (II) as defined under Section DGKzeta inhibitors Embodiments C.

In another preferred embodiment, the inhibitor of DGKalpha is a compound as listed in Section DGKalpha inhibitors Embodiments D and the inhibitor of DGKzeta is a compound as listed in Section DGKzeta inhibitors Embodiments D.

In a highly preferred embodiment, the inhibitor of DGKalpha is DGKalpha inhibitor A and the inhibitor of DGKzeta is DGKzeta inhibitor A′. For example, the inhibitor of DGKalpha is DGKalpha inhibitor A, or a tautomer, an N-oxide, a hydrate, a solvate, or a salt thereof, or a mixture of same and/or the inhibitor of DGKzeta is DGKzeta inhibitor A′, or a stereoisomer, a tautomer, an N-oxide, a hydrate, a solvate, or a salt thereof, or a mixture of same.

In a highly preferred embodiment, the inhibitor of DGKalpha is DGKalpha inhibitor A and the inhibitor of DGKzeta is DGKzeta inhibitor A′.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 20:1 to 1:20.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 12:1 to 1:12.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 12:1 to 8:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 10:1 to 1:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 10:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 4:1 to 2:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 3:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 2:1 to 1:2.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 1:1.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 1:2 to 1:4.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 1:3.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 1:8 to 1:12.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is in the range from 1:1 to 1:10.

In another preferred embodiment, the inhibitor of DGKalpha is DGKalpha inhibitor A and the inhibitor of DGKzeta is DGKzeta inhibitor A′, wherein the dose ratio (mg or mg/kg of DGKalpha inhibitor A:mg or mg/kg of DGKzeta inhibitor A′) is approximately 1:10.

### Embodiments of Combinations According to the Present Invention Use in Methods of the Treatment or Prophylaxis of a Disease

### Aspect 6

In accordance with a sixth aspect, the present invention provides the combination of the present invention as described supra for use in the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra.

In another embodiment, the present invention provides the use of the combination of the present invention as described supra for the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra.

In another embodiment, the present invention provides the use of such combinations as described supra for the preparation of a medicament for the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra.

In another embodiment, the present invention provides methods for the treatment and/or prophylaxis of a disease, preferably a condition with dysregulated immune responses, particularly cancer, or a viral infection or another disorder associated with aberrant DGKalpha and/or DGKzeta signaling, in mammals, including humans, as described supra using an effective amount of the combination of the present invention as described supra.

In a preferred embodiment, the present invention provides combinations of DGKalpha inhibitor A and DGKzeta inhibitor A′ as described herein, or a stereoisomer, a tautomer, an N-oxide, a hydrate, a solvate, or a salt thereof, or a mixture of same, for use in a method of the treatment or prophylaxis of a disease in a patient, preferably a condition with dysregulated immune responses, particularly cancer, or a viral infection or another disorder associated with aberrant DGKalpha and/or DGKzeta signaling, in mammals, including humans, as described supra.

In another preferred embodiment, the present invention provides combinations of DGKalpha inhibitor A and DGKzeta inhibitor A′ as described herein, for use in a method of the treatment or prophylaxis of a disease in a patient, preferably a condition with dysregulated immune responses, particularly cancer, or a viral infection or another disorder associated with aberrant DGKalpha and/or DGKzeta signaling, in mammals, including humans, as described supra.

In another preferred embodiment, the present invention provides the use of combinations of DGKalpha inhibitor A and DGKzeta inhibitor A′ as described herein, or a stereoisomer, a tautomer, an N-oxide, a hydrate, a solvate, or a salt thereof, or a mixture of same, in a method of the treatment or prophylaxis of a disease in a patient, preferably a condition with dysregulated immune responses, particularly cancer, or a viral infection or another disorder associated with aberrant DGKalpha and/or DGKzeta signaling, in mammals, including humans, as described supra.

In another preferred embodiment, the present invention provides the use of combinations of DGKalpha inhibitor A and DGKzeta inhibitor A′ as described herein, in a method of the treatment or prophylaxis of a disease in a patient, preferably a condition with dysregulated immune responses, particularly cancer, or a viral infection or another disorder associated with aberrant DGKalpha and/or DGKzeta signaling, in mammals, including humans, as described supra.

In another preferred embodiment, the present invention provides the use of combinations of DGKalpha inhibitor A and DGKzeta inhibitor A′ as described herein, or a stereoisomer, a tautomer, an N-oxide, a hydrate, a solvate, or a salt thereof, or a mixture of same for the preparation of a medicament for the treatment or prophylaxis of a disease in a patient, preferably a condition with dysregulated immune responses, particularly cancer, or a viral infection or another disorder associated with aberrant DGKalpha and/or DGKzeta signaling, in mammals, including humans, as described supra.

In another preferred embodiment, the present invention provides the use of combinations of DGKalpha inhibitor A and DGKzeta inhibitor A′ as described herein, for the preparation of a medicament for the treatment or prophylaxis of a disease in a patient, preferably a condition with dysregulated immune responses, particularly cancer, or a viral infection or another disorder associated with aberrant DGKalpha and/or DGKzeta signaling, in mammals, including humans, as described supra.

In another preferred embodiment, the present invention provides methods for the treatment and/or prophylaxis of a disease, preferably a condition with dysregulated immune responses, particularly cancer, or a viral infection or another disorder associated with aberrant DGKalpha and/or DGKzeta signaling, in mammals, including humans, as described supra, using an effective amount of the combination of DGKalpha inhibitor A and DGKzeta inhibitor A′ as described herein, or a stereoisomer, a tautomer, an N-oxide, a hydrate, a solvate, or a salt thereof, or a mixture of same.

In another preferred embodiment, the present invention provides methods for the treatment and/or prophylaxis of a disease, preferably a condition with dysregulated immune responses, particularly cancer, or a viral infection or another disorder associated with aberrant DGKalpha and/or DGKzeta signaling, in mammals, including humans, as described supra, using an effective amount of the combination of DGKalpha inhibitor A and DGKzeta inhibitor A′ as described herein.

### Embodiments of Kits According to the Present Invention Use in Methods of the Treatment or Prophylaxis of a Disease

### Aspect 7

In accordance with a seventh aspect, the present invention provides the kit as described supra for use in the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra.

In another embodiment, the present invention provides the use of the kit as described supra for the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra.

In another embodiment, the present invention provides the use of such kit as described supra for the preparation of a medicament for the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra.

In another embodiment, the present invention provides methods for the treatment and/or prophylaxis of a disease, preferably a condition with dysregulated immune responses, particularly cancer, or a viral infection or another disorder associated with aberrant DGKalpha and/or DGKzeta signaling, in mammals, including humans, as described supra, using an effective amount of the kit as described supra.

### Embodiments of Pharmaceutical Compositions According to the Present Invention Use in Methods of the Treatment or Prophylaxis of a Disease

### Aspect 8

In accordance with an eighth aspect, the present invention provides the pharmaceutical composition as described supra for use in the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra.

In another embodiment, the present invention provides the use of the pharmaceutical composition as described supra for the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra.

In another embodiment, the present invention provides the use of such pharmaceutical composition as described supra for the preparation of a medicament for the treatment or prophylaxis of a disease, preferably a condition with dysregulated immune responses, particularly cancer, or a viral infection or another disorder associated with aberrant DGKalpha and/or DGKzeta signaling, in mammals, including humans, as described supra.

In another embodiment, the present invention provides methods for the treatment and/or prophylaxis of a disease, preferably a condition with dysregulated immune responses, particularly cancer, or a viral infection or another disorder associated with aberrant DGKalpha and/or DGKzeta signaling, in mammals, including humans, as described supra, using an effective amount of the pharmaceutical composition as described supra.

### Further Embodiments of Methods of the Treatment or Prophylaxis of a Disease According to the Present Invention

### Aspect 9

In accordance with a ninth aspect, the present invention provides a method of the treatment or prophylaxis of a disease in a patient, preferably a condition with dysregulated immune responses, particularly cancer, or a viral infection or another disorder associated with aberrant DGKalpha and/or DGKzeta signaling, in mammals, including humans, as described supra, comprising
a) administering DGKalpha inhibitor A as described herein, or a tautomer, an N-oxide, a hydrate, a solvate, or a salt thereof, or a mixture of same, and
b) administering and DGKzeta inhibitor A′, as described herein, or a stereoisomer, a tautomer, an N-oxide, a hydrate, a solvate, or a salt thereof, or a mixture of same.

In a preferred embodiment, the present invention provides a method of the treatment or prophylaxis of a disease in a patient, preferably a condition with dysregulated immune responses, particularly cancer, or a viral infection or another disorder associated with aberrant DGKalpha and/or DGKzeta signaling, in mammals, including humans, as described supra, comprising
a) administering DGKalpha inhibitor A as described herein, and
b) administering and DGKzeta inhibitor A′, as described herein.

In another preferred embodiment, in said method of the treatment or prophylaxis of a disease in a patient the dose ratio (mg or mg/kg of DGKalpha inhibitor A:mg or mg/kg of DGKzeta inhibitor A′) is in the range from 20:1 to 1:20.

In another preferred embodiment, in said method of the treatment or prophylaxis of a disease in a patient the dose ratio (mg or mg/kg of DGKalpha inhibitor A:mg or mg/kg of DGKzeta inhibitor A′) is in the range from 12:1 to 1:12.

In another preferred embodiment, in said method of the treatment or prophylaxis of a disease in a patient the dose ratio (mg or mg/kg of DGKalpha inhibitor A:mg or mg/kg of DGKzeta inhibitor A′) is in the range from 12:1 to 8:1.

In another preferred embodiment, in said method of the treatment or prophylaxis of a disease in a patient the dose ratio (mg or mg/kg of DGKalpha inhibitor A:mg or mg/kg of DGKzeta inhibitor A′) is in the range from 10:1 to 1:1.

In another preferred embodiment, in said method of the treatment or prophylaxis of a disease in a patient the dose ratio (mg or mg/kg of DGKalpha inhibitor A:mg or mg/kg of DGKzeta inhibitor A′) is approximately 10:1.

In another preferred embodiment, in said method of the treatment or prophylaxis of a disease in a patient the dose ratio (mg or mg/kg of DGKalpha inhibitor A:mg or mg/kg of DGKzeta inhibitor A′) is in the range from 4:1 to 2:1.

In another preferred embodiment, in said method of the treatment or prophylaxis of a disease in a patient the dose ratio (mg or mg/kg of DGKalpha inhibitor A:mg or mg/kg of DGKzeta inhibitor A′) is approximately 3:1.

In another preferred embodiment, in said method of the treatment or prophylaxis of a disease in a patient the dose ratio (mg or mg/kg of DGKalpha inhibitor A:mg or mg/kg of DGKzeta inhibitor A′) is in the range from 2:1 to 1:2.

In another preferred embodiment, in said method of the treatment or prophylaxis of a disease in a patient the dose ratio (mg or mg/kg of DGKalpha inhibitor A:mg or mg/kg of DGKzeta inhibitor A′) is approximately 1:1.

In another preferred embodiment, in said method of the treatment or prophylaxis of a disease in a patient the dose ratio (mg or mg/kg of DGKalpha inhibitor A:mg or mg/kg of DGKzeta inhibitor A′) is in the range from 1:2 to 1:4.

In another preferred embodiment, in said method of the treatment or prophylaxis of a disease in a patient the dose ratio (mg or mg/kg of DGKalpha inhibitor A:mg or mg/kg of DGKzeta inhibitor A′) is approximately 1:3.

In another preferred embodiment, in said method of the treatment or prophylaxis of a disease in a patient the dose ratio (mg or mg/kg of DGKalpha inhibitor A:mg or mg/kg of DGKzeta inhibitor A′) is in the range from 1:8 to 1:12.

In another preferred embodiment, in said method of the treatment or prophylaxis of a disease in a patient the dose ratio (mg or mg/kg of DGKalpha inhibitor A:mg or mg/kg of DGKzeta inhibitor A′) is in the range from 1:1 to 1:10.

In another preferred embodiment, in said method of the treatment or prophylaxis of a disease in a patient the dose ratio (mg or mg/kg of DGKalpha inhibitor A:mg or mg/kg of DGKzeta inhibitor A′) is approximately 1:10.

### Dose and Administration

Based upon standard laboratory techniques known to evaluate compounds useful for the treatment of hyper-proliferative diseases and angiogenic diseases, by standard toxicity tests and by standard pharmacological assays for the determination of treatment of the conditions identified above in mammals, and by comparison of these results with the results of known medicaments that are used to treat these conditions, the effective dosage of the compounds, kits and combinations of this invention can readily be determined for treatment of each desired indication. The amount of the active ingredients to be administered in the treatment of one of these conditions can vary widely according to such considerations as the particular component and dosage unit employed, the mode of administration, the period of treatment, the age and sex of the patient treated, and the nature and extent of the condition treated.

The total amount of the active ingredients to be administered will generally range from about 0.001 mg/kg to about 200 mg/kg body weight per day, and preferably from about 0.01 mg/kg to about 50 mg/kg body weight per day. Clinically useful dosing schedules of a compound will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient(s), and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compounds employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

The dose(s), administration regime(s) and administration route(s) may have to be adapted according to, inter alia, the indication, the indication stage, the patient age and/or the patient gender, among other factors. Such adaptations can be readily determined by standard techniques known to the skilled person. For both, for the DGK inhibitors, particularly DGKalpha inhibitor A and DGKzeta inhibitor A′, the administered dosage of the compound(s) may be modified depending on any superior or unexpected results which may be obtained as routinely determined with this invention.

The DGK inhibitor(s) as described herein can be administered to a patient orally, topically, parenterally, rectally, by inhalation, and by injection. Administration by injection includes intravenous, intramuscular, subcutaneous, and parenterally as well as by infusion techniques. The agents can be administered by any of the conventional routes of administration for these compounds. The preferred route of administration for the DGK inhibitor(s) is typically orally.

Any of the routes and regimens of administration may be modified depending on any superior or unexpected results which may be obtained as routinely determined with this invention.

### DESCRIPTION OF THE FIGURES

FIG. 1 shows the tumor cell viability of human Colo-800 cells as cell index, also referred to as tumor cell index TCI in corresponding Example 2, infra, in the following groups (from left to right):
Human Colo-800 cells in medium alone,
Human Colo-800 cells incubated with DGKalpha inhibitor A (50 nM) and DGKzeta inhibitor A′ (50 nM), but without the addition of tumor antigen-specific MART1 T cells,
“Vehicle”, that is, human Colo-800 cells with the addition of tumor antigen-specific MART1 T cells but without addition of DGK inhibitor,
Human Colo-800 cells with the addition of tumor antigen-specific MART1 T cells, and with combination treatment with DGKalpha inhibitor A and DGKzeta inhibitor A′ in increasing concentrations (1 nM each, 10 nM each, 20 nM each, 30 nM each, and 50 nM each),
Human Colo-800 cells with the addition of tumor antigen-specific MART1 T cells, and with treatment with DGKalpha inhibitor A (monotherapy) in increasing concentrations (1 nM, 10 nM, and 50 nM),
Human Colo-800 cells with the addition of tumor antigen-specific MART1 T cells, and with treatment with DGKzeta inhibitor A′ (monotherapy) in increasing concentrations (1 nM, 10 nM, and 50 nM).

FIG. 2 shows the corresponding levels of IFN-γ secretions, in pg(mL), in the treatment groups (from left to right):
Human Colo-800 cells in medium alone,
Human Colo-800 cells incubated with DGKalpha inhibitor A (50 nM) and DGKzeta inhibitor A′ (50 nM), but without the addition of tumor antigen-specific MART1 T cells
“Vehicle”, that is, human Colo-800 cells with the addition of tumor antigen-specific MART1 T cells but without addition of DGK inhibitor,
Human Colo-800 cells with the addition of tumor antigen-specific MART1 T cells, and with combination treatment with DGKalpha inhibitor A and DGKzeta inhibitor A′ in increasing concentrations (1 nM each, 10 nM each, 20 nM each, 30 nM each, and 50 nM each),
Human Colo-800 cells with the addition of tumor antigen-specific MART1 T cells, and with treatment with DGKalpha inhibitor A (monotherapy) in increasing concentrations (1 nM, 10 nM, and 50 nM),
Human Colo-800 cells with the addition of tumor antigen-specific MART1 T cells, and with treatment with DGKzeta inhibitor A′ (monotherapy) in increasing concentrations (1 nM, 10 nM, and 50 nM).
   For a discussion of the results shown in FIGS. 1 and 2, the reader is referred to Example 2, infra.

FIGS. 3 and 4 show the activation of PBMC cells, determined by measuring the proportion of CD25+ cells within the CD8+-naïve T-cells in the respective samples, as a function of treatment of said PBMC cells with DGKalpha inhibitor A and DGKzeta inhibitor A′ at different concentrations.

FIG. 3 shows the PBMC activation displayed in ten plots, one each for a given concentration of DGKzeta inhibitor A′ (referred to in FIG. 3 as “DGKzi”) ascending from plot to plot, as a function of the concentration of DGKalpha inhibitor A (referred to as “DGKai” in FIG. 3), which increases from left to right within each individual plot.

FIG. 4 shows the same set of data as a 3-D plot, in which E represents the proportion of CD25+ cells within the CD8+-naïve T-cells.

The data shown in FIGS. 3 and 4, together with simulated data, are also displayed in table format in Example 3. For a discussion of the results, the reader is also referred to Example 3, infra.

FIG. 5A shows the time course of tumor growth in the syngeneic EMT6 murine breast carcinoma model following treatment with DGKzeta inhibitor A′, with DGKalpha inhibitor A and with anti-PD-L1 antibody, and combinations thereof, in the treatment groups, according to Example 4, as follows: (-∘-: Vehicle, QD, isotype control 5 mg/kg, Q3/4D; -♦-: DGKzeta inhibitor A′, 5 mg/kg (in vehicle), QD, plus isotype control 5 mg/kg, Q3/4D; -▴-: DGKalpha inhibitor A, 3 mg/kg (in vehicle), QD, plus isotype control 5 mg/kg, Q3/4D; -□-: DGKzeta inhibitor A′, 5 mg/kg (in vehicle) and DGKalpha inhibitor A, 3 mg/kg (in vehicle), QD, plus isotype control 5 mg/kg, Q3/4D; -●-: Vehicle QD, plus anti-PD-L1 antibody, 5 mg/kg, Q3/4D; -▪-: DGKzeta inhibitor A′, 5 mg/kg (in vehicle), QD, plus anti-PD-L1 antibody 5 mg/kg, Q3/4D); -▾-: DGKalpha inhibitor A, 3 mg/kg (in vehicle), QD, plus anti-PD-L1 antibody 5 mg/kg, Q3/4D; -∇-: DGKzeta inhibitor A′, 5 mg/kg (in vehicle) and DGKalpha inhibitor A, 3 mg/kg (in vehicle), QD, plus anti-PD-L1 antibody 5 mg/kg, Q3/4D).

FIG. 5B shows data from the same experiment, however only as far as relevant for the combinations of the present invention, such as the combinations of DGKalpha inhibitor A and DGKzeta inhibitor A′: (-∘-: Vehicle, QD, isotype control 5 mg/kg, Q3/4D; -♦-: DGKzeta inhibitor A′, 5 mg/kg (in vehicle), QD, plus isotype control 5 mg/kg, Q3/4D; -▴-: DGKalpha inhibitor A, 3 mg/kg (in vehicle), QD, plus isotype control 5 mg/kg, Q3/4D; -□-: DGKzeta inhibitor A′, 5 mg/kg (in vehicle) and DGKalpha inhibitor A, 3 mg/kg (in vehicle), QD, plus isotype control 5 mg/kg, Q3/4D).

EMT6 murine breast carcinoma cells were inoculated s.c. into Balb/c mice (n=10/group). Treatments with vehicle, DGKalpha inhibitor A, DGKzeta inhibitor A′, isotype control, and anti-PD-L1 antibody were started on day 8 and the last treatment doses were given on day 23. Q3/4D, every third or fourth day; QD, once daily.

The data show a surprisingly much stronger tumor inhibition resulting from combination treatment with DGKalpha inhibitor A and DGKzeta inhibitor A′, as compared to the only moderate tumor growth inhibitions resulting from monotherapy treatment with either DGKalpha inhibitor A or DGKzeta inhibitor A′.

FIG. 6 shows data obtained from the experiment described in detail in Example 5, in which the effects of combination therapies of DGKalpha inhibitor A (referred to as “Cpd A” in FIG. 6), and DGKzeta inhibitor A′ (referred to as “Cpd A′” in FIG. 6) in different dose ratios were studied versus the respective monotherapies, and in which the concentration of DGKzeta inhibitor A′ was titrated down, as described in the treatment groups below:
[symbol] : Vehicle
[symbol] : DGKalpha inhibitor A (3 mg/kg) p.o., q.d.
[symbol] : DGKzeta inhibitor A′ (3 mg/kg) p.o., q.d.
[symbol] : DGKzeta inhibitor A′ (0.3 mg/kg) p.o., q.d.+DGKalpha inhibitor A (3 mg/kg) q.d.
[symbol] : DGKzeta inhibitor A′ (1 mg/kg) p.o., q.d.+DGKalpha inhibitor A (3 mg/kg) q.d. [symbol]
[symbol] : DGKzeta inhibitor A′ (3 mg/kg) p.o., q.d.+DGKalpha inhibitor A (3 mg/kg) q.d.

FIG. 7 shows data obtained from the experiment described in detail in Example 5, in which the effects of combination therapies of DGKalpha inhibitor A (referred to as “Cpd A” in FIG. 7), and DGKzeta inhibitor A′ (referred to as “Cpd A′” or “Cmpd A” in FIG. 7) in different dose ratios were studied versus the respective monotherapies, and in which the concentration of DGKalpha inhibitor A was titrated down, as described in the treatment groups below:
[symbol] : Vehicle
[symbol] : DGKalpha inhibitor A (3 mg/kg) p.o., q.d.
[symbol] : DGKzeta inhibitor A′ (3 mg/kg) p.o., q.d.
[symbol] : DGKzeta inhibitor A′ (3 mg/kg) p.o., q.d.+DGKalpha inhibitor A (0.3 mg/kg) q.d.
[symbol] : DGKzeta inhibitor A′ (1 mg/kg) p.o., q.d.+DGKalpha inhibitor A (1 mg/kg) q.d.
[symbol] : DGKzeta inhibitor A′ (3 mg/kg) p.o., q.d.+DGKalpha inhibitor A (3 mg/kg) q.d.

For a discussion of the results shown in FIGS. 6 and 7, the reader is referred to Example 5, infra.

FIG. 8 shows the time course of tumor growth in the syngeneic MC38 murine breast carcinoma model following treatment with DGKzeta inhibitor A′, with DGKalpha inhibitor A, anti-PD-L1 antibody and anti-CCR8-antibody, and combinations thereof, in the treatment groups, according to Example 6, however only as far as relevant for the combinations of the present invention, such as the combinations of DGKalpha inhibitor A and DGKzeta inhibitor A′. The full set of data from this experiment is also presented in tables 6.1 and 6.2, with the data relevant for the combinations of the present invention being highlighted in bold.
-▾-: Vehicle & isotype controls (for aCCR8 and aPD-(L)1), isotype controls being administered i.p. at 3 mg/kg BIW×4
-●-: DGKalpha inhibitor A (referred to as DGKα inh in FIG. 8) administered p.o. at 3 mg/kg with a QD administration scheme
-▴-: DGKzeta inhibitor A′ (referred to as DGKz inh in FIG. 8) administered p.o. at 3 mg/kg with a QD administration scheme
••▪••: DGKalpha inhibitor A plus DGKzeta inhibitor A′ (referred to as DGKa inh+DGKz inh in FIG. 8) administered p.o. at 3 mg/kg each with a QD administration scheme

For a discussion of the results shown in FIG. 8, the reader is referred to Example 6.

FIG. 9 shows the probability of survival in the syngeneic MC38 murine breast carcinoma model following treatment with DGKzeta inhibitor A′, with DGKalpha inhibitor A, anti-PD-L1 antibody and anti-CCR8-antibody, and combinations thereof, in the treatment groups, according to Example 6, however only as far as relevant for the combinations of the present invention, such as the combinations of DGKalpha inhibitor A and DGKzeta inhibitor A′.
[symbol] : Vehicle & isotype controls (referred to “Iso Ctrl/vehicle” in FIG. 9) for aCCR8 and aPD-(L)1), isotype controls being administered i.p. at 3 mg/kg BIW×4
[symbol] : DGKalpha inhibitor A (referred to as DGKa inh in FIG. 9) administered p.o. at 3 mg/kg with a QD administration scheme
[symbol] : DGKzeta inhibitor A′ (referred to as DGKz inh in FIG. 9) administered p.o. at 3 mg/kg with a QD administration scheme
-: DGKalpha inhibitor A (referred to as DGKa inh in FIG. 9) plus DGKzeta inhibitor A′ (referred to as DGKz inh in FIG. 9) administered p.o. at 3 mg/kg each with a QD administration scheme.

For a discussion of the results shown in FIG. 9, the reader is referred to Example 6.

FIG. 10 shows tumor growth in the surviving animals at the end of the study described in Example 6 upon re-inoculation with MC38 tumor cells (i.e. the two animals which had received DGKalpha inhibitor A (referred to as DGKa inh in FIG. 10) plus DGKzeta inhibitor A′ (referred to as DGKz inh in FIG. 10) administered p.o. at 3 mg/kg each with a QD administration scheme), and in a control group.
[symbol] Control group
-∘- Animals which had received DGKalpha inhibitor A (referred to as DGKa inh in FIG. 10) plus DGKzeta inhibitor A′ (referred to as DGKz inh in FIG. 10) administered p.o. at 3 mg/kg each with a QD administration scheme, in the experiment described in Example 6.

For a discussion of the results shown in FIG. 10, the reader is referred to Example 6.

### EXPERIMENTAL SECTION

### Experimental Section—General

In this Experimental Section, the term “DGKalpha inhibitor A” is a preferred example of a DGKalpha inhibitor. DGKalpha inhibitor A is described in the International Patent Application PCT/EP2020/083198, published as WO 2021/105117 A1, Example 298. As shown herein DGKalpha inhibitor A is 6-fluoro-1-methyl-4-[4-(5-methyl-1,3-benzoxazol-2-yl)piperidin-1-yl]-2-oxo-1,2-dihydroquinoline-3-carboxamide, of structure:

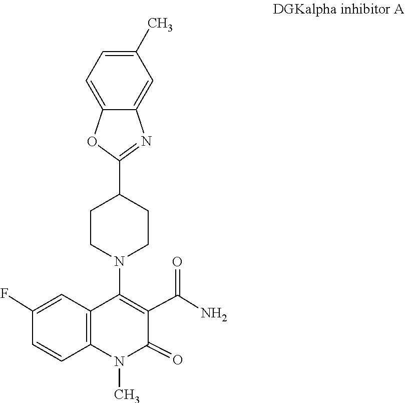 [SMILES: Cc1ccc2oc(C3CCN(c4c(C(N)=O)c(=O)n(C)c5ccc(F)cc45)CC3)nc2c1]

Further inhibitors of DGKalpha characterized in vitro as shown below in Example 1 are the following Example compounds described in the International Patent Application PCT/EP2020/083198, published as WO 2021/105117 A1:
4-[4-(6-fluoro-1,3-benzoxazol-2-yl)piperidin-1-yl]-1-methyl-2-oxo-1,2-dihydroquinoline-3-carbonitrile (Example 4)
1-methyl-4-[4-(5-methyl-1,3-benzoxazol-2-yl)piperidin-1-yl]-2-oxo-1,2-dihydroquinoline-3-carboxamide (Example 107)
4-[4-(4-methoxyphenyl)piperidin-1-yl]-1-methyl-2-oxo-1,2-dihydroquinoline-3-carboxamide (Example 112)
N,1-dimethyl-4-[4-(5-methyl-1,3-benzoxazol-2-yl)piperidin-1-yl]-2-oxo-1,2-dihydroquinoline-3-carboxamide (Example 448)

In this Experimental Section, the term “DGKzeta inhibitor A” is a preferred example of a DGKzeta inhibitor. DGKzeta inhibitor A′ is described in Example 62.2 of International Patent Application PCT/EP2021/060167, published as WO 2021/214019 A1. As shown herein DGKzeta inhibitor A′ is (R)-2-(N-[4-amino-5-(4-methoxybenzoyl)thiazol-2-yl]-4-fluoro-anilino)propanamide, of structure:

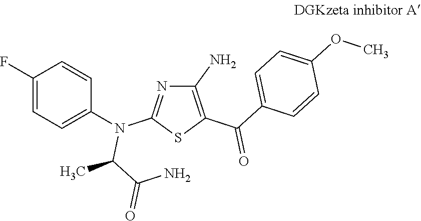 [SMILES: COc1ccc(C(=O)c2sc(N(c3ccc(F)cc3)[C@H](C)C(N)=O)nc2N)cc1]

Further inhibitors of DGKzeta characterized in vitro as shown below in Example 1 are the following Example compounds described in the International Patent Application PCT/EP2021/060167, published as WO 2021/214019 A1:
2-(N-[4-amino-5-[6-(trifluoromethyl)pyridine-3-carbonyl]thiazol-2-yl]-4-fluoro-anilino)propanamide (enantiomer 2) (Example 29.2)
2-(N-[4-amino-5-[4-(trifluoromethoxy)benzoyl]thiazol-2-yl]-4-fluoro-anilino)propanamide (enantiomer 2) (Example 40.2)
2-(N-[4-amino-5-[4-(difluoromethoxy)benzoyl]thiazol-2-yl]-4-fluoro-anilino)propanamide (enantiomer 2) (Example 44.2)
2-(N-[4-amino-5-(4-methoxybenzoyl)thiazol-2-yl]-3,4-difluoro-anilino)propanamide (enantiomer 1) (Example 51.1)

### Experimental Section—In Vitro Assays and Data

Compounds were tested in in vitro assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.
Compounds were synthesized one or more times. When synthesized more than once, data from biological assays represent average values or median values calculated utilizing data sets obtained from testing of one or more synthetic batch.
If more than one value is reported, the individual values represent one or more test results, as average value or as median value.

The in vitro activity of the compounds of the present invention was demonstrated in the following assays:

### Human DGKalpha Kinase Activity Inhibition Assay.

Human DGKalpha inhibitory activity of compounds of the present invention was quantified employing the human DGKalpha kinase activity assay as described in the following paragraphs. In essence, the enzyme activity was measured by quantification of the adenosine-di-phosphate (ADP) generated as a co-product of the enzyme reaction via the “ADP-Glo™ Kinase Assay” kit from the company Promega. This detection system works as follows: In a first step the ATP not consumed in the kinase reaction is quantitatively converted to cAMP employing an adenylate cyclase (“ADP-Glo-reagent”), then the adenylate cyclase is stopped and the ADP generated in the kinase reaction converted to ATP, which subsequently generates in a luciferase-based reaction a glow-luminescence signal (“Kinase Detection Reagent”).

C-terminally FLAG-tagged, recombinant full-length human DGKalpha (expressed in baculovirus infected insect cells, purified using anti-Flag pulldown and size exclusion chromatography as described below, DGKa_hu_1) was used as enzyme. As substrate for the kinase 1,2-dioleoyl-sn-glycerol, reconstituted in octyl-β-D-glucopyranoside micelles, was used. For the preparation of the micelles, 1 volume of a 16.1 mM solution of 1,2-dioleoyl-sn-glycerol (Avanti, Cat. #O8001-25G) in chloroform was slowly evaporated using a nitrogen stream. Subsequently, 22.55 volumes of a 510 mM solution of octyl-β-D-glucopyranoside (Sigma-Aldrich, Cat. #O8001-10G) in 50 mM MOPS buffer (pH 7.4) were added, and the mixture was sonicated in an ultrasonic bath for 20 s. Then 35 volumes of 50 mM MOPS buffer (pH 7.4) were added to yield a solution of 0.28 mM 1,2 dioleoyl-sn-glycerol and 200 mM octyl-β-D-glucopyranoside, which was aliquoted, flash-frozen in liquid nitrogen, and stored at −20° C. until use. For each experiment, a fresh aliquot was quickly thawed and diluted 24-fold with aqueous assay buffer (described below) containing 95.7 μM adenosine triphosphate (Promega) to yield a 1.67-fold concentrated substrate solution.

For the assay 50 nl of a 100-fold concentrated solution of the test compound in dimethyl sulfoxide (DMSO, Sigma) was pipetted into either a white 1536-well or a white low-volume 384-well microtiter plate (both Greiner Bio-One, Frickenhausen, Germany). Subsequently, 2 μl of a solution of human DGKα in aqueous assay buffer [50 mM (3-(N-morpholino)propanesulfonic acid (MOPS, pH 7.4, Sigma-Aldrich), 1 mM dithiothreitol (DTT, Sigma-Aldrich), 100 mM NaCl (Sigma-Aldrich), 10 mM MgCl₂ (Sigma-Aldrich), 0.1% (w/v) bovine gamma globulin (BGG, Sigma-Aldrich), 1 μM CaCl₂ (Sigma-Aldrich)] were added to the wells, and the mixture was incubated for 15 min at 22° C. to allow pre-binding of the test compounds to the enzyme. The reaction was initiated by the addition of 3 μl of substrate solution [preparation described above; 11.7 μM 1,2-dioleoyl-sn-glycerol (=>final conc. in the 5 μl assay volume is 7 μM), 8.33 mM octyl-β-D-glucopyranoside (=>final conc. in 5 μl assay volume is 5 mM), and 91.67 μM adenosine triphosphate (=>final conc. in 5 μl assay volume is 55 μM) in assay buffer] and the resulting mixture was incubated for a reaction time of 20 min at 22° C. The concentration of DGKα was adjusted depending of the activity of the enzyme lot and was chosen appropriate to have the assay in the linear range, a typical concentration is about 0.1 nM. The reaction was stopped by the addition of 2.5 al of “ADP-Glo-reagent” (1 to 1.5 diluted with water) and the resulting mixture was incubated at 22° C. for 1 h to convert the ATP not consumed in the kinase reaction completely to cAMP. Subsequently 2.5 μl of the “kinase detection reagent” (1.2-fold more concentrated than recommended by the producer) were added, the resulting mixture was incubated at 22° C. for 1 h and then the luminescence measured with a suitable measurement instrument (e.g. Viewlux™ from Perkin-Elmer). The amount of emitted light was taken as a measure for the amount of ADP generated and thereby for the activity of the DGKα.

The data were normalised (enzyme reaction without inhibitor=0% inhibition, all other assay components but no enzyme=100% inhibition). Usually the test compounds were tested on the same microtiterplate in 11 different concentrations in the range of 20 μM to 0.07 nM (20 μM, 5.7 μM, 1.6 μM, 0.47 μM, 0.13 aM, 38 nM, 11 nM, 3.1 nM, 0.9 nM, 0.25 nM and 0.07 nM, the dilution series prepared separately before the assay on the level of the 100-fold concentrated solutions in DMSO by serial dilutions, exact concentrations may vary depending pipettors used) in duplicate values for each concentration and IC₅₀ values were calculated using Genedata Screener™ software.

### Expression of DGKalpha in Insect Cells Using the Baculovirus System

### Expression Constructs:

The cDNA encoding the full length sequence of human DGKalpha (Uniprot P23743) was optimized for expression in eukaryotic cells and synthesized by the GeneArt Technology at Life Technologies.

The DNA sequence encoded the following sequence:

### Construct DGKa_hu Amino Acid M1 to S735

Additionally the expression construct encoded: a Kozak DNA sequence for translation initiation (GCCACC), at the C-terminus a Flag (DYKDDDDK) sequence followed by two stop codons and additionally 5′ and 3′ att-DNA sequences for Gateway Cloning.

The DGKα construct was subcloned using the Gateway Technology into the Destination vector pD-INS. The vector pD-INS is a Baculovirus transfer vector (based on vector pVL1393, Pharmingen) which enables the expression of the DGK-Flag protein. The respective protein was named DNA_hu_1.

Additionally the DNA construct DGKa_hu with C-terminal Flag tag was also subcloned in to the Destination vector pD-INSA. This Baculovirus transfer vector is designed to fuse a His6 tag +Avi tag protein sequence to N-terminus of the DGKa_hu-Flag protein. The complete encoded protein was designated DGKa_hu_1Avi. The Avi-tag sequence enables a site-specific in-vitro biotinylation of the DGKα protein.

### Generation of Recombinant Baculovirus

In separate approaches each of the two DGK transfer vectors was co-transfected in Sf9 cells with Baculovirus DNA (Flashbac Gold DNA, Oxford Expression Technologies) using Fugene HD (Roche). After 5 days the supernatant of the transfected cells containing the recombinant Baculovirus encoding the various DGK proteins was used for further infection of Sf9 cells for virus amplification whereby the virus titer was monitored using qPCR.

### DGK Expression in Sf9 Cells Using Bioreactor

Sf9 cells cultured (Insect-xpress medium, Lonza, 27° C.) in a Wave-bioreactor with a disposable culture bag were infected at a cell density of 10⁶ cells/mL with one of the recombinant baculovirus stocks at a multiplicity of infection of 1 and incubated for 72. Subsequently the cells were harvested by centrifugation (800×g) and cell pellet frozen at −80° C.

To produce biotinylated DGKa_hu_1Avi the Sf9 cells in the bioreactor were co-infected with the Baculovirus encoding DGKa_hu_1Avi as well as with a Baculovirus encoding the biotinylation enzyme BirA.

### Purification of the DGK-Flag Proteins:

Purification of the DGK-Flag proteins was achieved by a two-step chromatography procedure as follows.

The pelleted cells (from 8 L cell culture) were resuspended in Lysis-Buffer (50 mM Tris HCl 7.4; 150 mM NaCl; 10 mM MgCl₂; 1 μM CaCl₂; 1 mM DTT; 0.1% NP-40; 0.1% NP-40; Complete Protease Inhibitor Cocktail-(Roche)) and lysed by a freeze-thaw cycle followed by an incubation on ice for 60 min. The lysate was centrifuged at 63.000×g for 30 min. at 4° C. The soluble supernatant was than incubated with 25 mL anti-Flag M2 Agarose (Sigma) in a plastic flask rotating for 16 h at 4° C. for binding of the DGK-Flag proteins, subsequently rinsed with 10×25 mL Wash-Buffer (50 mM Tris HCl 7.4; 150 mM NaCl; 10 mM MgCl₂; 1 μM CaCl₂; 1 mM DTT) and finally the bound protein was eluted using Elusion-Buffer (Wash-Buffer with 300 μg/mL FLAG-Peptide, incubated 30 min. at 4° C. with 3×15 mL).

The elution fractions from the affinity chromatography were concentrated (using Amicon Ultra 15, Centrifugal Filters, 30 kDa MW cut-off; Millipore #UFC903024) to 10 mL and applied to a size exclusion chromatography column (S200 prep grade 26/60, GE Healthcare) and the resulting monomeric peak fraction was collected, pooled and again concentrated. Wash-buffer was used for size exclusion chromatography and the final concentrated sample. The final protein sample concentration was 5-10 mg/mL and the yield was 1-2 mg final protein per L cell culture.

For DGKa_hu_1Avi a biotinylation level of 100% was demonstrated by mass spectromentry.

### Human DGKzeta Kinase Activity Inhibition Assay.

Human diacylglycerol kinase zeta (DGKζ) inhibitory activity of compounds of the present invention was quantified employing the human DGKζ kinase activity assay as described in the following paragraphs. In essence, the enzyme activity was measured by quantification of the adenosine-di-phosphate (ADP) generated as a co-product of the enzyme reaction via the “ADP-Glo™ Kinase Assay” kit from the company Promega. This detection system works as follows: In a first step the adenosine-tri-phosphate (ATP) not consumed in the kinase reaction is quantitatively converted to cyclic adenosine-mono-phosphate (cAMP) employing an adenylate cyclase (“ADP-Glo-reagent”), then the adenylate cyclase is stopped and the ADP generated in the kinase reaction is converted to ATP, which subsequently generates in a luciferase-based reaction a glow-luminescence signal (“Kinase Detection Reagent”).

C-terminally FLAG-tagged, recombinant full-length human DGKζ (inhouse expressed in baculovirus infected insect cells, purified using anti-Flag pulldown and size exclusion chromatography) was used as enzyme. As an alternative, commercially available enzyme by Carnabio can be used. As substrate for the kinase, 1,2-dioleoyl-sn-glycerol, reconstituted in octyl-β-D-glucopyranoside micelles, was used. For the preparation of the micelles, 1 volume of a 16.1 mM solution of 1,2-dioleoyl-sn-glycerol (Avanti, Cat. #O8001-25G) in chloroform was slowly evaporated using a nitrogen stream. Subsequently, 22.55 volumes of a 510 mM solution of octyl-β-D-glucopyranoside (Sigma-Aldrich, Cat. #O8001-10G) in 50 mM MOPS buffer (pH 7.4) were added, and the mixture was sonicated in an ultrasonic bath for 20 s. Then 35 volumes of 50 mM MOPS buffer (pH 7.4) were added to yield a solution of 0.28 mM 1,2 dioleoyl-sn-glycerol and 200 mM octyl-β-D-glucopyranoside, which was aliquoted, flash-frozen in liquid nitrogen, and stored at −20° C. until use. For each experiment, a fresh aliquot was quickly thawed and diluted 24-fold with aqueous assay buffer (described below) containing 95.7 IM adenosine triphosphate (Promega) to yield a 1.67-fold concentrated substrate solution.

For the assay 50 nl of a 100-fold concentrated solution of the test compound in dimethyl sulfoxide (DMSO, Sigma) was pipetted into either a white 1536-well or a white low-volume 384-well microtiter plate (both Greiner Bio-One, Frickenhausen, Germany). Subsequently, 2 μl of a solution of human DGKζ in aqueous assay buffer [50 mM (3-(N-morpholino)propanesulfonic acid (MOPS, pH 7.4, Sigma-Aldrich), 1 mM dithiothreitol (DTT, Sigma-Aldrich), 100 mM NaCl (Sigma-Aldrich), 10 mM MgCl₂ (Sigma-Aldrich), 0.1% (w/v) bovine gamma globulin (BGG, Sigma-Aldrich), 1 μM CaCl₂ (Sigma-Aldrich)] were added to the wells, and the mixture was incubated for 15 min at 22° C. to allow pre-binding of the test compounds to the enzyme. The reaction was initiated by the addition of 3 μL of substrate solution [preparation described above; 11.7 μM 1,2-dioleoyl-sn-glycerol (=>final conc. in the 5 μL assay volume is 7 μM), 8.33 mM octyl-β-D-glucopyranoside (=>final conc. in 5 μL assay volume is 5 mM), and 91.67 μM adenosine triphosphate (=>final conc. in 5 μL assay volume is 55 μM) in assay buffer] and the resulting mixture was incubated for a reaction time of 20 min at 22° C. The concentration of DGKζ was adjusted depending of the activity of the enzyme lot and was chosen appropriate to have the assay in the linear range, a typical concentration is about 0.1 nM. The reaction was stopped by the addition of 2.5 μL of “ADP-Glo-reagent” (1 to 1.5 diluted with water) and the resulting mixture was incubated at 22° C. for 1 h to convert the ATP not consumed in the kinase reaction completely to cAMP. Subsequently 2.5 μl of the “kinase detection reagent” (1.2 fold more concentrated than recommended by the producer) were added, the resulting mixture was incubated at 22° C. for 1 h and then the luminescence measured with a suitable measurement instrument (e.g. Viewlux™ from Perkin-Elmer). The amount of emitted light was taken as a measure for the amount of ADP generated and thereby for the activity of the DGKζ.

The data were normalised (enzyme reaction without inhibitor=0% inhibition, all other assay components but no enzyme=100% inhibition). Usually the test compounds were tested on the same microtiterplate in 11 different concentrations in the range of 20 μM to 0.07 nM (20 paM, 5.7 μM, 1.6 μM, 0.47 μM, 0.13 μM, 38 nM, 11 nM, 3.1 nM, 0.9 nM, 0.25 nM and 0.07 nM, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

### Example 1: IC₅₀ Values of Examples in In Vitro Human DGKalpha and DGKzeta Kinase Activity Inhibition Assays

| Patent | Example | IC₅₀ | | IC₅₀ | |
|---|---|---|---|---|---|
| application | No | hDGKa | | hDGKz | |
| PCT/EP2021/060167 | 29.2 | >15000 | nM | 46 | nM |
| PCT/EP2021/060167 | 40.2 | >12200 | nM | 7.1 | nM |
| PCT/EP2021/060167 | 44.2 | >20000 | nM | 3.9 | nM |
| PCT/EP2021/060167 | 44.2 | | | 6.7 | nM |
| PCT/EP2021/060167 | 51.1 | >13300 | nM | 8.2 | nM |
| PCT/EP2021/060167 | 51.1 | | | 8.9 | nM |
| PCT/EP2021/060167 | 62.2 (DGKzeta | >3630 | nM | 14 | nM |
| | inhibitor A′) | | | | |
| PCT/EP2021/060167 | 62.2 (DGKzeta | | | 23 | nM |
| | inhibitor A′) | | | | |
| PCT/EP2020/083198 | 4 | 2.8 | nM | >20000 | nM |
| PCT/EP2020/083198 | 107 | 2.3 | nM | >20000 | nM |
| PCT/EP2020/083198 | 112 | 4.5 | nM | >20000 | nM |
| PCT/EP2020/083198 | 298 (DGKalpha | 0.25 | nM | 17200 | nM |
| | inhibitor A) | | | | |
| PCT/EP2020/083198 | 298 (DGKalpha | 0.5 | nM | | |
| | inhibitor A) | | | | |
| PCT/EP2020/083198 | 448 | 27 | nM | >20000 | nM |

### Example 2: Tumor Cell Killing Assay (Colo-800 MART1 Cells in xCELLigence #104 Assay)

Human Colo-800 melanoma cells were seeded in a 96-well E-Plate (2×10⁴ cells/well) for detection of cell adherence [=cell index (CI)], directly correlating with tumor cell viability and proliferation. After 24 hours, tumor antigen-specific MART1 T cells were added (here 6×10⁴ cells/well) with increasing concentrations of DGKalpha inhibitor A (Cpd A, 1 nM, 10 nM, 50 nM), DGKzeta inhibitor A′ (Cpd A′, 1 nM, 10 nM, 50 nM) or the equimolar combination of both (1 nM, 10 nM, 20 nM, 30 nM, 50 nM).

Control groups were (i) human Colo-800 cells in medium alone (i.e. without addition of tumor antigen-specific MART1 T cells and of DGK inhibitors), (ii) human Colo-800 cells with the addition of DGKalpha inhibitor A and DGKzeta inhibitor A′ (50 nM each) but without the addition of tumor antigen-specific MART1 T cells, and (iii) human Colo-800 cells with the addition of tumor antigen-specific MART1 T cells in medium “Vehicle”. Tumor cell index was quantified in five-minute intervals for a >90 hours period. Figure AA depicts the normalized cell index of the technical triplicates for each treatment group 46 hours after the start of co-culture. Figure BB displays the secretion levels of IFN-γ quantified 72 hours after start of co-culture via ELISA.

For details of the xCELLigence #104 assay, the reader is referred to J. Chou et al., Immunother. 2012 February; 35(2): 131-141, FIG. 6.

As shown in FIG. 1, monotherapy with DGKalpha inhibitor A and with DGKzeta inhibitor A′ results in a dose-dependent reduction of the TCI, with the stronger effect size for DGKzeta inhibitor A′, which effects killing of the majority of tumor cells at 10 nM and virtually complete tumor cell killing at 50 nM, whilst even at 50 nM the effect size of DGKalpha inhibitor A on the TCI is only moderate. In contrast, upon combined treatment with both DGKalpha inhibitor A and DGKzeta inhibitor A′ even at a concentration of 1 nM each a virtually complete killing of the tumor cells is effected, at which concentration neither DGKalpha inhibitor A nor DGKzeta inhibitor A′ exercise a major effect in monotherapy. As shown in FIG. 2, monotherapy even at a concentration of 50 nM effects only a slight (DGKalpha inhibitor A) or moderate (DGKzeta inhibitor A′) increase in IFN-γ secretion. In contrast, combined treatment with DGKalpha inhibitor A and DGKzeta inhibitor A′ at a concentration of only 10 nM each show a much more pronounced effect, and even at 1 nM each the effect is comparable to the maximal effect size of monotherapy with DGKzeta inhibitor A′ at 50 nM, and is superior over monotherapy with DGKalpha inhibitor A at 50 nM.

### Example 3: Activation of PBMC Cells as a Function of Treatment with DGKalpha Inhibitor a and DGKzeta Inhibitor A′ in Different Concentrations and Concentration Ratios

Activation of PBMC cells as a function of treatment with DGKalpha inhibitor A and DGKzeta inhibitor A′ in different concentrations was determined by measuring the proportion of CD25+ cells within the CD8+ naïve T-cells in the respective samples. In addition to the experimental determination according to the protocol provided below, said proportion of CD25+ cells withing the CD8+ naïve T-cells was simulated according to Meyer et al., 2019, Cell Systems 8, 97-108, Feb. 27, 2019.

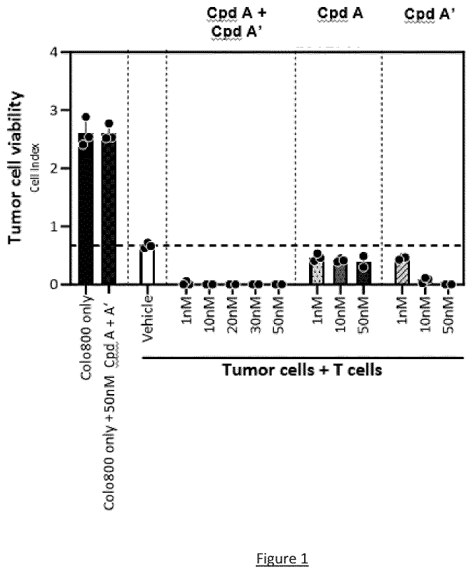

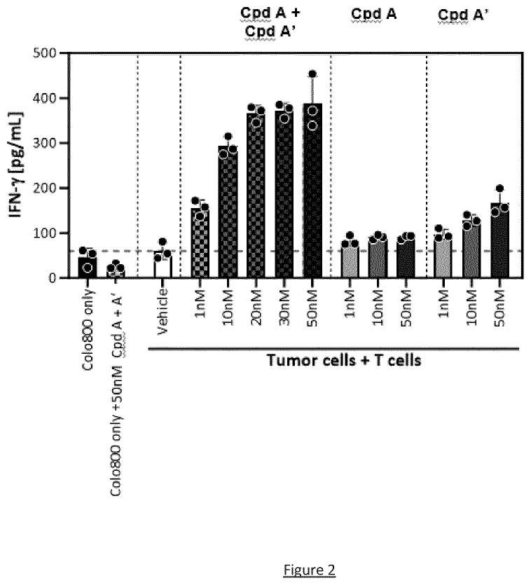

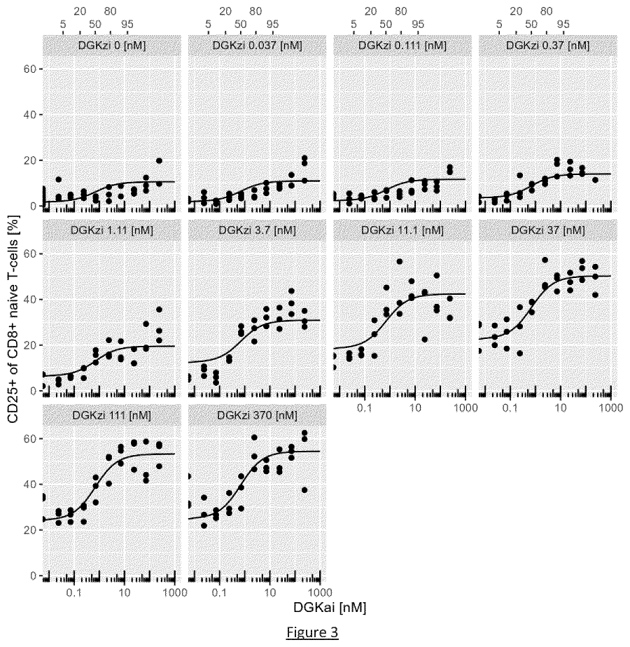

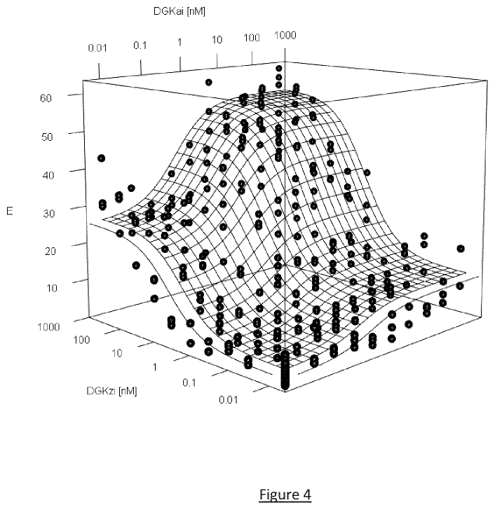

Table 3.1, below, provides the results of the experimental determination of the PBMC cell activation in triplicate, plus the result of said simulation, at a wide range of concentrations of DGKalpha inhibitor A and DGKzeta inhibitor A′. Table 3.2, below, provides control data obtained from subjecting the PBMC cells to the protocol below, however without adding DGKalpha inhibitor A and DGKzeta inhibitor A′. FIG. 3 shows the relative amount of activated (CD25+) naïve CD8 T cells (i.e. the observed activations as specified in tables 3.1 and 3.2) as a function of the unbound concentration of DGKalpha inhibitor A (referred to as DGKai [nM] in FIG. 3) at unbound concentrations of DGKzeta inhibitor A′ (referred to as DGKzi in FIG. 3) of 0 nM, 0.037 nM, 0.111 nM, 0.37 nM, 1.11 nM, 3.7 nM, 11.1 nM, 37 nM, 111 nM, and 370 nM. The numbers 5, 20, 50, 80 and 95 above the upper x-axis in FIG. 3 mark the concentrations needed for 5, 20, 50, 80 and 95% of DGKai inhibition. FIG. 4 shows the data visualized as concentration response surface.

Human PBMCs (ordered frozen from StemCell Technologies; Donor 1003762, Lot 1704070034) were plated in 384-Well plates with 70000 cells in 70 μl cell culture media and stimulated with ImmunoCult™ Human CD3/CD28 T Cell Activator (StemCell, #10971) in a final dilution of 1:320 with the addition of nine increasing concentrations of DGKalpha inhibitor A and DGKzeta inhibitor A′ alone or in combination. The relative amount of activated (CD25+) naïve CD8 T cells were analyzed by flow cytometry. Non-stimulated cells served as negative control, cells stimulated with 30 ng/ml aCD3/aCD28/IgG were used as positive control.

The data shown in tables 3.1 and 3.2, and visualized in FIGS. 3 and 4, clearly show that combined treatment of the PBMC cells with DGKalpha inhibitor A and DGKzeta inhibitor A′ allows to reach levels of PBMC activation which are not possible to reach by treatment with either DGKalpha inhibitor A or DGKzeta inhibitor A′, even at substantially increased concentration.

TABLE 3.1
Experimentally determined and simulated PBMC activation
by combined DGKalpha and DGKzeta inhibition at different
concentrations and concentration ratios of DGKalpha
inhibitor A and DGKzeta inhibitor A′.

| | |
|---|---|
| Unbound | CD25+ of CD8+ |
| concentration [nM] | naïve T-cells [%] |

| DGKai | DGKzi | Observed | | | Simulated |
|---|---|---|---|---|---|
| 0 | 0.037 | 2 | 2 | 3.2 | 1.9 |
| 0 | 0.11 | 4.7 | 3.6 | 5.3 | 2.3 |
| 0 | 0.37 | 1.7 | 1.6 | 4.2 | 3.6 |
| 0 | 1.1 | 7.2 | 2.1 | 7.1 | 6.5 |
| 0 | 3.7 | 6 | 4.9 | 6.4 | 12 |
| 0 | 11 | 15 | 10 | 15 | 19 |
| 0 | 37 | 29 | 18 | 29 | 23 |
| 0 | 111 | 34 | 25 | 35 | 24 |
| 0 | 370 | 32 | 44 | 31 | 25 |
| 0.024 | 0 | 4.1 | 3.1 | 12 | 2 |
| 0.024 | 0.037 | 1.2 | 6.1 | 3.6 | 2.2 |
| 0.024 | 0.11 | 1.1 | 5.5 | 3.1 | 2.6 |
| 0.024 | 0.37 | 1.5 | 3 | 2.9 | 3.9 |
| 0.024 | 1.1 | 4.6 | 2.8 | 5 | 6.9 |
| 0.024 | 3.7 | 6.5 | 9.3 | 11 | 13 |
| 0.024 | 11 | 17 | 14 | 15 | 19 |
| 0.024 | 37 | 29 | 24 | 20 | 24 |
| 0.024 | 111 | 28 | 27 | 23 | 25 |
| 0.024 | 370 | 27 | 34 | 22 | 26 |
| 0.072 | 0 | 4.8 | 3.7 | 5 | 2.6 |
| 0.072 | 0.037 | 2.4 | 0.9 | 2.3 | 2.8 |
| 0.072 | 0.11 | 4.6 | 3.8 | 3.2 | 3.2 |
| 0.072 | 0.37 | 4.7 | 5.4 | 2.3 | 4.6 |
| 0.072 | 1.1 | 6.2 | 6.6 | 5.6 | 7.7 |
| 0.072 | 3.7 | 8 | 6 | 3.6 | 14 |
| 0.072 | 11 | 16 | 18 | 16 | 21 |
| 0.072 | 37 | 26 | 31 | 19 | 25 |
| 0.072 | 111 | 27 | 24 | 29 | 27 |
| 0.072 | 370 | 27 | 25 | 29 | 28 |
| 0.24 | 0 | 3.4 | 5.1 | 6.4 | 4 |
| 0.24 | 0.037 | 5.4 | 3.8 | 2.6 | 4.3 |
| 0.24 | 0.11 | 3.5 | 6.1 | 2.9 | 4.7 |
| 0.24 | 0.37 | 5.7 | 13 | 3.9 | 6.3 |
| 0.24 | 1.1 | 5.6 | 10 | 9.8 | 9.9 |
| 0.24 | 3.7 | 13 | 13 | 15 | 17 |
| 0.24 | 11 | 31 | 25 | 15 | 25 |
| 0.24 | 37 | 28 | 37 | 16 | 30 |
| 0.24 | 111 | 31 | 30 | 24 | 32 |
| 0.24 | 370 | 29 | 27 | 36 | 32 |
| 0.72 | 0 | 3.6 | 4.3 | 2.3 | 6.3 |
| 0.72 | 0.037 | 5.4 | 3.3 | 4.3 | 6.6 |
| 0.72 | 0.11 | 7 | 6.6 | 3.6 | 7.1 |
| 0.72 | 0.37 | 9.7 | 9.4 | 6.8 | 8.9 |
| 0.72 | 1.1 | 18 | 16 | 12 | 13 |
| 0.72 | 3.7 | 26 | 28 | 25 | 22 |
| 0.72 | 11 | 36 | 45 | 34 | 31 |
| 0.72 | 37 | 39 | 39 | 34 | 37 |
| 0.72 | 111 | 39 | 43 | 32 | 39 |
| 0.72 | 370 | 29 | 39 | 44 | 40 |
| 2.4 | 0 | 8.3 | 2.1 | 5 | 8.6 |
| 2.4 | 0.037 | 7 | 4.1 | 3.7 | 9 |
| 2.4 | 0.11 | 6.5 | 3.9 | 5.2 | 9.6 |
| 2.4 | 0.37 | 11 | 9.6 | 12 | 12 |
| 2.4 | 1.1 | 22 | 15 | 16 | 17 |
| 2.4 | 3.7 | 22 | 27 | 31 | 27 |
| 2.4 | 11 | 34 | 39 | 57 | 37 |
| 2.4 | 37 | 57 | 46 | 45 | 44 |
| 2.4 | 111 | 52 | 52 | 40 | 47 |
| 2.4 | 370 | 47 | 61 | 52 | 48 |
| 7.2 | 0 | 4.2 | 8.8 | 8.8 | 9.8 |
| 7.2 | 0.037 | 7.6 | 10 | 5.6 | 10 |
| 7.2 | 0.11 | 11 | 6.7 | 5.8 | 11 |
| 7.2 | 0.37 | 20 | 13 | 18 | 13 |
| 7.2 | 1.1 | 15 | 22 | 14 | 18 |
| 7.2 | 3.7 | 32 | 36 | 28 | 29 |
| 7.2 | 11 | 41 | 41 | 48 | 40 |
| 7.2 | 37 | 49 | 51 | 44 | 48 |
| 7.2 | 111 | 55 | 56 | 49 | 51 |
| 7.2 | 370 | 51 | 46 | 47 | 52 |
| 24 | 0 | 6.8 | 5.4 | 7.3 | 10 |
| 24 | 0.037 | 9.8 | 7.6 | 8.5 | 11 |
| 24 | 0.11 | 11 | 9.6 | 7.3 | 11 |
| 24 | 0.37 | 13 | 19 | 16 | 14 |
| 24 | 1.1 | 12 | 12 | 18 | 19 |
| 24 | 3.7 | 31 | 36 | 27 | 30 |
| 24 | 11 | 23 | 43 | 43 | 42 |
| 24 | 37 | 52 | 48 | 44 | 50 |
| 24 | 111 | 58 | 46 | 58 | 52 |
| 24 | 370 | 45 | 47 | 55 | 54 |
| 72 | 0 | 9 | 6.7 | 12 | 11 |
| 72 | 0.037 | 9 | 14 | 8.9 | 11 |
| 72 | 0.11 | 6.7 | 8.5 | 11 | 12 |
| 72 | 0.37 | 14 | 17 | 15 | 14 |
| 72 | 1.1 | 29 | 18 | 19 | 19 |
| 72 | 3.7 | 38 | 34 | 44 | 31 |
| 72 | 11 | 37 | 35 | 51 | 42 |
| 72 | 37 | 49 | 57 | 54 | 50 |
| 72 | 111 | 42 | 44 | 59 | 53 |
| 72 | 370 | 52 | 55 | 56 | 54 |
| 240 | 0 | 20 | 9.7 | 20 | 11 |
| 240 | 0.037 | 11 | 19 | 21 | 11 |
| 240 | 0.11 | 15 | 15 | 17 | 12 |
| 240 | 0.37 | 11 | NA | NA | 14 |
| 240 | 1.1 | 22 | 26 | 36 | 20 |
| 240 | 3.7 | 31 | 35 | 28 | 31 |
| 240 | 11 | 32 | 32 | 40 | 42 |
| 240 | 37 | 54 | 42 | 50 | 50 |
| 240 | 111 | 58 | 57 | 48 | 53 |
| 240 | 370 | 38 | 63 | 60 | 54 |

TABLE 3.2
Vehicle control, unbound concentrations DGKalpha inhibitor
A(DGKai) = DGKzeta inhibitor A′ (DGKzi) =
0 nM and simulated CD25+ of CD8 naïve = 1.7 [%]

| CD25+ of | CD25+ of | CD25+ of | CD25+ of | CD25+ of |
|---|---|---|---|---|
| CD8+ naïve | CD8+ naïve | CD8+ naïve | CD8+ naïve | CD8+ naïve |
| T-cells [%] | T-cells [%] | T-cells [%] | T-cells [%] | T-cells [%] |
| Observed | Observed | Observed | Observed | Observed |
| 1.4 | 0.5 | 1.2 | 2.9 | 7.6 |
| 0.6 | 2.5 | 2.3 | 3.6 | 5.4 |
| 2.2 | 1.6 | 1.5 | 2.3 | 4.3 |
| 3 | 2.6 | 0.7 | 3.7 | 5.6 |
| 1.6 | 2.6 | 3.7 | 3.1 | 5.1 |
| 2 | 3.5 | 4.1 | 1.5 | 2.5 |
| 4.3 | 1.1 | 3.8 | 6.7 | 4.8 |
| 0.6 | 0.4 | 2.2 | 0.8 | |

### Experimental Section—In Vivo Experiments and Data

### Example 4

Effects of the DGKζ Inhibitor DGKzeta Inhibitor A′, the DGKα Inhibitor DGKalpha Inhibitor a, Anti PD-L1, and Combinations Thereof, in the Syngeneic EMT6 Murine Breast Carcinoma Model—Surprising Effect of Combination Therapy with a GKalpha Inhibitor and a DGKzeta Inhibitor

The objective of this study was to observe the effects of combination therapies with DGKzeta and/or &DGKalpha inhibitors and anti PD-L1 antibody on the tumor volume in a syngeneic mouse tumor model in treatment setting. Surprising effects, however, were also found upon combination therapy with DGKalpha inhibitor A and DGKzeta inhibitor A′, compared to the respective monotherapies. The following eight treatment groups are included:

### Vehicle for DGKalpha Inhibitor A and DGKzeta Inhibitor A′:

Polyethylene glycol 400 (PEG)/ethanol (EtOH)/water (60/10/30).
1. Anti-PD-L1 (TPP-3911, 5 mg/kg) i.p., q3/4d
2. DGKzeta inhibitor A′ (5 mg/kg) p.o., q.d.+anti PD-L1 (TPP 3911, 5 mg/kg) i.p., q3/4d
3. DGKzeta inhibitor A′ (5 mg/kg) p.o., q.d.+Isotype control (5 mg/kg) i.p., q3/4d
4. DGKalpha inhibitor A (3 mg/kg) p.o., q.d.+anti PD-L1 (TPP-3911, 5 mg/kg) i.p., q3/4d
5. DGKalpha inhibitor A (3 mg/kg) p.o., q.d.+Isotype control (5 mg/kg) i.p., q3/4d
6. DGKzeta inhibitor A′ (5 mg/kg) p.o., q.d.+DGKalpha inhibitor A (3 mg/kg) p.o., q.d.+anti PD-L1 (TPP-3911, 5 mg/kg) i.p., q3/4d
7. DGKzeta inhibitor A′ (5 mg/kg) p.o., q.d.+DGKalpha inhibitor A (3 mg/kg) p.o., q.d.+Isotype control (5 mg/kg) i.p., q3/4d
8. Vehicle as described supra, q.d.

Mice were assigned to a study at the age of 6-8 weeks. Animal husbandry, feeding and health conditions were according to animal welfare guidelines. EMT6 cell line was cultivated with appropriate medium and split at least 3 times before inoculation. Female Balb/c mice were inoculated subcutaneously with 5×10>5 tumor cells in a medium/matrigel mixture ratio of 1:1. After 8 days the mice were randomized, and therapeutic treatment started when tumors had reached a size of approx. 40-mm².

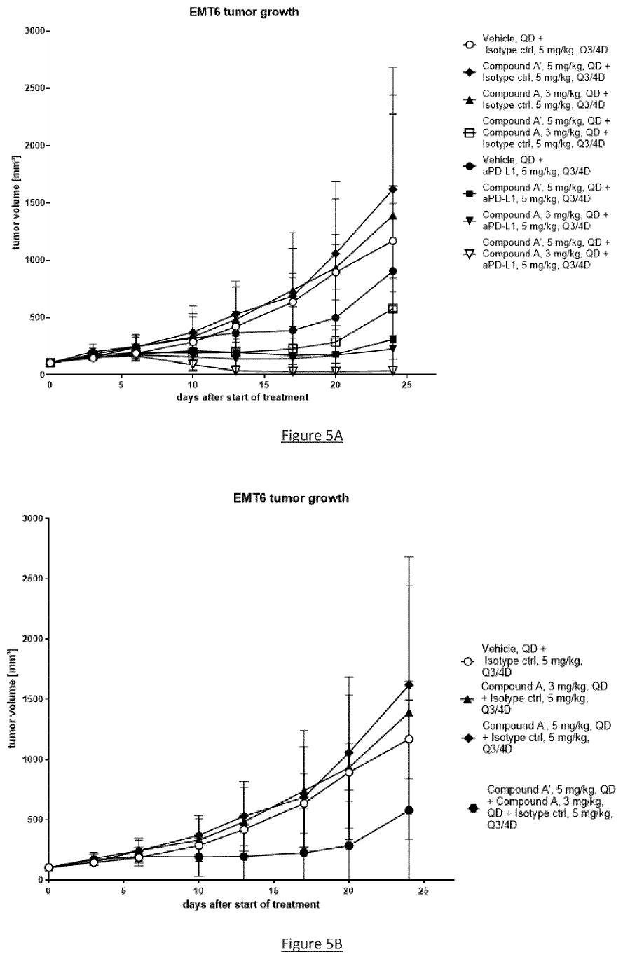

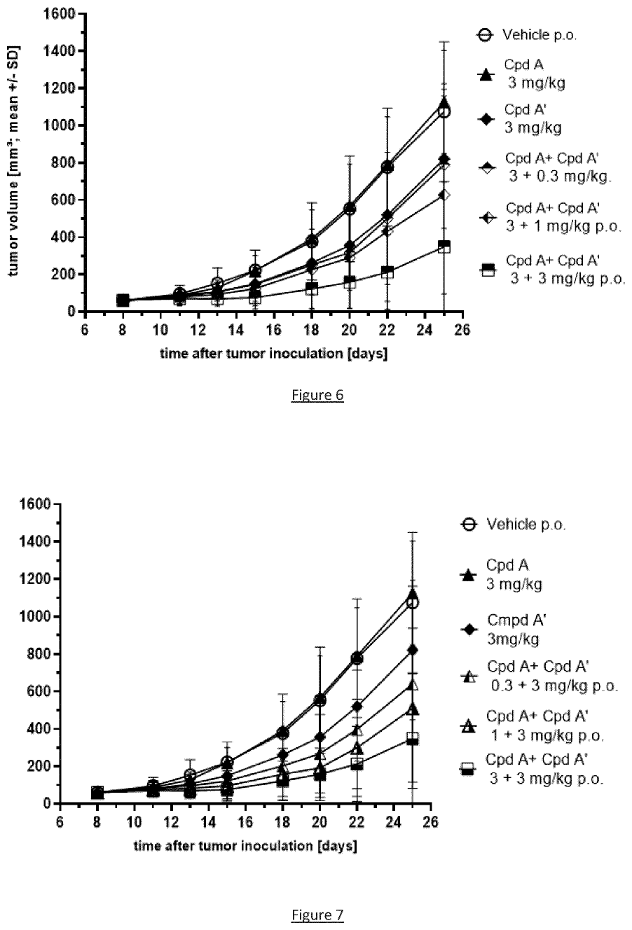

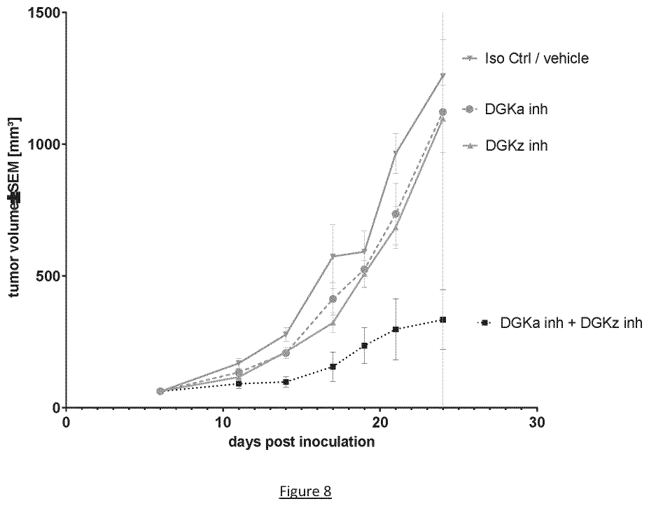

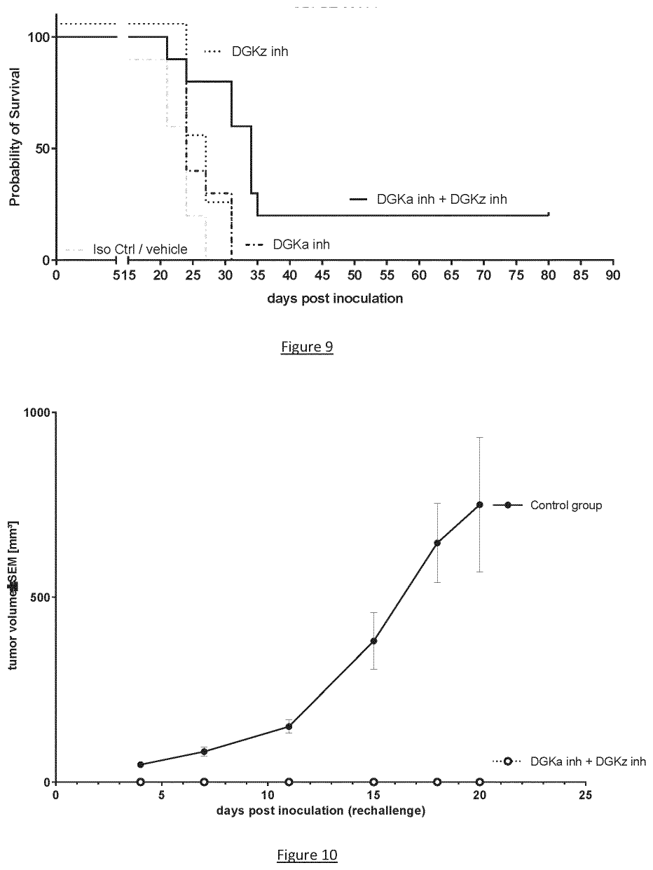

Tumor size was measured using calipers determining length (a) and width (b), for results see FIG. 5. Tumor volume was calculated according to:

$$v = \frac{a \times {b}^{2}}{2} .$$

The results are discussed in the description of FIGS. 5A and SB.

### Example 5: In Vivo Combination Therapy with an Inhibitor of DGKalpha and DGKzeta

The objective of this study was to observe the effects of combination therapies with DGKzeta and/or DGKalpha inhibitors on the tumor volume in the MC38 syngeneic mouse tumor model upon different dose combination ratios.

The following eight treatment groups are included:
1. Vehicle
2. DGKzeta inhibitor A′ (3 mg/kg) p.o., q.d.
3. DGKalpha inhibitor A (3 mg/kg) p.o., q.d.
4. DGKzeta inhibitor A′ (3 mg/kg) p.o., q.d.+DGKalpha inhibitor A (3 mg/kg) q.d.
5. DGKzeta inhibitor A′ (3 mg/kg) p.o., q.d.+DGKalpha inhibitor A (1 mg/kg) q.d.
6. DGKzeta inhibitor A′ (3 mg/kg) p.o., q.d.+DGKalpha inhibitor A (0.3 mg/kg) q.d.
7. DGKzeta inhibitor A′ (1 mg/kg) p.o., q.d.+DGKalpha inhibitor A (3 mg/kg) q.d.
8. DGKzeta inhibitor A′ (0.3 mg/kg) p.o., q.d.+DGKalpha inhibitor A (3 mg/kg) q.d.

Treatment was started on ~day 8 after tumor inoculation at ~80-100 mm³ tumor volume. Tumor growth inhibition was measured until tumors reached a size of 1100 mm³ (approx. Day 25 after tumor inoculation).

Surprisingly, and as shown in FIGS. 6 & 7, a pronounced effect was observed for the combination therapy with DGKalpha inhibitor A and DGKzeta inhibitor A′ over the respective monotherapies in this study. Whilst only moderate inhibition of tumor growth was observed in the two monotherapy groups receiving treatment with DGKalpha inhibitor A and DGKzeta inhibitor A′ (3 mg/kg each) a much stronger and more than additive inhibition of tumor growth was found upon combination therapy with DGKalpha inhibitor A and DGKzeta inhibitor A′ on day 25 after tumor inoculation (3 mg/kg+3 mg/kg).

FIG. 6 shows the tumor growth inhibition upon down titration of the DGKzeta inhibitor A′ (0.3 mg/kg and 1 mg/kg in combination with DGKalpha inhibitor A (3 mg/kg). Surprisingly, a 10-fold lower dose of DGKzeta inhibitor A′ (0.3 mg/kg) shows similar tumor growth inhibition in combination with DGKalpha inhibitor A (3 mg/kg) compared to DGKzeta inhibitor A′ as a single agent at the 3 mg/kg dose, while the DGKalpha inhibitor A (3 mg/kg) showed no single agent activity in this study.

A similar effect could be observed upon down-titration of DGKalpha inhibitor A (0.3 mg/kg and 1 mg/kg) in combination with DGKzeta inhibitor A′ (3 mg/kg) (FIG. 7)

### Example 6: Efficacy of Anti-CCR8 Antibodies in Combination Therapy with an Inhibitor of DGKalpha, DGKzeta, or with Both, and with a PD(L)1 Inhibitor in MC38 Mouse Model—Surprising Effect of Combination Therapy with a DGKalpha Inhibitor and a DGKzeta Inhibitor

In order to study triple combinations or quadruple combinations with anti-CCR8 antibodies, at least one DGK inhibitor and an anti-PD(L)1 antibody, multiple experiments were performed. In order to see the differences in efficacy, the administered amounts for each individual compound or antibody were reduced to 3 mg/kg to avoid full efficacy in a monotherapy setup and to be able to evaluate the efficacy for triple and quadruple combination therapy. As discussed in detail below, this study also brought up surprising effects the combination therapy with DGKalpha inhibitor A and DGKzeta inhibitor A′ over the respective monotherapies in this study.

Efficacy in a MC38 mouse model was analyzed in groups with 10 mice each and is shown in Table 6.1 and FIGS. 8, 9 and 10.
TPP-15285 (mIgG2a) is a surrogate antibody for anti-CCR8 antibody TPP-23411 and induces both, ADCC and ADCP. TPP-15285 was administered i.p. at 3 mg/kg with a BIWx4 administration scheme.
TPP-10748 (Iso Ctrl aCCR8) served as isotype control for TPP-15285 (mIgG2a) and was administered i.p. at 3 mg/kg with a BIWx4 administration scheme.
TPP-3911 (Atezo, mIgG1) was used as anti-PD(L)1 antibody (e.g. pembrolizumab surrogate) and was administered i.p. at 3 mg/kg with a BIWx4 administration scheme.
TPP-3267 or TPP-10149 (mIgG1) served as isotype control for anti-PD(L)1 antibody TPP-3911 (mIgGa) and were abbreviated Iso Ctrl aPD(L)1. The respective isotype control was administered i.p. at 3 mg/kg with a BIWx4 administration scheme.
DGKalpha inhibitor A (DGKα inh A, preferred example of a DGK alpha inhibitor) was administered p.o. at 3 mg/kg with a QD administration scheme.
DGKzeta inhibitor A′ (DGKz inh A′, preferred example of a DGK zeta inhibitor) was administered p.o. at 3 mg/kg with a QD administration scheme.

Antibody treatment was started on ~day 7 after tumor inoculation at ~80-100 mm³ tumor volume, i.e. antibody administration occurred on days 7, 10, 14 and 17, after tumor inoculation. DGK inhibitor treatment started two days after the first antibody administration, i.e. on ~day 9 after tumor inoculation, i.e. DGK inhibitor was administered on day 9, 10, 11, 12, 13, 14, 15, 16, 17, 18, 19, 20 and 21 after tumor inoculation. Tumor growth inhibition was measured until tumors reached a size of 1100 mm³ (approx. Day 20 after tumor inoculation).

Blood samples were drawn on day 11 and 18, 2-3 h after DGK inhibitor administration.

TABLE 6.1
Tumor volume in mm³ upon treatment with aCCR8. DGKalpha inh. DGKzeta inh or
aPD(L)1 monotherapy, any double, triple or quadruple combination thereof in MC38 mouse
model. Data relevant for the present invention are highlighted in bold.

| | | −1 days after | | | | | | |
|---|---|---|---|---|---|---|---|---|
| | | start of | | | | | | |
| | | treatment | | | | | | |
| | | (6 days after | | | | | | |
| | | start of | | | | | | |
| Group | Compounds | transplantation) | 4 | 7 | 10 | 12 | 14 | 17 |
| Iso ctrl for | TPP-10748 + | 61.3 | 168.5 | 277.6 | 573.6 | 591.6 | 965.1 | 1259.1 |
| aCCR8 + | vehicle + | | | | | | | |
| Vehicle + | TPP-3267 | | | | | | | |
| Iso Ctrl for | | | | | | | | |
| aPD(L)1 | | | | | | | | |
| aCCR8 | TPP-15285 | 61.3 | 139.6 | 161.5 | 211.6 | 277.2 | 348.5 | 587.0 |
| DGKa inh | DGKa inh A | 62.0 | 134.0 | 207.0 | 412.4 | 524.8 | 735.4 | 1122.5 |
| DGKz inh | DGKz inh | 62.4 | 115.9 | 211.9 | 322.0 | 508.0 | 684.4 | 1097.3 |
| | A′ | | | | | | | |
| DGKa inh + | DGKa inh A + | 61.8 | 90.4 | 97.3 | 155.3 | 235.5 | 297.5 | 333.5 |
| DGKz inh | DGKz inh | | | | | | | |
| | A′ | | | | | | | |
| aPD(L)1 | TPP-3911 | 65.2 | 116.8 | 97.4 | 155.5 | 221.2 | 279.5 | 442.1 |
| aCCR8 + | TPP-15285 + | 62.0 | 86.2 | 83.1 | 71.5 | 112.5 | 131.6 | 170.4 |
| aPD(L)1 | TPP-3911 | | | | | | | |
| DGKa inh + | DGKa inh A + | 62.1 | 86.7 | 68.1 | 95.3 | 117.3 | 145.2 | 188.8 |
| aPD(L)1) | TPP-3911 | | | | | | | |
| DGKz inh + | DGKz inh A′ + | 61.9 | 86.3 | 60.2 | 77.4 | 115.6 | 129.7 | 207.6 |
| aPD(L)1 | TPP-3911 | | | | | | | |
| DGKa inh + | DGKa inh A + | 62.7 | 100.5 | 53.1 | 47.6 | 65.1 | 61.7 | 109.0 |
| DGKz inh + | DGKz inh | | | | | | | |
| aPD(L)1 | A′ + TPP- | | | | | | | |
| | 3911 | | | | | | | |
| aCCR8 + | TPP-15285 + | 60.4 | 135.3 | 149.7 | 228.0 | 279.3 | 405.9 | 482.2 |
| DGKa inh | DGKa inh | | | | | | | |
| | A | | | | | | | |
| aCCR8 + | TPP-15285 + | 63.5 | 139.4 | 125.3 | 170.8 | 224.9 | 291.8 | 306.7 |
| DGKz inh | DGKz inh | | | | | | | |
| | A′ | | | | | | | |
| aCCR8 + | TPP-15285 + | 60.3 | 91.6 | 70.2 | 79.4 | 92.4 | 117.4 | 146.6 |
| DGKa inh + | DGKa inh | | | | | | | |
| DGKz inh | A + | | | | | | | |
| | DGKz inh | | | | | | | |
| | A′ | | | | | | | |
| aCCR8 + | TPP-15285 + | 57.9 | 59.9 | 39.6 | 35.3 | 30.9 | 35.5 | 46.8 |
| DGKa inh + | DGKa inh | | | | | | | |
| aPD(L)1 | A + | | | | | | | |
| | TPP-3911 | | | | | | | |
| aCCR8 + | TPP-15285 + | 61.1 | 61.8 | 50.8 | 61.0 | 53.3 | 59.7 | 86.4 |
| DGKz inh + | DGKz inh | | | | | | | |
| aPD(L)1 | A′ + | | | | | | | |
| | TPP-3911 | | | | | | | |
| aCCR8 + | TPP-15285 + | 60.3 | 64.5 | 51.0 | 60.5 | 54.0 | 48.1 | 91.0 |
| DGKa inh + | DGKa inh | | | | | | | |
| DGKz inh + | A + | | | | | | | |
| aPD(L)1 | DGKz inh | | | | | | | |
| | A′ + TPP- | | | | | | | |
| | 3911 | | | | | | | |

Surprisingly, and as shown in FIG. 8, also a pronounced effect was also observed for the combination therapy with DGKalpha inhibitor A and DGKzeta inhibitor A′ over the respective monotherapies in this study. FIG. 8 visualizes the data provided in Table 6.1 with a focus on DGKa inhibitor monotherapy, DGKz inhibitor monotherapy, and DGKa inhibitor+DGKz inhibitor combination therapy, respectively. Whilst only moderate inhibition of tumor growth was observed in the two monotherapy groups receiving treatment with DGKalpha inhibitor A and DGKzeta inhibitor A′ (e.g. 1122.5 mm³ and 1097.3 mm³ vs 1259.1 mm³ in the control group on day 17) a much stronger and more than additive inhibition of tumor growth (e.g. 333.5 mm³ on day 17) was found upon combination therapy with DGKalpha inhibitor A and DGKzeta inhibitor A′.

FIG. 9 shows the survival curves of the different groups according to the current example. The survival study ended at day 85. Whilst in the control and monotherapy groups no survival was observed, two of 10 animals receiving combination therapy with DGKalpha inhibitor A and DGKzeta inhibitor A′. survived until day 85. FIG. 10 shows that the two animals surviving the end of the study did not show any substantial tumor growth upon re-inoculation with MC38 tumor cells, suggesting their immunity as a result of the treatment.

### Example 7: Efficacy of Anti-CCR8 Antibodies in Combination Therapy with an Inhibitor of DGKalpha, DGKzeta, or with Both in MC38 Mouse Model—Surprising Effect of Combination Therapy with a DGKalpha Inhibitor and a DGKzeta Inhibitor

Various experiments were performed to analyze the efficacy of anti-CCR8 antibodies alone or in combination with inhibitors of DGKalpha, inhibitors of DGKzeta, or inhibitors of DGKalpha and inhibitors of DGKzeta.

As discussed in detail below, this study also brought up surprising effects the combination therapy with DGKalpha inhibitor A and DGKzeta inhibitor A′ over the respective monotherapies in this study.

Combination treatment with 5 mg/kg anti-mouse CCR8 surrogate antibody TPP-15285 (IgG2a) BIW (twice weekly, intraperitoneally) and 5 mg/kg DGKalpha inhibitor (DGKalpha inhibitor A), QD (daily, oral dosing) was performed in MC38 tumor bearing mice. While the antibody administration was started on day 7 after tumor inoculation, the DGK inhibitor treatment started two days later, on day 9 after tumor inoculation. This schedule was designed to allow the efficient depletion of the Tregs by the anti-mouse CCR8 surrogate antibody before the DGK inhibitor activates the T cells.

Combination treatment with 5 mg/kg anti-mouse CCR8 surrogate antibody TPP-15285 (IgG2a) BIW (twice weekly, intraperitoneally) and 3 mg/kg DGKzeta inhibitor (DGKzeta inhibitor A′), QD (daily, oral dosing) was performed in MC38 tumor bearing mice. While the antibody administration was started on day 7 after tumor inoculation, the DGK inhibitor treatment started two days later, on day 9 after tumor inoculation. This schedule was designed to allow the efficient depletion of the Tregs by the anti-mouse CCR8 surrogate antibody before the DGK inhibitor activates the T cells.

Combination treatment with 5 mg/kg anti-mouse CCR8 surrogate antibody TPP-15285 (IgG2a) BIW (twice weekly, intraperitoneally), 5 mg/kg DGKalpha inhibitor (DGKalpha inhibitor A), QD (daily, oral dosing) and 3 mg/kg DGKzeta inhibitor (DGKzeta inhibitor A′), QD (daily, oral dosing) was performed in MC38 tumor bearing mice. While the antibody administration was started on day 7 after tumor inoculation, the DGKalpha and DGKzeta inhibitor treatment started two days later, on day 9 after tumor inoculation. This schedule was designed to allow the efficient depletion of the Tregs by the anti-mouse CCR8 surrogate antibody before the DGK inhibitor activates the T cells.

Combination treatment with 5 mg/kg DGKalpha inhibitor (DGKalpha inhibitor A), QD (daily, oral dosing) and 3 mg/kg DGKzeta inhibitor (DGKzeta inhibitor A′), QD (daily, oral dosing) was performed in MC38 tumor bearing mice. The DGKalpha and DGKzeta inhibitor treatment started on day 9 after tumor inoculation.

As shown in Table 7.1 below, the combination of DGKalpha inhibitor A and DGKzeta inhibitor A′ was substantially superior over monotherapy with either DGKzeta inhibitor A′ or DGKalpha inhibitor A, thus confirming the findings as described in the previous examples.

TABLE 7.1
Mean tumor volume in mm³ upon treatment of MC38 mice with isotype control,
DGKalpha inhibitor A and DGKzeta inhibitor A′, anti-CCR8 antibody, anti-CCR8 antibody and
DGKalpha inhibitor A, anti-CCR8 antibody and DGKzeta inhibitor A′, as well as triple combination
(anti-CCR8 antibody, DGKalpha inhibitor A, and DGKzeta inhibitor A′). Data relevant for the
present invention are highlighted in bold.

| | Day 0 | | | | | |
|---|---|---|---|---|---|---|
| | (day 7 after | | | | | |
| | tumor | | | | | |
| Group | inoculation) | Day 4 | Day 7 | Day 11 | Day 14 | Day 18 |
| TPP-10748 + | 91.78 | 238.84 | 489.86 | 1077.61 | 1717.45 | 3200.11 |
| vehicle | | | | | | |
| DGKalpha inhibitor A | 91.74 | 216.59 | 483.4 | 1057.04 | 1585.28 | 2961.81 |
| +DGKzeta inhibitor A′ | 91.65 | 216.68 | 480.5 | 982.9 | 1448.04 | 2879.9 |
| TPP-10748 + | 91.70 | 233.16 | 500.51 | 882.51 | 1187.62 | 2140.91 |
| DGKalpha inhibitor A + | | | | | | |
| DGKzeta inhibitor A′ | | | | | | |
| TPP-15285 + | 91.74 | 252.99 | 474.66 | 787.12 | 1069.84 | 2250.90 |
| vehicle | | | | | | |
| TPP-15285 + | 91.71 | 231.82 | 408.26 | 538.06 | 686.91 | 1276.60 |
| DGKalpha inhibitor A | | | | | | |
| TPP-15285 + | 91.74 | 239.97 | 448.71 | 544.12 | 635.26 | 1185.12 |
| DGKzeta inhibitor A′ | | | | | | |
| TPP-15285 + | 91.75 | 251.51 | 423.55 | 561.56 | 607.11 | 1149.83 |
| DGKalpha inhibitor A + | | | | | | |
| DGKzeta inhibitor A′ | | | | | | |

TABLE 7.2
Standard error of the mean corresponding to the values in Table 7.1. Data relevant
for the present invention are highlighted in bold.

| | Day 0 | | | | | |
|---|---|---|---|---|---|---|
| | (day 7 after | | | | | |
| | tumor | | | | | |
| Group | inoculation) | Day 4 | Day 7 | Day 11 | Day 14 | Day 18 |
| TPP-10748 + | 4.59 | 11.57 | 43.95 | 88.48 | 185.24 | 287.78 |
| vehicle | | | | | | |
| DGKalpha inhibitor A | 4.6 | 15.57 | 51.14 | 99.2 | 138.78 | 237.01 |
| +DGKzeta inhibitor A′ | 4.0 | 14.0 | 47.0 | 114.0 | 196.0 | 300.0 |
| TPP-10748 + | 4.34 | 16.20 | 48.42 | 69.43 | 107.96 | 193.40 |
| DGKalpha inhibitor A + | | | | | | |
| DGKzeta inhibitor A′ | | | | | | |
| TPP-15285 + | 4.36 | 12.40 | 20.59 | 61.55 | 126.54 | 292.52 |
| vehicle | | | | | | |
| TPP-15285 + | 4.23 | 18.48 | 45.32 | 47.10 | 70.13 | 168.32 |
| DGKalpha inhibitor A | | | | | | |
| TPP-15285 + | 4.24 | 15.19 | 54.32 | 65.26 | 84.43 | 188.41 |
| DGKzeta inhibitor A′ | | | | | | |
| TPP-15285 + | 4.23 | 17.81 | 41.87 | 58.25 | 63.67 | 180.15 |
| DGKalpha inhibitor A + | | | | | | |
| DGKzeta inhibitor A′ | | | | | | |

### Example 8: Efficacy of Anti-CCR8 Antibodies in Combination Therapy with an Inhibitor of DGKalpha, DGKzeta, or with Both in Hepa 1-6 Mouse Model—Surprising Effect of Combination Therapy with a DGKalpha Inhibitor and a DGKzeta Inhibitor

Various experiments were performed to analyze the efficacy of anti-CCR8 antibodies alone or in combination with inhibitors of DGKalpha, inhibitors of DGKzeta, or inhibitors of DGKalpha and inhibitors of DGKzeta.

As discussed in detail below, this study also brought up surprising effects the combination therapy with DGKalpha inhibitor A and DGKzeta inhibitor A′ over the respective monotherapies in this study.

Combination treatment with 5 mg/kg anti-mouse CCR8 surrogate antibody TPP-15285 (IgG2a) BIW (twice weekly, intraperitoneally) and 5 mg/kg DGKalpha inhibitor (DGKalpha inhibitor A), QD (daily, oral dosing) was performed in Hepa 1-6 tumor bearing mice. While the antibody administration was started on day 7 after tumor inoculation, the DGK inhibitor treatment started two days later, on day 9 after tumor inoculation. This schedule was designed to allow the efficient depletion of the Tregs by the anti-mouse CCR8 surrogate antibody before the DGK inhibitor activates the T cells.

Combination treatment with 5 mg/kg anti-mouse CCR8 surrogate antibody TPP-15285 (IgG2a) BIW (twice weekly, intraperitoneally) and 3 mg/kg DGKzeta inhibitor (DGKzeta inhibitor A′), QD (daily, oral dosing) was performed in Hepa 1-6 tumor bearing mice. While the antibody administration was started on day 7 after tumor inoculation, the DGK inhibitor treatment started two days later, on day 9 after tumor inoculation. This schedule was designed to allow the efficient depletion of the Tregs by the anti-mouse CCR8 surrogate antibody before the DGK inhibitor activates the T cells.

Combination treatment with 5 mg/kg anti-mouse CCR8 surrogate antibody TPP-15285 (IgG2a) BIW (twice weekly, intraperitoneally), 5 mg/kg DGKalpha inhibitor (DGKalpha inhibitor A), QD (daily, oral dosing) and 3 mg/kg DGKzeta inhibitor (DGKzeta inhibitor A′), QD (daily, oral dosing) was performed in Hepa 1-6 tumor bearing mice. While the antibody administration was started on day 7 after tumor inoculation, the DGKalpha and DGKzeta inhibitor treatment started two days later, on day 9 after tumor inoculation. This schedule was designed to allow the efficient depletion of the Tregs by the anti-mouse CCR8 surrogate antibody before the DGK inhibitor activates the T cells.

Combination treatment with 5 mg/kg DGKalpha inhibitor (DGKalpha inhibitor A), QD (daily, oral dosing) and 3 mg/kg DGKzeta inhibitor (DGKzeta inhibitor A′), QD (daily, oral dosing) was performed in Hepa 1-6 tumor bearing mice. The DGKalpha and DGKzeta inhibitor treatment started on day 9 after tumor inoculation.

As shown in Table 8.1 below, the combination of DGKalpha inhibitor A and DGKzeta inhibitor A′ was substantially superior over monotherapy with either DGKzeta inhibitor A′ or DGKalpha inhibitor A, thus confirming the findings as described in the previous examples.

TABLE 8.1
Mean tumor volume in mm³ upon treatment of Hepa 1-6 mice with isotype control,
DGKalpha inhibitor and DGKzeta inhibitor, anti-CCR8 antibody, anti-CCR8 antibody and DGKalpha
inhibitor, anti-CCR8 antibody and DGKzeta inhibitor, as well as triple combination (anti-CCR8
antibody, DGKalpha inhibitor, and DGKzeta inhibitor). Data relevant for the present invention are
highlighted in bold.

| Group | Day 0 | Day 4 | Day 7 | Day 11 | Day 14 | Day 18 | Day 20 |
|---|---|---|---|---|---|---|---|
| TPP-10748 + | 89.00 | 208.55 | 424.44 | 636.74 | 823.78 | 1201.17 | 1455.90 |
| vehicle | | | | | | | |
| DGKalpha inhibitor A | 88.99 | 186.75 | 321.40 | 461.93 | 569.94 | 787.30 | 957.86 |
| +DGKzeta inhibitor A′ | 88.99 | 224.86 | 429.26 | 624.07 | 799.19 | 1001.75 | 1158.6 |
| TPP-10748 + | 89.01 | 179.32 | 272.46 | 326.80 | 378.51 | 433.18 | 532.53 |
| DGKalpha inhibitor A + | | | | | | | |
| DGKzeta inhibitor A′ | | | | | | | |
| TPP-15285 + | 88.97 | 258.03 | 413.71 | 521.44 | 573.80 | 583.45 | 646.96 |
| vehicle | | | | | | | |
| TPP-15285 + | 89.07 | 203.52 | 268.43 | 258.50 | 256.74 | 247.79 | 243.02 |
| DGKalpha inhibitor A | | | | | | | |
| TPP-15285 + | 88.97 | 239.02 | 356.37 | 433.67 | 474.49 | 431.20 | 435.23 |
| DGKzeta inhibitor A′ | | | | | | | |
| TPP-15285 + | 89.07 | 200.39 | 260.97 | 252.87 | 271.72 | 282.85 | 315.37 |
| DGKalpha inhibitor A + | | | | | | | |
| DGKzeta inhibitor A′ | | | | | | | |

TABLE 8.2
Standard error of the mean corresponding to the values in Table 8.1. Data relevant
for the present invention are highlighted in bold.

| Group | Day 0 | Day 4 | Day 7 | Day 11 | Day 14 | Day 18 | Day 20 |
|---|---|---|---|---|---|---|---|
| TPP-10748 + | 5.31 | 22.66 | 47.66 | 80.83 | 115.61 | 193.06 | 238.93 |
| vehicle | | | | | | | |
| DGKalpha inhibitor A | 5.3 | 18.62 | 37.21 | 70.53 | 107.6 | 159.69 | 208.4 |
| +DGKzeta inhibitor A′ | 5.26 | 19.75 | 52.61 | 79.85 | 100.65 | 147.61 | 169.17 |
| TPP-10748 + | 5.22 | 26.25 | 30.50 | 47.48 | 65.64 | 92.34 | 128.38 |
| DGKalpha inhibitor A + | | | | | | | |
| DGKzeta inhibitor A′ | | | | | | | |
| TPP-15285 + | 5.13 | 30.43 | 46.56 | 74.66 | 85.96 | 100.64 | 131.57 |
| vehicle | | | | | | | |
| TPP-15285 + | 5.15 | 20.72 | 31.37 | 40.10 | 58.12 | 65.47 | 73.11 |
| DGKalpha inhibitor A | | | | | | | |
| TPP-15285 + | 5.17 | 18.50 | 43.54 | 56.95 | 69.06 | 77.60 | 87.22 |
| DGKzeta inhibitor A′ | | | | | | | |
| TPP-15285 + | 5.23 | 19.44 | 28.47 | 49.02 | 55.46 | 70.12 | 85.99 |
| DGKalpha inhibitor A + | | | | | | | |
| DGKzeta inhibitor A′ | | | | | | | |

1. A combination comprising one or more inhibitors of DGKalpha and one or more inhibitors of DGKzeta.

2. The combination according to claim 1, comprising one inhibitor of DGKalpha and one inhibitor of DGKzeta.

3. The combination according to claim 1, comprising one DGKalpha inhibitor compound of formula (I), 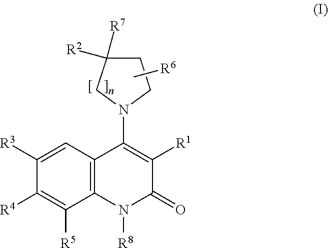
in which:
R¹ represents a group selected from the group consisting of cyano, —C(═O)NH₂, —C(═O)N(H)CH₃, —C(═O)N(H)C₂H₅, —C(═O)N(CH₃)₂ and —C(═O)OR¹⁵,
R² represents a group selected from the group consisting of phenyl, naphthyl and 5- to 10-membered heteroaryl,
which 5- to 10-membered heteroaryl group is connected to the rest of the molecule via a carbon atom of said 5- to 10-membered heteroaryl group, and
which phenyl, naphthyl and 5- to 10-membered heteroaryl group is optionally substituted, one, two, three or four times, each substituent independently selected from the group consisting of a halogen atom or a group selected from the group consisting of
C₁-C₆-alkyl, C₃-C₆-cycloalkyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂ alkoxy)-(C₁-C₆-alkoxy)-, C₁-C₆-haloalkoxy, C₃-C₆-cycloalkyloxy, phenoxy, —SR¹⁴, —S(═O)R¹⁴, —S(═O)₂R¹⁴, —P(═O)(R¹⁴)₂ cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
or two substituents of said phenyl group, when they are attached to adjacent ring atoms, are optionally linked to one another in such a way that they jointly form a group selected from the group consisting of
—(CH₂)₃—, —CH₂—CH(OH)—CH₂—, —(CH₂)₄—, —O—(CH₂)₂—, —(CH₂)₂—O—, —CH₂—CH(CH₃)—O—, —CH₂—O—CH₂—, —O—(CH₂)₃—, —(CH₂)₃—O—, —CH₂—O—(CH₂)₂—, —(CH₂)₂O—CH₂—, —O—CH₂—O—, —O—C(CH₃)₂—O—, —O—(CH₂)₂—O—, —N(R¹⁸)—C(═O)—(C(R¹⁸)(R¹⁹))ₘ—, —N(R¹⁸)—C(═O)—(C(CH₂)₃)—, —N(R¹⁸)—(C(R¹⁸)(R¹⁹))ₘ—, —N(R¹⁸)—C(═O)—O— and —N(R¹⁸)—C(═O)—N(R¹⁸)_,
wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
and
wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and
wherein said C₁-C₆-alkyl and C₁-C₆-alkoxy group is optionally substituted with a group selected from the group consisting of C₃-C₄-cycloalkyl, phenyl and 4- to 7-membered heterocycloalkyl,
wherein said 4- to 7-membered heterocycloalkyl group is connected to the rest of the molecule via a carbon atom of said 4- to 7-membered heterocycloalkyl group,
and
wherein said 4- to 7-membered heterocycloalkyl group is optionally substituted, one, two or three times, each substituent independently selected from the group consisting of a halogen atom or a group selected from the group consisting of C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and
which phenyl group is optionally substituted, one or two times, each substituent independently selected from the group consisting of a halogen atom or a group selected from the group consisting of C₁-C₂-alkyl, C₁-C₂-haloalkyl, cyano, hydroxy, C₁-C₂-alkoxy, C₃-C₄-cycloalkyl and —N(R⁹)(R¹⁰),
and
which C₃-C₄-cycloalkyl group is optionally substituted, one or two times, each substituent independently selected from the group consisting of a halogen atom or a group selected from the group consisting of
cyano and hydroxy,
and
wherein said C₃-C₆-cycloalkyl group is optionally substituted, one or two times, each substituent independently selected from the group consisting of a halogen atom or a C₁-C₄-alkyl group,
and
wherein said phenyl, phenoxy and 5- or 6-membered heteroaryl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R⁹)(R¹⁰),
R³ represents a hydrogen atom or a halogen atom or a group selected from the group consisting of
C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂ alkoxy)-(C₁-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
and
wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and
wherein said C₁-C₆-alkyl, C₂-C₆-alkenyl, C₂-C₆-alkynyl and C₁-C₆-alkoxy group is optionally substituted with a group selected from the group consisting of C₃-C₄-cycloalkyl, phenyl and 4- to 7-membered heterocycloalkyl,
wherein said 4- to 7-membered heterocycloalkyl group is connected to the rest of the molecule via a carbon atom of said 4- to 7-membered heterocycloalkyl group,
and
wherein said 4- to 7-membered heterocycloalkyl group is optionally substituted, one, two or three times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and
which phenyl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R⁹)(R¹⁰),
and
which C₃-C₄-cycloalkyl group is optionally substituted, one or two times, each substituent independently selected from the group consisting of a halogen atom or a group selected from the group consisting of cyano and hydroxy,
and
wherein said C₂-C₆-alkenyl group is optionally substituted with a C₁-C₄-haloalkyl group,
and
wherein said C₃-C₆-cycloalkyl and C₄-C₆-cycloalkenyl group is optionally substituted, one or two times, each substituent independently selected from the group consisting of a halogen atom or a group selected from the group consisting of C₁-C₄-alkyl and C₁-C₄-haloalkyl,
and
wherein said phenyl, phenoxy and 5- or 6-membered heteroaryl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R⁹)(R¹⁰),
R⁴ represents a hydrogen atom or a halogen atom or a group selected from the group consisting of
C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂ alkoxy)-(C₁-C₆-alkoxy)-, C₁-C₄-haloalkoxy, —O—(C₁-C₄-alkyl)-C(═O)OR¹⁵, —O—(C₁-C₄-alkyl)-C(═O)N(R⁹)(R¹⁰), C₃-C₆-cycloalkyloxy, —S(═O)R¹⁴, —S(═O)₂R¹⁴, cyano, nitro, hydroxy, —N(R⁹)(R¹⁰), —N(R¹⁶)(R¹⁷), —N(R¹⁶)(R²⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
and
wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and
wherein said C₁-C₆-alkyl, C₂-C₆-alkenyl, C₂-C₆-alkynyl and C₁-C₆-alkoxy group is optionally substituted with a group selected from the group consisting of C₃-C₄-cycloalkyl and 4- to 7-membered heterocycloalkyl,
wherein said 4- to 7-membered heterocycloalkyl group is connected to the rest of the molecule via a carbon atom of said 4- to 7-membered heterocycloalkyl group,
and
wherein said 4- to 7-membered heterocycloalkyl group is optionally substituted, one, two or three times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and
which phenyl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R⁹)(R¹⁰),
and
which C₃-C₄-cycloalkyl group is optionally substituted, one or two times, each substituent independently selected from the group consisting of a halogen atom or a group selected from the group consisting of cyano and hydroxy,
and
wherein said C₁-C₆-alkoxy group is optionally substituted with a oxiran-2-yl group,
and
wherein said C₃-C₆-cycloalkyl and C₄-C₆-cycloalkenyl group is optionally substituted, one or two times, each substituent independently selected from the group consisting of a halogen atom or a C₁-C₄-alkyl group,
and
wherein said phenyl and 5- or 6-membered heteroaryl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R⁹)(R¹⁰),
R⁵ represents a hydrogen atom or a halogen atom or a group selected from the group consisting of
C₁-C₆-alkyl, C₂-C₆-alkenyl, C₂-C₆-alkynyl, C₃-C₆-cycloalkyl, C₄-C₆-cycloalkenyl, C₁-C₆-hydroxyalkyl, C₁-C₆-haloalkyl, (C₁-C₂-alkoxy)-(C₁-C₆-alkyl)-, C₁-C₆-alkoxy, (C₁-C₂ alkoxy)-(C₂-C₆-alkoxy)-, C₁-C₄-haloalkoxy, C₃-C₆-cycloalkyloxy, phenoxy, —SR¹⁴, —S(═O)R¹⁴, —S(═O)₂R¹⁴, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy, phenyl and 5- or 6-membered heteroaryl,
wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
and
wherein said 4- to 7-membered heterocycloalkyl group, 5- to 7-membered heterocycloalkenyl group and (4- to 7-membered heterocycloalkyl)oxy group is optionally substituted, one, two or three times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and
wherein said C₁-C₆-alkyl, C₂-C₆-alkenyl, C₂-C₆-alkynyl and C₁-C₆-alkoxy group is optionally substituted with a group selected from the group consisting of C₃-C₄-cycloalkyl, phenyl and 4- to 7-membered heterocycloalkyl,
wherein said 4- to 7-membered heterocycloalkyl group is connected to the rest of the molecule via a carbon atom of said 4- to 7-membered heterocycloalkyl group,
and
wherein said 4- to 7-membered heterocycloalkyl group is optionally substituted, one, two or three times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl, —N(R⁹)(R¹⁰) and oxo,
and
which phenyl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R⁹)(R¹⁰),
and
which C₃-C₄-cycloalkyl group is optionally substituted, one or two times, each substituent independently selected from the group consisting of a halogen atom or a group selected from the group consisting of cyano and hydroxy,
and
wherein said C₃-C₆-cycloalkyl and C₄-C₆-cycloalkenyl group is optionally substituted, one or two times, each substituent independently selected from the group consisting of a halogen atom or a C₁-C₄-alkyl group,
and
wherein said phenyl, phenoxy and 5- or 6-membered heteroaryl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R⁹)(R¹⁰),
R⁶ represents a hydrogen atom, or a fluorine atom or a group selected from the group consisting of
C₁-C₄-alkyl, C₁-C₄-hydroxyalkyl, C₁-C₄-alkoxy, hydroxy and oxo,
R⁷ represents a hydrogen atom or a halogen atom or a group selected from the group consisting of
C₁-C₄-alkyl, C₁-C₄-alkoxy, hydroxy and cyano,
R⁸ represents a group selected from the group consisting of methyl and ethyl,
R⁹ and R¹⁰ represent, independently from each occurrence, a hydrogen atom or a group selected from the group consisting of
C₁-C₄-alkyl, C₂-C₄-hydroxyalkyl, N═C—(C₁-C₄-alkyl)-, (C₁-C₄-alkoxy)-(C₂-C₄-alkyl)-, C₃-C₄-cycloalkyl and C₂-C₄-haloalkyl,
or
R⁹ and R¹⁰ together with the nitrogen to which they are attached represent a
nitrogen containing 4- to 7-membered heterocycloalkyl group,
wherein said nitrogen containing 4- to 7-membered heterocycloalkyl group is optionally substituted, one, two or three times, each substituent independently selected from the group consisting of a halogen atom or a group selected from the group consisting of
C₁-C₄-alkyl, C₃-C₄-cycloalkyl, hydroxy and oxo,
or
two substituents, which are attached to the same carbon atom of said nitrogen containing 4- to 7-membered heterocycloalkyl group, together with the carbon atom to which they are attached, represent a 4- to 7-membered heterocycloalkyl group,
wherein said 4- to 7-membered heterocycloalkyl group is optionally substituted, one or two times, each substituent independently selected from the group consisting of a halogen atom or a group selected from the group consisting of
C₁-C₄-alkyl, C₃-C₄-cycloalkyl, C₁-C₄-haloalkyl, hydroxy and oxo,
R¹¹ represents a hydrogen atom or group selected from the group consisting of C₁-C₄-alkyl, C₁-C₄-hydroxyalkyl, C₁-C₄-haloalkyl, phenyl and 5- or 6-membered heteroaryl,
wherein said phenyl group and 5- or 6-membered heteroaryl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R⁹)(R¹⁰),
R¹² represents a hydrogen atom or a C₁-C₄-alkyl group,
R¹³ represents a hydrogen atom or a group selected from the group consisting of
C₁-C₆-alkyl, phenyl and 5- or 6-membered heteroaryl,
wherein said phenyl group and 5- or 6-membered heteroaryl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R⁹)(R¹⁰),
R¹⁴ represents a group selected from the group consisting of C₁-C₆-alkyl, C₁-C₆-haloalkyl, C₃-C₆-cycloalkyl, phenyl and 5- or 6-membered heteroaryl,
wherein said phenyl group and 5- or 6-membered heteroaryl group is optionally substituted, one or two times, each substituent independently selected from the group consisting of a halogen atom or a group selected from the group consisting of
C₁-C₂-alkyl, C₁-C₂-haloalkyl, cyano, hydroxy, C₁-C₂-alkoxy, C₃-C₄-cycloalkyl and —N(R′)(R¹⁰),
R¹⁵ represents a hydrogen atom or a C₁-C₄-alkyl group,
R¹⁶ represents a hydrogen atom or a group selected from the group consisting of C₁-C₄-alkyl, C₃-C₄-cycloalkyl and C₂-C₄-haloalkyl,
R¹⁷ represents a 4- to 7-membered heterocycloalkyl group,
wherein said 4- to 7-membered heterocycloalkyl group is optionally substituted, one, two or three times, each substituent independently selected from the group consisting of a halogen atom or a group selected from the group consisting of C₁-C₄-alkyl, C₃-C₄-cycloalkyl, C₁-C₄-alkoxy, hydroxy and oxo, and
wherein said 4- to 7-membered heterocycloalkyl group is connected to the rest of the molecule via a carbon atom of the 4- to 7-membered heterocycloalkyl group,
R¹⁸ represents a hydrogen atom or a group selected from the group consisting of methyl and ethyl,
R¹⁹ represents a hydrogen atom or a group selected from the group consisting of methyl and ethyl,
R²⁰ represents a (4- to 7-membered heterocycloalkyl)-(C₁-C₄-alkyl)- group,
wherein the (4- to 7-membered heterocycloalkyl) part of said group is optionally substituted, one, two or three times, each substituent independently selected from the group consisting of a halogen atom or a group selected from the group consisting of
C₁-C₄-alkyl, C₃-C₄-cycloalkyl, C₁-C₄-alkoxy, hydroxy and oxo,
m represents an integer selected from the group consisting of 1, 2 and 3, and
n represents an integer selected from the group consisting of 1, 2 and 3,
or a stereoisomer, a tautomer, an N-oxide, a hydrate, a solvate, or a salt thereof, or a mixture of same;
and
one DGKζ inhibitor compound of formula (II), 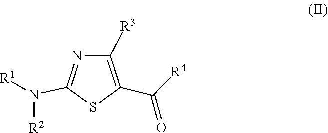
in which:
R¹ represents a phenyl or 6-membered heteroaryl group optionally substituted, one, two, or three times, each substituent independently selected from the group consisting of a halogen atom or a group selected from the group consisting of hydroxy, cyano, nitro, C₁-C₆-alkyl, (phenyl)-(C₁-C₃-alkyl)-, C₁-C₆-haloalkyl, C₁-C₆-alkoxy, (phenyl)-(C₁-C₃-alkoxy)-, C₁-C₆-haloalkoxy, —N(R⁵)(R⁶),
wherein the phenyl groups in said (phenyl)-(C₁-C₃-alkyl)- and (phenyl)-(C₁-C₃-alkoxy)- groups are optionally substituted one or two times, each substituent independently selected from the group consisting of a halogen atom or a group selected from the group consisting of cyano, methyl, ethyl, trifluoromethyl, methoxy, ethoxy, dimethylamino and trifluoromethoxy,
or two substituents attached to adjacent carbon atoms of said phenyl or 6-membered heteroaryl group together form a bivalent group selected from the group consisting of —(CH₂)₃—, —(CH₂)₄—, —(CH₂)₂—O—, —(CH₂)₃—O—, —CH₂—O—CH₂—, —(CH₂)₂O—CH₂—, —O—CH₂—O—, —O—CH₂—CH₂—O—, —O—CF₂—O—, —O—CH₂—CF₂—O—, and —O—CF₂—CF₂—O—,
or
R¹ represents a 5-membered heteroaryl group optionally substituted one or two times, each substituent independently selected from the group consisting of a halogen atom or a group selected from the group consisting of cyano, C₁-C₃-alkyl, and C₁-C₃-alkoxy;
R² represents a group 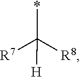
wherein “*” indicates the point of attachment to the nitrogen atom to which R² is attached;
R³ represents a group selected from the group consisting of methyl and —NH₂;
R⁴ represents a phenyl or 6-membered heteroaryl group optionally substituted, one, two, or three times, each substituent independently selected from the group consisting of a halogen atom or a group selected from the group consisting of cyano, nitro, C₁-C₆-alkyl, (phenyl)-(C₁-C₃-alkyl)-, (5- or 6-membered heteroaryl)-(C₁-C₃-alkyl)-, (C₃-C₇-cycloalkyl)-(C₁-C₃-alkyl)-, ((R⁹)O)—(C₁-C₆-alkyl)-, C₁-C₆-haloalkyl, C₃-C₇-cycloalkyl, —OR⁹, —N(R¹⁰)(R¹¹), ((R¹⁰)(R¹¹)N)—(C₁-C₃-alkyl)-, —C(═O)—N(R¹²)(R¹³), —S(═O)ₙ—R¹⁴, —C(═O)R¹⁴, —C(═O)—OR¹⁷, and a 5- or 6-membered heteroaryl group which itself is optionally substituted with one or two substituents selected from the group consisting of a halogen atom and a methyl group,
or two substituents attached to adjacent carbon atoms of said phenyl or 6-membered heteroaryl group together form a bivalent group selected from the group consisting of —(CH₂)₃—, —(CH₂)₄—, —(CH₂)₂—O—, —(CH₂)₃—O—, —CH₂—O—CH₂—, —(CH₂)₂O—CH₂—, —O—CH₂—O—, —O—CH₂—CH₂—O—, —O—CF₂—O—, —O—CH₂—CF₂—O—, and —O—CF₂—CF₂—O—;
R⁵ and R⁶ represent, independently from each occurrence, a hydrogen atom or a group selected from the group consisting of C₁-C₄-alkyl, (C₁-C₄-alkyl)-C(═O)—, C₃-C₄-cycloalkyl and (phenyl)-(C₁-C₃-alkyl)-,
or
R⁵ and R⁶, together with the nitrogen atom to which they are attached, represent a monocyclic nitrogen containing 4- to 7-membered heterocycloalkyl group which is optionally substituted one, two or three times, each substituent independently selected from the group consisting of a halogen atom or a group selected from the group consisting of oxo, hydroxy, C₁-C₄-alkyl, (C₁-C₄-alkyl)-C(═O)—, C₃-C₄-cycloalkyl and C₁-C₄-alkoxy;
R⁷ represents a hydrogen atom or a C₁-C₂-alkyl group;
R⁸ represents a group selected from the group consisting of —C(═O)—NH₂ and —S(═O)₂—NH₂;
R⁹ represents a hydrogen atom or a group selected from the group consisting of C₁-C₆-alkyl, (5- or 6-membered heteroaryl)-(C₁-C₃-alkyl)-, (phenyl)-(C₁-C₃-alkyl)-, C₁-C₆-haloalkyl, C₂-C₄-hydroxyalkyl, (C₁-C₃-alkoxy)-C₂-C₃-alkyl-, ((C₁-C₃-alkyl)-C(═O)—O)—C₂-C₃-alkyl-, —C(R¹⁸)(R¹⁹)—C(═O)—OR¹⁷, —C(R¹⁸)(R¹⁹)—C(═O)—N(R²⁰)(R²¹), —C(═O)—N(R²⁰)(R²¹), phenyl and 5- or 6-membered heteroaryl group,
wherein the phenyl group within said (phenyl)-(C₁-C₃-alkyl)- group and said phenyl group itself, and the 5- or 6-membered heteroaryl group within said (5- or 6-membered heteroaryl)-(C₁-C₃-alkyl)- group and said 5- or 6-membered heteroaryl group itself are optionally substituted one or two times, each substituent independently selected from the group consisting of a halogen atom or a group selected from the group consisting of cyano, methyl, ethyl, trifluoromethyl, methoxy, ethoxy, dimethylamino and trifluoromethoxy;
R¹⁰ and R¹¹ represent, independently from each occurrence, a hydrogen atom or a group selected from the group consisting of C₁-C₄-alkyl, C₁-C₄-haloalkyl, C₂-C₄-hydroxyalkyl, (C₁-C₃-alkoxy)-C₂-C₃-alkyl-, ((R²²)(R²³)N)—C₂-C₃-alkyl, (C₃-C₇-cycloalkyl)-(C₁-C₃-alkyl)-, (C₁-C₄-alkyl)-C(═O)—, C₃-C₇-cycloalkyl, (C₃-C₇-cycloalkyl)-C(═O)—, (phenyl)-(C₁-C₃-alkyl)-, (phenyl)-(C₁-C₃-alkyl)-C(═O)—, (phenyl)-(C₁-C₃-alkyl)-O—C(═O)—, phenyl and a 5- or 6-membered heteroaryl group,
wherein C₃-C₇-cycloalkyl, and the C₃-C₇-cycloalkyl within said (C₃-C₇-cycloalkyl)-(C₁-C₃-alkyl)- and (C₃-C₇-cycloalkyl)-C(═O)— groups are optionally substituted one or two times, each substituent independently selected from the group consisting of a fluorine atom or a group selected from the group consisting of cyano, C₁-C₂-alkyl and C₁-C₂-haloalkyl,
and wherein said phenyl and said 5- or 6-membered heteroaryl group, and the phenyl groups within said (phenyl)-(C₁-C₃-alkyl)-, (phenyl)-(C₁-C₃-alkyl)-C(═O)— and (phenyl)-(C₁-C₃-alkyl)-O—C(═O)— groups, are optionally substituted one or two times, each substituent independently selected from the group consisting of a halogen atom or a group selected from the group consisting of cyano, methyl, ethyl, trifluoromethyl, methoxy, ethoxy, dimethylamino and trifluoromethoxy,
or
R¹⁰ and R¹¹, together with the nitrogen atom to which they are attached, represent a monocyclic nitrogen containing 4- to 7-membered heterocycloalkyl group, or a bicyclic nitrogen containing 5- to 11-membered heterocycloalkyl group, which are optionally substituted one, two or three times, each substituent independently selected from the group consisting of a halogen atom or a group selected from the group consisting of cyano, oxo, hydroxy, C₁-C₄-alkyl, C₁-C₄-haloalkyl, (C₁-C₄-alkyl)-C(═O)—, C₃-C₇-cycloalkyl, C₁-C₄-alkoxy, —N(R²²)(R²³), and a monocyclic 4- to 7-membered heterocycloalkyl group;
R¹² and R¹³ represent, independently from each occurrence, a hydrogen atom or a group selected from the group consisting of C₁-C₄-alkyl, C₁-C₄-haloalkyl, C₁-C₄-hydroxyalkyl, (C₁-C₄-alkoxy)-C₂-C₃-alkyl-, (C₁-C₄-haloalkoxy)-C₂-C₃-alkyl-, (phenoxy)-C₂-C₃-alkyl-, C₃-C₇-cycloalkyl, monocyclic 4- to 7-membered heterocycloalkyl and (phenyl)-(C₁-C₃-alkyl)-,
wherein C₃-C₇-cycloalkyl and monocyclic 4- to 7-membered heterocycloalkyl are optionally substituted one, two or three times, each substituent independently selected from the group consisting of a halogen atom or a group selected from the group consisting of cyano, oxo, hydroxy, C₁-C₄-alkyl, (C₁-C₄-alkyl)-C(═O)—, C₃-C₄-cycloalkyl and C₁-C₄-alkoxy,
and wherein the phenyl groups within said (phenoxy)-C₂-C₃-alkyl- group and said (phenyl)-(C₁-C₃-alkyl)- group are optionally substituted one or two times, each substituent independently selected from the group consisting of a halogen atom or a group selected from the group consisting of cyano, methyl, ethyl, trifluoromethyl, methoxy, ethoxy, dimethylamino and trifluoromethoxy,
or
R¹² and R¹³, together with the nitrogen atom to which they are attached, represent a monocyclic nitrogen containing 4- to 7-membered heterocycloalkyl group which is optionally substituted one, two or three times, each substituent independently selected from the group consisting of a halogen atom or a group selected from the group consisting of cyano, oxo, hydroxy, C₁-C₄-alkyl, (C₁-C₄-alkyl)-C(═O)—, C₃-C₄-cycloalkyl and C₁-C₄-alkoxy;
R¹⁴ represents a group selected from the group consisting of C₁-C₄-alkyl, C₁-C₄-haloalkyl and phenyl,
wherein the phenyl group is optionally substituted one or two times, each substituent independently selected from the group consisting of a halogen atom or a group selected from the group consisting of cyano, methyl, ethyl, trifluoromethyl, methoxy, ethoxy, dimethylamino and trifluoromethoxy;
R¹⁷ represents a C₁-C₄-alkyl group;
R¹⁸ and R¹⁹ represent, independently from each occurrence, a hydrogen atom or a C₁-C₄-alkyl group;
R²⁰ represents a hydrogen atom or a group selected from the group consisting of C₁-C₆-alkyl, C₃-C₄-alkenyl, C₃-C₄-alkynyl, C₁-C₃-alkoxy, C₃-C₇-cycloalkyl, bicyclic C₅-C₁₁-cycloalkyl, adamantyl, monocyclic 4- to 7-membered heterocycloalkyl, bicyclic 5- to 11-membered heterocycloalkyl, phenyl, naphthyl, and 5- to 10-membered heteroaryl,
wherein said C₁-C₆-alkyl group is optionally substituted one, two or three times, each substituent independently selected from the group consisting of a halogen atom or a group selected from the group consisting of hydroxy, cyano, C₁-C₃-alkoxy, —N(R²²)(R²³), C₃-C₇-cycloalkyl, bicyclic C₅-C₁₁-cycloalkyl, adamantyl, monocyclic 4- to 7-membered heterocycloalkyl, bicyclic 5- to 11-membered heterocycloalkyl, phenyl, and 5- to 10-membered heteroaryl, said phenyl and 5- to 10-membered heteroaryl substituents themselves being optionally substituted one or two times, each substituent independently selected from the group consisting of a halogen atom or a group selected from the group consisting of cyano, methyl, ethyl, trifluoromethyl, methoxy, ethoxy, dimethylamino and trifluoromethoxy,
and wherein C₃-C₇-cycloalkyl, bicyclic C₅-C₁₁-cycloalkyl, adamantyl, monocyclic 4- to 7-membered heterocycloalkyl and bicyclic 5- to 11-membered heterocycloalkyl are optionally substituted one, two or three times, each substituent independently selected from the group consisting of a halogen atom or a group selected from the group consisting of cyano, oxo, hydroxy, C₁-C₄-alkyl, (C₁-C₄-alkyl)-C(═O)—, C₃-C₄-cycloalkyl and C₁-C₄-alkoxy,
and wherein said phenyl, naphthyl and 5- to 10-membered heteroaryl groups are optionally substituted one, two or three times, each substituent independently selected from the group consisting of a halogen atom or a group selected from the group consisting of cyano, C₁-C₄-alkyl, C₁-C₄-haloalkyl, C₁-C₄-alkoxy, C₁-C₄-haloalkoxy, —N(R²²)(R²³) and —C(═O)—N(R²⁴)(R²⁵),
R²¹ represents a hydrogen atom or a C₁-C₄-alkyl group,
or
R²⁰ and R²¹, together with the nitrogen atom to which they are attached, represent a monocyclic nitrogen containing 4- to 7-membered heterocycloalkyl group which is optionally benzocondensed, and which is optionally substituted one, two or three times, each substituent independently selected from the group consisting of a halogen atom or a group selected from the group consisting of cyano, oxo, hydroxy, C₁-C₄-alkyl, C₁-C₄-haloalkyl, (phenyl)-(C₁-C₃-alkyl)-, (C₁-C₄-alkyl)-C(═O)—, C₃-C₄-cycloalkyl, C₁-C₄-alkoxy, C₁-C₃-haloalkoxy, —N(R²²)(R²³) and —C(═O)—N(R²⁴)(R²⁵);
R²² and R²³ represent, independently from each occurrence, a hydrogen atom or a group selected from the group consisting of C₁-C₂-alkyl and (C₁-C₂-alkyl)-C(═O)—;
R²⁴ and R²⁵ represent, independently from each occurrence, a hydrogen atom or a C₁-C₄-alkyl group, and
n represents an integer 0, 1, or 2,
or a stereoisomer, a tautomer, an N-oxide, a hydrate, a solvate, or a salt thereof, or a mixture of same.

4. The combination according to claim 1, comprising one DGKalpha inhibitor compound of formula (I),
in which
R¹ represents a group selected from the group consisting of cyano, —C(═O)NH₂, —C(═O)N(H)CH₃ and —C(═O)N(CH₃)₂,
R² represents a group selected from the group consisting of phenyl, naphthyl and 5- to 10-membered heteroaryl,
which 5- to 10-membered heteroaryl group is connected to the rest of the molecule via a carbon atom of said 5- to 10-membered heteroaryl group, and
which phenyl, naphthyl and 5- to 10-membered heteroaryl group is optionally substituted, one, two, three or four times, each substituent independently selected from the group consisting of a halogen atom or a group selected from the group consisting of
C₁-C₆-alkyl, C₃-C₅-cycloalkyl, C₁-C₄-haloalkyl, (C₁-C₂-alkoxy)-(C₁-C₂-alkyl)-, C₁-C₄-alkoxy, C₁-C₄-haloalkoxy, phenoxy, —S(═O)₂R¹⁴, —P(═O)(R¹⁴)₂, cyano, hydroxy, —N(R⁹)(R¹⁰), —C(═O)N(R⁹)(R¹⁰), —C(═O)R¹¹, —N(R¹²)C(═O)R¹³, —N(R¹²)S(═O)₂R¹⁴, —N═S(═O)(R¹⁴)₂, 4- to 7-membered heterocycloalkyl, phenyl and 5- or 6-membered heteroaryl,
or two substituents of said phenyl group, when they are attached to adjacent ring atoms, are optionally linked to one another in such a way that they jointly form a group selected from the group consisting of
—CH₂—CH(OH)—CH₂—, —CH₂—CH(CH₃)—O—, —O—C(CH₃)₂—O—, —N(R¹⁸)—C(═O)—(C(R¹⁸)(R¹⁹))ₘ—, —N(R¹⁸)—C(═O)—(C(CH₂)₃)—, —N(R¹⁸)—(C(R¹⁸)(R¹⁹))ₘ—, —N(R¹⁸)—C(═O)—O— and —N(R¹⁸)—C(═O)—N(R¹⁸)—, wherein said 4- to 7-membered heterocycloalkyl group is connected to the rest of the molecule via a carbon atom of said 4- to 7-membered heterocycloalkyl group,
and
wherein said C₁-C₄-alkoxy group is optionally substituted with a group selected from the group consisting of 4- to 7-membered heterocycloalkyl and phenyl,
wherein said 4- to 7-membered heterocycloalkyl group is connected to the rest of the molecule via a carbon atom of said 4- to 7-membered heterocycloalkyl group,
and
wherein said phenyl and 5- or 6-membered heteroaryl group is optionally substituted, one or two times, each substituent independently selected from the group consisting of a halogen atom or a group selected from the group consisting of
C₁-C₂-alkyl, C₁-C₂-haloalkyl and C₁-C₂-alkoxy,
R³ represents a hydrogen atom or a halogen atom or a group selected from the group consisting of
C₁-C₆-alkyl, C₂-C₄-alkenyl, C₃-C₅-cycloalkyl, (C₁-C₂-alkoxy)-(C₁-C₄-alkyl)-, C₁-C₄-alkoxy, C₁-C₄-haloalkoxy, C₃-C₅-cycloalkyloxy, —S(═O)₂R¹⁴, cyano, hydroxy, —N(R⁹)(R¹⁰), —C(═O)N(R⁹)(R¹⁰), —P(═O)(R¹⁴)₂, 4- to 7-membered heterocycloalkyl, 5- to 7-membered heterocycloalkenyl, (4- to 7-membered heterocycloalkyl)oxy and phenyl,
wherein said 4- to 7-membered heterocycloalkyl group and 5- to 7-membered heterocycloalkenyl group is connected to the rest of the molecule via a carbon atom of said 4- to 7-membered heterocycloalkyl group and 5- to 7-membered heterocycloalkenyl group,
and
wherein said C₁-C₆-alkyl and C₁-C₄-alkoxy group is optionally substituted with a group selected from the group consisting of
C₃-C₄-cycloalkyl and 4- to 7-membered heterocycloalkyl,
wherein said 4- to 7-membered heterocycloalkyl group is connected to the rest of the molecule via a carbon atom of said 4- to 7-membered heterocycloalkyl group,
and
which C₃-C₄-cycloalkyl group is optionally substituted, one or two times, with a cyano group,
and
wherein said C₂-C₄-alkenyl group is optionally substituted with a C₁-C₄-haloalkyl group,
and
wherein said C₃-C₅-cycloalkyl group is optionally substituted, one or two times, each substituent independently selected from the group consisting of a halogen atom or a group selected from the group consisting of C₁-C₄-alkyl and C₁-C₄-haloalkyl,
R⁴ represents a hydrogen atom or a halogen atom or a group selected from the group consisting of
C₁-C₆-alkyl, C₁-C₆-haloalkyl, C₃-C₅-cycloalkyl, (C₁-C₂-alkoxy)-(C₁-C₄-alkyl)-, C₁-C₄-alkoxy, (C₁-C₂ alkoxy)-(C₁-C₄-alkoxy)-, —O—(C₁-C₄-alkyl)-C(═O)OR¹⁵, —O—(C₁-C₄-alkyl)-C(═O)N(R⁹)(R¹⁰), C₃-C₅-cycloalkyloxy, —S(═O)₂R¹⁴, cyano, nitro, hydroxy, —N(R⁹)(R¹⁰), —N(R¹⁶)(R¹⁷), —N(R¹⁶)(R²⁰), —N═S(═NH)(R¹⁴)₂, —N═S(═O)(R¹⁴)₂, —P(═O)(R¹⁴)₂, 4- to 7-membered heterocycloalkyl, 5- to 7-membered heterocycloalkenyl, (4- to 7-membered heterocycloalkyl)oxy and phenyl,
wherein said 4- to 7-membered heterocycloalkyl group and, 5- to 7-membered heterocycloalkenyl group is connected to the rest of the molecule via a carbon atom of said 4- to 7-membered heterocycloalkyl group and 5- to 7-membered heterocycloalkenyl group,
and
wherein said C₁-C₆-alkyl and C₁-C₄-alkoxy group is optionally substituted with a group selected from the group consisting of
C₃-C₄-cycloalkyl and 4- to 7-membered heterocycloalkyl,
wherein said 4- to 7-membered heterocycloalkyl group is connected to the rest of the molecule via a carbon atom of said 4- to 7-membered heterocycloalkyl group,
and
which C₃-C₄-cycloalkyl group is optionally substituted, one or two times, each substituent independently selected from the group consisting of a halogen atom or a group selected from the group consisting of cyano and hydroxy,
and
wherein said C₁-C₄-alkoxy group is optionally substituted with a oxiran-2-yl group,
and
wherein said C₃-C₅-cycloalkyl is optionally substituted, one or two times, each substituent independently selected from the group consisting of a halogen atom or a C₁-C₄-alkyl group,
R⁵ represents a hydrogen atom or a halogen atom or a group selected from the group consisting of
C₁-C₅-alkyl, C₃-C₅-cycloalkyl, C₁-C₄-alkoxy, C₃-C₅-cycloalkyloxy, —S(═O)₂R¹⁴, cyano, hydroxy, —N(R⁹)(R¹⁰), 4- to 7-membered heterocycloalkyl and (4- to 7-membered heterocycloalkyl)oxy,
wherein said 4- to 7-membered heterocycloalkyl group is connected to the rest of the molecule via a carbon atom of said 4- to 7-membered heterocycloalkyl group,
R⁶ represents a hydrogen atom or a group selected from the group consisting of C₁-C₄-alkyl and C₁-C₄-hydroxyalkyl,
R⁷ represents a hydrogen atom or a halogen atom or a group selected from the group consisting of
C₁-C₄-alkyl, C₁-C₄-alkoxy and hydroxy,
R⁸ represents a group selected from the group consisting of methyl and ethyl,
R⁹ and R¹⁰ represent, independently from each occurrence, a hydrogen atom or a group selected from the group consisting of C₁-C₄-alkyl, C₂-C₄-hydroxyalkyl, N≡C—(C₁-C₄-alkyl)-, (C₁-C₄-alkoxy)-(C₂-C₄-alkyl)- and C₃-C₄-cycloalkyl,
or
R⁹ and R¹⁰ together with the nitrogen to which they are attached represent a
nitrogen containing 4- to 7-membered heterocycloalkyl group,
wherein said nitrogen containing 4- to 7-membered heterocycloalkyl group is optionally substituted, one, two or three times, each substituent independently selected from the group consisting of a halogen atom or a group selected from the group consisting of
C₁-C₄-alkyl, hydroxy and oxo,
or
two substituents, which are attached to the same carbon atom of said nitrogen containing 4- to 7-membered heterocycloalkyl group, together with the carbon atom to which they are attached, represent a 4- to 7-membered heterocycloalkyl group,
wherein said 4- to 7-membered heterocycloalkyl group is optionally substituted, one or two times, with a C₁-C₄-alkyl group,
R¹¹ represents a group selected from the group consisting of C₁-C₄-alkyl and C₁-C₄-haloalkyl,
R¹² represents a hydrogen atom,
R¹³ represents a phenyl group,
R¹⁴ represents a group selected from the group consisting of C₁-C₄-alkyl and phenyl,
R¹⁵ represents a hydrogen atom or a C₁-C₄-alkyl group,
R¹⁶ represents a hydrogen atom or a C₁-C₄-alkyl group,
R¹⁷ represents a 4- to 7-membered heterocycloalkyl group,
wherein said 4- to 7-membered heterocycloalkyl group is optionally substituted, one or two times, with a C₁-C₄-alkyl group,
and
wherein said 4- to 7-membered heterocycloalkyl group is connected to the rest of the molecule via a carbon atom of the 4- to 7-membered heterocycloalkyl group,
R¹⁸ represents a hydrogen atom or a methyl group,
R¹⁹ represents a hydrogen atom or a methyl group,
R²⁰ represents a (4- to 7-membered heterocycloalkyl)-(C₁-C₄-alkyl)- group,
wherein the (4- to 7-membered heterocycloalkyl) part of said group is optionally substituted, one or two times, with a C₁-C₄-alkyl group,
m represents an integer selected from the group consisting of 1 and 2,
wherein said 4- to 7-membered heterocycloalkyl group is optionally substituted, one or two times, with a C₁-C₄-alkyl group,
and
n represents an integer selected from the group consisting of 1, 2 and 3,
or a stereoisomer, a tautomer, an N-oxide, a hydrate, a solvate, or a salt thereof, or a mixture of same;
and
one DGKζ inhibitor compound of formula (II),
in which:
R¹ represents a phenyl or pyridinyl group optionally substituted, one, two, or three times, each substituent independently selected from the group consisting of a fluorine atom, a chlorine atom and a bromine atom, or a group selected from the group consisting of hydroxy, cyano, C₁-C₄-alkyl, C₁-C₂-fluoroalkyl, C₁-C₂-alkoxy, (phenyl)-(C₁-C₂-alkoxy)-, C₁-C₂-fluoroalkoxy and —N(R⁵)(R⁶),
or two substituents attached to adjacent carbon atoms of said phenyl or pyridinyl group together form a bivalent group selected from the group consisting of —(CH₂)₃—, —O—CH₂—O— and —O—CF₂—O—,
or
R¹ represents a pyrazolyl group optionally substituted with one methyl group;
R² represents a group 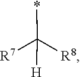
wherein “*” indicates the point of attachment to the nitrogen atom to which R² is attached;
R³ represents a group selected from the group consisting of methyl and —NH₂;
R⁴ represents a phenyl or pyridinyl group optionally substituted, one, two, or three times, each substituent independently selected from the group consisting of a halogen atom or a group selected from the group consisting of cyano, C₁-C₃-alkyl, ((R⁹)O)—(C₁-C₃-alkyl)-, C₁-C₃-fluoroalkyl, —OR⁹, —N(R¹⁰)(R¹¹), —C(═O)—N(R¹²)(R¹³), S(═O)ₙ—R¹⁴ and —C(═O)—OR¹⁷,
or two substituents attached to adjacent carbon atoms of said phenyl or pyridinyl group together form a bivalent group selected from the group consisting of —(CH₂)₃—, —O—CH₂—O— and —O—CF₂—O—;
R⁵ and R⁶ represent, independently from each occurrence, a hydrogen atom or a C₁-C₂-alkyl group,
or
R⁵ and R⁶, together with the nitrogen atom to which they are attached, represent a monocyclic nitrogen containing 4- to 7-membered heterocycloalkyl group which is optionally substituted one or two times, each substituent independently selected from the group consisting of a fluorine atom or a group selected from the group consisting of hydroxy and C₁-C₂-alkyl;
R⁷ represents a hydrogen atom or a C₁-C₂-alkyl group;
R⁸ represents a —C(═O)—NH₂ group;
R⁹ represents a hydrogen atom or a group selected from the group consisting of C₁-C₂-alkyl, benzyl, C₁-C₂-fluoroalkyl, C₂-hydroxyalkyl, (C₁-C₂-alkoxy)-C₂-alkyl-, ((C₁-C₂-alkyl)-C(═O)—O)—C₂-alkyl-, —C(R¹⁸)(R¹⁹)—C(═O)—OR¹⁷, —C(R¹⁸)(R¹⁹)—C(═O)—N(R²⁰)(R²¹), —C(═O)—N(R²⁰)(R²¹) and phenyl,
wherein the phenyl group within said benzyl group and said phenyl group itself are optionally substituted one or two times, each substituent independently selected from the group consisting of a fluorine atom and a chlorine atom, or a group selected from the group consisting of cyano and methyl;
R¹⁰ and R¹¹ represent, independently from each occurrence, a hydrogen atom or a group selected from the group consisting of C₁-C₂-alkyl, C₁-C₂-fluoroalkyl, (C₃-C₅-cycloalkyl)-(C₁-C₂-alkyl)- (C₁-C₂-alkyl)-C(═O)—, C₃-C₇-cycloalkyl, C₃-C₇-cycloalkyl-(C═O)—, (phenyl)-(C₁-C₂-alkyl)-, (phenyl)-(C₁-C₂-alkyl)-C(═O)— and (phenyl)-(C₁-C₂-alkyl)-O—C(═O)—,
wherein C₃-C₇-cycloalkyl, and the C₃-C₅-cycloalkyl within said (C₃-C₅-cycloalkyl)-(C₁-C₂-alkyl)- and the C₃-C₇-cycloalkyl within the C₃-C₇-cycloalkyl-(C═O)— groups are optionally substituted one or two times, each substituent independently selected from the group consisting of a fluorine atom or a group selected from the group consisting of cyano, C₁-C₂-alkyl and C₁-C₂-fluoroalkyl,
and wherein the phenyl groups within said (phenyl)-(C₁-C₂-alkyl)-, (phenyl)-(C₁-C₂-alkyl)-C(═O)— and (phenyl)-(C₁-C₂-alkyl)-O—C(═O)— groups are optionally substituted one or two times, each substituent independently selected from the group consisting of a fluorine atom, a chlorine atom and a methyl group,
or
R¹⁰ and R¹¹, together with the nitrogen atom to which they are attached, represent a monocyclic nitrogen containing 4- to 7-membered heterocycloalkyl group which is optionally substituted one or two times, each substituent independently selected from the group consisting of a fluorine atom or a group selected from the group consisting of cyano, oxo, C₁-C₂-alkyl, C₁-C₂-fluoroalkyl and (C₁-C₂-alkyl)-C(═O)—;
R¹² and R¹³ represent, independently from each occurrence, a hydrogen atom or a group selected from the group consisting of C₁-C₄-alkyl, C₁-C₄-fluoroalkyl, C₁-C₄-hydroxyalkyl, (C₁-C₄-alkoxy)-C₂-C₃-alkyl-, (C₁-C₂-fluoroalkoxy)-C₂-C₃-alkyl-, (phenoxy)-C₂-C₃-alkyl-, C₃-C₇-cycloalkyl, monocyclic 4- to 7-membered heterocycloalkyl and (phenyl)-(C₁-C₂-alkyl)-,
wherein C₃-C₇-cycloalkyl and monocyclic 4- to 7-membered heterocycloalkyl are optionally substituted one or two times, each substituent independently selected from the group consisting of a fluorine atom or a group selected from the group consisting of oxo, C₁-C₂-alkyl and (C₁-C₂-alkyl)-C(═O)—,
and wherein the phenyl groups within said (phenoxy)-C₂-C₃-alkyl- group and said (phenyl)-(C₁-C₂-alkyl)- group are optionally substituted one or two times, each substituent independently selected from the group consisting of a fluorine atom and a chlorine atom, or a group selected from the group consisting of methyl, trifluoromethyl and methoxy,
or
R¹² and R¹³, together with the nitrogen atom to which they are attached, represent a monocyclic nitrogen containing 4- to 7-membered heterocycloalkyl group which is optionally substituted one or two times, each substituent independently selected from the group consisting of a halogen atom or a group selected from the group consisting of oxo, C₁-C₂-alkyl and (C₁-C₂-alkyl)-C(═O)—;
R¹⁴ represents a group selected from the group consisting of methyl and trifluoromethyl;
R¹⁷ represents a C₁-C₂-alkyl group;
R¹⁸ and R¹⁹ represent, independently from each occurrence, a hydrogen atom or a methyl group;
R²⁰ represents a hydrogen atom or a group selected from the group consisting of optionally substituted C₁-C₃-alkyl, unsubstituted C₄-C₆-alkyl, prop-2-ynyl, methoxy, C₃-C₆-cycloalkyl, adamantyl, monocyclic 4- to 7-membered heterocycloalkyl, phenyl, and 5- to 10-membered heteroaryl,
wherein said C₁-C₃-alkyl group is optionally substituted one, two or three times, each substituent independently selected from the group consisting of a halogen atom or a group selected from the group consisting of hydroxy, cyano, C₁-C₃-alkoxy, —N(R²²)(R²³), C₃-C₆-cycloalkyl, adamantyl, monocyclic 4- to 7-membered heterocycloalkyl, phenyl, and 5- to 10-membered heteroaryl, said phenyl and 5- to 10-membered heteroaryl substituents themselves being optionally substituted one or two times, each substituent independently selected from the group consisting of a fluorine atom, a chlorine atom and a methyl group,
and wherein said C₃-C₆-cycloalkyl, adamantyl and monocyclic 4- to 7-membered heterocycloalkyl groups are optionally substituted one or two or three times, each substituent independently selected from the group consisting of a fluorine atom or a group selected from the group consisting of oxo, C₁-C₂-alkyl and (C₁-C₂-alkyl)-C(═O)—,
and wherein said phenyl and 5- to 10-membered heteroaryl groups are optionally substituted one, two or three times, each substituent independently selected from the group consisting of a fluorine atom and a chlorine atom or a group selected from the group consisting of cyano, C₁-C₂-alkyl, C₁-C₂-fluoroalkyl, C₁-C₂-alkoxy, C₁-C₂-fluoroalkoxy, —N(R²²)(R²³) and —C(═O)—N(R²⁴)(R²⁵),
R²¹ represents a hydrogen atom or a C₁-C₂-alkyl group,
or
R²⁰ and R²¹, together with the nitrogen atom to which they are attached, represent a monocyclic nitrogen containing 4- to 7-membered heterocycloalkyl group which is optionally benzocondensed, and which is optionally substituted one, two or three times, each substituent independently selected from the group consisting of a halogen atom or a group selected from the group consisting of cyano, oxo, hydroxy, C₁-C₂-alkyl, C₁-C₂-fluoroalkyl, benzyl, (C₁-C₂-alkyl)-C(═O)—, C₃-C₄-cycloalkyl, C₁-C₂-alkoxy, C₁-C₂-fluoroalkoxy, —N(R²²)(R²³) and —C(═O)—N(R²⁴)(R²⁵);
R²² and R²³ represent, independently from each occurrence, a hydrogen atom or a group selected from the group consisting of C₁-C₂-alkyl and (C₁-C₂-alkyl)-C(═O)—;
R²⁴ and R²⁵ represent, independently from each occurrence, a hydrogen atom or a C₁-C₂-alkyl group, and
n represents an integer 2,
or a stereoisomer, a tautomer, an N-oxide, a hydrate, a solvate, or a salt thereof, or a mixture of same.

5. The combination according to claim 1, comprising one DGKalpha inhibitor compound of formula (I),
in which
R¹ represents a group selected from the group consisting of cyano, —C(═O)NH₂, —C(═O)N(H)CH₃ and —C(═O)N(CH₃)₂,
R² represents a group selected from the group consisting of phenyl, 1-naphthyl, 2-naphthyl, 1H-pyrazol-3-yl, 1H-pyrazol-4-yl, 1H-pyrazol-5-yl, 1,2,4-oxadiazol-5-yl, 1,3,4-oxadiazol-2-yl, 1H-1,2,3-triazol-4-yl, 2H-1,2,3-triazol-4-yl, 1,3-thiazol-2-yl, pyridin-3-yl, pyrazin-2-yl, 1H-indol-5-yl, 1-benzofuran-4-yl, 1-benzofuran-7-yl, 1H-indol-6-yl, benzothiophen-2-yl, 1,3-benzoxazol-2-yl, 1,3-benzoxazol-5-yl, 1,3-benzoxazol-6-yl, 1,3-benzoxazol-7-yl, 1H-indazol-5-yl, 1H-benzimidazol-2-yl, 1H-benzimidazol-4-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1H-pyrrolo[2,3-b]pyridin-3-yl, quinolin-2-yl, quinolin-4-yl, quinolin-6-yl, quinolin-7-yl, isoquinolin-5-yl, isoquinolin-7-yl, isoquinolin-8-yl, quinoxalin-2-yl, quinoxalin-5-yl and 1,3-thiazolo[5,4-b]pyridin-2-yl,
which group is optionally substituted, one or two times, each substituent independently selected from the group consisting of a fluorine, chlorine or bromine atom or a group selected from the group consisting of
methyl, propyl, isopropyl, tert-butyl, cyclopropyl, difluoromethyl, trifluoromethyl, methoxy, ethoxy, propoxy, (propan-2-yl)oxy, methoxymethyl, 2-methoxyethyl, benzyloxy, trifluormethoxy, 2,2,2-trifluoroethoxy, phenoxy, (oxolan-2-yl)methoxy, (tetrahydrofuran-2-yl)methoxy, methanesulfonyl, dimethylphosphoryl, cyano, hydroxy, dimethylamino, oxetan-3-yl, 2-oxopyrrolidin-1-yl, 4-methyl-2-oxopiperazin-1-yl, 4-methyl-3-oxopiperazin-1-yl, morpholino-4-yl, 7-oxo-2-oxa-6-azaspiro[3.4]octan-6-yl, 8-methyl-3-oxo-2,8-diazaspiro[4.5]decan-2-yl, carbamoyl, acetyl, trifluoroacetyl, benzamido, benzenesulfonamido, [dimethyl(oxido)-λ⁶-sulfanylidene]amino, phenyl, 3-chlorophenyl, 4-chlorophenyl, 2-methylphenyl, 3-methylphenyl, 4-methylphenyl, 3-trifluoromethylphenyl, 2-methoxyphenyl, 3-methoxyphenyl, 4-methoxyphenyl and pyridin-3-yl,
or
two substituents of said phenyl group, when they are attached to adjacent ring atoms, are linked to one another in such a way that they jointly form a group selected from the group consisting of
—CH₂—CH(OH)—CH₂—, —CH₂—CH(CH₃)—O—, —O—C(CH₃)₂—O—, —NH—C(═O)—CH(CH₃)—, —N(CH₃)—C(═O)—C(CH₃)₂—, —NH—C(═O)—(C(CH₂)₃)—, —NH—CH₂—C(CH₃)₂—, —N(CH₃)—C(═O)—O— and —N(CH₃)—C(═O)—N(CH₃)—,
R³ represents a hydrogen atom or a fluorine, chlorine or bromine atom or a group selected from the group consisting of methyl, sec-butyl, (oxetan-3-yl)methyl, 3,3,3-trifluoroprop-1-en-2-yl, cyclopropyl, (trifluoromethyl)cyclopropyl, cyclobutyl, 2,2-dimethylcyclobutyl, 3,3-difluorocyclobutyl, methoxymethyl, methoxy, ethoxy, propoxy, 2,2-difluoroethoxy, 2,2-difluoropropoxy, cyclopropylmethoxy, (1-cyanocyclopropyl)methoxy, cyclopropyloxy, cyclobutyloxy, methanesulfonyl, cyano, hydroxy, 4-hydroxy-2-oxo-pyrrolidin-1-yl, 7-oxo-2-oxa-6-azaspiro[3.4]octan-6-yl, carbamoyl, dimethylphosphoryl, oxetan-3-yl, 3,6-dihydro-2H-pyran-4-yl, (oxetan-3-yl)oxy and phenyl,
R⁴ represents a hydrogen atom or a fluorine, chlorine or bromine atom or a group selected from the group consisting of methyl, sec-butyl, (oxetan-3-yl)methyl, trifluoromethyl, cyclopropyl, 3,3-difluorocyclobutyl, methoxymethyl, methoxy, propoxy, 2-methoxyethoxy, (1-hydroxycyclopropyl)methoxy, (1-cyanocyclopropyl)methoxy, (oxiran-2-yl)methoxy, carboxymethoxy, 2-tert-butoxy-2-oxo-ethoxy, 2-amino-2-oxo-ethoxy, cyclopropyloxy, cyclobutyloxy, methanesulfonyl, dimethylphosphoryl, cyano, nitro, hydroxy, (cyanomethyl)(methyl)amino, (2-hydroxyethyl)amino, (2-hydroxyethyl)(methyl)amino, (2-methoxyethyl)amino, (2-methoxyethyl)(methyl)amino, cyclopropylamino, (oxetan-3-yl)amino, methyl(oxetan-3-yl)amino, methyl(oxolan-3-yl)amino, 3-hydroxyazetidin-1-yl, 2-oxopyrrolidin-1-yl, morpholino, 1,1-dioxidothiomorpholin-4-yl, 4-hydroxy-2-oxo-pyrrolidin-1-yl, 7-oxo-2-oxa-6-azaspiro[3.4]octan-6-yl, 2,2-dimethyl-2λ⁶-diazathia-1,2-dien-1-yl, [dimethyl(oxido)-λ⁶-sulfanylidene]amino, methyl(tetrahydrofuran-3-yl)amino, tetrahydrofuran-3-ylmethoxy, (tetrahydrofuran-3-ylmethyl)amino, oxetan-3-yl, 3,6-dihydro-2H-pyran-4-yl, (oxetan-3-yl)oxy, (tetrahydrofuran-3-yl)oxy, (tetrahydro-2H-pyran-3-yl)oxy, (tetrahydro-2H-pyran-4-yl)oxy and phenyl,
R⁵ represents a hydrogen atom or a fluorine, chlorine or bromine atom or a group selected from the group consisting of methyl, cyclopropyl, methoxy, propoxy, cyclopropyloxy, methanesulfonyl, cyano, hydroxy, oxetan-3-yl and oxetan-3-yloxy,
R⁶ represents a hydrogen atom or a group selected from the group consisting of methyl and hydroxymethyl,
R⁷ represents a hydrogen atom or a fluorine atom or a group selected from the group consisting of methyl, ethyl, methoxy and hydroxy,
R⁸ represents a group selected from the group consisting of methyl and ethyl,
and
n represents an integer selected from the group consisting of 1, 2 and 3, or a stereoisomer, a tautomer, an N-oxide, a hydrate, a solvate, or a salt thereof, or a mixture of same;
and
one DGKζ inhibitor compound of formula (II),
in which: R¹ represents a group 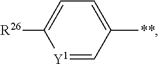
wherein “**” indicates the point of attachment to the nitrogen atom to which R¹ is attached;
R² represents a group 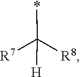
wherein “*” indicates the point of attachment to the nitrogen atom to which R² is attached;
R³ represents a group selected from the group consisting of methyl and —NH₂;
R⁴ represents a group 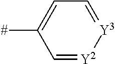
wherein “#” indicates the point of attachment to the carbonyl group to which R⁴ is attached;
R⁷ represents a hydrogen atom or a C₁-C₂-alkyl group;
R⁸ represents a —C(═O)—NH₂ group;
R⁹ represents a hydrogen atom or a group selected from the group consisting of C₁-C₂-alkyl, benzyl, C₁-C₂-fluoroalkyl, (C₁-C₂-alkoxy)-C₂-alkyl-, ((C₁-C₂-alkyl)-C(═O)—O)—C₂-alkyl-, —C(R¹⁸)(R¹⁹)—C(═O)—N(R²⁰)(R²¹), —C(═O)—N(R²⁰)(R²¹) and phenyl,
wherein the phenyl group within said benzyl group and said phenyl group itself are optionally substituted one or two times, each substituent independently selected from the group consisting of a fluorine atom and a chlorine atom, or a group selected from the group consisting of cyano and methyl;
R¹⁰ and R¹¹ represent, independently from each occurrence, a hydrogen atom or a group selected from the group consisting of C₁-C₂-alkyl, C₃-C₇-cycloalkyl and (benzyl)-O—C(═O)—,
wherein C₃-C₇-cycloalkyl is optionally substituted one or two times, each substituent independently selected from the group consisting of a fluorine atom or a group selected from the group consisting of methyl and trifluoromethyl,
and wherein the phenyl group within said (benzyl)-O—C(═O)— group is optionally substituted one or two times, each substituent independently selected from the group consisting of a fluorine atom, a chlorine atom and a methyl group,
or
R¹⁰ and R¹¹, together with the nitrogen atom to which they are attached, represent a monocyclic nitrogen containing 4- to 7-membered heterocycloalkyl group which is optionally substituted one or two times, each substituent independently selected from the group consisting of a fluorine atom or a group selected from the group consisting of cyano, methyl and trifluoromethyl;
R¹² and R¹³ represent, independently from each occurrence, a hydrogen atom or a group selected from the group consisting of C₁-C₄-alkyl, C₁-C₂-fluoroalkyl, C₁-C₂-hydroxyalkyl, (C₁-C₄-alkoxy)-C₂-alkyl-, (C₁-C₂-fluoroalkoxy)-C₂-alkyl-, (phenoxy)-C₂-alkyl-, C₃-C₇-cycloalkyl and (phenyl)-(C₁-C₂-alkyl)-,
wherein the phenyl groups within said (phenoxy)-C₂-alkyl- group and said (phenyl)-(C₁-C₂-alkyl)- group are optionally substituted one or two times, each substituent independently selected from the group consisting of fluorine atom and a chlorine atom, or a group selected from the group consisting of methyl, trifluoromethyl and methoxy;
R¹⁷ represents a C₁-C₂-alkyl group;
R¹⁸ and R¹⁹ represent, independently from each occurrence, a hydrogen atom or a methyl group;
R²⁰ represents a group selected from the group consisting of benzyl and phenyl,
wherein said phenyl group, and the phenyl group within said benzyl group, is optionally substituted one or two times, each substituent independently selected from the group consisting of a fluorine atom, a chlorine atom and a methyl group,
R²¹ represents a hydrogen atom or a methyl group,
Y¹ represents —C(H)═, —C(F)═, —C(Cl)═, —C(CN)═ or —N═;
Y² represents —C(H)═ or —N═;
Y³ represents —C(R²⁷)═ or —N═,
with the proviso that if Y² represents —N═, Y³ represents —C(R²⁷)═, and if Y³ represents —N═, Y² represents —C(H)═;
R²⁶ represents a fluorine atom, a chlorine atom or a bromine atom, or a group selected from the group consisting of methyl, difluoromethyl, trifluoromethyl, methoxy, benzyloxy, difluoromethoxy and trifluoromethoxy, and
R²⁷ represents a halogen atom or a group selected from the group consisting of C₁-C₂-alkyl, C₁-C₂-fluoroalkyl, —OR⁹, —N(R¹⁰)(R¹¹), —C(═O)—N(R¹²)(R¹³) and —C(═O)—OR¹⁷,
or a stereoisomer, a tautomer, an N-oxide, a hydrate, a solvate, or a salt thereof, or a mixture of same.

6. The combination according to claim 1, comprising one DGKalpha inhibitor compound of formula (I) selected from the group consisting of:
4-[4-(1,3-benzoxazol-2-yl)-4-methylpiperidin-1-yl]-1-methyl-2-oxo-1,2-dihydroquinoline-3-carbonitrile,
4-[4-(1,3-benzoxazol-2-yl)piperidin-1-yl]-1-methyl-2-oxo-1,2-dihydroquinoline-3-carbonitrile,
4-[4-(7-fluoro-1,3-benzoxazol-2-yl)piperidin-1-yl]-1-methyl-2-oxo-1,2-dihydroquinoline-3-carbonitrile,
4-[4-(6-fluoro-1,3-benzoxazol-2-yl)piperidin-1-yl]-1-methyl-2-oxo-1,2-dihydroquinoline-3-carbonitrile,
1-methyl-4-[4-(5-methyl-1,3-benzoxazol-2-yl)piperidin-1-yl]-2-oxo-1,2-dihydroquinoline-3-carbonitrile,
4-[4-(5-fluoro-1,3-benzoxazol-2-yl)piperidin-1-yl]-1-methyl-2-oxo-1,2-dihydroquinoline-3-carbonitrile,
1-methyl-4-[4-(6-methyl-1,3-benzoxazol-2-yl)piperidin-1-yl]-2-oxo-1,2-dihydroquinoline-3-carbonitrile,
1-methyl-2-oxo-4-{4-[5-(propan-2-yl)-1,3-benzoxazol-2-yl]piperidin-1-yl}-1,2-dihydroquinoline-3-carbonitrile,
4-[4-(6-chloro-1,3-benzoxazol-2-yl)piperidin-1-yl]-1-methyl-2-oxo-1,2-dihydroquinoline-3-carbonitrile,
4-[4-(5-bromo-1,3-benzoxazol-2-yl)piperidin-1-yl]-1-methyl-2-oxo-1,2-dihydroquinoline-3-carbonitrile,
4-[4-(5-chloro-1,3-benzoxazol-2-yl)piperidin-1-yl]-1-methyl-2-oxo-1,2-dihydroquinoline-3-carbonitrile,
4-[4-(1,3-benzothiazol-2-yl)piperidin-1-yl]-1-methyl-2-oxo-1,2-dihydroquinoline-3-carbonitrile,
4-[4-(5-fluoro-1,3-benzothiazol-2-yl)piperidin-1-yl]-1-methyl-2-oxo-1,2-dihydroquinoline-3-carbonitrile,
4-[4-(6-bromo-1,3-benzothiazol-2-yl)piperidin-1-yl]-1-methyl-2-oxo-1,2-dihydroquinoline-3-carbonitrile,
1-methyl-4-[4-(7-methyl-1,3-benzoxazol-2-yl)piperidin-1-yl]-2-oxo-1,2-dihydroquinoline-3-carbonitrile,
4-[4-(1,3-benzothiazol-2-yl)-4-fluoropiperidin-1-yl]-1-methyl-2-oxo-1,2-dihydroquinoline-3-carbonitrile,
1-methyl-4-[4-(4-methyl-1,3-benzoxazol-2-yl)piperidin-1-yl]-2-oxo-1,2-dihydroquinoline-3-carbonitrile,
4-[4-(5-chloro-1,3-benzothiazol-2-yl)piperidin-1-yl]-1-methyl-2-oxo-1,2-dihydroquinoline-3-carbonitrile,
4-[4-(5-chloro[1,3]thiazolo[5,4-b]pyridin-2-yl)piperidin-1-yl]-1-methyl-2-oxo-1,2-dihydroquinoline-3-carbonitrile,
1-methyl-4-{4-methyl-4-[6-(trifluoromethoxy)-1,3-benzoxazol-2-yl]piperidin-1-yl}-2-oxo-1,2-dihydroquinoline-3-carbonitrile,
1-methyl-4-{4-methyl-4-[5-(propan-2-yl)-1,3-benzoxazol-2-yl]piperidin-1-yl}-2-oxo-1,2-dihydroquinoline-3-carbonitrile,
4-[4-(5,6-difluoro-1,3-benzoxazol-2-yl)piperidin-1-yl]-1-methyl-2-oxo-1,2-dihydroquinoline-3-carbonitrile,
1-methyl-2-oxo-4-{4-[5-(trifluoromethyl)-1,3-benzothiazol-2-yl]piperidin-1-yl}-1,2-dihydroquinoline-3-carbonitrile,
4-[4-(5-tert-butyl-1,3-benzoxazol-2-yl)-4-methylpiperidin-1-yl]-1-methyl-2-oxo-1,2-dihydroquinoline-3-carbonitrile,
4-{4-[5-(methanesulfonyl)-1,3-benzoxazol-2-yl]-4-methylpiperidin-1-yl}-1-methyl-2-oxo-1,2-dihydroquinoline-3-carbonitrile,
4-[4-(4-fluoro-1,3-benzoxazol-2-yl)piperidin-1-yl]-1-methyl-2-oxo-1,2-dihydroquinoline-3-carbonitrile,
4-[4-(1,3-benzoxazol-2-yl)piperidin-1-yl]-7-bromo-1-methyl-2-oxo-1,2-dihydroquinoline-3-carbonitrile,
7-bromo-1-methyl-4-{4-methyl-4-[6-(trifluoromethoxy)-1,3-benzoxazol-2-yl]piperidin-1-yl}-2-oxo-1,2-dihydroquinoline-3-carbonitrile,
7-bromo-4-[4-(5-chloro-1,3-benzoxazol-2-yl)piperidin-1-yl]-1-methyl-2-oxo-1,2-dihydroquinoline-3-carbonitrile,
7-bromo-1-methyl-2-oxo-4-{4-[5-(propan-2-yl)-1,3-benzoxazol-2-yl]piperidin-1-yl}-1,2-dihydroquinoline-3-carbonitrile,
7-bromo-4-[4-(5,6-difluoro-1,3-benzoxazol-2-yl)piperidin-1-yl]-1-methyl-2-oxo-1,2-dihydroquinoline-3-carbonitrile,
7-bromo-4-[4-(6-chloro-1,3-benzoxazol-2-yl)piperidin-1-yl]-1-methyl-2-oxo-1,2-dihydroquinoline-3-carbonitrile,
4-[4-(1,3-benzothiazol-2-yl)piperidin-1-yl]-7-bromo-1-methyl-2-oxo-1,2-dihydroquinoline-3-carbonitrile,
7-bromo-1-methyl-4-{4-methyl-4-[5-(propan-2-yl)-1,3-benzoxazol-2-yl]piperidin-1-yl}-2-oxo-1,2-dihydroquinoline-3-carbonitrile,
4-[4-(1,3-benzoxazol-2-yl)piperidin-1-yl]-1-methyl-2-oxo-7-(2-oxopyrrolidin-1-yl)-1,2-dihydroquinoline-3-carbonitrile,
4-[4-(1,3-benzothiazol-2-yl)piperidin-1-yl]-1-methyl-2-oxo-7-(2-oxopyrrolidin-1-yl)-1,2-dihydroquinoline-3-carbonitrile,
1-methyl-2-oxo-4-{4-[4-(2,2,2-trifluoroethoxy)phenyl]piperidin-1-yl}-1,2-dihydroquinoline-3-carbonitrile,
1-methyl-2-oxo-4-(4-{4-[(propan-2-yl)oxy]phenyl}piperidin-1-yl)-1,2-dihydroquinoline-3-carbonitrile,
4-[4-(4-ethoxyphenyl)piperidin-1-yl]-1-methyl-2-oxo-1,2-dihydroquinoline-3-carbonitrile,
4-[4-(4-cyclopropylphenyl)piperidin-1-yl]-1-methyl-2-oxo-1,2-dihydroquinoline-3-carbonitrile,
4-[4-(4-methoxyphenyl)piperidin-1-yl]-1-methyl-2-oxo-1,2-dihydroquinoline-3-carbonitrile,
1-methyl-2-oxo-4-[4-(4-propoxyphenyl)piperidin-1-yl]-1,2-dihydroquinoline-3-carbonitrile,
1-methyl-2-oxo-4-{4-[4-(trifluoromethoxy)phenyl]piperidin-1-yl}-1,2-dihydroquinoline-3-carbonitrile,
N-{4-[1-(3-cyano-1-methyl-2-oxo-1,2-dihydroquinolin-4-yl)piperidin-4-yl]phenyl}benzenesulfonamide,
4-[4-(3-cyclopropylphenyl)piperidin-1-yl]-1-methyl-2-oxo-1,2-dihydroquinoline-3-carbonitrile,
4-{4-[4-(dimethylamino)phenyl]piperidin-1-yl}-1-methyl-2-oxo-1,2-dihydroquinoline-3-carbonitrile,
1-methyl-2-oxo-4-{4-[4-(propan-2-yl)phenyl]piperidin-1-yl}-1,2-dihydroquinoline-3-carbonitrile,
4-{4-[4-(benzyloxy)phenyl]piperidin-1-yl}-1-methyl-2-oxo-1,2-dihydroquinoline-3-carbonitrile,
N-{4-[1-(3-cyano-1-methyl-2-oxo-1,2-dihydroquinolin-4-yl)piperidin-4-yl]phenyl}benzamide,
1-methyl-4-[4-(1-methyl-1H-indol-5-yl)piperidin-1-yl]-2-oxo-1,2-dihydroquinoline-3-carbonitrile,
4-[4-(3-fluoro-5-methylphenyl)piperidin-1-yl]-1-methyl-2-oxo-1,2-dihydroquinoline-3-carbonitrile,
4-[4-(2-methoxyphenyl)piperidin-1-yl]-1-methyl-2-oxo-1,2-dihydroquinoline-3-carbonitrile,
4-[4-([1,1′-biphenyl]-4-yl)piperidin-1-yl]-1-methyl-2-oxo-1,2-dihydroquinoline-3-carbonitrile,
4-[4-(4-chlorophenyl)piperidin-1-yl]-1-methyl-2-oxo-1,2-dihydroquinoline-3-carbonitrile,
4-[4-(3-methoxyphenyl)piperidin-1-yl]-1-methyl-2-oxo-1,2-dihydroquinoline-3-carbonitrile,
1-methyl-2-oxo-4-[4-(4-phenoxyphenyl)piperidin-1-yl]-1,2-dihydroquinoline-3-carbonitrile,
1-methyl-2-oxo-4-(4-phenylpiperidin-1-yl)-1,2-dihydroquinoline-3-carbonitrile,
1-methyl-4-(4-methyl-4-phenylpiperidin-1-yl)-2-oxo-1,2-dihydroquinoline-3-carbonitrile,
1-methyl-2-oxo-4-{4-[4-(2-oxopyrrolidin-1-yl)phenyl]piperidin-1-yl}-1,2-dihydroquinoline-3-carbonitrile,
4-[4-(2-fluorophenyl)piperidin-1-yl]-1-methyl-2-oxo-1,2-dihydroquinoline-3-carbonitrile,
1-methyl-2-oxo-4-{4-[4-(trifluoromethyl)phenyl]piperidin-1-yl}-1,2-dihydroquinoline-3-carbonitrile,
4-[4-(3-fluorophenyl)piperidin-1-yl]-1-methyl-2-oxo-1,2-dihydroquinoline-3-carbonitrile,
1-methyl-4-{4-[3-(morpholin-4-yl)phenyl]piperidin-1-yl}-2-oxo-1,2-dihydroquinoline-3-carbonitrile,
4-[4-(3-cyano-2-methylphenyl)piperidin-1-yl]-1-methyl-2-oxo-1,2-dihydroquinoline-3-carbonitrile,
4-{4-[4-(methanesulfonyl)phenyl]piperidin-1-yl}-1-methyl-2-oxo-1,2-dihydroquinoline-3-carbonitrile,
1-methyl-4-{4-[4-(4-methyl-2-oxopiperazin-1-yl)phenyl]piperidin-1-yl}-2-oxo-1,2-dihydroquinoline-3-carbonitrile,
4-[4-(1,3-benzoxazol-5-yl)piperidin-1-yl]-1-methyl-2-oxo-1,2-dihydroquinoline-3-carbonitrile,
N-{3-[1-(3-cyano-1-methyl-2-oxo-1,2-dihydroquinolin-4-yl)piperidin-4-yl]phenyl}benzenesulfonamide,
4-[4-(3-{[dimethyl(oxo)-λ6-sulfanylidene]amino}phenyl)piperidin-1-yl]-1-methyl-2-oxo-1,2-dihydroquinoline-3-carbonitrile,
1-methyl-4-[4-(naphthalen-1-yl)piperidin-1-yl]-2-oxo-1,2-dihydroquinoline-3-carbonitrile,
4-[4-(1,3-benzothiazol-4-yl)piperidin-1-yl]-1-methyl-2-oxo-1,2-dihydroquinoline-3-carbonitrile,
1-methyl-4-{4-[1-methyl-3-(trifluoroacetyl)-1H-indol-5-yl]piperidin-1-yl}-2-oxo-1,2-dihydroquinoline-3-carbonitrile,
4-[4-(1-benzofuran-7-yl)piperidin-1-yl]-1-methyl-2-oxo-1,2-dihydroquinoline-3-carbonitrile,
4-[4-(isoquinolin-7-yl)piperidin-1-yl]-1-methyl-2-oxo-1,2-dihydroquinoline-3-carbonitrile,
4-[4-(1,3-benzothiazol-7-yl)piperidin-1-yl]-1-methyl-2-oxo-1,2-dihydroquinoline-3-carbonitrile,
N-{3-[1-(3-cyano-1-methyl-2-oxo-1,2-dihydroquinolin-4-yl)piperidin-4-yl]phenyl}benzamide,
4-[4-(isoquinolin-8-yl)piperidin-1-yl]-1-methyl-2-oxo-1,2-dihydroquinoline-3-carbonitrile,
4-[4-(isoquinolin-5-yl)piperidin-1-yl]-1-methyl-2-oxo-1,2-dihydroquinoline-3-carbonitrile,
1-methyl-2-oxo-4-[4-(quinoxalin-5-yl)piperidin-1-yl]-1,2-dihydroquinoline-3-carbonitrile,
4-{4-[3-(methanesulfonyl)phenyl]piperidin-1-yl}-1-methyl-2-oxo-1,2-dihydroquinoline-3-carbonitrile,
4-[4-(4-fluorophenyl)piperidin-1-yl]-1-methyl-2-oxo-1,2-dihydroquinoline-3-carbonitrile,
1-methyl-4-[4-(2-methylphenyl)piperidin-1-yl]-2-oxo-1,2-dihydroquinoline-3-carbonitrile,
1-methyl-4-[4-(4-methylphenyl)piperidin-1-yl]-2-oxo-1,2-dihydroquinoline-3-carbonitrile,
4-[4-(3,5-dichlorophenyl)piperidin-1-yl]-1-methyl-2-oxo-1,2-dihydroquinoline-3-carbonitrile,
4-[4-(3-bromophenyl)piperidin-1-yl]-1-methyl-2-oxo-1,2-dihydroquinoline-3-carbonitrile,
4-[4-(4-cyanophenyl)piperidin-1-yl]-1-methyl-2-oxo-1,2-dihydroquinoline-3-carbonitrile,
4-{4-[3-(difluoromethyl)phenyl]piperidin-1-yl}-1-methyl-2-oxo-1,2-dihydroquinoline-3-carbonitrile,
4-[4-(4-bromophenyl)piperidin-1-yl]-1-methyl-2-oxo-1,2-dihydroquinoline-3-carbonitrile,
7-bromo-4-[4-(4-methoxyphenyl)piperidin-1-yl]-1-methyl-2-oxo-1,2-dihydroquinoline-3-carbonitrile,
7-bromo-1-methyl-2-oxo-4-(4-phenylpiperidin-1-yl)-1,2-dihydroquinoline-3-carbonitrile,
4-[4-(4-methoxyphenyl)piperidin-1-yl]-1,7-dimethyl-2-oxo-1,2-dihydroquinoline-3-carbonitrile,
4-[4-(4-methoxyphenyl)piperidin-1-yl]-1-methyl-2-oxo-7-phenyl-1,2-dihydroquinoline-3-carbonitrile,
4-[4-(4-methoxyphenyl)piperidin-1-yl]-1-methyl-2-oxo-1,2-dihydroquinoline-3,7-dicarbonitrile,
7-cyclopropyl-4-[4-(4-methoxyphenyl)piperidin-1-yl]-1-methyl-2-oxo-1,2-dihydroquinoline-3-carbonitrile,
7-(2,2-dimethyl-2λ⁶-diazathia-1,2-dien-1-yl)-4-[4-(4-methoxyphenyl)piperidin-1-yl]-1-methyl-2-oxo-1,2-dihydroquinoline-3-carbonitrile,
4-[4-(4-methoxyphenyl)piperidin-1-yl]-1-methyl-2-oxo-7-(2-oxopyrrolidin-1-yl)-1,2-dihydroquinoline-3-carbonitrile,
4-[4-(4-methoxyphenyl)piperidin-1-yl]-1-methyl-7-(oxetan-3-yl)-2-oxo-1,2-dihydroquinoline-3-carbonitrile,
7-(methanesulfonyl)-4-[4-(4-methoxyphenyl)piperidin-1-yl]-1-methyl-2-oxo-1,2-dihydroquinoline-3-carbonitrile,
7-{[dimethyl(oxo)-λ6-sulfanylidene]amino}-4-[4-(4-methoxyphenyl)piperidin-1-yl]-1-methyl-2-oxo-1,2-dihydroquinoline-3-carbonitrile,
7-(3,6-dihydro-2H-pyran-4-yl)-4-[4-(4-methoxyphenyl)piperidin-1-yl]-1-methyl-2-oxo-1,2-dihydroquinoline-3-carbonitrile,
4-[4-(1-benzofuran-4-yl)piperidin-1-yl]-1-methyl-2-oxo-1,2-dihydroquinoline-3-carbonitrile,
1-methyl-2-oxo-4-(4-{4-[(propan-2-yl)oxy]phenyl}piperidin-1-yl)-1,2-dihydroquinoline-3-carboxamide,
1-methyl-4-{4-methyl-4-[5-(propan-2-yl)-1,3-benzoxazol-2-yl]piperidin-1-yl}-2-oxo-1,2-dihydroquinoline-3-carboxamide,
4-[4-(1,3-benzoxazol-2-yl)piperidin-1-yl]-7-bromo-1-methyl-2-oxo-1,2-dihydroquinoline-3-carboxamide,
4-[4-(1,3-benzoxazol-2-yl)-4-methylpiperidin-1-yl]-1-methyl-2-oxo-1,2-dihydroquinoline-3-carboxamide,
4-[4-(1,3-benzoxazol-2-yl)-4-ethylpiperidin-1-yl]-1-methyl-2-oxo-1,2-dihydroquinoline-3-carbonitrile,
1-methyl-4-[4-(5-methyl-1,3-benzoxazol-2-yl)piperidin-1-yl]-2-oxo-1,2-dihydroquinoline-3-carboxamide,
1-methyl-2-oxo-4-{4-[4-(trifluoromethoxy)phenyl]piperidin-1-yl}-1,2-dihydroquinoline-3-carboxamide,
4-[4-(5,6-difluoro-1,3-benzoxazol-2-yl)piperidin-1-yl]-1-methyl-2-oxo-1,2-dihydroquinoline-3-carboxamide,
7-bromo-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[3-methyl-4-(5-methyl-1,3-benzoxazol-2-yl)piperidin-1-yl]-2-oxo-1,2-dihydroquinoline-3-carbonitrile, mixture of stereoisomers,
4-[4-(4-methoxyphenyl)piperidin-1-yl]-1-methyl-2-oxo-1,2-dihydroquinoline-3-carboxamide,
4-[4-(1,3-benzothiazol-2-yl)piperidin-1-yl]-1-methyl-2-oxo-1,2-dihydroquinoline-3-carboxamide,
1-methyl-2-oxo-4-(4-phenylpiperidin-1-yl)-1,2-dihydroquinoline-3-carboxamide,
4-[4-(4-methoxyphenyl)piperidin-1-yl]-1-methyl-7-(oxetan-3-yl)-2-oxo-1,2-dihydroquinoline-3-carboxamide,
4-[4-(1,3-benzothiazol-2-yl)piperidin-1-yl]-1-methyl-2-oxo-7-(2-oxopyrrolidin-1-yl)-1,2-dihydroquinoline-3-carboxamide,
(rac)-1-methyl-2-oxo-4-{4-[4-(propan-2-yl)phenyl]azepan-1-yl}-1,2-dihydroquinoline-3-carbonitrile,
1-methyl-2-oxo-4-{(4S)-4-[4-(propan-2-yl)phenyl]azepan-1-yl}-1,2-dihydroquinoline-3-carbonitrile,
1-methyl-2-oxo-4-{(4R)-4-[4-(propan-2-yl)phenyl]azepan-1-yl}-1,2-dihydroquinoline-3-carbonitrile,
(rac)-4-[4-(1,3-benzoxazol-2-yl)azepan-1-yl]-1-methyl-2-oxo-1,2-dihydroquinoline-3-carbonitrile,
1-methyl-2-oxo-4-[4-{4-[(propan-2-yl)oxy]phenyl}azepan-1-yl]-1,2-dihydroquinoline-3-carbonitrile,
(rac)-4-[4-(4-methoxyphenyl)azepan-1-yl]-1-methyl-2-oxo-1,2-dihydroquinoline-3-carbonitrile,
4-[(4R)-4-(4-methoxyphenyl)azepan-1-yl]-1-methyl-2-oxo-1,2-dihydroquinoline-3-carbonitrile,
4-[(4S)-4-(4-methoxyphenyl)azepan-1-yl]-1-methyl-2-oxo-1,2-dihydroquinoline-3-carbonitrile,
(rac)-4-[4-(1,3-benzoxazol-2-yl)azepan-1-yl]-7-bromo-1-methyl-2-oxo-1,2-dihydroquinoline-3-carbonitrile,
4-[(4R)-4-(1,3-benzoxazol-2-yl)azepan-1-yl]-7-bromo-1-methyl-2-oxo-1,2-dihydroquinoline-3-carbonitrile,
4-[(4S)-4-(1,3-benzoxazol-2-yl)azepan-1-yl]-7-bromo-1-methyl-2-oxo-1,2-dihydroquinoline-3-carbonitrile,
(rac)-4-[4-(4-chlorophenyl)azepan-1-yl]-1-methyl-2-oxo-1,2-dihydroquinoline-3-carbonitrile,
4-[(4R)-4-(4-chlorophenyl)azepan-1-yl]-1-methyl-2-oxo-1,2-dihydroquinoline-3-carbonitrile,
4-[(4SR)-4-(4-chlorophenyl)azepan-1-yl]-1-methyl-2-oxo-1,2-dihydroquinoline-3-carbonitrile,
(rac)-1-methyl-2-oxo-4-[4-phenylazepan-1-yl]-1,2-dihydroquinoline-3-carbonitrile,
(rac)-7-bromo-1-methyl-2-oxo-4-[4-phenylazepan-1-yl]-1,2-dihydroquinoline-3-carbonitrile,
(rac)-1-methyl-7-(oxetan-3-yl)-2-oxo-4-[4-phenylazepan-1-yl]-1,2-dihydroquinoline-3-carbonitrile,
(rac)-1-methyl-7-(morpholin-4-yl)-2-oxo-4-[4-phenylazepan-1-yl]-1,2-dihydroquinoline-3-carbonitrile,
(rac)-1-methyl-2-oxo-7-(2-oxopyrrolidin-1-yl)-4-[4-phenylazepan-1-yl]-1,2-dihydroquinoline-3-carbonitrile,
(rac)-7-(1,1-dioxo-1λ⁶-thiomorpholin-4-yl)-1-methyl-2-oxo-4-[4-phenylazepan-1-yl]-1,2-dihydroquinoline-3-carbonitrile,
(rac)-7-bromo-1-methyl-2-oxo-4-[4-phenylazepan-1-yl]-1,2-dihydroquinoline-3-carboxamide,
7-bromo-1-methyl-2-oxo-4-[(4S)-4-phenylazepan-1-yl]-1,2-dihydroquinoline-3-carboxamide,
7-bromo-1-methyl-2-oxo-4-[(4S)-4-phenylazepan-1-yl]-1,2-dihydroquinoline-3-carboxamide,
4-[4-ethyl-4-(5-methyl-1,3-benzoxazol-2-yl)piperidin-1-yl]-1-methyl-2-oxo-1,2-dihydroquinoline-3-carbonitrile,
7-bromo-4-[4-ethyl-4-(5-methyl-1,3-benzoxazol-2-yl)piperidin-1-yl]-1-methyl-2-oxo-1,2-dihydroquinoline-3-carbonitrile,
4-[4-(6-methoxypyridin-3-yl)piperidin-1-yl]-1-methyl-2-oxo-1,2-dihydroquinoline-3-carbonitrile,
1-methyl-4-[4-(6-methylpyridin-3-yl)piperidin-1-yl]-2-oxo-1,2-dihydroquinoline-3-carbonitrile,
1-methyl-2-oxo-4-[4-(pyridin-3-yl)piperidin-1-yl]-1,2-dihydroquinoline-3-carbonitrile,
4-[4-(1,3-benzothiazol-2-yl)piperidin-1-yl]-N,1-dimethyl-2-oxo-1,2-dihydroquinoline-3-carboxamide,
4-[4-(1,3-benzothiazol-2-yl)piperidin-1-yl]-N,N,1-trimethyl-2-oxo-1,2-dihydroquinoline-3-carboxamide,
1-methyl-4-[4-(1-methyl-1H-benzimidazol-2-yl)piperidin-1-yl]-2-oxo-1,2-dihydroquinoline-3-carbonitrile,
1-methyl-2-oxo-4-[4-(3-propyl-1,2,4-oxadiazol-5-yl)piperidin-1-yl]-1,2-dihydroquinoline-3-carbonitrile,
1-methyl-4-[4-(1-methyl-1H-pyrazol-5-yl)piperidin-1-yl]-2-oxo-1,2-dihydroquinoline-3-carbonitrile,
1-methyl-2-oxo-4-[4-(pyrazin-2-yl)piperidin-1-yl]-1,2-dihydroquinoline-3-carbonitrile,
4-[4-(4-chlorophenyl)-4-hydroxypiperidin-1-yl]-1-methyl-2-oxo-1,2-dihydroquinoline-3-carbonitrile,
4-{4-hydroxy-4-[3-(trifluoromethyl)phenyl]piperidin-1-yl}-1-methyl-2-oxo-1,2-dihydroquinoline-3-carbonitrile,
8-fluoro-4-[4-(4-methoxyphenyl)piperidin-1-yl]-1-methyl-2-oxo-1,2-dihydroquinoline-3-carbonitrile,
4-[4-(1,3-benzothiazol-2-yl)piperidin-1-yl]-8-fluoro-1-methyl-2-oxo-1,2-dihydroquinoline-3-carbonitrile,
4-[4-(1,3-benzoxazol-2-yl)piperidin-1-yl]-8-fluoro-1-methyl-2-oxo-1,2-dihydroquinoline-3-carbonitrile,
4-[4-(1,3-benzoxazol-2-yl)-4-methylpiperidin-1-yl]-8-bromo-1-methyl-2-oxo-1,2-dihydroquinoline-3-carbonitrile,
8-bromo-4-[4-(6-chloro-1,3-benzoxazol-2-yl)piperidin-1-yl]-1-methyl-2-oxo-1,2-dihydroquinoline-3-carbonitrile,
8-bromo-1-methyl-4-[4-methyl-4-(5-methyl-1,3-benzoxazol-2-yl)piperidin-1-yl]-2-oxo-1,2-dihydroquinoline-3-carbonitrile,
8-bromo-4-[4-(5-chloro-1,3-benzoxazol-2-yl)piperidin-1-yl]-1-methyl-2-oxo-1,2-dihydroquinoline-3-carbonitrile,
8-bromo-4-[4-(5,6-difluoro-1,3-benzoxazol-2-yl)piperidin-1-yl]-1-methyl-2-oxo-1,2-dihydroquinoline-3-carbonitrile,
4-[4-(1,3-benzoxazol-2-yl)piperidin-1-yl]-8-bromo-1-methyl-2-oxo-1,2-dihydroquinoline-3-carbonitrile,
4-[4-(1,3-benzoxazol-2-yl)piperidin-1-yl]-8-chloro-1-methyl-2-oxo-1,2-dihydroquinoline-3-carbonitrile,
1-methyl-4-[4-methyl-4-(5-methyl-1,3-benzoxazol-2-yl)piperidin-1-yl]-2-oxo-1,2-dihydroquinoline-3,8-dicarbonitrile,
8-(methanesulfonyl)-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)piperidin-1-yl]-1,6-dimethyl-2-oxo-1,2-dihydroquinoline-3-carbonitrile,
1,6-dimethyl-4-[4-(5-methyl-1,3-benzoxazol-2-yl)piperidin-1-yl]-2-oxo-1,2-dihydroquinoline-3-carbonitrile,
6-bromo-4-[4-(4-methoxyphenyl)piperidin-1-yl]-1-methyl-2-oxo-1,2-dihydroquinoline-3-carbonitrile,
4-[4-(1,3-benzoxazol-2-yl)piperidin-1-yl]-6-bromo-1-methyl-2-oxo-1,2-dihydroquinoline-3-carbonitrile,
4-[4-(1,3-benzothiazol-2-yl)piperidin-1-yl]-6-bromo-1-methyl-2-oxo-1,2-dihydroquinoline-3-carbonitrile,
6-bromo-1-methyl-2-oxo-4-{4-[5-(propan-2-yl)-1,3-benzoxazol-2-yl]piperidin-1-yl}-1,2-dihydroquinoline-3-carbonitrile,
6-bromo-1-methyl-4-[4-methyl-4-(5-methyl-1,3-benzoxazol-2-yl)piperidin-1-yl]-2-oxo-1,2-dihydroquinoline-3-carbonitrile,
4-[4-(4-methoxyphenyl)piperidin-1-yl]-1,6-dimethyl-2-oxo-1,2-dihydroquinoline-3-carbonitrile,
4-[4-(4-methoxyphenyl)piperidin-1-yl]-1-methyl-2-oxo-1,2-dihydroquinoline-3,6-dicarbonitrile,
4-[4-(1,3-benzothiazol-2-yl)piperidin-1-yl]-1,6-dimethyl-2-oxo-1,2-dihydroquinoline-3-carbonitrile,
4-[4-(1,3-benzoxazol-2-yl)piperidin-1-yl]-1-methyl-2-oxo-1,2-dihydroquinoline-3,6-dicarbonitrile,
4-[4-(1,3-benzothiazol-2-yl)piperidin-1-yl]-1-methyl-2-oxo-1,2-dihydroquinoline-3,6-dicarbonitrile,
6-cyclopropyl-4-[4-(4-methoxyphenyl)piperidin-1-yl]-1-methyl-2-oxo-1,2-dihydroquinoline-3-carbonitrile,
6-(methanesulfonyl)-4-[4-(4-methoxyphenyl)piperidin-1-yl]-1-methyl-2-oxo-1,2-dihydroquinoline-3-carbonitrile,
4-[4-(4-methoxyphenyl)piperidin-1-yl]-1-methyl-6-(oxetan-3-yl)-2-oxo-1,2-dihydroquinoline-3-carbonitrile,
6-(3,6-dihydro-2H-pyran-4-yl)-4-[4-(4-methoxyphenyl)piperidin-1-yl]-1-methyl-2-oxo-1,2-dihydroquinoline-3-carbonitrile,
4-[4-(4-methoxyphenyl)piperidin-1-yl]-1-methyl-2-oxo-6-phenyl-1,2-dihydroquinoline-3-carbonitrile,
6-(methanesulfonyl)-1-methyl-4-[4-methyl-4-(5-methyl-1,3-benzoxazol-2-yl)piperidin-1-yl]-2-oxo-1,2-dihydroquinoline-3-carbonitrile,
4-[4-(6-chloro-1,3-benzoxazol-2-yl)piperidin-1-yl]-1,6-dimethyl-2-oxo-1,2-dihydroquinoline-3-carbonitrile,
1,6-dimethyl-4-[4-(6-methyl-1,3-benzoxazol-2-yl)piperidin-1-yl]-2-oxo-1,2-dihydroquinoline-3-carbonitrile,
4-[4-(5-chloro-1,3-benzoxazol-2-yl)piperidin-1-yl]-1,6-dimethyl-2-oxo-1,2-dihydroquinoline-3-carbonitrile,
1,6-dimethyl-2-oxo-4-{4-[5-(propan-2-yl)-1,3-benzoxazol-2-yl]piperidin-1-yl}-1,2-dihydroquinoline-3-carbonitrile,
1,6-dimethyl-4-[4-methyl-4-(5-methyl-1,3-benzoxazol-2-yl)piperidin-1-yl]-2-oxo-1,2-dihydroquinoline-3-carbonitrile,
1,6-dimethyl-4-[4-methyl-4-(6-methyl-1,3-benzoxazol-2-yl)piperidin-1-yl]-2-oxo-1,2-dihydroquinoline-3-carbonitrile,
1,6-dimethyl-4-{4-methyl-4-[5-(propan-2-yl)-1,3-benzoxazol-2-yl]piperidin-1-yl}-2-oxo-1,2-dihydroquinoline-3-carbonitrile,
6-methoxy-1-methyl-4-[4-(6-methyl-1,3-benzoxazol-2-yl)piperidin-1-yl]-2-oxo-1,2-dihydroquinoline-3-carbonitrile,
6-methoxy-1-methyl-4-[4-methyl-4-(6-methyl-1,3-benzoxazol-2-yl)piperidin-1-yl]-2-oxo-1,2-dihydroquinoline-3-carbonitrile,
6-chloro-1-methyl-4-[4-methyl-4-(6-methyl-1,3-benzoxazol-2-yl)piperidin-1-yl]-2-oxo-1,2-dihydroquinoline-3-carbonitrile,
4-[4-(1,3-benzoxazol-2-yl)piperidin-1-yl]-1,6-dimethyl-2-oxo-1,2-dihydroquinoline-3-carboxamide,
1,6-dimethyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)-4-methylpiperidin-1-yl]-1,6-dimethyl-2-oxo-1,2-dihydroquinoline-3-carboxamide,
1,6-dimethyl-4-[4-(6-methyl-1,3-benzoxazol-2-yl)piperidin-1-yl]-2-oxo-1,2-dihydroquinoline-3-carboxamide,
1,6-dimethyl-4-[4-methyl-4-(6-methyl-1,3-benzoxazol-2-yl)piperidin-1-yl]-2-oxo-1,2-dihydroquinoline-3-carboxamide,
1,6-dimethyl-4-[4-methyl-4-(5-methyl-1,3-benzoxazol-2-yl)piperidin-1-yl]-2-oxo-1,2-dihydroquinoline-3-carboxamide,
1,6-dimethyl-4-{4-methyl-4-[5-(propan-2-yl)-1,3-benzoxazol-2-yl]piperidin-1-yl}-2-oxo-1,2-dihydroquinoline-3-carboxamide,
1,6-dimethyl-2-oxo-4-{4-[5-(propan-2-yl)-1,3-benzoxazol-2-yl]piperidin-1-yl}-1,2-dihydroquinoline-3-carboxamide,
4-[4-(5-chloro-1,3-benzoxazol-2-yl)piperidin-1-yl]-1,6-dimethyl-2-oxo-1,2-dihydroquinoline-3-carboxamide,
4-[4-(6-chloro-1,3-benzoxazol-2-yl)piperidin-1-yl]-1,6-dimethyl-2-oxo-1,2-dihydroquinoline-3-carboxamide,
6-bromo-1-methyl-4-[4-methyl-4-(5-methyl-1,3-benzoxazol-2-yl)piperidin-1-yl]-2-oxo-1,2-dihydroquinoline-3-carboxamide,
4-[4-(1,3-benzoxazol-2-yl)azepan-1-yl]-1,6-dimethyl-2-oxo-1,2-dihydroquinoline-3-carbonitrile,
4-[4-(1,3-benzoxazol-2-yl)azepan-1-yl]-1,6-dimethyl-2-oxo-1,2-dihydroquinoline-3-carboxamide,
4-[4-(1,3-benzoxazol-2-yl)piperidin-1-yl]-6-fluoro-1-methyl-2-oxo-1,2-dihydroquinoline-3-carbonitrile,
1-methyl-4-[4-methyl-4-(5-methyl-1,3-benzoxazol-2-yl)piperidin-1-yl]-2-oxo-1,2-dihydroquinoline-3,6-dicarbonitrile,
6-cyano-1-methyl-4-[4-methyl-4-(5-methyl-1,3-benzoxazol-2-yl)piperidin-1-yl]-2-oxo-1,2-dihydroquinoline-3-carboxamide,
6-(3,3-difluorocyclobutyl)-1-methyl-4-[4-methyl-4-(5-methyl-1,3-benzoxazol-2-yl)piperidin-1-yl]-2-oxo-1,2-dihydroquinoline-3-carbonitrile,
6-cyclopropyl-1-methyl-4-[4-methyl-4-(5-methyl-1,3-benzoxazol-2-yl)piperidin-1-yl]-2-oxo-1,2-dihydroquinoline-3-carbonitrile,
6-(butan-2-yl)-1-methyl-4-[4-methyl-4-(5-methyl-1,3-benzoxazol-2-yl)piperidin-1-yl]-2-oxo-1,2-dihydroquinoline-3-carbonitrile,
7-(methoxymethyl)-1-methyl-4-[4-methyl-4-(5-methyl-1,3-benzoxazol-2-yl)piperidin-1-yl]-2-oxo-1,2-dihydroquinoline-3-carbonitrile,
6-(methoxymethyl)-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-1,6-dimethyl-2-oxo-1,2-dihydroquinoline-3-carbonitrile,
6-fluoro-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-6-fluoro-1-methyl-2-oxo-1,2-dihydroquinoline-3-carbonitrile,
6-fluoro-1-methyl-4-[4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-1,7-dimethyl-2-oxo-1,2-dihydroquinoline-3-carbonitrile,
4-[4-(1,3-benzoxazol-2-yl)piperidin-1-yl]-1,7-dimethyl-2-oxo-1,2-dihydroquinoline-3-carbonitrile,
1,7-dimethyl-4-[4-methyl-4-(5-methyl-1,3-benzoxazol-2-yl)piperidin-1-yl]-2-oxo-1,2-dihydroquinoline-3-carbonitrile,
1,7-dimethyl-4-[4-(5-methyl-1,3-benzoxazol-2-yl)piperidin-1-yl]-2-oxo-1,2-dihydroquinoline-3-carbonitrile,
4-[4-(1,3-benzothiazol-2-yl)piperidin-1-yl]-1,7-dimethyl-2-oxo-1,2-dihydroquinoline-3-carbonitrile,
4-[4-(1,3-benzoxazol-2-yl)-4-ethylpiperidin-1-yl]-7-bromo-1-methyl-2-oxo-1,2-dihydroquinoline-3-carbonitrile,
4-[4-(1,3-benzoxazol-2-yl)-4-methylpiperidin-1-yl]-7-bromo-1-methyl-2-oxo-1,2-dihydroquinoline-3-carbonitrile,
8-bromo-1,6-dimethyl-4-[4-methyl-4-(6-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-7-chloro-1-methyl-2-oxo-1,2-dihydroquinoline-3-carbonitrile,
4-[4-(1,3-benzoxazol-2-yl)piperidin-1-yl]-7-chloro-1-methyl-2-oxo-1,2-dihydroquinoline-3-carbonitrile,
7-chloro-1-methyl-4-[4-methyl-4-(5-methyl-1,3-benzoxazol-2-yl)piperidin-1-yl]-2-oxo-1,2-dihydroquinoline-3-carbonitrile,
7-chloro-1-methyl-4-[4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-8-chloro-1-methyl-2-oxo-1,2-dihydroquinoline-3-carbonitrile,
8-chloro-1-methyl-4-[4-methyl-4-(5-methyl-1,3-benzoxazol-2-yl)piperidin-1-yl]-2-oxo-1,2-dihydroquinoline-3-carbonitrile,
8-chloro-1-methyl-4-[4-(5-methyl-1,3-benzoxazol-2-yl)piperidin-1-yl]-2-oxo-1,2-dihydroquinoline-3-carbonitrile,
8-bromo-1-methyl-4-{4-methyl-4-[5-methyl-4-(trifluoromethyl)-1,3-benzoxazol-2-yl]piperidin-1-yl}-2-oxo-1,2-dihydroquinoline-3-carbonitrile,
1-methyl-4-[(2S,4S)-2-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)-4-methylpiperidin-1-yl]-7-fluoro-1-methyl-2-oxo-1,2-dihydroquinoline-3-carbonitrile,
4-[4-(1,3-benzoxazol-2-yl)piperidin-1-yl]-7-fluoro-1-methyl-2-oxo-1,2-dihydroquinoline-3-carbonitrile,
7-fluoro-1-methyl-4-[4-methyl-4-(5-methyl-1,3-benzoxazol-2-yl)piperidin-1-yl]-2-oxo-1,2-dihydroquinoline-3-carbonitrile,
7-fluoro-1-methyl-4-[4-(5-methyl-1,3-benzoxazol-2-yl)piperidin-1-yl]-2-oxo-1,2-dihydroquinoline-3-carbonitrile,
7-[(1-hydroxycyclopropyl)methoxy]-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzothiazol-2-yl)piperidin-1-yl]-7-methoxy-1-methyl-2-oxo-1,2-dihydroquinoline-3-carbonitrile,
1-methyl-4-[4-methyl-4-(5-methyl-1,3-benzoxazol-2-yl)piperidin-1-yl]-7-[(oxetan-3-yl)oxy]-2-oxo-1,2-dihydroquinoline-3-carbonitrile,
6-hydroxy-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)piperidin-1-yl]-7-methoxy-1-methyl-2-oxo-1,2-dihydroquinoline-3-carbonitrile,
7-(cyclopropyloxy)-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-8-(oxetan-3-yl)-2-oxo-1,2-dihydroquinoline-3-carbonitrile,
6-methoxy-1-methyl-4-[4-methyl-4-(5-methyl-1,3-benzoxazol-2-yl)piperidin-1-yl]-2-oxo-1,2-dihydroquinoline-3-carbonitrile,
7-(cyclobutyloxy)-1-methyl-4-[4-methyl-4-(5-methyl-1,3-benzoxazol-2-yl)piperidin-1-yl]-2-oxo-1,2-dihydroquinoline-3-carbonitrile,
7-methoxy-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-2-oxo-1,2-dihydroquinoline-3,7-dicarbonitrile,
7-cyclopropyl-1-methyl-4-[4-methyl-4-(5-methyl-1,3-benzoxazol-2-yl)piperidin-1-yl]-2-oxo-1,2-dihydroquinoline-3-carbonitrile,
7-[(1-cyanocyclopropyl)methoxy]-1-methyl-4-[4-methyl-4-(5-methyl-1,3-benzoxazol-2-yl)piperidin-1-yl]-2-oxo-1,2-dihydroquinoline-3-carbonitrile,
7-(3,3-difluorocyclobutyl)-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-8-[(oxetan-3-yl)oxy]-2-oxo-1,2-dihydroquinoline-3-carbonitrile,
4-[4-(5,6-difluoro-1,3-benzoxazol-2-yl)piperidin-1-yl]-7-methoxy-1-methyl-2-oxo-1,2-dihydroquinoline-3-carbonitrile,
1,7-dimethyl-2-oxo-4-(4-phenylpiperidin-1-yl)-1,2-dihydroquinoline-3-carbonitrile,
7-methoxy-1-methyl-2-oxo-4-(4-phenylpiperidin-1-yl)-1,2-dihydroquinoline-3-carbonitrile,
1-methyl-4-[4-methyl-4-(5-methyl-1,3-benzoxazol-2-yl)piperidin-1-yl]-2-oxo-7-propoxy-1,2-dihydroquinoline-3-carbonitrile,
4-[4-(6-chloro-1,3-benzoxazol-2-yl)piperidin-1-yl]-7-methoxy-1-methyl-2-oxo-1,2-dihydroquinoline-3-carbonitrile,
4-[4-(5-chloro-1,3-benzoxazol-2-yl)piperidin-1-yl]-7-methoxy-1-methyl-2-oxo-1,2-dihydroquinoline-3-carbonitrile,
8-methoxy-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-7-(oxetan-3-yl)-2-oxo-1,2-dihydroquinoline-3-carbonitrile,
8-(cyclopropyloxy)-1-methyl-4-[4-methyl-4-(5-methyl-1,3-benzoxazol-2-yl)piperidin-1-yl]-2-oxo-1,2-dihydroquinoline-3-carbonitrile,
1,8-dimethyl-4-[4-methyl-4-(5-methyl-1,3-benzoxazol-2-yl)piperidin-1-yl]-2-oxo-1,2-dihydroquinoline-3-carbonitrile,
7-(cyclopropyloxy)-1-methyl-2-oxo-4-(4-phenylpiperidin-1-yl)-1,2-dihydroquinoline-3-carbonitrile,
4-{4-[4-(2-methoxyethyl)-1,3-benzoxazol-2-yl]piperidin-1-yl}-1-methyl-2-oxo-1,2-dihydroquinoline-3-carbonitrile,
7-[(2S)-butan-2-yl]-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-6-[(oxetan-3-yl)oxy]-2-oxo-1,2-dihydroquinoline-3-carbonitrile,
6-[(1-cyanocyclopropyl)methoxy]-1-methyl-4-[4-methyl-4-(5-methyl-1,3-benzoxazol-2-yl)piperidin-1-yl]-2-oxo-1,2-dihydroquinoline-3-carbonitrile,
7-[(4R)-4-hydroxy-2-oxopyrrolidin-1-yl]-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzothiazol-2-yl)piperidin-1-yl]-1-methyl-2-oxo-7-propoxy-1,2-dihydroquinoline-3-carbonitrile,
8-hydroxy-1-methyl-4-[4-methyl-4-(5-methyl-1,3-benzoxazol-2-yl)piperidin-1-yl]-2-oxo-1,2-dihydroquinoline-3-carbonitrile,
8-cyclopropyl-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-2-oxo-8-propoxy-1,2-dihydroquinoline-3-carbonitrile,
6-(cyclopropyloxy)-1-methyl-4-[4-methyl-4-(5-methyl-1,3-benzoxazol-2-yl)piperidin-1-yl]-2-oxo-1,2-dihydroquinoline-3-carbonitrile,
4-[4-(1,3-benzoxazol-2-yl)piperidin-1-yl]-1-methyl-2-oxo-1,2-dihydroquinoline-3,7-dicarbonitrile,
4-[4-(1,3-benzothiazol-2-yl)piperidin-1-yl]-6-cyclopropyl-1-methyl-2-oxo-1,2-dihydroquinoline-3-carbonitrile,
6-[(4R)-4-hydroxy-2-oxopyrrolidin-1-yl]-1-methyl-4-[4-methyl-4-(5-methyl-1,3-benzoxazol-2-yl)piperidin-1-yl]-2-oxo-1,2-dihydroquinoline-3-carbonitrile,
6-(cyclobutyloxy)-1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-methyl-4-(5-methyl-1,3-benzoxazol-2-yl)piperidin-1-yl]-2-oxo-6-propoxy-1,2-dihydroquinoline-3-carbonitrile,
6-[(4R)-4-hydroxy-2-oxopyrrolidin-1-yl]-1-methyl-4-[4-methyl-4-(5-methyl-1,3-benzoxazol-2-yl)piperidin-1-yl]-2-oxo-1,2-dihydroquinoline-3-carboxamide,
7-bromo-1-methyl-4-[4-(5-methyl-1,3-benzoxazol-2-yl)piperidin-1-yl]-2-oxo-1,2-dihydroquinoline-3-carbonitrile,
7-(dimethylphosphoryl)-1-methyl-4-[4-(5-methyl-1,3-benzoxazol-2-yl)piperidin-1-yl]-2-oxo-1,2-dihydroquinoline-3-carbonitrile,
4-{4-[5-(2-methoxyethyl)-1,3-benzoxazol-2-yl]piperidin-1-yl}-1-methyl-2-oxo-1,2-dihydroquinoline-3-carbonitrile,
4-{4-[6-(2-methoxyethyl)-1,3-benzoxazol-2-yl]piperidin-1-yl}-1-methyl-2-oxo-1,2-dihydroquinoline-3-carbonitrile,
1-methyl-4-[4-methyl-4-(5-methyl-1,3-benzoxazol-2-yl)piperidin-1-yl]-7-[(oxetan-3-yl)methyl]-2-oxo-1,2-dihydroquinoline-3-carbonitrile,
1-methyl-4-[4-methyl-4-(5-methyl-1,3-benzoxazol-2-yl)piperidin-1-yl]-6-[(oxetan-3-yl)methyl]-2-oxo-1,2-dihydroquinoline-3-carbonitrile,
1-methyl-4-{4-[5-(oxetan-3-yl)-1,3-benzoxazol-2-yl]piperidin-1-yl}-2-oxo-1,2-dihydroquinoline-3-carbonitrile,
1-methyl-4-[4-methyl-4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-2-oxo-7-(trifluoromethyl)-1,2-dihydroquinoline-3-carbonitrile,
1-methyl-4-[4-methyl-4-(5-methyl-1,3-benzoxazol-2-yl)piperidin-1-yl]-2-oxo-7-(trifluoromethyl)-1,2-dihydroquinoline-3-carbonitrile,
4-[4-(1,3-benzoxazol-2-yl)piperidin-1-yl]-1-methyl-2-oxo-7-(trifluoromethyl)-1,2-dihydroquinoline-3-carbonitrile,
4-[4-(1,3-benzoxazol-2-yl)-4-methylpiperidin-1-yl]-1-methyl-2-oxo-7-(trifluoromethyl)-1,2-dihydroquinoline-3-carbonitrile,
7-hydroxy-1-methyl-4-[4-(5-methyl-1,3-benzoxazol-2-yl)piperidin-1-yl]-2-oxo-1,2-dihydroquinoline-3-carbonitrile,
4-[4-(6-bromo-1,3-benzoxazol-2-yl)piperidin-1-yl]-1-methyl-2-oxo-1,2-dihydroquinoline-3-carbonitrile,
4-{4-[5-(2-methoxyethyl)-1,3-benzoxazol-2-yl]piperidin-1-yl}-1-methyl-2-oxo-1,2-dihydroquinoline-3-carboxamide,
4-[4-(1,3-benzoxazol-2-yl)-4-methylpiperidin-1-yl]-6-fluoro-1-methyl-2-oxo-1,2-dihydroquinoline-3-carboxamide,
4-[4-(1,3-benzoxazol-2-yl)-4-methylpiperidin-1-yl]-7-chloro-1-methyl-2-oxo-1,2-dihydroquinoline-3-carboxamide,
6-fluoro-1-methyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)piperidin-1-yl]-6-fluoro-1-methyl-2-oxo-1,2-dihydroquinoline-3-carboxamide,
4-[4-(1,3-benzoxazol-2-yl)piperidin-1-yl]-7-chloro-1-methyl-2-oxo-1,2-dihydroquinoline-3-carboxamide,
7-chloro-1-methyl-4-[4-(5-methyl-1,3-benzoxazol-2-yl)piperidin-1-yl]-2-oxo-1,2-dihydroquinoline-3-carboxamide,
1-methyl-4-[4-methyl-4-(5-methyl-1,3-benzoxazol-2-yl)piperidin-1-yl]-2-oxo-1,2-dihydroquinoline-3-carboxamide,
4-[4-(1,3-benzoxazol-2-yl)-4-methylpiperidin-1-yl]-7-fluoro-1-methyl-2-oxo-1,2-dihydroquinoline-3-carboxamide,
7-fluoro-1-methyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)piperidin-1-yl]-7-fluoro-1-methyl-2-oxo-1,2-dihydroquinoline-3-carboxamide,
4-[4-(1,3-benzoxazol-2-yl)-4-methylpiperidin-1-yl]-1-methyl-2-oxo-7-(trifluoromethyl)-1,2-dihydroquinoline-3-carboxamide,
1-methyl-4-[4-(5-methyl-1,3-benzoxazol-2-yl)piperidin-1-yl]-2-oxo-7-(trifluoromethyl)-1,2-dihydroquinoline-3-carboxamide,
4-[4-(1,3-benzoxazol-2-yl)piperidin-1-yl]-1-methyl-2-oxo-7-(trifluoromethyl)-1,2-dihydroquinoline-3-carboxamide,
4-[4-(1,3-benzoxazol-2-yl)piperidin-1-yl]-8-chloro-1-methyl-2-oxo-1,2-dihydroquinoline-3-carboxamide,
4-[4-(1,3-benzoxazol-2-yl)-4-methylpiperidin-1-yl]-8-chloro-1-methyl-2-oxo-1,2-dihydroquinoline-3-carboxamide,
4-[4-(1,3-benzoxazol-2-yl)-4-methylpiperidin-1-yl]-1,7-dimethyl-2-oxo-1,2-dihydroquinoline-3-carboxamide,
1,7-dimethyl-4-[4-methyl-4-(5-methyl-1,3-benzoxazol-2-yl)piperidin-1-yl]-2-oxo-1,2-dihydroquinoline-3-carboxamide,
1,7-dimethyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)piperidin-1-yl]-1,7-dimethyl-2-oxo-1,2-dihydroquinoline-3-carboxamide,
4-{4-[4-(2-methoxyethyl)-1,3-benzoxazol-2-yl]piperidin-1-yl}-1-methyl-2-oxo-1,2-dihydroquinoline-3-carboxamide,
4-[4-(1,3-benzoxazol-2-yl)piperidin-1-yl]-1-methyl-2-oxo-1,2-dihydroquinoline-3-carboxamide,
4-{4-[6-(2-methoxyethyl)-1,3-benzoxazol-2-yl]piperidin-1-yl}-1-methyl-2-oxo-1,2-dihydroquinoline-3-carboxamide,
4-[4-(1,3-benzoxazol-2-yl)piperidin-1-yl]-1-methyl-2-oxo-7-(2-oxopyrrolidin-1-yl)-1,2-dihydroquinoline-3-carboxamide,
8-chloro-1-methyl-4-[4-(5-methyl-1,3-benzoxazol-2-yl)piperidin-1-yl]-2-oxo-1,2-dihydroquinoline-3-carboxamide,
4-[4-(4,5-dimethyl-1,3-thiazol-2-yl)piperidin-1-yl]-1-methyl-2-oxo-1,2-dihydroquinoline-3-carbonitrile,
1-methyl-2-oxo-4-[4-(quinoxalin-2-yl)piperidin-1-yl]-1,2-dihydroquinoline-3-carbonitrile,
1-methyl-2-oxo-4-[4-(1H-pyrazol-3-yl)piperidin-1-yl]-1,2-dihydroquinoline-3-carbonitrile,
1-methyl-4-[4-(1-methyl-1H-pyrazol-4-yl)piperidin-1-yl]-2-oxo-1,2-dihydroquinoline-3-carbonitrile,
4-[4-(3,4-dimethoxyphenyl)piperidin-1-yl]-1-methyl-2-oxo-1,2-dihydroquinoline-3-carbonitrile,
4-[4-(3-chlorophenyl)piperidin-1-yl]-1-methyl-2-oxo-1,2-dihydroquinoline-3-carbonitrile,
1-methyl-4-[(2S,4S)-2-methyl-4-(5-methyl-1,3-benzoxazol-2-yl)-1-piperidyl]-2-oxo-quinoline-3-carbonitrile,
4-[4-(3-cyanophenyl)piperidin-1-yl]-1-methyl-2-oxo-1,2-dihydroquinoline-3-carbonitrile,
8-fluoro-1-methyl-2-oxo-4-[(4S)-4-phenylazepan-1-yl]-1,2-dihydroquinoline-3-carbonitrile,
1-methyl-4-[rac-(2R,3S)-2-methyl-3-phenylpyrrolidin-1-yl]-2-oxo-1,2-dihydroquinoline-3-carbonitrile,
4-[1-(3-cyano-1-methyl-2-oxo-1,2-dihydroquinolin-4-yl)piperidin-4-yl]benzamide,
4-[4-hydroxy-4-(2-methoxyphenyl)piperidin-1-yl]-1-methyl-2-oxo-1,2-dihydroquinoline-3-carbonitrile,
4-[4-(1,3-benzoxazol-2-yl)piperidin-1-yl]-1-ethyl-2-oxo-1,2-dihydroquinoline-3-carbonitrile,
4-[4-(4-chlorophenyl)piperidin-1-yl]-1-ethyl-2-oxo-1,2-dihydroquinoline-3-carbonitrile,
1-methyl-2-oxo-4-{4-[5-(2-oxopyrrolidin-1-yl)-1,3-benzoxazol-2-yl]piperidin-1-yl}-1,2-dihydroquinoline-3-carbonitrile,
4-[4-(4-acetylphenyl)-4-methylpiperidin-1-yl]-1-methyl-2-oxo-1,2-dihydroquinoline-3-carbonitrile,
4-[4-(3-chlorophenyl)-4-methoxypiperidin-1-yl]-1-methyl-2-oxo-1,2-dihydroquinoline-3-carbonitrile,
6-(dimethylphosphoryl)-1-methyl-4-[4-methyl-4-(5-methyl-1,3-benzoxazol-2-yl)piperidin-1-yl]-2-oxo-1,2-dihydroquinoline-3-carbonitrile,
6-(dimethylphosphoryl)-1-methyl-4-[4-(5-methyl-1,3-benzoxazol-2-yl)piperidin-1-yl]-2-oxo-1,2-dihydroquinoline-3-carbonitrile,
6-(methanesulfonyl)-1-methyl-4-[4-(5-methyl-1,3-benzoxazol-2-yl)piperidin-1-yl]-2-oxo-1,2-dihydroquinoline-3-carbonitrile,
4-[4-(1-benzothiophen-2-yl)-4-hydroxypiperidin-1-yl]-1-methyl-2-oxo-1,2-dihydroquinoline-3-carbonitrile,
4-[4-(1-benzothiophen-2-yl)-4-methoxypiperidin-1-yl]-1-methyl-2-oxo-1,2-dihydroquinoline-3-carbonitrile,
4-[4-(1-benzothiophen-2-yl)-4-methylpiperidin-1-yl]-1-methyl-2-oxo-1,2-dihydroquinoline-3-carbonitrile,
4-[4-(6-methoxynaphthalen-2-yl)piperidin-1-yl]-1-methyl-2-oxo-1,2-dihydroquinoline-3-carbonitrile,
6-[1-(3-cyano-1-methyl-2-oxo-1,2-dihydroquinolin-4-yl)piperidin-4-yl]-2-methylquinoline-4-carbonitrile,
1-methyl-4-[4-(2-methyl-1,3-benzoxazol-5-yl)piperidin-1-yl]-2-oxo-1,2-dihydroquinoline-3-carbonitrile,
1-methyl-4-[4-(2-methyl-1,3-benzothiazol-5-yl)piperidin-1-yl]-2-oxo-1,2-dihydroquinoline-3-carbonitrile,
(rac)-1-methyl-4-[4-(3-methyl-2-oxo-2,3-dihydro-1H-indol-5-yl)piperidin-1-yl]-2-oxo-1,2-dihydroquinoline-3-carbonitrile,
1-methyl-4-{4-[3-(2-methylphenyl)-1,2,4-oxadiazol-5-yl]piperidin-1-yl}-2-oxo-1,2-dihydroquinoline-3-carbonitrile,
4-{4-[3-(4-methoxyphenyl)-1,2,4-oxadiazol-5-yl]piperidin-1-yl}-1-methyl-2-oxo-1,2-dihydroquinoline-3-carbonitrile,
1-methyl-4-{4-[3-(3-methylphenyl)-1,2,4-oxadiazol-5-yl]piperidin-1-yl}-2-oxo-1,2-dihydroquinoline-3-carbonitrile,
1-methyl-4-{4-[5-(2-methylphenyl)-1,3,4-oxadiazol-2-yl]piperidin-1-yl}-2-oxo-1,2-dihydroquinoline-3-carbonitrile,
1-methyl-4-{4-[5-(4-methylphenyl)-1,3,4-oxadiazol-2-yl]piperidin-1-yl}-2-oxo-1,2-dihydroquinoline-3-carbonitrile,
1-methyl-4-{4-[5-(3-methylphenyl)-1,3,4-oxadiazol-2-yl]piperidin-1-yl}-2-oxo-1,2-dihydroquinoline-3-carbonitrile,
4-{4-[5-(3-chlorophenyl)-1,3,4-oxadiazol-2-yl]piperidin-1-yl}-1-methyl-2-oxo-1,2-dihydroquinoline-3-carbonitrile,
4-{4-[5-(3-chlorophenyl)-1,3,4-oxadiazol-2-yl]-4-methylpiperidin-1-yl}-1-methyl-2-oxo-1,2-dihydroquinoline-3-carbonitrile,
4-[4-(3,3-dimethyl-2,3-dihydro-1H-indol-5-yl)piperidin-1-yl]-1-methyl-2-oxo-1,2-dihydroquinoline-3-carbonitrile,
1-methyl-4-{4-[(2R)-2-methyl-2,3-dihydro-1-benzofuran-5-yl]piperidin-1-yl}-2-oxo-1,2-dihydroquinoline-3-carbonitrile,
1-methyl-2-oxo-4-[4-(1,3,3-trimethyl-2-oxo-2,3-dihydro-1H-indol-5-yl)piperidin-1-yl]-1,2-dihydroquinoline-3-carbonitrile,
4-[4-(2-hydroxy-2,3-dihydro-1H-inden-5-yl)piperidin-1-yl]-1-methyl-2-oxo-1,2-dihydroquinoline-3-carbonitrile,
4-[4-(2,2-dimethyl-2H-1,3-benzodioxol-5-yl)piperidin-1-yl]-1-methyl-2-oxo-1,2-dihydroquinoline-3-carbonitrile,
1-methyl-2-oxo-4-[4-(2′-oxo-1′,2′-dihydrospiro[cyclobutane-1,3′-indol]-5′-yl)piperidin-1-yl]-1,2-dihydroquinoline-3-carbonitrile,
1-methyl-4-[4-(3-methyl-2-oxo-2,3-dihydro-1,3-benzoxazol-6-yl)piperidin-1-yl]-2-oxo-1,2-dihydroquinoline-3-carbonitrile,
4-[4-(1,3-dimethyl-2-oxo-2,3-dihydro-1H-benzimidazol-5-yl)piperidin-1-yl]-1-methyl-2-oxo-1,2-dihydroquinoline-3-carbonitrile,
1-methyl-4-[4-methyl-4-(4-methylquinolin-2-yl)piperidin-1-yl]-2-oxo-1,2-dihydroquinoline-3-carbonitrile,
4-[4-(4-fluoro-1-methyl-1H-indol-6-yl)piperidin-1-yl]-1-methyl-2-oxo-1,2-dihydroquinoline-3-carbonitrile,
1-methyl-4-{4-[1-(3-methylphenyl)-1H-pyrazol-3-yl]piperidin-1-yl}-2-oxo-1,2-dihydroquinoline-3-carbonitrile,
1-methyl-4-{4-[1-(2-methylphenyl)-1H-pyrazol-3-yl]piperidin-1-yl}-2-oxo-1,2-dihydroquinoline-3-carbonitrile,
4-{4-[1-(3-chlorophenyl)-1H-pyrazol-3-yl]piperidin-1-yl}-1-methyl-2-oxo-1,2-dihydroquinoline-3-carbonitrile,
4-{4-[1-(4-chlorophenyl)-1H-pyrazol-3-yl]piperidin-1-yl}-1-methyl-2-oxo-1,2-dihydroquinoline-3-carbonitrile,
1-methyl-2-oxo-4-{4-[3-(pyridin-3-yl)-1,2,4-oxadiazol-5-yl]piperidin-1-yl}-1,2-dihydroquinoline-3-carbonitrile,
1-methyl-4-[4-(2-methylquinolin-6-yl)piperidin-1-yl]-2-oxo-1,2-dihydroquinoline-3-carbonitrile,
4-{4-[4-(3-methoxyphenyl)-1,3-thiazol-2-yl]piperidin-1-yl}-1-methyl-2-oxo-1,2-dihydroquinoline-3-carbonitrile,
4-{4-[4-(2-methoxyphenyl)-1,3-thiazol-2-yl]piperidin-1-yl}-1-methyl-2-oxo-1,2-dihydroquinoline-3-carbonitrile,
1-methyl-4-{4-[4-(4-methylphenyl)-1,3-thiazol-2-yl]piperidin-1-yl}-2-oxo-1,2-dihydroquinoline-3-carbonitrile,
4-[4-(1,3-dimethyl-1H-indazol-5-yl)piperidin-1-yl]-1-methyl-2-oxo-1,2-dihydroquinoline-3-carbonitrile,
1-methyl-4-[4-(2-methyl-1,3-benzoxazol-6-yl)piperidin-1-yl]-2-oxo-1,2-dihydroquinoline-3-carbonitrile,
1-methyl-4-[4-(2-methyl-1,3-benzothiazol-6-yl)piperidin-1-yl]-2-oxo-1,2-dihydroquinoline-3-carbonitrile,
4-{4-[3-(difluoromethyl)quinolin-7-yl]piperidin-1-yl}-1-methyl-2-oxo-1,2-dihydroquinoline-3-carbonitrile,
(rac)-1-methyl-4-[4-(5-methyl-1,3-benzoxazol-2-yl)piperidin-1-yl]-2-oxo-7-[(oxolan-3-yl)oxy]-1,2-dihydroquinoline-3-carbonitrile,
1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carbonitrile
1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carbonitrile
1-methyl-4-[4-(5-methyl-1,3-benzoxazol-2-yl)piperidin-1-yl]-7-[(oxan-4-yl)oxy]-2-oxo-1,2-dihydroquinoline-3-carbonitrile,
7-(2-methoxyethoxy)-1-methyl-4-[4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-7-{[oxiran-2-yl]methoxy}-2-oxo-1,2-dihydroquinoline-3-carbonitrile,
1-methyl-4-[4-(5-methyl-1,3-benzoxazol-2-yl)piperidin-1-yl]-7-[(oxetan-3-yl)oxy]-2-oxo-1,2-dihydroquinoline-3-carbonitrile,
4-[4-(5-methoxy-1,3-benzoxazol-2-yl)piperidin-1-yl]-1-methyl-2-oxo-1,2-dihydroquinoline-3-carbonitrile,
1-methyl-2-oxo-4-(4-{5-[(oxolan-2-yl)methoxy]-1,3-benzoxazol-2-yl}piperidin-1-yl)-1,2-dihydroquinoline-3-carbonitrile,
1-methyl-4-{4-[6-(oxetan-3-yl)-1,3-benzoxazol-2-yl]piperidin-1-yl}-2-oxo-1,2-dihydroquinoline-3-carbonitrile,
4-[4-(1,3-benzoxazol-2-yl)-4-fluoropiperidin-1-yl]-1-methyl-2-oxo-1,2-dihydroquinoline-3-carbonitrile,
4-[4-(1,3-benzoxazol-2-yl)-4-methoxypiperidin-1-yl]-1-methyl-2-oxo-1,2-dihydroquinoline-3-carbonitrile,
4-{4-[5-(methoxymethyl)-1,3-benzoxazol-2-yl]-4-methylpiperidin-1-yl}-1-methyl-2-oxo-1,2-dihydroquinoline-3-carbonitrile,
4-{4-[6-(methoxymethyl)-1,3-benzoxazol-2-yl]-4-methylpiperidin-1-yl}-1-methyl-2-oxo-1,2-dihydroquinoline-3-carbonitrile,
4-[4-fluoro-4-(5-methoxy-1,3-benzoxazol-2-yl)piperidin-1-yl]-1-methyl-2-oxo-1,2-dihydroquinoline-3-carbonitrile,
4-[4-(5-cyclopropyl-1,3-benzoxazol-2-yl)piperidin-1-yl]-1-methyl-2-oxo-1,2-dihydroquinoline-3-carbonitrile,
4-[4-(6-cyclopropyl-1,3-benzoxazol-2-yl)piperidin-1-yl]-1-methyl-2-oxo-1,2-dihydroquinoline-3-carbonitrile,
6-bromo-1-methyl-4-[4-(5-methyl-1,3-benzoxazol-2-yl)piperidin-1-yl]-2-oxo-1,2-dihydroquinoline-3-carbonitrile,
8-bromo-1-methyl-4-[4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-7-[methyl(oxetan-3-yl)amino]-2-oxo-1,2-dihydroquinoline-3-carbonitrile,
7-[(2-hydroxyethyl)(methyl)amino]-1-methyl-4-[4-(5-methyl-1,3-benzoxazol-2-yl)piperidin-1-yl]-2-oxo-1,2-dihydroquinoline-3-carbonitrile,
(rac)-1-methyl-4-[4-(5-methyl-1,3-benzoxazol-2-yl)piperidin-1-yl]-7-[methyl(oxolan-3-yl)amino]-2-oxo-1,2-dihydroquinoline-3-carbonitrile,
7-[(cyanomethyl)(methyl)amino]-1-methyl-4-[4-(5-methyl-1,3-benzoxazol-2-yl)piperidin-1-yl]-2-oxo-1,2-dihydroquinoline-3-carbonitrile,
6-methoxy-1-methyl-4-[4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-2-oxo-1,2-dihydroquinoline-3,6-dicarbonitrile,
3-cyano-1-methyl-4-[4-(5-methyl-1,3-benzoxazol-2-yl)piperidin-1-yl]-2-oxo-1,2-dihydroquinoline-6-carboxamide,
6-ethoxy-1-methyl-4-[4-(5-methyl-1,3-benzoxazol-2-yl)piperidin-1-yl]-2-oxo-1,2-dihydroquinoline-3-carbonitrile,
6-(2,2-difluoropropoxy)-1-methyl-4-[4-(5-methyl-1,3-benzoxazol-2-yl)piperidin-1-yl]-2-oxo-1,2-dihydroquinoline-3-carbonitrile,
6-(2,2-difluoroethoxy)-1-methyl-4-[4-(5-methyl-1,3-benzoxazol-2-yl)piperidin-1-yl]-2-oxo-1,2-dihydroquinoline-3-carbonitrile,
6-(cyclopropylmethoxy)-1-methyl-4-[4-(5-methyl-1,3-benzoxazol-2-yl)piperidin-1-yl]-2-oxo-1,2-dihydroquinoline-3-carbonitrile,
6-cyclobutyl-1-methyl-4-[4-(5-methyl-1,3-benzoxazol-2-yl)piperidin-1-yl]-2-oxo-1,2-dihydroquinoline-3-carbonitrile,
6-[2,2-dimethylcyclobutyl]-1-methyl-4-[4-(5-methyl-1,3-benzoxazol-2-yl)piperidin-1-yl]-2-oxo-1,2-dihydroquinoline-3-carbonitrile,
1-methyl-4-[4-(5-methyl-1,3-benzoxazol-2-yl)piperidin-1-yl]-2-oxo-6-(3,3,3-trifluoroprop-1-en-2-yl)-1,2-dihydroquinoline-3-carbonitrile,
1-methyl-4-[4-(5-methyl-1,3-benzoxazol-2-yl)piperidin-1-yl]-2-oxo-6-[1-(trifluoromethyl)cyclopropyl]-1,2-dihydroquinoline-3-carbonitrile,
2-({3-cyano-1-methyl-4-[4-(5-methyl-1,3-benzoxazol-2-yl)piperidin-1-yl]-2-oxo-1,2-dihydroquinolin-7-yl}oxy)acetamide,
1-methyl-4-[4-(5-methyl-1,3-benzoxazol-2-yl)piperidin-1-yl]-7-{I[oxan-3-yl]oxy}-2-oxo-1,2-dihydroquinoline-3-carbonitrile,
(rac)-tert-butyl ({3-cyano-1-methyl-4-[4-(5-methyl-1,3-benzoxazol-2-yl)piperidin-1-yl]-2-oxo-1,2-dihydroquinolin-7-yl}oxy)acetate,
({3-cyano-1-methyl-4-[4-(5-methyl-1,3-benzoxazol-2-yl)piperidin-1-yl]-2-oxo-1,2-dihydroquinolin-7-yl}oxy)acetic acid,
(rac)-4-[4-fluoro-4-(5-methyl-1,3-benzoxazol-2-yl)piperidin-1-yl]-1-methyl-2-oxo-7-(tetrahydrofuran-3-yloxy)-1,2-dihydroquinoline-3-carbonitrile,
7-(cyclopropylamino)-1-methyl-4-[4-methyl-4-(5-methyl-1,3-benzoxazol-2-yl)piperidin-1-yl]-2-oxo-1,2-dihydroquinoline-3-carbonitrile,
7-methoxy-1,6-dimethyl-4-[4-(5-methyl-1,3-benzoxazol-2-yl)piperidin-1-yl]-2-oxo-1,2-dihydroquinoline-3-carbonitrile,
6-bromo-4-[4-fluoro-4-(5-methyl-1,3-benzoxazol-2-yl)piperidin-1-yl]-7-methoxy-1-methyl-2-oxo-1,2-dihydroquinoline-3-carbonitrile,
1,6-dimethyl-4-[4-(5-methyl-1,3-benzoxazol-2-yl)piperidin-1-yl]-7-nitro-2-oxo-1,2-dihydroquinoline-3-carbonitrile,
6-bromo-7-hydroxy-1-methyl-4-[4-(5-methyl-1,3-benzoxazol-2-yl)piperidin-1-yl]-2-oxo-1,2-dihydroquinoline-3-carbonitrile,
6-bromo-7-hydroxy-1-methyl-4-[4-methyl-4-(5-methyl-1,3-benzoxazol-2-yl)piperidin-1-yl]-2-oxo-1,2-dihydroquinoline-3-carbonitrile,
(rac)-6-bromo-1-methyl-4-[4-(5-methyl-1,3-benzoxazol-2-yl)piperidin-1-yl]-2-oxo-7-[(oxolan-3-yl)oxy]-1,2-dihydroquinoline-3-carbonitrile,
(rac)-1-methyl-4-[4-(5-methyl-1,3-benzoxazol-2-yl)piperidin-1-yl]-2-oxo-7-[tetrahydrofuran-3-yloxy]-1,2-dihydroquinoline-3,6-dicarbonitrile,
7-hydroxy-1-methyl-4-[4-methyl-4-(5-methyl-1,3-benzoxazol-2-yl)piperidin-1-yl]-2-oxo-1,2-dihydroquinoline-3,6-dicarbonitrile (rac)-6-ethoxy-1-methyl-4-[4-(5-methyl-1,3-benzoxazol-2-yl)piperidin-1-yl]-2-oxo-7-[tetrahydrofuran-3-yloxy]-1,2-dihydroquinoline-3-carbonitrile,
7-hydroxy-1,6-dimethyl-4-[4-(5-methyl-1,3-benzoxazol-2-yl)piperidin-1-yl]-2-oxo-1,2-dihydroquinoline-3-carbonitrile,
(rac)-1,6-dimethyl-4-[4-(5-methyl-1,3-benzoxazol-2-yl)piperidin-1-yl]-2-oxo-7-[tetrahydrofuran-3-yloxy]-1,2-dihydroquinoline-3-carbonitrile,
(rac)-6-(2,2-difluoroethoxy)-1-methyl-4-[4-(5-methyl-1,3-benzoxazol-2-yl)piperidin-1-yl]-2-oxo-7-[tetrahydrofuran-3-yloxy]-1,2-dihydroquinoline-3-carbonitrile,
(rac)-4-[4-fluoro-4-(5-methyl-1,3-benzoxazol-2-yl)piperidin-1-yl]-6-methoxy-1-methyl-2-oxo-7-[tetrahydrofuran-3-yloxy]-1,2-dihydroquinoline-3-carbonitrile,
(rac)-1-methyl-4-[4-methyl-4-(5-methyl-1,3-benzoxazol-2-yl)piperidin-1-yl]-2-oxo-7-[(oxolan-3-yl)oxy]-1,2-dihydroquinoline-3,6-dicarbonitrile,
(rac)-1-methyl-4-[4-(5-methyl-1,3-benzoxazol-2-yl)piperidin-1-yl]-2-oxo-7-[(oxolan-3-yl)oxy]-6-(3,3,3-trifluoroprop-1-en-2-yl)-1,2-dihydroquinoline-3-carbonitrile,
(rac)-1-methyl-4-[4-(5-methyl-1,3-benzoxazol-2-yl)piperidin-1-yl]-2-oxo-7-[(oxolan-3-yl)oxy]-6-[1-(trifluoromethyl)cyclopropyl]-1,2-dihydroquinoline-3-carbonitrile,
6-bromo-1-methyl-2-oxo-4-(4-{5-[3-(trifluoromethyl)phenyl]-1,3,4-oxadiazol-2-yl}piperidin-1-yl)-1,2-dihydroquinoline-3-carbonitrile,
6-methoxy-1-methyl-4-{4-[3-(3-methylphenyl)-1,2,4-oxadiazol-5-yl]piperidin-1-yl}-2-oxo-1,2-dihydroquinoline-3-carbonitrile,
6-methoxy-1-methyl-2-oxo-4-[4-(3-phenyl-1,2,4-oxadiazol-5-yl)piperidin-1-yl]-1,2-dihydroquinoline-3-carbonitrile,
6-methoxy-1-methyl-4-{4-[3-(2-methylphenyl)-1,2,4-oxadiazol-5-yl]piperidin-1-yl}-2-oxo-1,2-dihydroquinoline-3-carbonitrile,
6-methoxy-1-methyl-2-oxo-4-[4-(5-phenyl-1,3,4-oxadiazol-2-yl)piperidin-1-yl]-1,2-dihydroquinoline-3-carbonitrile,
6-bromo-1-methyl-2-oxo-4-[4-(3-phenyl-1,2,4-oxadiazol-5-yl)piperidin-1-yl]-1,2-dihydroquinoline-3-carbonitrile,
6-bromo-1-methyl-2-oxo-4-[4-(5-phenyl-1,3,4-oxadiazol-2-yl)piperidin-1-yl]-1,2-dihydroquinoline-3-carbonitrile,
6-bromo-1-methyl-4-{4-[5-(2-methylphenyl)-1,3,4-oxadiazol-2-yl]piperidin-1-yl}-2-oxo-1,2-dihydroquinoline-3-carbonitrile,
6-bromo-1-methyl-4-{4-[5-(3-methylphenyl)-1,3,4-oxadiazol-2-yl]piperidin-1-yl}-2-oxo-1,2-dihydroquinoline-3-carbonitrile,
7-hydroxy-1-methyl-4-{4-[5-(2-methylphenyl)-1,3,4-oxadiazol-2-yl]piperidin-1-yl}-2-oxo-1,2-dihydroquinoline-3-carbonitrile,
6-bromo-7-hydroxy-1-methyl-4-{4-[5-(2-methylphenyl)-1,3,4-oxadiazol-2-yl]piperidin-1-yl}-2-oxo-1,2-dihydroquinoline-3-carbonitrile,
(rac)-1-methyl-4-{4-[5-(2-methylphenyl)-1,3,4-oxadiazol-2-yl]piperidin-1-yl}-7-{[oxan-3-yl]oxy}-2-oxo-1,2-dihydroquinoline-3-carbonitrile,
4-[4-(1,3-benzoxazol-2-yl)piperidin-1-yl]-N,1-dimethyl-2-oxo-1,2-dihydroquinoline-3-carboxamide,
N,1-dimethyl-4-[4-(5-methyl-1,3-benzoxazol-2-yl)piperidin-1-yl]-2-oxo-1,2-dihydroquinoline-3-carboxamide,
4-[4-(1,3-benzoxazol-2-yl)-4-methylpiperidin-1-yl]-N,1-dimethyl-2-oxo-1,2-dihydroquinoline-3-carboxamide,
1-methyl-2-oxo-4-{4-[5-(2-oxopyrrolidin-1-yl)-1,3-benzoxazol-2-yl]piperidin-1-yl}-1,2-dihydroquinoline-3-carboxamide,
4-[4-(3-chlorophenyl)-4-methoxypiperidin-1-yl]-1-methyl-2-oxo-1,2-dihydroquinoline-3-carboxamide,
6-(dimethylphosphoryl)-1-methyl-4-[4-methyl-4-(5-methyl-1,3-benzoxazol-2-yl)piperidin-1-yl]-2-oxo-1,2-dihydroquinoline-3-carboxamide,
6-(dimethylphosphoryl)-1-methyl-4-[4-(5-methyl-1,3-benzoxazol-2-yl)piperidin-1-yl]-2-oxo-1,2-dihydroquinoline-3-carboxamide,
6-(methanesulfonyl)-1-methyl-4-[4-(5-methyl-1,3-benzoxazol-2-yl)piperidin-1-yl]-2-oxo-1,2-dihydroquinoline-3-carboxamide,
4-[4-(1-benzothiophen-2-yl)-4-methoxypiperidin-1-yl]-1-methyl-2-oxo-1,2-dihydroquinoline-3-carboxamide,
4-[4-(1-benzothiophen-2-yl)-4-hydroxypiperidin-1-yl]-1-methyl-2-oxo-1,2-dihydroquinoline-3-carboxamide,
4-[4-(6-methoxynaphthalen-2-yl)piperidin-1-yl]-1-methyl-2-oxo-1,2-dihydroquinoline-3-carboxamide,
1-methyl-4-{4-[3-(2-methylphenyl)-1,2,4-oxadiazol-5-yl]piperidin-1-yl}-2-oxo-1,2-dihydroquinoline-3-carboxamide,
4-{4-[3-(4-methoxyphenyl)-1,2,4-oxadiazol-5-yl]piperidin-1-yl}-1-methyl-2-oxo-1,2-dihydroquinoline-3-carboxamide,
1-methyl-4-{4-[3-(3-methylphenyl)-1,2,4-oxadiazol-5-yl]piperidin-1-yl}-2-oxo-1,2-dihydroquinoline-3-carboxamide,
1-methyl-4-{4-[5-(2-methylphenyl)-1,3,4-oxadiazol-2-yl]piperidin-1-yl}-2-oxo-1,2-dihydroquinoline-3-carboxamide,
1-methyl-4-{4-[5-(4-methylphenyl)-1,3,4-oxadiazol-2-yl]piperidin-1-yl}-2-oxo-1,2-dihydroquinoline-3-carboxamide,
1-methyl-4-{4-[5-(3-methylphenyl)-1,3,4-oxadiazol-2-yl]piperidin-1-yl}-2-oxo-1,2-dihydroquinoline-3-carboxamide,
4-{4-[5-(3-chlorophenyl)-1,3,4-oxadiazol-2-yl]piperidin-1-yl}-1-methyl-2-oxo-1,2-dihydroquinoline-3-carboxamide,
4-{4-[5-(3-chlorophenyl)-1,3,4-oxadiazol-2-yl]-4-methylpiperidin-1-yl}-1-methyl-2-oxo-1,2-dihydroquinoline-3-carboxamide,
1-methyl-4-{4-[2-methyl-2,3-dihydro-1-benzofuran-5-yl]piperidin-1-yl}-2-oxo-1,2-dihydroquinoline-3-carboxamide,
4-[4-(2-hydroxy-2,3-dihydro-1H-inden-5-yl)piperidin-1-yl]-1-methyl-2-oxo-1,2-dihydroquinoline-3-carboxamide,
4-[4-(2,2-dimethyl-2H-1,3-benzodioxol-5-yl)piperidin-1-yl]-1-methyl-2-oxo-1,2-dihydroquinoline-3-carboxamide,
1-methyl-2-oxo-4-[4-(2′-oxo-1′,2′-dihydrospiro[cyclobutane-1,3′-indol]-5′-yl)piperidin-1-yl]-1,2-dihydroquinoline-3-carboxamide,
1-methyl-4-[4-methyl-4-(4-methylquinolin-2-yl)piperidin-1-yl]-2-oxo-1,2-dihydroquinoline-3-carboxamide,
4-[4-(4-fluoro-1-methyl-1H-indol-6-yl)piperidin-1-yl]-1-methyl-2-oxo-1,2-dihydroquinoline-3-carboxamide,
1-methyl-4-{4-[1-(3-methylphenyl)-1H-pyrazol-3-yl]piperidin-1-yl}-2-oxo-1,2-dihydroquinoline-3-carboxamide,
1-methyl-4-{4-[1-(2-methylphenyl)-1H-pyrazol-3-yl]piperidin-1-yl}-2-oxo-1,2-dihydroquinoline-3-carboxamide,
4-{4-[1-(3-chlorophenyl)-1H-pyrazol-3-yl]piperidin-1-yl}-1-methyl-2-oxo-1,2-dihydroquinoline-3-carboxamide,
1-methyl-2-oxo-4-{4-[3-(pyridin-3-yl)-1,2,4-oxadiazol-5-yl]piperidin-1-yl}-1,2-dihydroquinoline-3-carboxamide,
4-{4-[4-(3-methoxyphenyl)-1,3-thiazol-2-yl]piperidin-1-yl}-1-methyl-2-oxo-1,2-dihydroquinoline-3-carboxamide,
4-{4-[4-(2-methoxyphenyl)-1,3-thiazol-2-yl]piperidin-1-yl}-1-methyl-2-oxo-1,2-dihydroquinoline-3-carboxamide,
1-methyl-4-{4-[4-(4-methylphenyl)-1,3-thiazol-2-yl]piperidin-1-yl}-2-oxo-1,2-dihydroquinoline-3-carboxamide,
1-methyl-4-[4-(5-methyl-1,3-benzoxazol-2-yl)piperidin-1-yl]-7-[(oxan-4-yl)oxy]-2-oxo-1,2-dihydroquinoline-3-carboxamide,
7-(2-methoxyethoxy)-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yl]-7-1{[oxiran-2-yl]methoxy}-2-oxo-1,2-dihydroquinoline-3-carboxamide,
(rac)-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
(−)-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carboxamide,
(+)-1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carboxamide,
1-methyl-4-[4-(5-methyl-1,3-benzoxazol-2-yl)piperidin-1-yl]-7-[(oxetan-3-yl)oxy]-2-oxo-1,2-dihydroquinoline-3-carboxamide,
4-[4-(5-methoxy-1,3-benzoxazol-2-yl)piperidin-1-yl]-1-methyl-2-oxo-1,2-dihydroquinoline-3-carboxamide,
1-methyl-2-oxo-4-(4-{5-[(oxolan-2-yl)methoxy]-1,3-benzoxazol-2-yl}piperidin-1-yl)-1,2-dihydroquinoline-3-carboxamide,
4-[4-(1,3-benzoxazol-2-yl)-4-fluoropiperidin-1-yl]-1-methyl-2-oxo-1,2-dihydroquinoline-3-carboxamide,
4-[4-(1,3-benzoxazol-2-yl)-4-methoxypiperidin-1-yl]-1-methyl-2-oxo-1,2-dihydroquinoline-3-carboxamide,
4-{4-[5-(methoxymethyl)-1,3-benzoxazol-2-yl]-4-methylpiperidin-1-yl}-1-methyl-2-oxo-1,2-dihydroquinoline-3-carboxamide,
4-{4-[6-(methoxymethyl)-1,3-benzoxazol-2-yl]-4-methylpiperidin-1-yl}-1-methyl-2-oxo-1,2-dihydroquinoline-3-carboxamide,
6-bromo-1-methyl-4-[4-(5-methyl-1,3-benzoxazol-2-yl)piperidin-1-yl]-2-oxo-1,2-dihydroquinoline-3-carboxamide,
8-bromo-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yl]-7-[methyl(oxetan-3-yl)amino]-2-oxo-1,2-dihydroquinoline-3-carboxamide,
7-[(2-hydroxyethyl)(methyl)amino]-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yl]-7-{methyl[(3R)-oxolan-3-yl]amino}-2-oxo-1,2-dihydroquinoline-3-carboxamide,
1-methyl-4-[4-(5-methyl-1,3-benzoxazol-2-yl)piperidin-1-yl]-7-{methyl[(3S)-oxolan-3-yl]amino}-2-oxo-1,2-dihydroquinoline-3-carboxamide,
6-cyano-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yl]-2-oxo-1,2-dihydroquinoline-3,6-dicarboxamide,
7-[(cyanomethyl)(methyl)amino]-1-methyl-4-[4-(5-methyl-1,3-benzoxazol-2-yl)piperidin-1-yl]-2-oxo-1,2-dihydroquinoline-3-carboxamide,
6-methoxy-1-methyl-4-[4-(5-methyl-1,3-benzoxazol-2-yl)piperidin-1-yl]-2-oxo-1,2-dihydroquinoline-3-carboxamide,
6-ethoxy-1-methyl-4-[4-(5-methyl-1,3-benzoxazol-2-yl)piperidin-1-yl]-2-oxo-1,2-dihydroquinoline-3-carboxamide,
6-(2,2-difluoropropoxy)-1-methyl-4-[4-(5-methyl-1,3-benzoxazol-2-yl)piperidin-1-yl]-2-oxo-1,2-dihydroquinoline-3-carboxamide,
6-(2,2-difluoroethoxy)-1-methyl-4-[4-(5-methyl-1,3-benzoxazol-2-yl)piperidin-1-yl]-2-oxo-1,2-dihydroquinoline-3-carboxamide,
6-(cyclopropylmethoxy)-1-methyl-4-[4-(5-methyl-1,3-benzoxazol-2-yl)piperidin-1-yl]-2-oxo-1,2-dihydroquinoline-3-carboxamide,
6-cyclobutyl-1-methyl-4-[4-(5-methyl-1,3-benzoxazol-2-yl)piperidin-1-yl]-2-oxo-1,2-dihydroquinoline-3-carboxamide,
1-methyl-4-[4-(5-methyl-1,3-benzoxazol-2-yl)piperidin-1-yl]-2-oxo-6-[1-(trifluoromethyl)cyclopropyl]-1,2-dihydroquinoline-3-carboxamide,
7-(2-amino-2-oxoethoxy)-1-methyl-4-[4-(5-methyl-1,3-benzoxazol-2-yl)piperidin-1-yl]-2-oxo-1,2-dihydroquinoline-3-carboxamide,
4-[4-fluoro-4-(5-methyl-1,3-benzoxazol-2-yl)piperidin-1-yl]-1-methyl-2-oxo-7-[(oxolan-3-yl)oxy]-1,2-dihydroquinoline-3-carboxamide,
1,6-dimethyl-4-[4-(5-methyl-1,3-benzoxazol-2-yl)piperidin-1-yl]-7-nitro-2-oxo-1,2-dihydroquinoline-3-carboxamide,
6-bromo-7-hydroxy-1-methyl-4-[4-(5-methyl-1,3-benzoxazol-2-yl)piperidin-1-yl]-2-oxo-1,2-dihydroquinoline-3-carboxamide,
6-bromo-7-hydroxy-1-methyl-4-[4-methyl-4-(5-methyl-1,3-benzoxazol-2-yl)piperidin-1-yl]-2-oxo-1,2-dihydroquinoline-3-carboxamide,
(rac)-6-bromo-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
(rac)-6-cyano-1-methyl-4-[4-methyl-4-(5-methyl-1,3-benzoxazol-2-yl)piperidin-1-yl]-2-oxo-7-[(oxolan-3-yl)oxy]-1,2-dihydroquinoline-3-carboxamide,
6-cyano-1-methyl-4-[4-methyl-4-(5-methyl-1,3-benzoxazol-2-yl)piperidin-1-yl]-2-oxo-7-{[(3R)-oxolan-3-yl]oxy}-1,2-dihydroquinoline-3-carboxamide,
6-cyano-1-methyl-4-[4-methyl-4-(5-methyl-1,3-benzoxazol-2-yl)piperidin-1-yl]-2-oxo-7-{[(3S)-oxolan-3-yl]oxy}-1,2-dihydroquinoline-3-carboxamide,
(rac)-6-(2,2-difluoroethoxy)-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
(rac)-1-methyl-4-[4-(5-methyl-1,3-benzoxazol-2-yl)piperidin-1-yl]-2-oxo-7-[(oxolan-3-yl)oxy]-6-[1-(trifluoromethyl)cyclopropyl]-1,2-dihydroquinoline-3-carboxamide,
6-bromo-1-methyl-2-oxo-4-(4-{5-[3-(trifluoromethyl)phenyl]-1,3,4-oxadiazol-2-yl}piperidin-1-yl)-1,2-dihydroquinoline-3-carboxamide,
6-methoxy-1-methyl-4-{4-[3-(3-methylphenyl)-1,2,4-oxadiazol-5-yl]piperidin-1-yl}-2-oxo-1,2-dihydroquinoline-3-carboxamide,
6-methoxy-1-methyl-2-oxo-4-[4-(3-phenyl-1,2,4-oxadiazol-5-yl)piperidin-1-yl]-1,2-dihydroquinoline-3-carboxamide,
6-methoxy-1-methyl-4-{4-[3-(2-methylphenyl)-1,2,4-oxadiazol-5-yl]piperidin-1-yl}-2-oxo-1,2-dihydroquinoline-3-carboxamide,
6-bromo-1-methyl-2-oxo-4-[4-(3-phenyl-1,2,4-oxadiazol-5-yl)piperidin-1-yl]-1,2-dihydroquinoline-3-carboxamide,
6-bromo-1-methyl-2-oxo-4-[4-(5-phenyl-1,3,4-oxadiazol-2-yl)piperidin-1-yl]-1,2-dihydroquinoline-3-carboxamide,
6-bromo-1-methyl-4-{4-[5-(2-methylphenyl)-1,3,4-oxadiazol-2-yl]piperidin-1-yl}-2-oxo-1,2-dihydroquinoline-3-carboxamide,
6-bromo-1-methyl-4-{4-[5-(3-methylphenyl)-1,3,4-oxadiazol-2-yl]piperidin-1-yl}-2-oxo-1,2-dihydroquinoline-3-carboxamide,
1-methyl-4-{4-[5-(2-methylphenyl)-1,3,4-oxadiazol-2-yl]piperidin-1-yl}-7-{[(3R)-oxan-3-yl]oxy}-2-oxo-1,2-dihydroquinoline-3-carboxamide,
1-methyl-4-{4-[5-(2-methylphenyl)-1,3,4-oxadiazol-2-yl]piperidin-1-yl}-7-{[(3S)-oxan-3-yl]oxy}-2-oxo-1,2-dihydroquinoline-3-carboxamide,
6-bromo-7-hydroxy-1-methyl-4-{4-[5-(2-methylphenyl)-1,3,4-oxadiazol-2-yl]piperidin-1-yl}-2-oxo-1,2-dihydroquinoline-3-carboxamide,
6-bromo-7-methoxy-1-methyl-4-{4-[3-(2-methylphenyl)-1,2,4-oxadiazol-5-yl]piperidin-1-yl}-2-oxo-1,2-dihydroquinoline-3-carbonitrile,
6-bromo-7-hydroxy-1-methyl-4-{4-[3-(2-methylphenyl)-1,2,4-oxadiazol-5-yl]piperidin-1-yl}-2-oxo-1,2-dihydroquinoline-3-carbonitrile,
(rac)-6-bromo-1-methyl-4-{4-[3-(2-methylphenyl)-1,2,4-oxadiazol-5-yl]piperidin-1-yl}-2-oxo-7-[(oxolan-3-yl)oxy]-1,2-dihydroquinoline-3-carbonitrile,
(rac)-6-bromo-1-methyl-4-{4-[3-(2-methylphenyl)-1,2,4-oxadiazol-5-yl]piperidin-1-yl}-2-oxo-7-[(oxolan-3-yl)oxy]-1,2-dihydroquinoline-3-carboxamide,
(rac)-6-cyano-1-methyl-4-{4-[3-(2-methylphenyl)-1,2,4-oxadiazol-5-yl]piperidin-1-yl}-2-oxo-7-[(oxolan-3-yl)oxy]-1,2-dihydroquinoline-3-carboxamide,
6-cyano-1-methyl-4-{4-[3-(2-methylphenyl)-1,2,4-oxadiazol-5-yl]piperidin-1-yl}-2-oxo-7-{[(3R)-oxolan-3-yl]oxy}-1,2-dihydroquinoline-3-carboxamide,
6-cyano-1-methyl-4-{4-[3-(2-methylphenyl)-1,2,4-oxadiazol-5-yl]piperidin-1-yl}-2-oxo-7-{[(3S)-oxolan-3-yl]oxy}-1,2-dihydroquinoline-3-carboxamide,
(−)-6-bromo-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carbonitrile,
(rac)-6-cyano-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
6-cyano-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carboxamide,
6-cyano-1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carboxamide,
6-methoxy-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carbonitrile,
6-methoxy-1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carbonitrile,
6-methoxy-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
(rac)-1,6-dimethyl-4-[4-(5-methyl-1,3-benzoxazol-2-yl)piperidin-1-yl]-2-oxo-7-[(oxolan-3-yl)oxy]-1,2-dihydroquinoline-3-carboxamide,
1,6-dimethyl-4-[4-(5-methyl-1,3-benzoxazol-2-yl)piperidin-1-yl]-2-oxo-7-{[(3R)-oxolan-3-yl]oxy}-1,2-dihydroquinoline-3-carboxamide,
1,6-dimethyl-4-[4-(5-methyl-1,3-benzoxazol-2-yl)piperidin-1-yl]-2-oxo-7-{[(3S)-oxolan-3-yl]oxy}-1,2-dihydroquinoline-3-carboxamide,
7-[(2-methoxyethyl)amino]-1,6-dimethyl-4-[4-(5-methyl-1,3-benzoxazol-2-yl)piperidin-1-yl]-2-oxo-1,2-dihydroquinoline-3-carbonitrile,
7-(3-hydroxyazetidin-1-yl)-1,6-dimethyl-4-[4-(5-methyl-1,3-benzoxazol-2-yl)piperidin-1-yl]-2-oxo-1,2-dihydroquinoline-3-carbonitrile,
1,6-dimethyl-4-[4-(5-methyl-1,3-benzoxazol-2-yl)piperidin-1-yl]-7-[(oxetan-3-yl)amino]-2-oxo-1,2-dihydroquinoline-3-carbonitrile,
7-[(2-hydroxyethyl)amino]-1,6-dimethyl-4-[4-(5-methyl-1,3-benzoxazol-2-yl)piperidin-1-yl]-2-oxo-1,2-dihydroquinoline-3-carbonitrile,
7-[(2-hydroxyethyl)(methyl)amino]-1,6-dimethyl-4-[4-(5-methyl-1,3-benzoxazol-2-yl)piperidin-1-yl]-2-oxo-1,2-dihydroquinoline-3-carbonitrile,
7-[(2-methoxyethyl)amino]-1,6-dimethyl-4-[4-(5-methyl-1,3-benzoxazol-2-yl)piperidin-1-yl]-2-oxo-1,2-dihydroquinoline-3-carboxamide,
7-(3-hydroxyazetidin-1-yl)-1,6-dimethyl-4-[4-(5-methyl-1,3-benzoxazol-2-yl)piperidin-1-yl]-2-oxo-1,2-dihydroquinoline-3-carboxamide,
(rac)-1,6-dimethyl-4-{4-[3-(2-methylphenyl)-1,2,4-oxadiazol-5-yl]piperidin-1-yl}-2-oxo-7-[(oxolan-3-yl)oxy]-1,2-dihydroquinoline-3-carbonitrile,
1,6-dimethyl-4-{4-[3-(2-methylphenyl)-1,2,4-oxadiazol-5-yl]piperidin-1-yl}-2-oxo-7-{[(3R)-oxolan-3-yl]oxy}-1,2-dihydroquinoline-3-carbonitrile,
1,6-dimethyl-4-{4-[3-(2-methylphenyl)-1,2,4-oxadiazol-5-yl]piperidin-1-yl}-2-oxo-7-{[(3S)-oxolan-3-yl]oxy}-1,2-dihydroquinoline-3-carbonitrile,
1,6-dimethyl-4-{4-[3-(2-methylphenyl)-1,2,4-oxadiazol-5-yl]piperidin-1-yl}-2-oxo-7-{[(3R)-oxolan-3-yl]oxy}-1,2-dihydroquinoline-3-carboxamide,
1,6-dimethyl-4-{4-[3-(2-methylphenyl)-1,2,4-oxadiazol-5-yl]piperidin-1-yl}-2-oxo-7-{[(3S)-oxolan-3-yl]oxy}-1,2-dihydroquinoline-3-carboxamide,
7-[(2-hydroxyethyl)amino]-1,6-dimethyl-4-{4-[3-(2-methylphenyl)-1,2,4-oxadiazol-5-yl]piperidin-1-yl}-2-oxo-1,2-dihydroquinoline-3-carbonitrile,
7-bromo-1,6-dimethyl-4-{4-[1-(2-methylphenyl)-1H-pyrazol-3-yl]piperidin-1-yl}-2-oxo-1,2-dihydroquinoline-3-carbonitrile,
7-[(2-hydroxyethyl)amino]-1,6-dimethyl-4-{4-[1-(2-methylphenyl)-1H-pyrazol-3-yl]piperidin-1-yl}-2-oxo-1,2-dihydroquinoline-3-carbonitrile,
6-bromo-7-fluoro-1-methyl-4-[4-(5-methyl-1,3-benzoxazol-2-yl)piperidin-1-yl]-2-oxo-1,2-dihydroquinoline-3-carboxamide,
6-cyano-7-fluoro-1-methyl-4-[4-(5-methyl-1,3-benzoxazol-2-yl)piperidin-1-yl]-2-oxo-1,2-dihydroquinoline-3-carboxamide,
(rac)-6-cyano-1-methyl-4-[4-(5-methyl-1,3-benzoxazol-2-yl)piperidin-1-yl]-2-oxo-7-{[(oxolan-3-yl)methyl]amino}-1,2-dihydroquinoline-3-carboxamide,
6-cyano-1-methyl-4-[4-(5-methyl-1,3-benzoxazol-2-yl)piperidin-1-yl]-2-oxo-7-({[(3R)-oxolan-3-yl]methyl}amino)-1,2-dihydroquinoline-3-carboxamide,
6-cyano-1-methyl-4-[4-(5-methyl-1,3-benzoxazol-2-yl)piperidin-1-yl]-2-oxo-7-({[(3S)-oxolan-3-yl]methyl}amino)-1,2-dihydroquinoline-3-carboxamide,
6-chloro-7-methoxy-1-methyl-4-[4-(5-methyl-1,3-benzoxazol-2-yl)piperidin-1-yl]-2-oxo-1,2-dihydroquinoline-3-carbonitrile,
6-chloro-7-hydroxy-1-methyl-4-[4-(5-methyl-1,3-benzoxazol-2-yl)piperidin-1-yl]-2-oxo-1,2-dihydroquinoline-3-carbonitrile,
(rac)-6-chloro-1-methyl-4-[4-(5-methyl-1,3-benzoxazol-2-yl)piperidin-1-yl]-2-oxo-7-[(oxolan-3-yl)oxy]-1,2-dihydroquinoline-3-carbonitrile,
(rac)-6-chloro-1-methyl-4-[4-(5-methyl-1,3-benzoxazol-2-yl)piperidin-1-yl]-2-oxo-7-[(oxolan-3-yl)oxy]-1,2-dihydroquinoline-3-carboxamide,
6-chloro-1-methyl-4-[4-(5-methyl-1,3-benzoxazol-2-yl)piperidin-1-yl]-2-oxo-7-{[(3R)-oxolan-3-yl]oxy}-1,2-dihydroquinoline-3-carboxamide,
6-chloro-1-methyl-4-[4-(5-methyl-1,3-benzoxazol-2-yl)piperidin-1-yl]-2-oxo-7-{[(3S)-oxolan-3-yl]oxy}-1,2-dihydroquinoline-3-carboxamide,
(rac)-6-chloro-1-methyl-4-[4-(5-methyl-1,3-benzoxazol-2-yl)piperidin-1-yl]-2-oxo-7-[(oxolan-3-yl)methoxy]-1,2-dihydroquinoline-3-carbonitrile,
(rac)-6-chloro-1-methyl-4-[4-(5-methyl-1,3-benzoxazol-2-yl)piperidin-1-yl]-2-oxo-7-[(oxolan-3-yl)methoxy]-1,2-dihydroquinoline-3-carboxamide,
6-chloro-1-methyl-4-[4-(5-methyl-1,3-benzoxazol-2-yl)piperidin-1-yl]-2-oxo-7-{[(3R)-oxolan-3-yl]methoxy}-1,2-dihydroquinoline-3-carboxamide,
6-chloro-1-methyl-4-[4-(5-methyl-1,3-benzoxazol-2-yl)piperidin-1-yl]-2-oxo-7-{[(3S)-oxolan-3-yl]methoxy}-1,2-dihydroquinoline-3-carboxamide,
(rac)-6-bromo-1-methyl-2-oxo-7-[(oxolan-3-yl)oxy]-4-{4-[2-(pyridin-3-yl)-2H-1,2,3-triazol-4-yl]piperidin-1-yl}-1,2-dihydroquinoline-3-carbonitrile,
(rac)-6-bromo-1-methyl-4-{4-[1-(2-methylphenyl)-1H-1,2,3-triazol-4-yl]piperidin-1-yl}-2-oxo-7-[(oxolan-3-yl)oxy]-1,2-dihydroquinoline-3-carbonitrile,
(rac)-1,6-dimethyl-4-{4-[1-(2-methylphenyl)-1H-1,2,3-triazol-4-yl]piperidin-1-yl}-2-oxo-7-[(oxolan-3-yl)oxy]-1,2-dihydroquinoline-3-carbonitrile,
7-bromo-1,6-dimethyl-4-{4-[1-(2-methylphenyl)-1H-pyrazol-4-yl]piperidin-1-yl}-2-oxo-1,2-dihydroquinoline-3-carbonitrile, and
(rac)-1,6-dimethyl-4-{4-[1-(2-methylphenyl)-1H-pyrazol-4-yl]piperidin-1-yl}-2-oxo-7-[(oxolan-3-yl)oxy]-1,2-dihydroquinoline-3-carbonitrile,
or a stereoisomer, a tautomer, an N-oxide, a hydrate, a solvate, or a salt thereof, or a mixture of same;
and
one DGKzeta inhibitor compound of formula (II) selected from the group consisting of:
rac-2-(N-(4-amino-5-benzoyl-thiazol-2-yl)-4-methoxy-2-methyl-anilino)propanamide,
rac-2-[N-(4-amino-5-benzoyl-thiazol-2-yl)-4-(dimethylamino)anilino]propanamide,
rac-2-(N-(4-amino-5-benzoyl-thiazol-2-yl)-4-isopropoxy-anilino)propanamide,
rac-2-(N-(4-amino-5-benzoyl-thiazol-2-yl)-2,4,6-trifluoro-anilino)propanamide,
rac-2-(N-(4-amino-5-benzoyl-thiazol-2-yl)-2-bromo-4-fluoro-anilino)propanamide,
rac-2-(N-[4-amino-5-(6-methylpyridine-3-carbonyl)thiazol-2-yl]-4-fluoro-anilino)propanamide,
rac-2-(N-[4-amino-5-(pyridine-4-carbonyl)thiazol-2-yl]-4-fluoro-anilino)propanamide,
rac-2-(N-[4-amino-5-(2-fluorobenzoyl)thiazol-2-yl]-4-fluoro-anilino)propanamide,
rac-2-(N-(4-amino-5-benzoyl-thiazol-2-yl)-4-cyano-anilino)propanamide,
rac-2-(N-[4-amino-5-[4-chloro-3-(trifluoromethyl)benzoyl]thiazol-2-yl]-4-fluoro-anilino)propanamide,
rac-2-(N-(4-amino-5-benzoyl-thiazol-2-yl)-2-chloro-4-fluoro-anilino)propanamide,
rac-2-(N-[4-amino-5-(3-cyanobenzoyl)thiazol-2-yl]-4-fluoro-anilino)propanamide,
rac-2-[N-(4-amino-5-benzoyl-thiazol-2-yl)-3-chloro-4-(dimethylamino)anilino]propanamide,
rac-2-(N-[4-amino-5-(6-methoxypyridine-3-carbonyl)thiazol-2-yl]-4-fluoro-anilino)propanamide,
rac-4-[4-amino-2-(N-[2-amino-1-methyl-2-oxo-ethyl]-4-fluoro-anilino)thiazole-5-carbonyl]-N-methyl-benzamide,
rac-2-(N-[4-amino-5-(3-fluorobenzoyl)thiazol-2-yl]-4-fluoro-anilino)propanamide,
rac-2-(N-(4-amino-5-benzoyl-thiazol-2-yl)-3-chloro-4-methoxy-anilino)propanamide,
rac-2-(N-[4-amino-5-(4-fluorobenzoyl)thiazol-2-yl]-4-methoxy-anilino)propanamide,
rac-2-(N-[4-amino-5-(4-methylsulfonylbenzoyl)thiazol-2-yl]-4-fluoro-anilino)propanamide,
rac-2-(N-[4-amino-5-(4-imidazol-1-ylbenzoyl)thiazol-2-yl]-4-fluoro-anilino)propanamide,
rac-2-(N-[4-amino-5-(4-cyano-3-fluoro-benzoyl)thiazol-2-yl]-4-fluoro-anilino)propanamide,
(R)-2-(N-[4-amino-5-(4-cyano-3-fluoro-benzoyl)thiazol-2-yl]-4-fluoro-anilino)propanamide,
(S)-2-(N-[4-amino-5-(4-cyano-3-fluoro-benzoyl)thiazol-2-yl]-4-fluoro-anilino)propanamide,
rac-2-(N-[4-amino-5-(4-cyano-2-fluoro-benzoyl)thiazol-2-yl]-4-fluoro-anilino)propanamide,
rac-2-(N-[4-amino-5-(2-fluoro-4-methoxy-benzoyl)thiazol-2-yl]-4-fluoro-anilino)propanamide,
rac-2-[N-(4-amino-5-benzoyl-thiazol-2-yl)-4-(trifluoromethoxy)anilino]propanamide,
rac-2-(N-[4-amino-5-(3,4-difluorobenzoyl)thiazol-2-yl]-4-fluoro-anilino)propanamide,
rac-2-(N-[4-amino-5-(3,4-dichlorobenzoyl)thiazol-2-yl]-4-fluoro-anilino)propanamide,
rac-2-[N-[4-amino-5-(4-fluorobenzoyl)thiazol-2-yl]-4-(trifluoromethoxy)anilino]propanamide,
(R)-2-[N-[4-amino-5-(4-fluorobenzoyl)thiazol-2-yl]-4-(trifluoromethoxy)anilino]propanamide,
(S)-2-[N-[4-amino-5-(4-fluorobenzoyl)thiazol-2-yl]-4-(trifluoromethoxy)anilino]propanamide,
rac-2-[N-(4-amino-5-benzoyl-thiazol-2-yl)-4-(trifluoromethyl)anilino]propanamide,
rac-2-(N-[4-amino-5-[6-(trifluoromethyl)pyridine-3-carbonyl]thiazol-2-yl]-4-fluoro-anilino)propanamide,
(R)-2-(N-[4-amino-5-[6-(trifluoromethyl)pyridine-3-carbonyl]thiazol-2-yl]-4-fluoro-anilino)propanamide,
(S)-2-(N-[4-amino-5-[6-(trifluoromethyl)pyridine-3-carbonyl]thiazol-2-yl]-4-fluoro-anilino)propanamide,
rac-2-(N-(4-amino-5-benzoyl-thiazol-2-yl)-4-chloro-2-fluoro-anilino)propanamide,
rac-2-(N-[4-amino-5-(indane-5-carbonyl)thiazol-2-yl]-4-fluoro-anilino)propanamide,
rac-2-(N-(4-amino-5-benzoyl-thiazol-2-yl)-3,4-difluoro-anilino)propanamide,
rac-2-(N-[4-amino-5-(4-cyanobenzoyl)thiazol-2-yl]-4-fluoro-anilino)propanamide,
(R)-2-(N-[4-amino-5-(4-cyanobenzoyl)thiazol-2-yl]-4-fluoro-anilino)propanamide,
(S)-2-(N-[4-amino-5-(4-cyanobenzoyl)thiazol-2-yl]-4-fluoro-anilino)propanamide,
rac-2-(N-(4-amino-5-benzoyl-thiazol-2-yl)-4-chloro-anilino)propanamide,
rac-2-(N-(4-amino-5-benzoyl-thiazol-2-yl)-3,4-dichloro-anilino)propanamide,
rac-2-(N-(4-amino-5-benzoyl-thiazol-2-yl)-4-chloro-3-fluoro-anilino)propanamide,
rac-2-(N-[4-amino-5-(4-fluorobenzoyl)thiazol-2-yl]-4-chloro-anilino)propanamide,
(R)-2-(N-[4-amino-5-(4-fluorobenzoyl)thiazol-2-yl]-4-chloro-anilino)propanamide,
(S)-2-(N-[4-amino-5-(4-fluorobenzoyl)thiazol-2-yl]-4-chloro-anilino)propanamide,
rac-2-(N-[4-amino-5-(4-chlorobenzoyl)thiazol-2-yl]-4-fluoro-anilino)propanamide,
(R)-2-(N-[4-amino-5-(4-chlorobenzoyl)thiazol-2-yl]-4-fluoro-anilino)propanamide,
(S)-2-(N-[4-amino-5-(4-chlorobenzoyl)thiazol-2-yl]-4-fluoro-anilino)propanamide,
rac-ethyl 2-[4-[4-amino-2-(N-[2-amino-1-methyl-2-oxo-ethyl]-4-fluoro-anilino)thiazole-5-carbonyl]phenoxy]-2-methyl-propanoate,
rac-2-(N-[4-amino-5-[4-(trifluoromethoxy)benzoyl]thiazol-2-yl]-4-fluoro-anilino)propanamide,
(R)-2-(N-[4-amino-5-[4-(trifluoromethoxy)benzoyl]thiazol-2-yl]-4-fluoro-anilino)propanamide,
(S)-2-(N-[4-amino-5-[4-(trifluoromethoxy)benzoyl]thiazol-2-yl]-4-fluoro-anilino)propanamide,
rac-2-(N-[4-amino-5-(4-chlorobenzoyl)thiazol-2-yl]-4-chloro-anilino)propanamide,
(R)-2-(N-[4-amino-5-(4-chlorobenzoyl)thiazol-2-yl]-4-chloro-anilino)propanamide,
(S)-2-(N-[4-amino-5-(4-chlorobenzoyl)thiazol-2-yl]-4-chloro-anilino)propanamide,
rac-2-(N-[4-amino-5-[4-(trifluoromethyl)benzoyl]thiazol-2-yl]-4-fluoro-anilino)propanamide,
rac-2-[N-[4-amino-5-[4-(difluoromethoxy)benzoyl]thiazol-2-yl]-4-(trifluoromethyl)anilino]propanamide,
R)-2-[N-[4-amino-5-[4-(difluoromethoxy)benzoyl]thiazol-2-yl]-4-(trifluoromethyl)anilino]propanamide,
(S)-2-[N-[4-amino-5-[4-(difluoromethoxy)benzoyl]thiazol-2-yl]-4-(trifluoromethyl)anilino]propanamide,
rac-2-(N-[4-amino-5-[4-(difluoromethoxy)benzoyl]thiazol-2-yl]-4-fluoro-anilino)propanamide,
(R)-2-(N-[4-amino-5-[4-(difluoromethoxy)benzoyl]thiazol-2-yl]-4-fluoro-anilino)propanamide,
(S)-2-(N-[4-amino-5-[4-(difluoromethoxy)benzoyl]thiazol-2-yl]-4-fluoro-anilino)propanamide,
2-(N-[4-amino-5-[4-[2-amino-1-methyl-2-oxo-ethoxy]benzoyl]thiazol-2-yl]anilino)propanamide (mixture of stereoisomers),
(2R)—(N-[4-amino-5-[4-[2-amino-(1R)-methyl-2-oxo-ethoxy]benzoyl]thiazol-2-yl]anilino)propanamide,
(2R)—(N-[4-amino-5-[4-[2-amino-(1S)-methyl-2-oxo-ethoxy]benzoyl]thiazol-2-yl]anilino)propanamide,
(2S)—(N-[4-amino-5-[4-[2-amino-(1R)-methyl-2-oxo-ethoxy]benzoyl]thiazol-2-yl]anilino)propanamide,
(2S)—(N-[4-amino-5-[4-[2-amino-(1S)-methyl-2-oxo-ethoxy]benzoyl]thiazol-2-yl]anilino)propanamide,
rac-2-(N-[4-amino-5-[4-(difluoromethoxy)benzoyl]thiazol-2-yl]-4-chloro-2-fluoro-anilino)propanamide,
(R)-2-(N-[4-amino-5-[4-(difluoromethoxy)benzoyl]thiazol-2-yl]-4-chloro-2-fluoro-anilino)propanamide,
(S)-2-(N-[4-amino-5-[4-(difluoromethoxy)benzoyl]thiazol-2-yl]-4-chloro-2-fluoro-anilino)propanamide,
rac-2-(N-[4-amino-5-[4-(difluoromethoxy)benzoyl]thiazol-2-yl]-4-chloro-anilino)propanamide,
(R)-2-(N-[4-amino-5-[4-(difluoromethoxy)benzoyl]thiazol-2-yl]-4-chloro-anilino)propanamide,
(S)-2-(N-[4-amino-5-[4-(difluoromethoxy)benzoyl]thiazol-2-yl]-4-chloro-anilino)propanamide,
rac-2-(N-[4-amino-5-[4-(difluoromethoxy)benzoyl]thiazol-2-yl]-4-chloro-3-fluoro-anilino)propanamide,
(R)-2-(N-[4-amino-5-[4-(difluoromethoxy)benzoyl]thiazol-2-yl]-4-chloro-3-fluoro-anilino)propanamide,
(S)-2-(N-[4-amino-5-[4-(difluoromethoxy)benzoyl]thiazol-2-yl]-4-chloro-3-fluoro-anilino)propanamide,
rac-2-(N-[4-amino-5-[4-(difluoromethoxy)benzoyl]thiazol-2-yl]-3,4-difluoro-anilino)propanamide,
(R)-2-(N-[4-amino-5-[4-(difluoromethoxy)benzoyl]thiazol-2-yl]-3,4-difluoro-anilino)propanamide,
(S)-2-(N-[4-amino-5-[4-(difluoromethoxy)benzoyl]thiazol-2-yl]-3,4-difluoro-anilino)propanamide,
rac-2-(N-[4-amino-5-(pyridine-4-carbonyl)thiazol-2-yl]-3,4-difluoro-anilino)propanamide,
(R)-2-(N-[4-amino-5-(pyridine-4-carbonyl)thiazol-2-yl]-3,4-difluoro-anilino)propanamide,
(S)-2-(N-[4-amino-5-(pyridine-4-carbonyl)thiazol-2-yl]-3,4-difluoro-anilino)propanamide,
rac-2-(N-[4-amino-5-(4-methoxybenzoyl)thiazol-2-yl]-3,4-difluoro-anilino)propanamide,
(R)-2-(N-[4-amino-5-(4-methoxybenzoyl)thiazol-2-yl]-3,4-difluoro-anilino)propanamide,
(S)-2-(N-[4-amino-5-(4-methoxybenzoyl)thiazol-2-yl]-3,4-difluoro-anilino)propanamide,
rac-2-(N-[4-amino-5-(6-methoxypyridine-3-carbonyl)thiazol-2-yl]-3,4-difluoro-anilino)propanamide,
(R)-2-(N-[4-amino-5-(6-methoxypyridine-3-carbonyl)thiazol-2-yl]-3,4-difluoro-anilino)propanamide,
(S)-2-(N-[4-amino-5-(6-methoxypyridine-3-carbonyl)thiazol-2-yl]-3,4-difluoro-anilino)propanamide,
rac-2-(N-[4-amino-5-[6-(trifluoromethyl)pyridine-3-carbonyl]thiazol-2-yl]-3,4-difluoro-anilino)propanamide,
(R)-2-(N-[4-amino-5-[6-(trifluoromethyl)pyridine-3-carbonyl]thiazol-2-yl]-3,4-difluoro-anilino)propanamide,
(S)-2-(N-[4-amino-5-[6-(trifluoromethyl)pyridine-3-carbonyl]thiazol-2-yl]-3,4-difluoro-anilino)propanamide,
rac-2-(N-[5-[4-(difluoromethoxy)benzoyl]-4-methyl-thiazol-2-yl]-4-fluoro-anilino)propanamide,
rac-2-(N-[5-[4-(difluoromethoxy)benzoyl]-4-methyl-thiazol-2-yl]-3,4-difluoro-anilino)propanamide,
(R)-2-(N-[5-[4-(difluoromethoxy)benzoyl]-4-methyl-thiazol-2-yl]-3,4-difluoro-anilino)propanamide,
(S)-2-(N-[5-[4-(difluoromethoxy)benzoyl]-4-methyl-thiazol-2-yl]-3,4-difluoro-anilino)propanamide,
rac-2-(N-[4-amino-5-(pyridine-4-carbonyl)thiazol-2-yl]-4-chloro-3-fluoro-anilino)propanamide,
(R)-2-(N-[4-amino-5-(pyridine-4-carbonyl)thiazol-2-yl]-4-chloro-3-fluoro-anilino)propanamide,
(S)-2-(N-[4-amino-5-(pyridine-4-carbonyl)thiazol-2-yl]-4-chloro-3-fluoro-anilino)propanamide,
rac-2-(N-[4-amino-5-(pyridine-4-carbonyl)thiazol-2-yl]-4-chloro-anilino)propanamide,
(R)-2-(N-[4-amino-5-(pyridine-4-carbonyl)thiazol-2-yl]-4-chloro-anilino)propanamide,
(S)-2-(N-[4-amino-5-(pyridine-4-carbonyl)thiazol-2-yl]-4-chloro-anilino)propanamide,
rac-2-(N-[4-amino-5-(4-methoxybenzoyl)thiazol-2-yl]-4-chloro-anilino)propanamide,
(R)-2-(N-[4-amino-5-(4-methoxybenzoyl)thiazol-2-yl]-4-chloro-anilino)propanamide,
(S)-2-(N-[4-amino-5-(4-methoxybenzoyl)thiazol-2-yl]-4-chloro-anilino)propanamide,
rac-2-(N-[4-amino-5-(4-methoxybenzoyl)thiazol-2-yl]-4-chloro-3-fluoro-anilino)propanamide,
(R)-2-(N-[4-amino-5-(4-methoxybenzoyl)thiazol-2-yl]-4-chloro-3-fluoro-anilino)propanamide,
(S)-2-(N-[4-amino-5-(4-methoxybenzoyl)thiazol-2-yl]-4-chloro-3-fluoro-anilino)propanamide
rac-2-(N-(4-amino-5-benzoyl-thiazol-2-yl)anilino)propanamide,
rac-2-(N-(4-amino-5-benzoyl-thiazol-2-yl)-4-fluoro-anilino)propanamide,
(R)-2-(N-(4-amino-5-benzoyl-thiazol-2-yl)-4-fluoro-anilino)propanamide,
(S)-2-(N-(4-amino-5-benzoyl-thiazol-2-yl)-4-fluoro-anilino)propanamide,
rac-2-(N-[4-amino-5-(4-methoxybenzoyl)thiazol-2-yl]-4-fluoro-anilino)propanamide,
(R)-2-(N-[4-amino-5-(4-methoxybenzoyl)thiazol-2-yl]-4-fluoro-anilino)propanamide,
(S)-2-(N-[4-amino-5-(4-methoxybenzoyl)thiazol-2-yl]-4-fluoro-anilino)propanamide,
rac-2-(N-[4-amino-5-(4-methylbenzoyl)thiazol-2-yl]-4-fluoro-anilino)propanamide,
(R)-2-(N-[4-amino-5-(4-methylbenzoyl)thiazol-2-yl]-4-fluoro-anilino)propanamide,
(S)-2-(N-[4-amino-5-(4-methylbenzoyl)thiazol-2-yl]-4-fluoro-anilino)propanamide,
rac-2-(N-[4-amino-5-(4-fluorobenzoyl)thiazol-2-yl]-4-fluoro-anilino)propanamide,
rac-2-(N-(4-amino-5-benzoyl-thiazol-2-yl)-2,4-difluoro-anilino)propanamide,
rac-2-(N-(4-amino-5-benzoyl-thiazol-2-yl)-4-methoxy-anilino)propanamide,
rac-2-[(4-amino-5-benzoyl-1,3-thiazol-2-yl)(phenyl)amino]butanamide,
rac-2-[(4-amino-5-benzoyl-1,3-thiazol-2-yl)(4-fluorophenyl)amino]butanamide,
2-(N-[4-amino-5-[4-(2-amino-1-methyl-2-oxo-ethoxy)benzoyl]thiazol-2-yl]-4-fluoro-anilino)propanamide (mixture of stereoisomers),
rac-2-{[4-amino-5-(4-methoxybenzoyl)-1,3-thiazol-2-yl](4-fluorophenyl)amino}butanamide,
2-(N-(4-amino-5-benzoyl-thiazol-2-yl)-4-fluoro-anilino)acetamide,
2-(N-[4-amino-5-(4-methylbenzoyl)thiazol-2-yl]-4-fluoro-anilino)acetamide,
rac-2-(N-(4-amino-5-benzoyl-thiazol-2-yl)-4-methyl-anilino)propanamide,
(R)-2-(N-(4-amino-5-benzoyl-thiazol-2-yl)-4-methyl-anilino)propanamide,
(S)-2-(N-(4-amino-5-benzoyl-thiazol-2-yl)-4-methyl-anilino)propanamide,
rac-2-(N-(4-amino-5-benzoyl-thiazol-2-yl)-3-fluoro-anilino)propanamide,
(R)-2-(N-(4-amino-5-benzoyl-thiazol-2-yl)-3-fluoro-anilino)propanamide,
(S)-2-(N-(4-amino-5-benzoyl-thiazol-2-yl)-3-fluoro-anilino)propanamide,
rac-2-(N-(4-amino-5-benzoyl-thiazol-2-yl)-2-fluoro-anilino)propanamide,
(R)-2-(N-(4-amino-5-benzoyl-thiazol-2-yl)-2-fluoro-anilino)propanamide,
(S)-2-(N-(4-amino-5-benzoyl-thiazol-2-yl)-2-fluoro-anilino)propanamide,
rac-2-(N-(4-amino-5-benzoyl-thiazol-2-yl)-3-methyl-anilino)propanamide,
(R)-2-(N-(4-amino-5-benzoyl-thiazol-2-yl)-3-methyl-anilino)propanamide,
(S)-2-(N-(4-amino-5-benzoyl-thiazol-2-yl)-3-methyl-anilino)propanamide,
rac-2-(N-(4-amino-5-benzoyl-thiazol-2-yl)-2-methyl-anilino)propanamide,
(R)-2-(N-(4-amino-5-benzoyl-thiazol-2-yl)-2-methyl-anilino)propanamide,
(S)-2-(N-(4-amino-5-benzoyl-thiazol-2-yl)-2-methyl-anilino)propanamide,
rac-2-[(4-amino-5-benzoyl-thiazol-2-yl)-(1-methylpyrazol-4-yl)amino]propanamide,
(R)-2-[(4-amino-5-benzoyl-thiazol-2-yl)-(1-methylpyrazol-4-yl)amino]propanamide,
(S)-2-[(4-amino-5-benzoyl-thiazol-2-yl)-(1-methylpyrazol-4-yl)amino]propanamide,
rac-2-[(4-amino-5-benzoyl-thiazol-2-yl)-(3-pyridyl)amino]propanamide,
(R)-2-[(4-amino-5-benzoyl-thiazol-2-yl)-(3-pyridyl)amino]propanamide,
(S)-2-[(4-amino-5-benzoyl-thiazol-2-yl)-(3-pyridyl)amino]propanamide,
rac-2-(N-[4-amino-5-(4-bromobenzoyl)thiazol-2-yl]-4-fluoro-anilino)propanamide,
rac-ethyl 2-[4-[4-amino-2-(4-fluoro-N-[2-amino-1-methyl-2-oxo-ethyl]anilino)thiazole-5-carbonyl]phenoxy]acetate,
(R)-ethyl 2-[4-[4-amino-2-(4-fluoro-N-[2-amino-1-methyl-2-oxo-ethyl]anilino)thiazole-5-carbonyl]phenoxy]acetate,
(S)-ethyl 2-[4-[4-amino-2-(4-fluoro-N-[2-amino-1-methyl-2-oxo-ethyl]anilino)thiazole-5-carbonyl]phenoxy]acetate,
rac-2-(N-[4-amino-5-[4-[2-(isopropylamino)-2-oxo-ethoxy]benzoyl]thiazol-2-yl]-4-fluoro-anilino)propanamide,
rac-2-(N-[4-amino-5-[4-[2-(m-tolylmethylamino)-2-oxo-ethoxy]benzoyl]thiazol-2-yl]-4-fluoro-anilino)propanamide,
rac-2-(N-[4-amino-5-[4-[2-(o-tolylmethylamino)-2-oxo-ethoxy]benzoyl]thiazol-2-yl]-4-fluoro-anilino)propanamide,
rac-2-(N-[4-amino-5-[4-[2-[(3-chlorophenyl)methylamino]-2-oxo-ethoxy]benzoyl]thiazol-2-yl]-4-fluoro-anilino)propanamide,
rac-2-(N-[4-amino-5-[4-[2-(4-methylpiperazin-1-yl)-2-oxo-ethoxy]benzoyl]thiazol-2-yl]-4-fluoro-anilino)propanamide,
rac-2-(N-[4-amino-5-[4-[2-(3-methylanilino)-2-oxo-ethoxy]benzoyl]thiazol-2-yl]-4-fluoro-anilino)propanamide,
rac-2-(N-[4-amino-5-[4-(2-morpholino-2-oxo-ethoxy)benzoyl]thiazol-2-yl]-4-fluoro-anilino)propanamide,
rac-2-(N-[4-amino-5-[4-[2-oxo-2-[2-(1-piperidyl)ethylamino]ethoxy]benzoyl]thiazol-2-yl]-4-fluoro-anilino)propanamide,
rac-2-(N-[4-amino-5-[4-[2-(4-benzyl-1-piperidyl)-2-oxo-ethoxy]benzoyl]thiazol-2-yl]-4-fluoro-anilino)propanamide,
rac-2-(N-[4-amino-5-[4-[2-(2-methoxyethylamino)-2-oxo-ethoxy]benzoyl]thiazol-2-yl]-4-fluoro-anilino)propanamide,
rac-2-(N-[4-amino-5-[4-[2-(4-cyanoanilino)-2-oxo-ethoxy]benzoyl]thiazol-2-yl]-4-fluoro-anilino)propanamide,
rac-2-(N-[4-amino-5-[4-[2-[methyl(prop-2-ynyl)amino]-2-oxo-ethoxy]benzoyl]thiazol-2-yl]-4-fluoro-anilino)propanamide,
rac-2-(N-[4-amino-5-[4-[2-[(2-methoxyphenyl)methylamino]-2-oxo-ethoxy]benzoyl]thiazol-2-yl]-4-fluoro-anilino)propanamide,
rac-2-(N-[4-amino-5-[4-[2-[(3-methoxyphenyl)methylamino]-2-oxo-ethoxy]benzoyl]thiazol-2-yl]-4-fluoro-anilino)propanamide,
rac-2-(N-[4-amino-5-[4-[2-[(2-fluorophenyl)methylamino]-2-oxo-ethoxy]benzoyl]thiazol-2-yl]-4-fluoro-anilino)propanamide,
rac-2-(N-[4-amino-5-[4-[2-[(4-fluorophenyl)methylamino]-2-oxo-ethoxy]benzoyl]thiazol-2-yl]-4-fluoro-anilino)propanamide,
rac-2-(N-[4-amino-5-[4-[2-(1H-benzimidazol-2-ylmethylamino)-2-oxo-ethoxy]benzoyl]thiazol-2-yl]-4-fluoro-anilino)propanamide,
rac-2-(N-[4-amino-5-[4-[2-oxo-2-(2,2,2-trifluoroethylamino)ethoxy]benzoyl]thiazol-2-yl]-4-fluoro-anilino)propanamide,
rac-2-(N-[4-amino-5-[4-[2-[methyl(2-pyridyl)amino]-2-oxo-ethoxy]benzoyl]thiazol-2-yl]-4-fluoro-anilino)propanamide,
rac-2-(N-[4-amino-5-[4-[2-[methyl-(1-methyl-4-piperidyl)amino]-2-oxo-ethoxy]benzoyl]thiazol-2-yl]-4-fluoro-anilino)propanamide,
rac-2-(N-[4-amino-5-[4-[2-(methoxyamino)-2-oxo-ethoxy]benzoyl]thiazol-2-yl]-4-fluoro-anilino)propanamide,
rac-2-(N-[4-amino-5-[4-[2-[(5-methylisoxazol-3-yl)amino]-2-oxo-ethoxy]benzoyl]thiazol-2-yl]-4-fluoro-anilino)propanamide,
rac-2-(N-[4-amino-5-[4-[2-(ethylamino)-2-oxo-ethoxy]benzoyl]thiazol-2-yl]-4-fluoro-anilino)propanamide,
rac-2-(N-[4-amino-5-[4-[2-(4-methylanilino)-2-oxo-ethoxy]benzoyl]thiazol-2-yl]-4-fluoro-anilino)propanamide,
rac-2-(N-[4-amino-5-[4-[2-(cyclohexylamino)-2-oxo-ethoxy]benzoyl]thiazol-2-yl]-4-fluoro-anilino)propanamide,
rac-3-[[2-[4-[4-amino-2-(N-[2-amino-1-methyl-2-oxo-ethyl]-4-fluoro-anilino)thiazole-5-carbonyl]phenoxy]acetyl]amino]benzamide,
rac-2-(N-[4-amino-5-[4-[2-oxo-2-(6-quinolylamino)ethoxy]benzoyl]thiazol-2-yl]-4-fluoro-anilino)propanamide,
rac-4-[[2-[4-[4-amino-2-(N-[2-amino-1-methyl-2-oxo-ethyl]-4-fluoro-anilino)thiazole-5-carbonyl]phenoxy]acetyl]amino]benzamide,
(2S)-1-[2-[4-[4-amino-2-(N-[2-amino-(1RS)-methyl-2-oxo-ethyl]-4-fluoro-anilino)thiazole-5-carbonyl]phenoxy]acetyl]pyrrolidine-2-carboxamide (mixture of two diastereomers),
rac-2-(N-[4-amino-5-[4-[2-[ethyl(methyl)amino]-2-oxo-ethoxy]benzoyl]thiazol-2-yl]-4-fluoro-anilino)propanamide,
rac-2-(N-[4-amino-5-[4-[2-[(3-methylisoxazol-5-yl)amino]-2-oxo-ethoxy]benzoyl]thiazol-2-yl]-4-fluoro-anilino)propanamide,
rac-2-(N-[4-amino-5-[4-[2-[3-(dimethylamino)propyl-methyl-amino]-2-oxo-ethoxy]benzoyl]thiazol-2-yl]-4-fluoro-anilino)propanamide,
rac-2-(N-[5-[4-[2-(4-acetylpiperazin-1-yl)-2-oxo-ethoxy]benzoyl]-4-amino-thiazol-2-yl]-4-fluoro-anilino)propanamide,
rac-2-(N-[4-amino-5-[4-[2-oxo-2-(3-pyridylmethylamino)ethoxy]benzoyl]thiazol-2-yl]-4-fluoro-anilino)propanamide,
2-(N-[4-amino-5-[4-[2-(2,3-dihydroxypropylamino)-2-oxo-ethoxy]benzoyl]thiazol-2-yl]-4-fluoro-anilino)propanamide (mixture of stereoisomers),
rac-2-(N-[4-amino-5-[4-[2-(4-methoxyanilino)-2-oxo-ethoxy]benzoyl]thiazol-2-yl]-4-fluoro-anilino)propenamide,
rac-2-(N-[4-amino-5-[4-[2-[benzyl(methyl)amino]-2-oxo-ethoxy]benzoyl]thiazol-2-yl]-4-fluoro-anilino)propanamide,
rac-2-(N-[4-amino-5-[4-[2-(4-chloroanilino)-2-oxo-ethoxy]benzoyl]thiazol-2-yl]-4-fluoro-anilino)propanamide,
rac-2-(N-[4-amino-5-[4-[2-[(2-chlorophenyl)methylamino]-2-oxo-ethoxy]benzoyl]thiazol-2-yl]-4-fluoro-anilino)propanamide,
rac-2-(N-[4-amino-5-[4-[2-[(4-chlorophenyl)methylamino]-2-oxo-ethoxy]benzoyl]thiazol-2-yl]-4-fluoro-anilino)propanamide,
rac-2-(N-[4-amino-5-[4-[2-(4-fluoroanilino)-2-oxo-ethoxy]benzoyl]thiazol-2-yl]-4-fluoro-anilino)propanamide,
rac-2-(N-[4-amino-5-[4-[2-(azepan-1-yl)-2-oxo-ethoxy]benzoyl]thiazol-2-yl]-4-fluoro-anilino)propanamide,
rac-2-(N-[4-amino-5-[4-[2-[(4-methoxyphenyl)methylamino]-2-oxo-ethoxy]benzoyl]thiazol-2-yl]-4-fluoro-anilino)propanamide,
2-(N-[4-amino-5-[4-[2-oxo-2-(1-phenylethylamino)ethoxy]benzoyl]thiazol-2-yl]-4-fluoro-anilino)propanamide (mixture of stereoisomers),
rac-2-(N-[4-amino-5-[4-[2-oxo-2-(p-tolylmethylamino)ethoxy]benzoyl]thiazol-2-yl]-4-fluoro-anilino)propenamide,
rac-2-(N-[4-amino-5-[4-[2-[methyl(2-phenylethyl)amino]-2-oxo-ethoxy]benzoyl]thiazol-2-yl]-4-fluoro-anilino)propanamide,
2-(N-[4-amino-5-[4-[2-(3-methyl-1-piperidyl)-2-oxo-ethoxy]benzoyl]thiazol-2-yl]-4-fluoro-anilino)propanamide (mixture of stereoisomers),
rac-2-(N-[4-amino-5-[4-[2-(4-methyl-1-piperidyl)-2-oxo-ethoxy]benzoyl]thiazol-2-yl]-4-fluoro-anilino)propanamide,
rac-2-(N-[5-[4-[2-(4-acetamidoanilino)-2-oxo-ethoxy]benzoyl]-4-amino-thiazol-2-yl]-4-fluoro-anilino)propanamide,
rac-2-(N-[4-amino-5-[4-[2-oxo-2-(1H-pyrazolo[3,4-d]pyrimidin-4-ylamino)ethoxy]benzoyl]thiazol-2-yl]-4-fluoro-anilino)propanamide,
rac-2-(N-[4-amino-5-[4-[2-(cyclopentylamino)-2-oxo-ethoxy]benzoyl]thiazol-2-yl]-4-fluoro-anilino)propanamide,
rac-2-(N-[4-amino-5-[4-[2-(3,4-dihydro-1H-isoquinolin-2-yl)-2-oxo-ethoxy]benzoyl]thiazol-2-yl]-4-fluoro-anilino)propanamide,
rac-2-(N-[4-amino-5-[4-(2-isoindolin-2-yl-2-oxo-ethoxy)benzoyl]thiazol-2-yl]-4-fluoro-anilino)propanamide,
rac-2-(N-[4-amino-5-[4-[2-[2-furylmethyl(methyl)amino]-2-oxo-ethoxy]benzoyl]thiazol-2-yl]-4-fluoro-anilino)propanamide,
rac-2-(N-[4-amino-5-[4-[2-[4-(dimethylamino)-1-piperidyl]-2-oxo-ethoxy]benzoyl]thiazol-2-yl]-4-fluoro-anilino)propanamide,
rac-2-(N-[4-amino-5-[4-[2-[methyl(3-pyridylmethyl)amino]-2-oxo-ethoxy]benzoyl]thiazol-2-yl]-4-fluoro-anilino)propanamide,
rac-2-(N-[4-amino-5-[4-[2-(N,2-dimethylanilino)-2-oxo-ethoxy]benzoyl]thiazol-2-yl]-4-fluoro-anilino)propanamide,
rac-2-(N-[4-amino-5-[4-[2-(N,4-dimethylanilino)-2-oxo-ethoxy]benzoyl]thiazol-2-yl]-4-fluoro-anilino)propanamide,
rac-2-(N-[4-amino-5-[4-[2-(N,3-dimethylanilino)-2-oxo-ethoxy]benzoyl]thiazol-2-yl]-4-fluoro-anilino)propanamide,
rac-2-(N-[4-amino-5-[4-[2-(2,2-dimethylpropylamino)-2-oxo-ethoxy]benzoyl]thiazol-2-yl]-4-fluoro-anilino)propanamide,
2-(N-[5-[4-[2-(1-adamantylamino)-2-oxo-ethoxy]benzoyl]-4-amino-thiazol-2-yl]-4-fluoro-anilino)propanamide (single stereoisomer),
2-(N-[5-[4-[2-(1-adamantylmethylamino)-2-oxo-ethoxy]benzoyl]-4-amino-thiazol-2-yl]-4-fluoro-anilino)propanamide (single stereoisomer),
2-(N-[5-[4-[2-[2-(1-adamantyl)ethylamino]-2-oxo-ethoxy]benzoyl]-4-amino-thiazol-2-yl]-4-fluoro-anilino)propanamide (single stereoisomer),
2-(N-[4-amino-5-[4-[2-(4-chloroanilino)-2-oxo-ethoxy]benzoyl]thiazol-2-yl]-4-fluoro-anilino)propanamide (single stereoisomer),
4-[[2-[4-[4-amino-2-(4-fluoro-N-[2-amino-1-methyl-2-oxo-ethyl]anilino)thiazole-5-carbonyl]phenoxy]acetyl]amino]benzamide (single stereoisomer),
2-(N-[4-amino-5-[4-[2-((2RS),3-dihydroxypropylamino)-2-oxo-ethoxy]benzoyl]thiazol-2-yl]-4-fluoro-anilino)propanamide (mixture of two diastereomers),
2-(N-[4-amino-5-[4-[2-oxo-2-[2-(1-piperidyl)ethylamino]ethoxy]benzoyl]thiazol-2-yl]-4-fluoro-anilino)propanamide (single stereoisomer),
2-(N-[4-amino-5-[4-(2-amino-2-oxo-ethoxy)benzoyl]thiazol-2-yl]-4-fluoro-anilino)propanamide (single stereoisomer),
(R)-2-(N-[4-amino-5-[4-[2-(methylamino)-2-oxo-ethoxy]benzoyl]thiazol-2-yl]-4-fluoro-anilino)propanamide,
(S)-2-(N-[4-amino-5-[4-[2-(methylamino)-2-oxo-ethoxy]benzoyl]thiazol-2-yl]-4-fluoro-anilino)propanamide,
(R)-2-(N-[4-amino-5-[4-[2-(isopropylamino)-2-oxo-ethoxy]benzoyl]thiazol-2-yl]-4-fluoro-anilino)propanamide,
(S)-2-(N-[4-amino-5-[4-[2-(isopropylamino)-2-oxo-ethoxy]benzoyl]thiazol-2-yl]-4-fluoro-anilino)propanamide,
rac-2-[4-[4-amino-2-(N-(2-amino-1-methyl-2-oxo-ethyl)-4-fluoro-anilino)thiazole-5-carbonyl]phenoxy]-N-isopropyl-2-methyl-propanamide,
rac-2-[4-[4-amino-2-(N-(2-amino-1-methyl-2-oxo-ethyl)-4-fluoro-anilino)thiazole-5-carbonyl]phenoxy]-2-methyl-propanamide,
rac-2-(N-(5-benzoyl-4-methyl-thiazol-2-yl)-4-fluoro-anilino)propanamide,
rac-2-(N-[4-amino-5-(4-hydroxybenzoyl)thiazol-2-yl]-4-fluoro-anilino)propanamide,
rac-2-(N-[4-amino-5-(4-benzyloxybenzoyl)thiazol-2-yl]-4-fluoro-anilino)propanamide,
rac-2-(N-[4-amino-5-[6-(difluoromethyl)pyridine-3-carbonyl]thiazol-2-yl]-4-fluoro-anilino)propanamide,
rac-2-(N-[4-amino-5-(pyridine-4-carbonyl)thiazol-2-yl]-3-chloro-4-fluoro-anilino)propanamide,
(R)-2-(N-[4-amino-5-(pyridine-4-carbonyl)thiazol-2-yl]-3-chloro-4-fluoro-anilino)propanamide,
(S)-2-(N-[4-amino-5-(pyridine-4-carbonyl)thiazol-2-yl]-3-chloro-4-fluoro-anilino)propanamide,
rac-2-(N-[4-amino-5-(4-methoxybenzoyl)thiazol-2-yl]-3-chloro-4-fluoro-anilino)propanamide,
(R)-2-(N-[4-amino-5-(4-methoxybenzoyl)thiazol-2-yl]-3-chloro-4-fluoro-anilino)propanamide,
(S)-2-(N-[4-amino-5-(4-methoxybenzoyl)thiazol-2-yl]-3-chloro-4-fluoro-anilino)propanamide,
rac-2-(N-[4-amino-5-[6-(difluoromethoxy)pyridine-3-carbonyl]thiazol-2-yl]-4-fluoro-anilino)propanamide,
(R)-2-(N-[4-amino-5-[6-(difluoromethoxy)pyridine-3-carbonyl]thiazol-2-yl]-4-fluoro-anilino)propanamide,
(S)-2-(N-[4-amino-5-[6-(difluoromethoxy)pyridine-3-carbonyl]thiazol-2-yl]-4-fluoro-anilino)propanamide,
rac-2-(N-[4-amino-5-[6-(difluoromethoxy)pyridine-3-carbonyl]thiazol-2-yl]-3,4-difluoro-anilino)propanamide,
(R)-(N-[4-amino-5-[6-(difluoromethoxy)pyridine-3-carbonyl]thiazol-2-yl]-3,4-difluoro-anilino)propanamide,
(S)-(N-[4-amino-5-[6-(difluoromethoxy)pyridine-3-carbonyl]thiazol-2-yl]-3,4-difluoro-anilino)propanamide,
rac-2-(N-[4-amino-5-[6-(difluoromethoxy)pyridine-3-carbonyl]thiazol-2-yl]-4-chloro-3-fluoro-anilino)propanamide,
(R)-2-(N-[4-amino-5-[6-(difluoromethoxy)pyridine-3-carbonyl]thiazol-2-yl]-4-chloro-3-fluoro-anilino)propanamide,
(S)-2-(N-[4-amino-5-[6-(difluoromethoxy)pyridine-3-carbonyl]thiazol-2-yl]-4-chloro-3-fluoro-anilino)propanamide,
rac-2-(N-[4-amino-5-(4-benzyloxybenzoyl)thiazol-2-yl]-3,4-difluoro-anilino)propanamide,
(R)-2-(N-[4-amino-5-(4-benzyloxybenzoyl)thiazol-2-yl]-3,4-difluoro-anilino)propanamide,
(S)-2-(N-[4-amino-5-(4-benzyloxybenzoyl)thiazol-2-yl]-3,4-difluoro-anilino)propanamide,
rac-2-(N-[4-amino-5-(4-benzyloxybenzoyl)thiazol-2-yl]-4-chloro-3-fluoro-anilino)propanamide,
(R)-2-(N-[4-amino-5-(4-benzyloxybenzoyl)thiazol-2-yl]-4-chloro-3-fluoro-anilino)propanamide,
(S)-2-(N-[4-amino-5-(4-benzyloxybenzoyl)thiazol-2-yl]-4-chloro-3-fluoro-anilino)propanamide,
rac-benzyl N-[4-[4-amino-2-(N-(2-amino-1-methyl-2-oxo-ethyl)-3,4-difluoro-anilino)thiazole-5-carbonyl]phenyl]carbamate,
(R)-benzyl N-[4-[4-amino-2-(N-(2-amino-1-methyl-2-oxo-ethyl)-3,4-difluoro-anilino)thiazole-5-carbonyl]phenyl]carbamate,
(S)-benzyl N-[4-[4-amino-2-(N-(2-amino-1-methyl-2-oxo-ethyl)-3,4-difluoro-anilino)thiazole-5-carbonyl]phenyl]carbamate,
rac-2-[4-[4-amino-2-(N-(2-amino-1-methyl-2-oxo-ethyl)-4-fluoro-anilino)thiazole-5-carbonyl]phenoxy]ethyl acetate,
rac-benzyl N-[4-[4-amino-2-(N-(2-amino-1-methyl-2-oxo-ethyl)-4-fluoro-anilino)thiazole-5-carbonyl]phenyl]carbamate,
(R)-benzyl N-[4-[4-amino-2-(N-(2-amino-1-methyl-2-oxo-ethyl)-4-fluoro-anilino)thiazole-5-carbonyl]phenyl]carbamate,
(S)-benzyl N-[4-[4-amino-2-(N-(2-amino-1-methyl-2-oxo-ethyl)-4-fluoro-anilino)thiazole-5-carbonyl]phenyl]carbamate,
rac-2-(N-[4-amino-5-(4-benzyloxybenzoyl)thiazol-2-yl]-4-fluoro-anilino)propanamide,
(R)-2-(N-[4-amino-5-(4-benzyloxybenzoyl)thiazol-2-yl]-4-fluoro-anilino)propanamide,
(S)-2-(N-[4-amino-5-(4-benzyloxybenzoyl)thiazol-2-yl]-4-fluoro-anilino)propanamide,
rac-2-(N-[4-amino-5-(4-iodobenzoyl)thiazol-2-yl]-4-fluoro-anilino)propanamide,
rac-2-(N-[4-amino-5-(6-methoxypyridine-3-carbonyl)thiazol-2-yl]-4-chloro-3-fluoro-anilino)propanamide,
(R)-2-(N-[4-amino-5-(6-methoxypyridine-3-carbonyl)thiazol-2-yl]-4-chloro-3-fluoro-anilino)propanamide,
(S)-2-(N-[4-amino-5-(6-methoxypyridine-3-carbonyl)thiazol-2-yl]-4-chloro-3-fluoro-anilino)propanamide,
rac-2-(N-[4-amino-5-(4-phenoxybenzoyl)thiazol-2-yl]-4-fluoro-anilino)propanamide,
rac-2-(N-[4-amino-5-[4-(difluoromethoxy)benzoyl]thiazol-2-yl]-4-methoxy-anilino)propanamide,
(R)-2-(N-[4-amino-5-[4-(difluoromethoxy)benzoyl]thiazol-2-yl]-4-methoxy-anilino)propanamide,
(S)-2-(N-[4-amino-5-[4-(difluoromethoxy)benzoyl]thiazol-2-yl]-4-methoxy-anilino)propanamide,
rac-2-(N-[4-amino-5-(4-nitrobenzoyl)thiazol-2-yl]-4-fluoro-anilino)propanamide,
rac-2-(4-fluoro-N-[5-(4-methoxybenzoyl)-4-methyl-thiazol-2-yl]anilino)propanamide,
(R)-2-(4-fluoro-N-[5-(4-methoxybenzoyl)-4-methyl-thiazol-2-yl]anilino)propanamide,
(S)-2-(4-fluoro-N-[5-(4-methoxybenzoyl)-4-methyl-thiazol-2-yl]anilino)propanamide,
rac-4-[4-[4-amino-2-(N-(2-amino-1-methyl-2-oxo-ethyl)-4-fluoro-anilino)thiazole-5-carbonyl]phenyl]cyclopropanecarboxamide,
rac-2-(N-[4-amino-5-(4-morpholinobenzoyl)thiazol-2-yl]-4-fluoro-anilino)propanamide,
rac-2-(N-[4-amino-5-[4-(pyrazol-1-ylmethyl)benzoyl]thiazol-2-yl]-4-fluoro-anilino)propanamide,
rac-2-(N-[4-amino-5-[4-(dimethylamino)benzoyl]thiazol-2-yl]-4-fluoro-anilino)propanamide,
rac-2-(N-[4-amino-5-(4-pyrrolidin-1-ylbenzoyl)thiazol-2-yl]-4-fluoro-anilino)propanamide,
rac-2-(N-[4-amino-5-[4-(difluoromethoxy)benzoyl]thiazol-2-yl]-4-benzyloxy-anilino)propanamide,
(R)-2-(N-[4-amino-5-[4-(difluoromethoxy)benzoyl]thiazol-2-yl]-4-benzyloxy-anilino)propanamide,
(S)-2-(N-[4-amino-5-[4-(difluoromethoxy)benzoyl]thiazol-2-yl]-4-benzyloxy-anilino)propanamide,
rac-2-(N-[4-amino-5-[3-(difluoromethoxy)benzoyl]thiazol-2-yl]-4-fluoro-anilino)propanamide,
rac-2-(N-[4-amino-5-(pyridine-3-carbonyl)thiazol-2-yl]-3,4-difluoro-anilino)propanamide,
(R)-2-(N-[4-amino-5-(pyridine-3-carbonyl)thiazol-2-yl]-3,4-difluoro-anilino)propanamide,
(S)-2-(N-[4-amino-5-(pyridine-3-carbonyl)thiazol-2-yl]-3,4-difluoro-anilino)propanamide,
rac-2-(N-[5-(4-acetamidobenzoyl)-4-amino-thiazol-2-yl]-4-fluoro-anilino)propanamide,
rac-2-(N-[4-amino-5-(2-chloropyridine-4-carbonyl)thiazol-2-yl]-4-fluoro-anilino)propanamide,
rac-2-(N-[4-amino-5-(2-methylpyridine-4-carbonyl)thiazol-2-yl]-4-fluoro-anilino)propanamide,
(R)-2-(N-[4-amino-5-(2-methylpyridine-4-carbonyl)thiazol-2-yl]-4-fluoro-anilino)propanamide,
(S)-2-(N-[4-amino-5-(2-methylpyridine-4-carbonyl)thiazol-2-yl]-4-fluoro-anilino)propanamide,
rac-2-(N-[4-amino-5-[2-(difluoromethyl)pyridine-4-carbonyl]thiazol-2-yl]-4-fluoro-anilino)propanamide,
rac-2-[(4-amino-5-benzoyl-thiazol-2-yl)-(4-pyridyl)amino]propanamide,
rac-2-(N-[4-amino-5-(2-methoxypyridine-4-carbonyl)thiazol-2-yl]-4-fluoro-anilino)propanamide,
rac-2-(N-(4-amino-5-benzoyl-thiazol-2-yl)-3-fluoro-4-methoxy-anilino)propanamide,
rac-2-(N-(4-amino-5-benzoyl-thiazol-2-yl)-4-cyano-3-fluoro-anilino)propanamide,
rac-2-(N-(4-amino-5-benzoyl-thiazol-2-yl)-4-bromo-anilino)propanamide,
rac-2-[N-(4-amino-5-benzoyl-thiazol-2-yl)-3-chloro-4-(difluoromethoxy)anilino]propanamide,
rac-2-(N-(4-amino-5-benzoyl-thiazol-2-yl)-4-ethoxy-anilino)propanamide,
rac-2-[(4-amino-5-benzoyl-thiazol-2-yl)-(1,3-benzodioxol-5-yl)amino]propanamide,
rac-2-[(4-amino-5-benzoyl-thiazol-2-yl)-(2,2-difluoro-1,3-benzodioxol-5-yl)amino]propanamide,
rac-2-[N-(4-amino-5-benzoyl-thiazol-2-yl)-4-(difluoromethoxy)-3-fluoro-anilino]propanamide,
rac-2-(N-(4-amino-5-benzoyl-thiazol-2-yl)-4-benzyloxy-anilino)propanamide,
rac-2-[N-[4-amino-5-(4-methoxybenzoyl)thiazol-2-yl]-3-chloro-4-(difluoromethoxy)anilino]propanamide,
rac-2-[N-[4-amino-5-[4-(difluoromethoxy)benzoyl]thiazol-2-yl]-3-chloro-4-(difluoromethoxy)anilino]propanamide,
rac-2-[N-[4-amino-5-(4-chlorobenzoyl)thiazol-2-yl]-3-chloro-4-(difluoromethoxy)anilino]propanamide,
rac-2-[N-[4-amino-5-(pyridine-4-carbonyl)thiazol-2-yl]-3-chloro-4-(difluoromethoxy)anilino]propanamide,
rac-2-[N-[4-amino-5-[6-(difluoromethoxy)pyridine-3-carbonyl]thiazol-2-yl]-3-chloro-4-(difluoromethoxy)anilino]propanamide,
rac-2-[N-[4-amino-5-(4-methoxybenzoyl)thiazol-2-yl]-4-(difluoromethoxy)-3-fluoro-anilino]propanamide,
rac-2-[N-[4-amino-5-(4-chlorobenzoyl)thiazol-2-yl]-4-(difluoromethoxy)-3-fluoro-anilino]propanamide,
rac-2-[N-[4-amino-5-[4-(difluoromethoxy)benzoyl]thiazol-2-yl]-4-(difluoromethoxy)-3-fluoro-anilino]propanamide,
rac-2-[N-[4-amino-5-(pyridine-4-carbonyl)thiazol-2-yl]-4-(difluoromethoxy)-3-fluoro-anilino]propanamide,
rac-2-[N-[4-amino-5-[6-(difluoromethoxy)pyridine-3-carbonyl]thiazol-2-yl]-4-(difluoromethoxy)-3-fluoro-anilino]propanamide,
rac-2-[N-(4-amino-5-benzoyl-thiazol-2-yl)-3-chloro-4-(trifluoromethoxy)anilino]propanamide,
rac-2-[[4-amino-5-(4-methoxybenzoyl)thiazol-2-yl]-(2,2-difluoro-1,3-benzodioxol-5-yl)amino]propanamide,
rac-2-[[4-amino-5-(4-chlorobenzoyl)thiazol-2-yl]-(2,2-difluoro-1,3-benzodioxol-5-yl)amino]propanamide,
rac-2-[[4-amino-5-[4-(difluoromethoxy)benzoyl]thiazol-2-yl]-(2,2-difluoro-1,3-benzodioxol-5-yl)amino]propanamide,
rac-2-[[4-amino-5-(pyridine-4-carbonyl)thiazol-2-yl]-(2,2-difluoro-1,3-benzodioxol-5-yl)amino]propanamide,
rac-2-[[4-amino-5-[6-(difluoromethoxy)pyridine-3-carbonyl]thiazol-2-yl]-(2,2-difluoro-1,3-benzodioxol-5-yl)amino]propanamide,
rac-2-[N-[4-amino-5-(4-methoxybenzoyl)thiazol-2-yl]-3-fluoro-4-(trifluoromethoxy)anilino]propanamide,
rac-2-[N-[4-amino-5-(4-chlorobenzoyl)thiazol-2-yl]-3-fluoro-4-(trifluoromethoxy)anilino]propanamide,
rac-2-[N-[4-amino-5-[4-(difluoromethoxy)benzoyl]thiazol-2-yl]-3-fluoro-4-(trifluoromethoxy)anilino]propanamide,
rac-2-[N-[4-amino-5-(pyridine-4-carbonyl)thiazol-2-yl]-3-fluoro-4-(trifluoromethoxy)anilino]propanamide,
rac-2-[N-[4-amino-5-[6-(difluoromethoxy)pyridine-3-carbonyl]thiazol-2-yl]-3-fluoro-4-(trifluoromethoxy)anilino]propanamide,
rac-2-[N-[4-amino-5-(4-methoxybenzoyl)thiazol-2-yl]-3-chloro-4-(trifluoromethoxy)anilino]propanamide,
rac-2-[N-[4-amino-5-(4-chlorobenzoyl)thiazol-2-yl]-3-chloro-4-(trifluoromethoxy)anilino]propanamide,
rac-2-[N-[4-amino-5-[4-(difluoromethoxy)benzoyl]thiazol-2-yl]-3-chloro-4-(trifluoromethoxy)anilino]propanamide,
rac-2-[N-[4-amino-5-(pyridine-4-carbonyl)thiazol-2-yl]-3-chloro-4-(trifluoromethoxy)anilino]propanamide,
rac-2-[N-[4-amino-5-[6-(difluoromethoxy)pyridine-3-carbonyl]thiazol-2-yl]-3-chloro-4-(trifluoromethoxy)anilino]propanamide,
rac-2-[(4-amino-5-benzoyl-thiazol-2-yl)-(6-methoxy-3-pyridyl)amino]propanamide,
rac-2-[(4-amino-5-benzoyl-thiazol-2-yl)-[6-(trifluoromethoxy)-3-pyridyl]amino]propanamide,
rac-2-[(4-amino-5-benzoyl-thiazol-2-yl)-[6-(difluoromethoxy)-3-pyridyl]amino]propanamide,
rac-2-[(4-amino-5-benzoyl-thiazol-2-yl)-[6-(trifluoromethyl)-3-pyridyl]amino]propanamide,
rac-2-[(4-amino-5-benzoyl-thiazol-2-yl)-[6-(difluoromethyl)-3-pyridyl]amino]propanamide,
rac-2-[(4-amino-5-benzoyl-thiazol-2-yl)-(6-chloro-3-pyridyl)amino]propanamide,
rac-2-[(4-amino-5-benzoyl-thiazol-2-yl)-(6-fluoro-3-pyridyl)amino]propanamide,
rac-2-[(4-amino-5-benzoyl-thiazol-2-yl)-(6-methyl-3-pyridyl)amino]propanamide,
rac-2-[N-(4-amino-5-benzoyl-thiazol-2-yl)-4-fluoro-3-(trifluoromethoxy)anilino]propanamide,
rac-2-[N-(4-amino-5-benzoyl-thiazol-2-yl)-4-chloro-3-(trifluoromethoxy)anilino]propanamide,
rac-2-[N-(4-amino-5-benzoyl-thiazol-2-yl)-3-(difluoromethoxy)-4-fluoro-anilino]propanamide,
rac-2-[N-(4-amino-5-benzoyl-thiazol-2-yl)-4-chloro-3-(difluoromethoxy)anilino]propanamide,
rac-2-[N-(4-amino-5-benzoyl-thiazol-2-yl)-2-fluoro-4-(trifluoromethoxy)anilino]propanamide,
rac-2-[N-(4-amino-5-benzoyl-thiazol-2-yl)-2-chloro-4-(trifluoromethoxy)anilino]propanamide,
rac-2-[N-(4-amino-5-benzoyl-thiazol-2-yl)-4-(difluoromethoxy)-2-fluoro-anilino]propanamide,
rac-2-[N-(4-amino-5-benzoyl-thiazol-2-yl)-2-chloro-4-(difluoromethoxy)anilino]propanamide,
rac-2-[N-[4-amino-5-(4-methoxybenzoyl)thiazol-2-yl]-4-(difluoromethyl)anilino]propanamide,
rac-2-[N-[4-amino-5-(4-chlorobenzoyl)thiazol-2-yl]-4-(difluoromethyl)anilino]propanamide,
rac-2-[N-[4-amino-5-[4-(difluoromethoxy)benzoyl]thiazol-2-yl]-4-(difluoromethyl)anilino]propanamide,
rac-2-[N-[4-amino-5-(pyridine-4-carbonyl)thiazol-2-yl]-4-(difluoromethyl)anilino]propanamide,
rac-2-[N-[4-amino-5-[6-(difluoromethoxy)pyridine-3-carbonyl]thiazol-2-yl]-4-(difluoromethyl)anilino]propanamide,
rac-2-[[4-amino-5-(4-methoxybenzoyl)thiazol-2-yl]-[6-(trifluoromethoxy)-3-pyridyl]amino]propanamide,
rac-2-[[4-amino-5-(4-chlorobenzoyl)thiazol-2-yl]-[6-(trifluoromethoxy)-3-pyridyl]amino]propanamide,
rac-2-[[4-amino-5-[4-(difluoromethoxy)benzoyl]thiazol-2-yl]-[6-(trifluoromethoxy)-3-pyridyl]amino]propanamide,
rac-2-[[4-amino-5-[6-(difluoromethoxy)pyridine-3-carbonyl]thiazol-2-yl]-[6-(trifluoromethoxy)-3-pyridyl]amino]propanamide,
rac-2-[[4-amino-5-(4-methoxybenzoyl)thiazol-2-yl]-[6-(difluoromethoxy)-3-pyridyl]amino]propanamide,
rac-2-[[4-amino-5-(4-chlorobenzoyl)thiazol-2-yl]-[6-(difluoromethoxy)-3-pyridyl]amino]propanamide,
rac-2-[[4-amino-5-[4-(difluoromethoxy)benzoyl]thiazol-2-yl]-[6-(difluoromethoxy)-3-pyridyl]amino]propanamide,
rac-2-[[4-amino-5-[6-(difluoromethoxy)pyridine-3-carbonyl]thiazol-2-yl]-[6-(difluoromethoxy)-3-pyridyl]amino]propanamide,
rac-benzyl N-[5-[4-amino-2-(N-(2-amino-1-methyl-2-oxo-ethyl)-4-fluoro-anilino)thiazole-5-carbonyl]-2-pyridyl]carbamate,
rac-Ethyl 4-[4-amino-2-(N-(2-amino-1-methyl-2-oxo-ethyl)-4-fluoro-anilino)thiazole-5-carbonyl]benzoate,
rac-Ethyl 4-[4-amino-2-(N-(2-amino-1-methyl-2-oxo-ethyl)-4-chloro-3-fluoro-anilino)thiazole-5-carbonyl]benzoate,
rac-Ethyl 2-[4-[4-amino-2-(N-(2-amino-1-methyl-2-oxo-ethyl)-4-fluoro-anilino)thiazole-5-carbonyl]phenoxy]-2-methyl-propanoate,
rac-4-[4-amino-2-(N-(2-amino-1-methyl-2-oxo-ethyl)-4-fluoro-anilino)thiazole-5-carbonyl]-N-cyclohexyl-benzamide,
rac-4-[4-amino-2-(N-(2-amino-1-methyl-2-oxo-ethyl)-4-fluoro-anilino)thiazole-5-carbonyl]-N-isopropyl-benzamide,
rac-4-[4-amino-2-(N-(2-amino-1-methyl-2-oxo-ethyl)-4-fluoro-anilino)thiazole-5-carbonyl]-N-benzyl-benzamide,
4-[4-amino-2-(N-(2-amino-1-methyl-2-oxo-ethyl)-4-fluoro-anilino)thiazole-5-carbonyl]-N-[(2S)-2-hydroxypropyl]benzamide (mixture of stereoisomers),
rac-4-[4-amino-2-(N-(2-amino-1-methyl-2-oxo-ethyl)-4-fluoro-anilino)thiazole-5-carbonyl]-N-(2-methoxyethyl)benzamide,
4-[4-amino-2-(N-(2-amino-1-methyl-2-oxo-ethyl)-4-fluoro-anilino)thiazole-5-carbonyl]-N-[(2R)-2-hydroxypropyl]benzamide (mixture of stereoisomers),
rac-4-[4-amino-2-(N-(2-amino-1-methyl-2-oxo-ethyl)-4-fluoro-anilino)thiazole-5-carbonyl]-N-cyclopropyl-benzamide,
rac-4-[4-amino-2-(N-(2-amino-1-methyl-2-oxo-ethyl)-4-fluoro-anilino)thiazole-5-carbonyl]-N-cyclopentyl-benzamide,
rac-4-[4-amino-2-(N-(2-amino-1-methyl-2-oxo-ethyl)-4-chloro-3-fluoro-anilino)thiazole-5-carbonyl]-N-(2-phenoxyethyl)benzamide,
rac-4-[4-amino-2-(N-(2-amino-1-methyl-2-oxo-ethyl)-4-chloro-3-fluoro-anilino)thiazole-5-carbonyl]-N-[2-(trifluoromethoxy)ethyl]benzamide,
rac-4-[4-amino-2-(N-(2-amino-1-methyl-2-oxo-ethyl)-4-chloro-3-fluoro-anilino)thiazole-5-carbonyl]-N-[2-(difluoromethoxy)ethyl]benzamide,
rac-4-[4-amino-2-(N-(2-amino-1-methyl-2-oxo-ethyl)-4-chloro-3-fluoro-anilino)thiazole-5-carbonyl]-N-(2-tert-butoxyethyl)benzamide,
rac-4-[4-amino-2-(N-(2-amino-1-methyl-2-oxo-ethyl)-4-chloro-3-fluoro-anilino)thiazole-5-carbonyl]-N-(2-methoxyethyl)benzamide,
rac-2-[4-[4-amino-2-(N-(2-amino-1-methyl-2-oxo-ethyl)-4-fluoro-anilino)thiazole-5-carbonyl]phenoxy]-N-[(4-chlorophenyl)methyl]-2-methyl-propanamide,
rac-2-(N-[4-amino-5-(6-bromopyridine-3-carbonyl)thiazol-2-yl]-4-fluoro-anilino)propanamide,
rac-2-(N-[4-amino-5-[6-[4-(trifluoromethyl)-1-piperidyl]pyridine-3-carbonyl]thiazol-2-yl]-4-fluoro-anilino)propanamide,
rac-2-(N-[4-amino-5-[6-(4-methyl-1-piperidyl)pyridine-3-carbonyl]thiazol-2-yl]-4-fluoro-anilino)propanamide,
rac-2-(N-[4-amino-5-[6-[4-(oxetan-3-yl)-1-piperidyl]pyridine-3-carbonyl]thiazol-2-yl]-4-fluoro-anilino)propanamide,
rac-2-(N-[4-amino-5-[6-(dimethylamino)pyridine-3-carbonyl]thiazol-2-yl]-4-fluoro-anilino)propanamide,
rac-2-(N-[4-amino-5-[6-(4,4-dimethyl-1-piperidyl)pyridine-3-carbonyl]thiazol-2-yl]-4-fluoro-anilino)propanamide,
rac-2-(N-[4-amino-5-[6-(3-azabicyclo[3.2.1]octan-3-yl)pyridine-3-carbonyl]thiazol-2-yl]-4-fluoro-anilino)propanamide,
rac-2-(N-[4-amino-5-[6-(3,5-dimethyl-1-piperidyl)pyridine-3-carbonyl]thiazol-2-yl]-4-fluoro-anilino)propanamide,
rac-2-(N-[4-amino-5-[6-(3-azabicyclo[3.1.0]hexan-3-yl)pyridine-3-carbonyl]thiazol-2-yl]-4-fluoro-anilino)propanamide,
rac-2-(N-[4-amino-5-[6-(1-piperidyl)pyridine-3-carbonyl]thiazol-2-yl]-4-fluoro-anilino)propanamide,
rac-2-(N-[4-amino-5-[6-(4,4-difluoro-1-piperidyl)pyridine-3-carbonyl]thiazol-2-yl]-4-fluoro-anilino)propanamide,
rac-2-(N-[4-amino-5-[6-(4-cyano-4-methyl-1-piperidyl)pyridine-3-carbonyl]thiazol-2-yl]-4-fluoro-anilino)propanamide,
(R)-2-(N-[4-amino-5-[6-(4-cyano-4-methyl-1-piperidyl)pyridine-3-carbonyl]thiazol-2-yl]-4-fluoro-anilino)propanamide,
(S)-2-(N-[4-amino-5-[6-(4-cyano-4-methyl-1-piperidyl)pyridine-3-carbonyl]thiazol-2-yl]-4-fluoro-anilino)propanamide,
2-(N-[4-amino-5-(4-hydroxybenzoyl)thiazol-2-yl]-4-fluoro-anilino)propanamide (single enantiomer),
(R)-2-(N-[4-amino-5-(4-hydroxybenzoyl)thiazol-2-yl]-3,4-difluoro-anilino)propanamide,
(S)-2-(N-[4-amino-5-(4-hydroxybenzoyl)thiazol-2-yl]-3,4-difluoro-anilino)propanamide,
2-(N-[4-amino-5-(4-hydroxybenzoyl)thiazol-2-yl]-4-chloro-3-fluoro-anilino)propanamide (enantiomer 1),
2-(N-[4-amino-5-(4-hydroxybenzoyl)thiazol-2-yl]-4-chloro-3-fluoro-anilino)propanamide (enantiomer 2),
2-(N-[4-amino-5-[4-(difluoromethoxy)benzoyl]thiazol-2-yl]-4-hydroxy-anilino)propanamide (single enantiomer), and
rac-2-(N-[4-amino-5-[4-(2-hydroxyethoxy)benzoyl]thiazol-2-yl]-4-fluoro-anilino)propanamide,
or a stereoisomer, a tautomer, an N-oxide, a hydrate, a solvate, or a salt thereof, or a mixture of same.

7. The combination according to claim 1, in which the DGKalpha inhibitor compound is of the formula 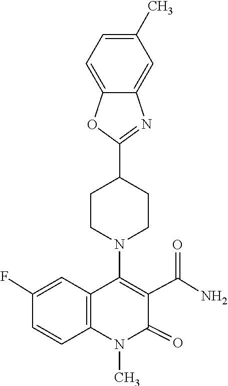 [SMILES: Cc1ccc2oc(C3CCN(c4c(C(N)=O)c(=O)n(C)c5ccc(F)cc45)CC3)nc2c1]
or a tautomer, an N-oxide, a hydrate, a solvate, or a salt thereof, or a mixture of same, and
in which the DGKzeta inhibitor compound is of the formula 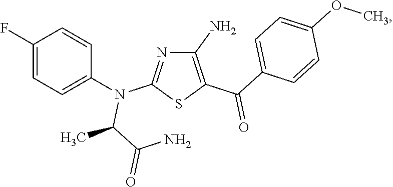 [SMILES: COc1ccc(C(=O)c2sc(N(c3ccc(F)cc3)[C@H](C)C(N)=O)nc2N)cc1]
or a stereoisomer, a tautomer, an N-oxide, a hydrate, a solvate, or a salt thereof, or a mixture of same.

8. The combination according to claim 1, in which the DGKalpha inhibitor compound is of the formula 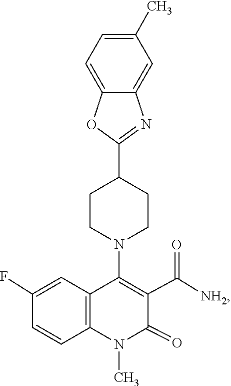 [SMILES: Cc1ccc2oc(C3CCN(c4c(C(N)=O)c(=O)n(C)c5ccc(F)cc45)CC3)nc2c1]
and
in which the DGKzeta inhibitor compound is of the formula

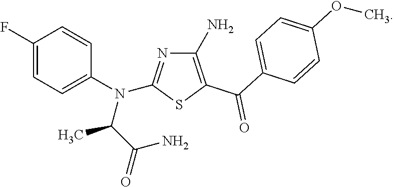 [SMILES: COc1ccc(C(=O)c2sc(N(c3ccc(F)cc3)[C@H](C)C(N)=O)nc2N)cc1]

9. (canceled)

10. (canceled)

11. A method of the treatment or prophylaxis of a disease in a patient, preferably a condition with dysregulated immune responses, or a viral infection or another disorder associated with aberrant DGKalpha and/or DGKzeta signaling, in mammals, said method comprising administering a combination according to claim 1.

12. The method according to claim 11, in which the mammal is a human.

13. (canceled)

14. (canceled)

15. A kit comprising
a DGKalpha inhibitor compound as defined in claim 1;
a DGKzeta inhibitor compound as defined in claim 1.

16. The kit according to claim 15, in which optionally both or either of said DGKalpha inhibitor compound and said DGKzeta inhibitor compound are in the form of a pharmaceutical composition which is ready for use to be administered simultaneously, concurrently, separately or sequentially.

17. The kit according to claim 15, in which said DGKalpha inhibitor compound and said DGKzeta inhibitor compound each are in the form of a pharmaceutical composition and in which said DGKalpha inhibitor compound is administered prior to said DGKzeta inhibitor compound.

18. A pharmaceutical composition comprising a combination as defined in claim 1 together with one or more pharmaceutically acceptable excipients.

19. The pharmaceutical composition according to claim 18, in which said DGKalpha inhibitor compound and said DGKzeta inhibitor compound are present in a joint formulation.

20. The pharmaceutical composition according to claim 19, in which said DGKalpha inhibitor compound and said DGKzeta inhibitor compound are present in separate formulations.

21. (canceled)

22. (canceled)

23. (canceled)

24. The method according to claim 11,
a. wherein the inhibitor of DGKalpha is DGKalpha inhibitor A, or a tautomer, an N-oxide, a hydrate, a solvate, or a salt thereof, or a mixture of same and/or
b. wherein the inhibitor of DGKzeta is DGKzeta inhibitor A′, or a stereoisomer, a tautomer, an N-oxide, a hydrate, a solvate, or a salt thereof, or a mixture of same.

25. The method according to claim 11,
a. wherein the inhibitor of DGKalpha is DGKalpha inhibitor A, and/or
b. wherein the inhibitor of DGKzeta is DGKzeta inhibitor A′.

26. The method according to claim 11, wherein the disease is cancer.

27. The method according to claim 26, wherein the cancer is selected from the group consisting of non-small cell lung cancer (NSCLC), triple-negative breast cancer (TNBC), head and neck squamous cell carcinoma (HNSCC), melanoma, skin cancer other than melanoma, gastric cancer, renal cell carcinoma, MSI high colorectal carcinoma, or gastroesophageal junction adenocarcinoma.
